(19) United States
(12) Patent Application Publication
(10) Pub. No.: US 2022/0062339 A1
(43) Pub. Date: Mar. 3, 2022

(54) USE OF AN ANTI-CD45 ANTIBODY DRUG CONJUGATE (ADC) IN CELL THERAPY

(71) Applicant: Magenta Therapeutics, Inc., Cambridge, MA (US)

(72) Inventors: Michael Cooke, Boston, MA (US); Geoffrey O. Gillard, Harvard, MA (US); Anthony Boitano, Newton, MA (US)

(21) Appl. No.: 17/368,815

(22) Filed: Jul. 6, 2021

Related U.S. Application Data
(63) Continuation of application No. PCT/US2020/012637, filed on Jan. 7, 2020.
(60) Provisional application No. 62/845,829, filed on May 9, 2019.
(60) Provisional application No. 62/789,462, filed on Jan. 7, 2019.

(51) Int. Cl. A61K 35/17; C07K 16/28; A61K 47/68; A61P 35/00

(52) U.S. Cl. CPC A61K 35/17; C07K 16/289; C07K 16/2815; A61P 35/00; C07K 16/2878; C07K 16/2893; A61K 47/6803; C07K 16/2812

(57) ABSTRACT

The invention provides methods of depleting CD45+ cells in human patients undergoing chimeric antigen receptor (CAR) immunotherapy in order to promote acceptance of CAR expressing immune cells. Anti-CD45 antibody drug conjugates (ADCs) are administered as a conditioning regimen to a human patient receiving autologous or allogeneic CAR expressing immune cells such that the CAR expressing immune cells are accepted by the human patient. Compositions and methods of the invention can be used in combination with CAR therapy to treat a variety of pathologies, including autoimmune diseases and cancer.

### RELATED APPLICATIONS

This application is a continuation of PCT/US2020/012637, filed on Jan. 7, 2020, which claims priority to U.S. Provisional Application No. 62/789,462, filed on Jan. 7, 2019, and U.S. Provisional Application No. 62/845,829, filed on May 9, 2019. The content of each of the priority applications is incorporated by reference herein.

### FIELD

The present invention generally relates to methods for promoting acceptance of an immune cell expressing a chimeric antigen receptor (CAR) in a human subject through the use of an anti-CD45 antibody-drug conjugate (ADC).

### BACKGROUND

Chimeric antigen receptor (CAR) therapy is an immunological treatment that uses lymphocytes, either from the patient or from an allogeneic donor, engineered to destroy cells expressing a specific antigen associated with a certain disease, such as cancer. In cancer, for example, CAR therapy enlists and strengthens the power of a patient's immune system to attack tumors. Over the past several years, this immunotherapy has emerged as a promising and revolutionary therapy. CAR therapy is based on an immune cell, such as a T cell, expressing a CAR which is generally a transmembrane fusion protein that combines an extracellular antigen binding domain, such as an scFv, with cytoplasmic activity signaling and “co-stimulatory” domains that signal the cell from the surface receptor. Thus, when immune cells, such as T-cells, express CARs, the immune cells are able to recognize and kill cells that express the antigen targeted by the antigen binding domain of the CAR (e.g., a tumor associated antigen) (Geyer and Brentjens (2016) Cytotherapy 18(11): 1393-1409).

While CAR therapy is an incredibly powerful technology, it does come with serious potential risks and adverse side effects (Kay and Turtle (2017) Drugs 77(3):237-245; Hill et al. (2018) Blood 131:121-130). Lymphodepleting chemotherapy is commonly used as a conditioning treatment in combination with CAR therapy in order to minimize the rejection of the CAR expressing cells by the patient receiving treatment (Wei et al. (2017) Exp Hematol Oncol. 6: 10). For example, the combination of lymphodepleting agents fludarabine and cyclophosphamide improved duration of CAR-T cells in recipient patients (Turtle et al. (2016) J Clinic Invest 126(6):2123; see also US 20170368101). While conditioning therapy has improved the efficacy of CAR-T cells, lymphodepleting chemotherapy often has serious negative side effects.

### SUMMARY

The present disclosure provides a conditioning regimen which can be used with chimeric antigen receptor (CAR) therapy to promote acceptance of CAR expressing immune cells. The methods described herein can be used to promote acceptance of either autologous CAR expressing immune cells or allogeneic CAR expressing immune cells. Traditionally acceptance of such cells has been achieved using lymphodepleting chemotherapeutic treatment. Described herein are improved methods of promoting acceptance of CAR expressing cells in a recipient patient.

In a first aspect, the present disclosure features a method of promoting acceptance of an immune cell expressing a chimeric antigen receptor (CAR) in a human subject having cancer or an autoimmune disease, by (a) administering an anti-CD45 antibody drug conjugate (ADC) to a human subject having cancer or an autoimmune disease, wherein the anti-CD45 ADC comprises an anti-CD45 antibody, or antigen-binding fragment thereof, conjugated to a cytotoxin via a linker; and (b) administering a therapeutically effective amount of an immune cell expressing a CAR to the human subject, wherein the CAR comprises an extracellular domain that binds to a tumor antigen expressed on the surface of a cell or an antigen associated with an autoimmune disease that is expressed on the surface of a cell, a transmembrane domain, and a cytoplasmic domain. In one embodiment, the human subject is not administered alemtuzumab prior to, concomitantly with, or following step (b). In another embodiment, the human subject is not administered a lymphodepleting chemotherapeutic agent prior to, concomitantly with, or following step (b). In yet other embodiments, the lymphodepleting chemotherapeutic agent is fludarabine, cyclophosphamide, bendamustine, and/or pentostatin.

In certain embodiments, the method involves administering an anti-CD45 ADC to the human subject prior to step (b).

In certain other embodiments, method involves administering the anti-CD45 ADC to the human subject about 12 hours to about 21 days (e.g., about 12 hours, about 13 hours, about 14 hours, about 15 hours, about 16 hours, about 17 hours, about 18 hours, about 19 hours, about 20 hours, about 21 hours, about 22 hours, about 23 hours, about 24 hours, about 2 days, about 3 days, about 4 days, about 5 days, about 6 days, about 7 days, about 8 days, about 9 days, about 10 days, about 11 days, about 12 days, about 13 days, about 14 days, about 15 days, about 16 days, about 17 days, about 18 days, about 19 days, about 20 days, or about 21 days) before step (b).

In certain embodiments, the immune cell is an allogeneic cell or an autologous cell. In yet another embodiment, the allogeneic cell is an allogeneic T cell or an allogeneic NK cell.

In certain embodiments, the therapeutically effective amount of the allogeneic cell expressing the CAR is about 1×10⁴ to about 1.0×10⁸ cells/kg (e.g., about 1×10⁴ to about 1×10⁸ cells/kg, about 1×10⁴ to about 1×10⁷ cells/kg, about 1×10⁴ to about 1×10⁶ cells/kg, about 1×10⁴ to about 1×10⁵ cells/kg, about 1×10⁵ to about 1×10⁸ cells/kg, about 1×10⁶ to about 1×10⁸ cells/kg, or about 1×10⁷ to about 1×10⁸ cells/kg).

In another aspect, the present disclosure features a method of treating a patient having a tumor by administering (i) an anti-CD45 ADC, wherein the anti-CD45 ADC comprises an anti-CD45 antibody, or antigen-binding fragment thereof, conjugated to a cytotoxin via a linker, and (ii) administering to the patient a therapeutically effective amount of from about 1×10⁶ to about 1×10⁸ engineered CAR T cells/kg (e.g., about 1×10⁶ to about 2×10⁶, about 2×10⁶ to about 3×10⁶ about, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 76×10⁷, about 8×10⁷ to about 9×10⁷, about 9×10⁷ to about 1×10⁸, about 1×10⁶, about 1×10⁷, or about 1×10⁸ engineered CART cells/kg). In one embodiment, the therapeutically effective amount of the engineered CAR T cells is about 1×10⁶ or about 2×10⁶ cells/kg. In yet another embodiment, the anti-CD45 ADC is administered to the patient as a single dose or as multiple doses.

In certain embodiments, the anti-CD45 antibody, or antigen-binding fragment thereof, comprises a heavy chain variable region comprising a CDR1, a CDR2, and a CDR3 having an amino acid sequence as set forth in SEQ ID NOs: 1, 2, and 3, respectively, and comprises a light chain variable region comprising a CDR1, a CDR2, and a CDR3 having an amino acid sequence as set forth in SEQ ID NOs: 4, 5, and 6, respectively. In another embodiment, the anti-CD45 antibody, or antigen-binding fragment thereof, is chimeric or humanized. In other embodiments, the anti-CD45 antibody, or antigen-binding fragment thereof, comprises a heavy chain variable region comprising the amino acid sequence as set forth in SEQ ID NO: 7, and comprises a light chain variable region comprising the amino acid sequence as set forth in SEQ ID NO: 8, respectively.

In certain embodiments, the anti-CD45 antibody, or antigen-binding fragment thereof, is an IgG1 isotype or an IgG4 isotype.

In certain embodiments, the cytotoxin is an antimitotic agent, a ribosome inactivating protein (RIP) (e.g., Shiga toxin), or an RNA polymerase inhibitor. In other embodiments, the RNA polymerase inhibitor is an amatoxin. In another embodiment, the RNA polymerase inhibitor is an amanitin. In another embodiment, the amanitin amatoxin is selected from the group consisting of α-amanitin, β-amanitin, γ-amanitin, ε-amanitin, amanin, amaninamide, amanullin, amanullinic acid, proamanullin and derivatives thereof.

In some embodiments of any of the above aspects, the cytotoxin is an amatoxin, and the antibody or the antigen-binding fragment thereof is conjugated to the amatoxin through a linker and chemical moiety to form an ADC represented by the formula Ab-Z-L-Am, wherein Ab is the antibody or antigen-binding fragment thereof, L is a linker, Z is a chemical moiety, and Am is the amatoxin. In some embodiments, the amatoxin is conjugated to a linker. In some embodiments, the amatoxin-linker conjugate Am-L-Z is represented by formula (I)

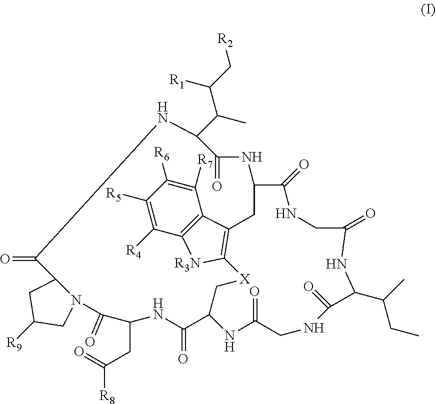

wherein R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, or optionally substituted heteroarylene; a peptide (e.g., a dipeptide), —(C═O)—, a disulfide, a hydrazone, a —(CH₂CH₂O)ₚ— group, wherein p is an integer from 1-6, a ((CH₂)ₘO)ₙ(CH₂)ₘ— group, where n and each m are each independently selected from 1, 2, 3, 4, 5, 6, 7, 8, 9, and 10; or a combination thereof; and

Z is a chemical moiety formed from a coupling reaction between a reactive substituent Z′ present on L and a reactive substituent present within an antibody, or an antigen-binding fragment thereof, that binds CD45.

In yet another embodiment, the antimitotic agent is a maytansine or an auristatin. In other embodiments, the auristatin is monomethyl auristatin F (MMAF) or monomethyl auristatin E (MMAE). In yet another embodiment, the antimitotic agent is a pyrrolobenzodiazepine (PBD) or a calicheamicin.

In certain embodiments, the linker of the ADC along with the reactive substituent Z′, is N-beta-maleimidopropyl-Val-Ala-para-aminobenzyl (BMP-Val-Ala-PAB).

In certain embodiments, the ADC has a serum half-life of 3 days or less.

In certain embodiments, the extracellular domain of the CAR comprises an scFv antibody or a single chain T cell receptor (scTCR).

In certain embodiments, the extracellular domain comprises a non-immunoglobulin scaffold protein.

In certain embodiments, the tumor antigen is an antigen selected from the group consisting of CD19, CD22, CD30, CD7, BCMA, CD137, CD22, CD20, AFP, GPC3, MUC1, mesothelin, CD38, PD1, EGFR (e.g., EGFRvIII), MG7, BCMA, TACI, CEA, PSCA, CEA, HER2, MUC1, CD33, ROR2, NKR-2, PSCA, CD28, TAA, NKG2D, or CD123.

In certain embodiments, the cytoplasmic domain of the CAR comprises a CD28 cytoplasmic signaling domain, a CD3 zeta cytoplasmic signaling domain, an OX40 cytoplasmic signaling domain, and/or a CD137 (4-1BB) cytoplasmic signaling domain.

In certain embodiments, the cytoplasmic domain of the CAR comprises a CD3 zeta cytoplasmic signaling domain.

In one embodiment, an anti-CD45 ADC is administered to the subject prior to CAR therapy in a therapeutically effective amount such that hematopoietic stem cell (HSC) levels are maintained in the patient while lymphocytes are depleted. In one embodiment, the level of HSCs in the subject is about 70% or more relative to the level of HSCs prior to anti-CD45 ADC treatment in the subject. In one embodiment, the level of HSCs in the subject is about 80% or more relative to the level of HSCs prior to anti-CD45 ADC treatment in the subject. In one embodiment, the level of HSCs in the subject is about 90% or more relative to the level of HSCs prior to anti-CD45 ADC treatment in the subject.

In certain embodiments, the human subject having cancer has a cancer selected from the group consisting of leukemia, adult advanced cancer, pancreatic cancer, non-resectable pancreatic cancer, colorectal cancer, metastatic colorectal cancer, ovarian cancer, triple-negative breast cancer, hematopoietic/lymphoid cancer, colon cancer liver metastasis, small cell lung cancer, non-small cell lung cancer, B-cell lymphoma, relapsed or refractory B-cell lymphoma, follicular lymphoma, mantle cell lymphoma, diffuse large cell lymphoma, relapsed or refractory diffuse large cell lymphoma, anaplastic large cell lymphoma, primary mediastinal B-cell lymphoma, recurrent mediastinal, refractory mediastinal large B-cell lymphoma, large B-cell lymphoma, Hodgkin lymphoma, non-Hodgkin lymphoma, relapsed or refractory non-Hodgkin lymphoma, refractory aggressive non-Hodgkin lymphoma, B-cell non-Hodgkin lymphoma, refractory non-Hodgkin lymphoma, colorectal carcinoma, gastric carcinoma, pancreatic carcinoma, triple-negative invasive breast carcinoma, renal cell carcinoma, lung squamous cell carcinoma, hepatocellularcarcinoma, urothelial carcinoma, leukemia, B-cell leukemia, B-cell acute lymphocytic leukemia, B-cell acute lymphoblastic leukemia, adult acute lymphoblastic leukemia, B-cell prolymphocytic leukemia, childhood acute lymphoblastic leukemia, refractory childhood acute lymphoblastic leukemia, acute leukemia, acute lymphoblastic leukemia, acute lymphocytic leukemia, prolymphocytic leukemia, chronic lymphocytic leukemia, acute myeloid leukemia, recurrent plasma cell myeloma, refractory plasma cell myeloma, multiple myeloma, relapsed or refractory multiple myeloma, multiple myeloma of bone, malignant glioma of brain, myelodysplastic syndrome, EGFR-positive colorectal cancer, glioblastoma multiforme, neoplasms, blastic plasmacytoid dendritic cell neoplasms, liver metastases, solid tumors, advanced solid tumors, mesothelin positive tumors, hematological malignancies, and other advanced malignancies.

In certain embodiments of any of the above aspects, the anti-CD45 antibody, or antigen-binding fragment thereof contains a combination of CDRs (i.e., CDR-H1, CDR-H2, CDR-H3, CDR-L1, CDR-L2, and CDR-L3 regions) as set forth in Table 4, below. In certain embodiments, the anti-CD45 antibody, or antigen-binding fragment thereof contains a combination of a heavy chain variable region and a light chain variable region as set forth in Table 4, below.

In certain embodiments of any of the above aspects, the anti-CD45 ADC is administered to the subject in a therapeutically effective amount such that hematopoietic stem cell (HSC) levels are maintained in the patient. In one embodiment, the level of HSCs in the subject is about 70% or more relative to the level of HSCs prior to anti-CD45 ADC treatment in the subject. In one embodiment, the level of HSCs in the subject is about 80% or more relative to the level of HSCs prior to anti-CD45 ADC treatment in the subject. In one embodiment, the level of HSCs in the subject is about 90% or more relative to the level of HSCs prior to anti-CD45 ADC treatment in the subject.

In certain embodiments of any of the above aspects, the anti-CD45 ADC treatment is administered in combination with a T cell depleting therapy. In one embodiment, the T cell depleting therapy is administered prior to, concomitantly with or following administration of the anti-CD45 ADC treatment. In one embodiment, the T cell depleting therapy comprises an agent that binds to an antigen expressed on the cell surface of a human T cell. In one embodiment, the T cell depleting therapy comprises an agent that binds to an antigen expressed on the cell surface of an activated human T cell. In one embodiment, the T cell depleting therapy comprises an anti-CD4 antibody. In one embodiment, the T cell depleting therapy comprises an anti-CD8 antibody. In one embodiment, the T cell depleting therapy comprises an anti-CD137 antibody.

In one embodiment, the T cell depleting therapy comprises an anti-CD52 antibody. In one embodiment, the T cell depleting therapy comprises an ant-CD4 antibody, an anti-CD8 antibody, an anti-CD137 antibody, and/or an anti-CD52 antibody. In one embodiment, the anti-CD52 antibody is alemtuzumab. In one embodiment, the antibody is a monoclonal antibody.

In one embodiment, the T cell depleting therapy comprises anti-thymocyte globublin (ATG). In one embodiment, the ATG is rabbit ATG (rATG). In one embodiment, the ATG is equine ATG (eATG).

In one embodiment, the T cell depleting therapy comprises total body irradiation (TBI).

In one embodiment, a lymphodepleting amount of the anti-CD45 ADC is administered.

In one embodiment, the human subject does not develop neutropenia following administration of the immune cell expressing the CAR. In certain embodiments, neutropenia is defined as the human subject having an absolute neutrophil count (ANC) of less than about 1500 per microliter (e.g., less than about 1500/μL, less than about 1400/μL, less than about 1300/μL, less than about 1200/μL, less than about 1100/μL, less than about 1000/μL, less than about 900/μL, less than about 800/μL, less than about 700/μL, or less than about 600/μL).

In one embodiment, the human subject does not develop severe neutropenia following administration of the immune cell expressing the CAR. In certain embodiments, severe neutropenia is defined as an ANC of less than about 500/μL (e.g., less than about 500/μL, less than about 450/μL, less than about 400/μL, less than about 350/μL, less than about 300/μL, less than about 250/μL, less than about 200/μL, less than about 150/μL, or less than about 100/μL).

In one embodiment, administration of the anti-CD45 ADC is effective to increase the levels of one or more CAR-T engrafting cytokines in the human subject. In certain embodiments, the CAR-T engrafting cytokine is IL-15 or IL-7.

In one embodiment, administration of the anti-CD45 ADC does not substantially increase the levels of one or more cytokine release syndrome (CRS)-cytokines in the human subject. In certain embodiments, the one or more CRS-cytokines is IFNγ, IL-10, IL-6, IL-8, MIP-1α, MIP-1β, or IL-10.

### BRIEF DESCRIPTION OF THE FIGURES

FIGS. 1A and 1B graphically depict results of cell killing assays using an anti-CD45 ADC and an isotype ADC control. FIG. 1A graphically depicts results from a human PBMC killing assay where PBMC viability was measured as the percentage of live cells in the presence of an anti-CD45-amatoxin ADC (“CD45-AM”) or an isotype-amatoxin ADC control (isotype-AM) (y-axis) as a function of anti-CD45-amatoxin ADC or control concentration (x-axis). Both ADCs had a DAR of 2. FIG. 1B graphically depicts the results of an in vitro cell killing assay that measured live human bone marrow CD34+ cells and their viability in the presence of an anti-CD45-amatoxin ADC (“CD45-AM”) or an isotype-amatoxin ADC control (isotype-AM). Both ADCs had a DAR of 2.

FIGS. 2A and 2B graphically depict results of cell killing assays in vitro using an anti-CD45 ADC. FIG. 2A graphically depicts the results of in vitro cell killing assays that shows human PBMC viability as determined by a CellTiter Glo (CTG) assay (as measured in luminescence (RLU) after 4 days of incubation with an anti-CD45-amatoxin ADC (“CD45-AM”) or an isotype-amatoxin ADC control (isotype-AM) (y-axis). Both ADCs had a DAR of 2. FIG. 2B graphically depicts the results of an in vitro cell killing assay that measured live human bone marrow CD90+ CD34+ cells and their viability in the presence of an anti-CD45-amatoxin ADC (“CD45-AM”) or an isotype-amatoxin ADC control (isotype-AM). Both ADCs had a DAR of 2.

FIG. 3 provides the FACS profile for CD45 expression on various cell subsets.

FIG. 4 graphically depicts the results of an assay detecting lymphocyte count (10³/μL) as a function of days post initial dose administration of ADC 1 (0.3 mg/kg) versus a control (i.e., PBS).

FIG. 5 graphically depicts the results of an assay detecting neutrophil count (10³/μL) as a function of days post initial dose administration of ADC 1 (0.3 mg/kg) versus a control (i.e., PBS).

FIG. 6 graphically depicts the results of an assay measuring the mean plasma concentration of ADC 1 as a function of hours post dose administration of ADC 1 (0.3 mg/kg).

FIGS. 7A-7C graphically depicts the results of an assay detecting levels of plasma alanine aminotransferase (ALT; in U/L) (FIG. 7A), total bilirubin levels (mg/dL) (FIG. 7B) and platelet count (10³/μL) (FIG. 7C) as a function of hours post dose administration of ADC 1 (0.3 mg/kg) versus a control (PBS).

FIGS. 8A and 8B graphically depict the results of an assay measuring the levels of IL-15 (pg/mL) (FIG. 8A) and IL-7 (pg/mL) (FIG. 8B) as a function of hours after ADC 1.

FIG. 9 graphically depicts the results of an assay measuring the plasma concentration (pg/mL; y-axis) for certain CRS cytokines (x-axis) at 72 hours after ADC 1.

FIG. 10 graphically depicts the results of an assay measuring conditioning efficiency in bone marrow (BM).

FIG. 11 graphically depicts the results of an assay determining donor chimerism at 3 weeks.

FIGS. 12A-12C graphically depict the results of an in vivo lymphodepletion assay measuring the levels of T cell depletion (FIG. 12A), B cell depletion (FIG. 12B), and myeloid cell depletion (FIG. 12C) in hNSG mice 14 days after administration the indicated dose of anti-CD45 ADC (“CD45-AM” comprising one of two amatoxins, notated as Amatoxin “A” or “B”) or isotype-ADC (“Isotype-AM” comprising Amatoxin “A”).

FIGS. 13A and 13B graphically depict the results of an in vivo depletion assay measuring the number of T cells (FIG. 13A) and HSCs (FIG. 13B) in the bone marrow of hNSG mice 14 days after administration the indicated dose of anti-CD45 ADC (“CD45-AM” comprising one of two amatoxins, notated as Amatoxin “A” or “B”)) or isotype-ADC (“Isotype-AM” comprising Amatoxin “A”).

### DETAILED DESCRIPTION

The present disclosure provides methods for promoting acceptance of an immune cell (either autologous or allogeneic) expressing a chimeric antigen receptor (CAR) in a human subject receiving CAR therapy by administering an anti-CD45 antibody drug conjugate (ADC) to the patient receiving the CAR therapy. The methods disclosed herein can be used to improve acceptance of autologous or allogeneic immune cells (e.g., T cells) without reliance on (or alternatively a reduced use of) lymphodepleting chemotherapy commonly used as a conditioning therapy to reduce rejection of the CAR expressing immune cells.

### I. Definitions

As used herein, the term “about” refers to a value that is within 5% above or below the value being described.

As used herein, the term “allogeneic”, when used in the context of transplantation, is used to define cells (or tissue or an organ) that are transplanted from a donor to a recipient of the same species but are not the same subject.

As used herein, the term “autologous” refers to cells or a graft where the donor and recipient are the same subject.

As used herein, the term “xenogeneic” refers to cells where the donor and recipient species are different.

As used herein, the term “immune cell” is intended to include, but is not limited to, a cell that is of hematopoietic origin and that plays a role in the immune response. Immune cells include, but are not limited to, T cells and natural killer (NK) cells. Natural killer cells are well known in the art. In one embodiment, natural killer cells include cell lines, such as NK-92 cells. Further examples of NK cell lines include NKG, YT, NK-YS, HANK-1, YTS cells, and NKL cells. An immune cell can be allogeneic or autologous. In one embodiment, an immune cell is a T cell.

An “engineered cell” means any cell of any organism that is modified, transformed, or manipulated by addition or modification of a gene, a DNA or RNA sequence, or protein or polypeptide. Isolated cells, host cells, and genetically engineered cells of the present disclosure include isolated immune cells, such as NK cells and T cells that contain the DNA or RNA sequences encoding a CAR and express the CAR on the cell surface. Isolated host cells and engineered cells may be used, for example, for enhancing an NK cell activity or a T lymphocyte activity, treatment of cancer, and treatment of autoimmune diseases. In an embodiment, the engineered cell includes immune cells, e.g., T-cells or Natural Killer (NK cells). A cell expressing a chimeric antigen receptor (CAR) is an example of an engineered cell.

As used herein, the term “antibody” refers to an immunoglobulin molecule that specifically binds to, or is immunologically reactive with, a particular antigen. An antibody includes, but is not limited to, monoclonal antibodies, polyclonal antibodies, multispecific antibodies (e.g., bispecific antibodies), chimeric antibodies, humanized antibodies, heteroconjugate antibodies (e.g., bi- tri- and quad-specific antibodies, diabodies, triabodies, and tetrabodies), and antibody fragments (i.e., antigen binding fragments of antibodies), including, for example, Fab′, F(ab′)₂, Fab, Fv, rlgG, and scFv fragments, so long as they exhibit the desired antigen-binding activity.

The antibodies of the present disclosure are generally isolated or recombinant. “Isolated,” when used herein refers to a polypeptide, e.g., an antibody, that has been identified and separated and/or recovered from a cell or cell culture from which it was expressed. Ordinarily, an isolated antibody will be prepared by at least one purification step. Thus, an “isolated antibody,” refers to an antibody which is substantially free of other antibodies having different antigenic specificities. For instance, an isolated antibody that specifically binds to CD45 is substantially free of antibodies that specifically bind antigens other than CD45.

Generally, an antibody comprises two heavy and two light chains containing antigen binding regions. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains each contain binding domains that interact with an antigen. The constant regions of the antibodies can mediate the binding of the immunoglobulin to host tissues or factors, including various cells of the immune system (e.g., effector cells) and the first component (Clq) of the classical complement system.

As used herein, the term “complementarity determining region” (CDR) refers to a hypervariable region found both in the light chain and the heavy chain variable domains of an antibody.

The more highly conserved portions of variable domains are referred to as framework regions (FRs). The amino acid positions that delineate a hypervariable region of an antibody can vary, depending on the context and the various definitions known in the art. Some positions within a variable domain may be viewed as hybrid hypervariable positions in that these positions can be deemed to be within a hypervariable region under one set of criteria while being deemed to be outside a hypervariable region under a different set of criteria. One or more of these positions can also be found in extended hypervariable regions. The antibodies described herein may contain modifications in these hybrid hypervariable positions. The variable domains of native heavy and light chains each contain four framework regions that primarily adopt a β-sheet configuration, connected by three CDRs, which form loops that connect, and in some cases form part of, the β-sheet structure. The CDRs in each chain are held together in close proximity by the framework regions in the order FR1-CDR1-FR2-CDR2-FR3-CDR3-FR4 and, with the CDRs from the other antibody chains, contribute to the formation of the target binding site of antibodies (see Kabat et al., Sequences of Proteins of Immunological Interest, National Institute of Health, Bethesda, Md., 1987). In certain embodiments, numbering of immunoglobulin amino acid residues is performed according to the immunoglobulin amino acid residue numbering system of Kabat et al., unless otherwise indicated (although any antibody numbering scheme, including, but not limited to IMGT and Chothia, can be utilized).

The term “antigen-binding fragment,” as used herein, refers to one or more portions of an antibody that retain the ability to specifically bind to a target antigen. The antigen-binding function of an antibody can be performed by fragments of a full-length antibody. The antibody fragments can be, for example, a Fab, F(ab′)2, scFv, diabody, a triabody, an affibody, a nanobody, an aptamer, or a domain antibody. Examples of binding fragments encompassed of the term “antigen-binding fragment” of an antibody include, but are not limited to: (i) a Fab fragment, a monovalent fragment consisting of the VL, VH, CL, and CH1 domains; (ii) a F(ab′)2 fragment, a bivalent fragment containing two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb including VH and VL domains; (vi) a dAb fragment that consists of a VH domain (see, e.g., Ward et al., Nature 341:544-546, 1989); (vii) a dAb which consists of a VH or a VL domain; (viii) an isolated complementarity determining region (CDR); and (ix) a combination of two or more (e.g., two, three, four, five, or six) isolated CDRs which may optionally be joined by a synthetic linker. Furthermore, although the two domains of the Fv fragment, VL and VH, are coded for by separate genes, they can be joined, using recombinant methods, by a linker that enables them to be made as a single protein chain in which the VL and VH regions pair to form monovalent molecules (known as single chain Fv (scFv); see, for example, Bird et al., Science 242:423-426, 1988 and Huston et al., Proc. Natl. Acad. Sci. USA 85:5879-5883, 1988). These antibody fragments can be obtained using conventional techniques known to those of skill in the art, and the fragments can be screened for utility in the same manner as intact antibodies. Antigen-binding fragments can be produced by recombinant DNA techniques, enzymatic or chemical cleavage of intact immunoglobulins, or, in certain cases, by chemical peptide synthesis procedures known in the art. In one embodiment, an antibody fragment comprises an Fc region.

As used herein, the term “diabody” refers to a bivalent antibody containing two polypeptide chains, in which each polypeptide chain includes V_{H} and V_{L} domains joined by a linker that is too short (e.g., a linker composed of five amino acids) to allow for intramolecular association of V_{H} and V_{L} domains on the same peptide chain. This configuration forces each domain to pair with a complementary domain on another polypeptide chain so as to form a homodimeric structure. Accordingly, the term “triabody” refers to trivalent antibodies containing three peptide chains, each of which contains one V_{H} domain and one V_{L} domain joined by a linker that is exceedingly short (e.g., a linker composed of 1-2 amino acids) to permit intramolecular association of V_{H} and V_{L} domains within the same peptide chain. In order to fold into their native structures, peptides configured in this way typically trimerize so as to position the V_{H} and V_{L} domains of neighboring peptide chains spatially proximal to one another (see, for example, Holliger et al., Proc. Natl. Acad. Sci. USA 90:6444-48, 1993).

As used herein, the term “bispecific antibody” refers to, for example, a monoclonal, e.g., a de-immunized or humanized antibody, that is capable of binding at least two different antigens or two different epitopes that can be on the same or different antigens. For instance, one of the binding specificities can be directed towards an epitope on a hematopoietic stem cell surface antigen, such as CD45, and the other can specifically bind an epitope on a different cell surface antigen or another cell surface protein, such as a receptor or receptor subunit involved in a signal transduction pathway that potentiates cell growth, among others. In some embodiments, the binding specificities can be directed towards unique, non-overlapping epitopes on the same target antigen (i.e., a biparatopic antibody).

An “intact” or “full length” antibody, as used herein, refers to an antibody having two heavy (H) chain polypeptides and two light (L) chain polypeptides interconnected by disulfide bonds. Each heavy chain is comprised of a heavy chain variable region (abbreviated herein as HCVR or VH) and a heavy chain constant region. The heavy chain constant region is comprised of three domains, CH1, CH2 and CH3. Each light chain is comprised of a light chain variable region (abbreviated herein as LCVR or VL) and a light chain constant region. The light chain constant region is comprised of one domain, CL. The VH, and VL regions can be further subdivided into regions of hypervariability, termed complementarity determining regions (CDR), interspersed with regions that are more conserved, termed framework regions (FR). Each VH and VL is composed of three CDRs and four FRs, arranged from amino-terminus to carboxyl-terminus in the following order: FR1, CDR1, FR2, CDR2, FR3, CDR3, FR4. The variable regions of the heavy and light chains contain a binding domain that interacts with an antigen.

Also provided are “conservative sequence modifications” of the sequences set forth in SEQ ID NOs described herein, i.e., nucleotide and amino acid sequence modifications which do not abrogate the binding of the antibody encoded by the nucleotide sequence or containing the amino acid sequence, to the antigen. Such conservative sequence modifications include conservative nucleotide and amino acid substitutions, as well as, nucleotide and amino acid additions and deletions. For example, modifications can be introduced into SEQ ID NOs described herein by standard techniques known in the art, such as site-directed mutagenesis and PCR-mediated mutagenesis. Conservative sequence modifications include conservative amino acid substitutions, in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine, tryptophan), nonpolar side chains (e.g., alanine, valine, leucine, isoleucine, proline, phenylalanine, methionine), beta-branched side chains (e.g., threonine, valine, isoleucine) and aromatic side chains (e.g., tyrosine, phenylalanine, tryptophan, histidine). Thus, a predicted nonessential amino acid residue in an anti-CD45 antibody is preferably replaced with another amino acid residue from the same side chain family. Methods of identifying nucleotide and amino acid conservative substitutions that do not eliminate antigen binding are well-known in the art (see, e.g., Brummell et al., Biochem. 32:1180-1187 (1993); Kobayashi et al. Protein Eng. 12(10):879-884 (1999); and Burks et al. Proc. Natl. Acad. Sci. USA 94:412-417 (1997)).

As used herein, the term “anti-CD45 antibody” or “an antibody that binds to CD45” or an “anti-CD45 ADC” or “an ADC that binds to CD45” refers to an antibody or ADC that specifically binds to human CD45. CD45 is found on the cell surface of cells, such as lymphocytes. The amino acid sequence of human CD45 to which an anti-CD45 antibody (or anti-CD45 ADC).

The term “specifically binds”, as used herein, refers to the ability of an antibody (or ADC) to recognize and bind to a specific protein structure (epitope) rather than to proteins generally. If an antibody is specific for epitope “A”, the presence of a molecule containing epitope A (or free, unlabeled A), in a reaction containing labeled “A” and the antibody, will reduce the amount of labeled A bound to the antibody. By way of example, an antibody “binds specifically” to a target if the antibody, when labeled, can be competed away from its target by the corresponding non-labeled antibody. In one embodiment, an antibody specifically binds to a target, e.g., CD45, if the antibody has a dissociation constant (K_{D}) for the target of at least about 10⁻⁴ M or less, about 10⁻⁵ M or less, about 10⁻⁶ M or less, about 10⁻⁷ M or less, about 10⁻⁸ M or less, about 10⁻⁹ M or less, about 10⁻¹⁰ M or less, about 10⁻¹¹ M, about 10⁻¹² M or less (less meaning a number that is less than 10⁻¹², e.g. 10⁻¹³). In one embodiment, the term “specific binding to CD45” or “specifically binds to CD45,” as used herein, refers to an antibody or that binds to CD45 and has a dissociation constant (K_{D}) of 1.0×10⁻⁷ M or less, as determined by surface plasmon resonance. In one embodiment, K_{D} is determined according to standard bio-layer interferometry (BLI). It shall be understood, however, that the antibody may be capable of specifically binding to two or more antigens which are related in sequence. For example, in one embodiment, an antibody can specifically bind to both human and a non-human (e.g., mouse or non-human primate) orthologs of CD45.

In some embodiments, the anti-CD45 antibody is able to specifically bind the extracellular domain of each one of the various isoforms of human CD45 (e.g., CD45RA (Uniprot Accession No: P08575-8; SEQ ID NO: 20), CD45RO (NCBI Accession No: NP_563578.2; SEQ ID NO: 21), CD45RB (NCBI Accession No: XP_006711537.1; SEQ ID NO: 22), CD45RAB (NCBI Accession No: XP_006711535.1; SEQ ID NO: 23), CD45RBC (NCBI Accession No: XP_006711536.1; SEQ ID NO: 24) and CD45RABC (NCBI Accession No. NP_002829.3; SEQ ID NO: 25)). Accordingly, in certain embodiments, the antibody herein is a pan-specific anti-CD45 antibody (i.e., an antibody that specifically binds to the extracellular region of all six human CD45 isoforms).

The term “monoclonal antibody” as used herein refers to an antibody derived from a single clone, including any eukaryotic, prokaryotic, or phage clone, by any means available or known in the art. Monoclonal antibodies useful with the present disclosure can be prepared using a wide variety of techniques known in the art including the use of hybridoma, recombinant, and phage display technologies, or a combination thereof. Unless otherwise indicated, the term “monoclonal antibody” (mAb) is meant to include both intact molecules, as well as antibody fragments (including, for example, Fab and F(ab′)₂ fragments) that are capable of specifically binding to a target protein.

The term “chimeric” antibody as used herein refers to an antibody having variable sequences derived from a non-human immunoglobulin, such as a rat or a mouse antibody, and human immunoglobulin constant regions, typically chosen from a human immunoglobulin template. Methods for producing chimeric antibodies are known in the art. See, e.g., Morrison, 1985, Science 229(4719):1202-7; Oi et al., 1986, BioTechniques 4:214-221; Gillies et al., 1985, J. Immunol. Methods 125:191-202; U.S. Pat. Nos. 5,807,715; 4,816,567; and 4,816,397.

As used herein, “drug-to-antibody ratio” or “DAR” refers to the average number of cytotoxins, e.g., amatoxin, conjugated to an antibody. Generally, the DAR of an ADC ranges from about 1 to about 8, although higher loads are also possible depending on the number of linkage sites on an antibody. Thus, in certain embodiments, an anti-CD45 ADC described herein has a DAR of 1, 2, 3, 4, 5, 6, 7, or 8.

“Humanized” forms of non-human (e.g., murine) antibodies are immunoglobulins that contain minimal sequences derived from non-human immunoglobulin. In general, a humanized antibody will comprise substantially all of at least one, and typically two, variable domains, in which all or substantially all of the CDR regions correspond to those of a non-human immunoglobulin and all or substantially all of the FR regions are those of a human immunoglobulin sequence. A humanized antibody can also comprise at least a portion of an immunoglobulin constant region (Fc), typically that of a human immunoglobulin consensus sequence. Methods of antibody humanization are known in the art. See, e.g., Riechmann et al., 1988, Nature 332:323-7; U.S. Pat. Nos. 5,530,101; 5,585,089; 5,693,761; 5,693,762; and U.S. Pat. No. 6,180,370 to Queen et al.; EP239400; PCT publication WO 91/09967; U.S. Pat. No. 5,225,539; EP592106; EP519596; Padlan, 1991, Mol. Immunol., 28:489-498; Studnicka et al., 1994, Prot. Eng. 7:805-814; Roguska et al., 1994, Proc. Natl. Acad. Sci. 91:969-973; and U.S. Pat. No. 5,565,332.

As used herein, the terms “chimeric antigen receptor” or “CAR” refer to a recombinant polypeptide comprising at least an extracellular domain capable of specifically binding an antigen, a transmembrane domain, and at least one intracellular signaling domain. Generally, a CAR is a genetically engineered receptor that redirects cytotoxicity of immune effector cells toward cells presenting the given antigen. CARs are molecules that combine antibody-based specificity for a desired antigen (e.g., a tumor antigen) with a T cell receptor-activating intracellular domain to generate a chimeric protein that exhibits a specific cellular immune activity. In particular embodiments, CARs comprise an extracellular domain (also referred to as a binding domain or antigen-specific binding domain), a transmembrane domain, and an intracellular (cytoplasmic) signaling domain. Engagement of the antigen binding domain of the CAR with the target antigen on the surface of a target cell results in clustering of the CAR and delivers an activation stimulus to the CAR-containing cell. A main characteristic of a CAR is its ability to redirect immune effector cell specificity, thereby triggering proliferation, cytokine production, phagocytosis or production of molecules that can mediate cell death of the target antigen expressing cell in a major histocompatibility (MHC) independent manner, exploiting the cell specific targeting abilities of monoclonal antibodies, soluble ligands or cell specific co-receptors. In various embodiments, a CAR comprises an extracellular binding domain that specifically binds human CD45; a transmembrane domain; and one or more intracellular signaling domains.

As used herein, the term “CAR therapy” refers to administration of an immune cell that has been engineered to express a CAR, to a human subject for the treatment of a given disease, e.g., cancer or an autoimmune disease. CAR therapy refers to the specific treatment of the patient with the engineered immune cells and is not intended to include therapies that commonly are used in conjunction with CAR cell treatment, e.g., lymphodepleting chemotherapy. Notably, where the term “cell” is used throughout, populations of cells are also included by the term unless otherwise specified. For example, as CAR therapy requires administration of a population of engineered cells.

As used herein, the term “combination” or “combination therapy” refers to the use of two (or more) therapies in a single human patient. The terms are not intended to refer to a combination composition. For example, described herein is a combination therapy comprising administering an anti-CD45 ADC and CAR therapy.

The term “conditioning” refers to the preparation of a patient in need of CAR therapy for a suitable condition. Conditioning as used herein includes, but is not limited to, reducing the number of endogenous lymphocytes, removing a cytokine sink, increasing a serum level of one or more homeostatic cytokines or pro-inflammatory factors, enhancing an effector function of T cells administered after the conditioning, enhancing antigen presenting cell activation and/or availability, or any combination thereof prior to a T cell therapy.

The term “deplete,” in the context of the effect of an anti-CD45 antibody or ADC on CD45-expressing cells, refers to a reduction in the number of or elimination of CD45-expressing cells.

The phrase “therapeutically effective amount” or “therapeutically effective dose”, used interchangeably herein, refers to the amount or dose of a therapeutic agent, e.g., an anti-CD45 ADC, which, upon single or multiple dose administration to a patient, provides the desired treatment, is sufficient to achieve the desired result, or to have an effect on an autoimmune disease or cancer. A “therapeutically effective amount” of a therapeutic agent may vary according to factors such as the disease state, age, sex, and weight of the individual, such that the amount is able to elicit a desired response in the individual. The term “therapeutically effective amount” includes an amount that is effective to “treat” a subject (e.g., a patient). When a therapeutic amount is indicated, the precise amount of the compositions of the present invention to be administered can be determined by a physician with consideration of individual differences in age, weight, tumor size, extent of infection or metastasis, and condition of the patient (subject). In one embodiment, a therapeutically effective amount of an anti-CD45 ADC is a lymphodepleting dose.

As used herein, the phrase “lymphodepleting dose” refers to an amount of a therapeutic agent, e.g., an anti-CD45 antibody or an anti-CD45 ADC, that is effective to deplete lymphocytes in a subject while not substantially depleting hematopoietic stem cells (HSCs) in the subject.

As used herein, the term “half-life” refers to the time it takes for the plasma concentration of the antibody drug in the body to be reduced by one half or 50% in a subject, e.g., a human subject. This 50% reduction in serum concentration reflects the amount of drug circulating.

The terms “Fc” “Fc region,” “Fc domain,” and “IgG Fc domain” as used herein refer to the portion of an immunoglobulin, e.g., an IgG molecule, which correlates to a crystallizable fragment obtained by papain digestion of an IgG molecule. The Fc region comprises the C-terminal half of two heavy chains of an IgG molecule that are linked by disulfide bonds. It has no antigen binding activity but contains the carbohydrate moiety and binding sites for complement and Fc receptors, including the FcRn receptor (see below). For example, an Fc domain contains the second constant domain CH2 (e.g., residues at EU positions 231-340 of human IgG1) and the third constant domain CH3 (e.g., residues at EU positions 341-447 of human IgG1). As used herein, the Fc domain includes the “lower hinge region” (e.g., residues at EU positions 233-239 of human IgG1).

Fc can refer to this region in isolation, or this region in the context of an antibody, antibody fragment, or Fc fusion protein. Polymorphisms have been observed at a number of positions in Fc domains, including but not limited to EU positions 270, 272, 312, 315, 356, and 358, and thus slight differences between the sequences presented in the instant application and sequences known in the art can exist. Thus, a “wild type IgG Fc domain” or “WT IgG Fc domain” refers to any naturally occurring IgG Fc region (i.e., any allele). The sequences of the heavy chains of human IgG1, IgG2, IgG3 and IgG4 can be found in a number of sequence databases, for example, at the Uniprot database (www.uniprot.org) under accession numbers P01857 (IGHG1_HUMAN), P01859 (IGHG2_HUMAN), P01860 (IGHG3_HUMAN), and P01861 (IGHG1_HUMAN), respectively.

The terms “modified Fc region” or “variant Fc region” as used herein refers to an IgG Fc domain comprising one or more amino acid substitutions, deletions, insertions or modifications introduced at any position within the Fc domain. In certain aspects a variant IgG Fc domain comprises one or more amino acid substitutions resulting in decreased or ablated binding affinity for an Fc gamma R and/or C1q as compared to the wild type Fc domain not comprising the one or more amino acid substitutions. Further, Fc binding interactions are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a variant Fc domain (e.g., an antibody, fusion protein or conjugate) can exhibit altered binding affinity for at least one or more Fc ligands (e.g., Fc gamma Rs) relative to a corresponding antibody otherwise having the same amino acid sequence but not comprising the one or more amino acid substitution, deletion, insertion or modifications such as, for example, an unmodified Fc region containing naturally occurring amino acid residues at the corresponding position in the Fc region.

Variant Fc domains are defined according to the amino acid modifications that compose them. For all amino acid substitutions discussed herein in regard to the Fc region, numbering is always according to the EU index as in Kabat. Thus, for example, D265C is an Fc variant with the aspartic acid (D) at EU position 265 substituted with cysteine (C) relative to the parent Fc domain. Likewise, e.g., D265C/L234A/L235A defines a variant Fc variant with substitutions at EU positions 265 (D to C), 234 (L to A), and 235 (L to A) relative to the parent Fc domain. A variant can also be designated according to its final amino acid composition in the mutated EU amino acid positions. For example, the L234A/L235A mutant can be referred to as “LALA”. As a further example, the E233P.L234V.L235A.delG236 (deletion of 236) mutant can be referred to as “EPLVLAdelG”. As yet another example, the I253A.H310A.H435A mutant can be referred to as “IHH”. It is noted that the order in which substitutions are provided is arbitrary.

The terms “Fc gamma receptor” or “Fc gamma R” as used herein refer to any member of the family of proteins that bind the IgG antibody Fc region and are encoded by the Fc gamma R genes. In humans this family includes but is not limited to Fcg amma RI (CD64), including isoforms Fc gamma RIa, Fc gamma RIb, and Fc gamma RIc; Fc gamma RII (CD32), including isoforms Fc gamma RIIa (including allotypes H131 and R131), Fc gamma RIIb (including Fc gamma RIIb-1 and Fc gamma RIIb-2), and Fc gamma RIIc; and Fc gamma RIII (CD16), including isoforms Fc gamma RIIIa (including allotypes V158 and F158) and Fc gamma RIIIb (including allotypes Fc gamma RIIIb-NA1 and Fc gamma RIIIb-NA2), as well as any undiscovered human Fc gamma Rs or Fc gamma R isoforms or allotypes. An Fc gamma R can be from any organism, including but not limited to humans, mice, rats, rabbits, and monkeys. Mouse Fc gamma Rs include but are not limited to Fc gamma RI (CD64), Fc gamma RII (CD32), Fc gamma RIII (CD16), and Fc gamma RIII-2 (CD16-2), as well as any undiscovered mouse Fc gamma Rs or Fc gamma R isoforms or allotypes.

The term “effector function” as used herein refers to a biochemical event that results from the interaction of an Fc domain with an Fc receptor. Effector functions include but are not limited to ADCC, ADCP, and CDC. By “effector cell” as used herein is meant a cell of the immune system that expresses or one or more Fc receptors and mediates one or more effector functions. Effector cells include but are not limited to monocytes, macrophages, neutrophils, dendritic cells, eosinophils, mast cells, platelets, B cells, large granular lymphocytes, Langerhans' cells, natural killer (NK) cells, and gamma delta T cells, and can be from any organism included but not limited to humans, mice, rats, rabbits, and monkeys.

The term “silent”, “silenced”, or “silencing” as used herein refers to an antibody having a modified Fc region described herein that has decreased binding to an Fc gamma receptor (FcγR) relative to binding of an identical antibody comprising an unmodified Fc region to the FcγR (e.g., a decrease in binding to a FcγR by at least 70%, at least 80%, at least 90%, at least 95%, at least 98%, at least 99%, or 100% relative to binding of the identical antibody comprising an unmodified Fc region to the FcγR as measured by, e.g., BLI). In some embodiments, the Fc silenced antibody has no detectable binding to an FcγR. Binding of an antibody having a modified Fc region to an FcγR can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE™ analysis or Octet™ analysis (forteBlO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmunoassay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

As used herein, the term “identical antibody comprising an unmodified Fc region” refers to an antibody that lacks the recited amino acid substitutions (e.g., D265C, H435A, L234A, and/or L235A), but otherwise has the same amino acid sequence as the Fc modified antibody to which it is being compared.

As used herein, the terms “subject” and “patient” refer to an organism, such as a human, that receives treatment for a particular disease or condition as described herein.

As used herein, the term “endogenous” describes a substance, such as a molecule, cell, tissue, or organ (e.g., CD45+ immune cells, such as endogenous lymphocytes) that is found naturally in a particular organism, such as a human patient.

As used herein, the term “sample” refers to a specimen (e.g., blood, blood component (e.g., serum or plasma), urine, saliva, amniotic fluid, cerebrospinal fluid, tissue (e.g., placental or dermal), pancreatic fluid, chorionic villus sample, and cells) taken from a subject.

As used herein, the phrase “substantially cleared from the blood” refers to a point in time following administration of a therapeutic agent (such as an anti-CD45 antibody, or antigen-binding fragment thereof) to a patient when the concentration of the therapeutic agent in a blood sample isolated from the patient is such that the therapeutic agent is not detectable by conventional means (for instance, such that the therapeutic agent is not detectable above the noise threshold of the device or assay used to detect the therapeutic agent). A variety of techniques known in the art can be used to detect antibodies, antibody fragments, and protein ligands, such as ELISA-based detection assays known in the art or described herein. Additional assays that can be used to detect antibodies, or antibody fragments, include immunoprecipitation techniques and immunoblot assays, among others known in the art.

As used herein “to treat” or “treatment”, refer to any improvement of any consequence of disease, such as prolonged survival, less morbidity, and/or a lessening of side effects which are the byproducts of an alternative therapeutic modality; as is readily appreciated in the art, full eradication of disease is a preferred but albeit not a requirement for a treatment act. Beneficial or desired clinical results include, but are not limited to, promoting acceptance of CAR expressing immune cells (allogeneic or autologous—both of which can cause immune reactions in a patient receiving CAR therapy). Insofar as the methods of the present disclosure are directed to preventing disorders, it is understood that the term “prevent” does not require that the disease state be completely thwarted. Rather, as used herein, the term preventing refers to the ability of the skilled artisan to identify a population that is susceptible to disorders, such that administration of the compounds of the present disclosure may occur prior to onset of a disease. The term does not imply that the disease state is completely avoided.

As used herein, the term “vector” includes a nucleic acid vector, such as a plasmid, a DNA vector, a plasmid, a RNA vector, virus, or other suitable replicon. Expression vectors described herein may contain a polynucleotide sequence as well as, for example, additional sequence elements used for the expression of proteins and/or the integration of these polynucleotide sequences into the genome of a mammalian cell. Certain vectors that can be used for the expression of CARs or antibodies include plasmids that contain regulatory sequences, such as promoter and enhancer regions, which direct gene transcription. Other useful vectors for antibody or CAR expression contain polynucleotide sequences that enhance the rate of translation of these genes or improve the stability or nuclear export of the mRNA that results from gene transcription. These sequence elements may include, for example, 5′ and 3′ untranslated regions and a polyadenylation signal site in order to direct efficient transcription of the gene carried on the expression vector. The expression vectors described herein may also contain a polynucleotide encoding a marker for selection of cells that contain such a vector. Examples of a suitable marker include genes that encode resistance to antibiotics, such as ampicillin, chloramphenicol, kanamycin, and nourseothricin.

As used herein, the term “antibody drug conjugate” or “ADC” refers to an antibody which is linked to a cytotoxin. An ADC is formed by the chemical bonding of a reactive functional group of one molecule, such as an antibody or antigen-binding fragment thereof, with an appropriately reactive functional group of another molecule, such as a cytotoxin described herein. Conjugates may include a linker between the two molecules bound to one another, e.g., between an antibody and a cytotoxin. Examples of linkers that can be used for the formation of a conjugate include peptide-containing linkers, such as those that contain naturally occurring or non-naturally occurring amino acids, such as D-amino acids. Linkers can be prepared using a variety of strategies described herein and known in the art. Depending on the reactive components therein, a linker may be cleaved, for example, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012).

As used herein, the term “microtubule-binding agent” refers to a compound which acts by disrupting the microtubular network that is essential for mitotic and interphase cellular function in a cell. Examples of microtubule-binding agents include, but are not limited to, maytasine, maytansinoids, and derivatives thereof, such as those described herein or known in the art, vinca alkaloids, such as vinblastine, vinblastine sulfate, vincristine, vincristine sulfate, vindesine, and vinorelbine, taxanes, such as docetaxel and paclitaxel, macrolides, such as discodermolides, cochicine, and epothilones, and derivatives thereof, such as epothilone B or a derivative thereof.

As used herein, the term “amatoxin” refers to a member of the amatoxin family of peptides produced by Amanita phalloides mushrooms, or derivative thereof, such as a variant or derivative thereof capable of inhibiting RNA polymerase II activity. Also included are synthetic amatoxins (see, e.g., U.S. Pat. No. 9,676,702, incorporated by reference herein). Amatoxins useful in conjunction with the compositions and methods described herein include compounds such as, but not limited to, amatoxins of Formulas (III), (IIIa), (IIIb), and (Mc) as described herein, (e.g., α-amanitin, β-amanitin, γ-amanitin, ε-amanitin, amanin, amaninamide, amanullin, amanullinic acid, or proamanullin, and derivatives thereof). As described herein, amatoxins may be conjugated to an antibody, or antigen-binding fragment thereof, for instance, by way of a linker moiety (L) (thus forming an ADC). Such ADCs are represented by the formula Ab-Z-L-Am, wherein Ab is the antibody or antigen-binding fragment thereof, L is a linker, Z is a chemical moiety, and Am is the amatoxin. In some embodiments, the amatoxin is conjugated to a linker. In some embodiments, the amatoxin-linker conjugate Am-L-Z is represented by formulae (I) or (IA), (IB), (IV), (IVA), or (IVB). Exemplary methods of amatoxin conjugation and linkers useful for such processes are described below. Exemplary linker-containing amatoxins useful for conjugation to an antibody, or antigen-binding fragment, in accordance with the compositions and methods are also described herein.

The term “acyl” as used herein refers to —C(═O)R, wherein R is hydrogen (“aldehyde”), alkyl (e.g., C₁-C₁₂ alkyl), alkenyl (e.g., C₂-C₁₂ alkenyl), alkynyl (e.g., C₂-C₁₂ alkynyl), carbocyclyl (e.g., C₃-C₇ carbocyclyl), aryl (e.g., C₆-C₂₀ aryl), heteroaryl (e.g., 5-10 membered heteroaryl), or heterocyclyl (e.g., 5-10 membered heterocyclyl), as defined herein. Non-limiting examples include formyl, acetyl, propanoyl, benzoyl, and acryloyl.

The term “alkyl” as used herein refers to a straight chain or branched, saturated hydrocarbon having from 1 to 12 carbon atoms. Representative C₁-C₁₂ alkyl groups include, but are not limited to, -methyl, -ethyl, -n-propyl, -n-butyl, -n-pentyl, and -n-hexyl; while branched C₁-C₁₂ alkyls include, but are not limited to, -isopropyl, -sec-butyl, -isobutyl, -tert-butyl, -isopentyl, and 2-methylbutyl. A C₁-C₁₂ alkyl group can be unsubstituted or substituted.

The term “alkenyl” as used herein refers to C₂-C₁₂ hydrocarbon containing normal, secondary, or tertiary carbon atoms with at least one site of unsaturation, i.e., a carbon-carbon, sp2 double bond. Examples include, but are not limited to: ethylene or vinyl, -allyl, -1-butenyl, -2-butenyl, -isobutylenyl, -1-pentenyl, -2-pentenyl, -3-methyl-1-butenyl, -2-methyl-2-butenyl, -2,3-dimethyl-2-butenyl, and the like. An alkenyl group can be unsubstituted or substituted.

“Alkynyl” as used herein refers to a C₂-C₁₂ hydrocarbon containing normal, secondary, or tertiary carbon atoms with at least one site of unsaturation, i.e., a carbon-carbon, sp triple bond. Examples include, but are not limited to acetylenic and propargyl. An alkynyl group can be unsubstituted or substituted.

“Aryl” as used herein refers to a C₆-C₂₀ carbocyclic aromatic group. Examples of aryl groups include, but are not limited to, phenyl, naphthyl and anthracenyl. An aryl group can be unsubstituted or substituted.

“Arylalkyl” as used herein refers to an acyclic alkyl radical in which one of the hydrogen atoms bonded to a carbon atom, typically a terminal or sp³ carbon atom, is replaced with an aryl radical. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. The arylalkyl group comprises 6 to 20 carbon atoms, e.g. the alkyl moiety, including alkanyl, alkenyl or alkynyl groups, of the arylalkyl group is 1 to 6 carbon atoms and the aryl moiety is 5 to 14 carbon atoms. An alkanyl group can be unsubstituted or substituted.

“Cycloalkyl” as used herein refers to a saturated carbocyclic radical, which may be mono- or bicyclic. Cycloalkyl groups include a ring having 3 to 7 carbon atoms as a monocycle or 7 to 12 carbon atoms as a bicycle. Examples of monocyclic cycloalkyl groups include cyclopropyl, cyclobutyl, cyclopentyl, cyclohexyl, cycloheptyl, and cyclooctyl. A cycloalkyl group can be unsubstituted or substituted.

“Cycloalkenyl” as used herein refers to an unsaturated carbocyclic radical, which may be mono- or bicyclic. Cycloalkenyl groups include a ring having 3 to 6 carbon atoms as a monocycle or 7 to 12 carbon atoms as a bicycle. Examples of monocyclic cycloalkenyl groups include 1-cyclopent-1-enyl, 1-cyclopent-2-enyl, 1-cyclopent-3-enyl, 1-cyclohex-1-enyl, 1-cyclohex-2-enyl, and 1-cyclohex-3-enyl. A cycloalkenyl group can be unsubstituted or substituted.

“Heteroaralkyl” as used herein refers to an acyclic alkyl radical in which one of the hydrogen atoms bonded to a carbon atom, typically a terminal or sp3 carbon atom, is replaced with a heteroaryl radical. Typical heteroarylalkyl groups include, but are not limited to, 2-benzimidazolylmethyl, 2-furylethyl, and the like. The heteroarylalkyl group comprises 6 to 20 carbon atoms, e.g. the alkyl moiety, including alkanyl, alkenyl or alkynyl groups, of the heteroarylalkyl group is 1 to 6 carbon atoms and the heteroaryl moiety is 5 to 14 carbon atoms and 1 to 3 heteroatoms selected from N, O, P, and S. The heteroaryl moiety of the heteroarylalkyl group may be a monocycle having 3 to 7 ring members (2 to 6 carbon atoms or a bicycle having 7 to 10 ring members (4 to 9 carbon atoms and 1 to 3 heteroatoms selected from N, O, P, and S), for example: a bicyclo[4,5], [5,5], [5,6], or [6,6] system.

“Heteroaryl” and “heterocycloalkyl” as used herein refer to an aromatic or non-aromatic ring system, respectively, in which one or more ring atoms is a heteroatom, e.g. nitrogen, oxygen, and sulfur. The heteroaryl or heterocycloalkyl radical comprises 2 to 20 carbon atoms and 1 to 3 heteroatoms selected from N, O, P, and S. A heteroaryl or heterocycloalkyl may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo[4,5], [5,5], [5,6], or [6,6] system. Heteroaryl and heterocycloalkyl can be unsubstituted or substituted.

Heteroaryl and heterocycloalkyl groups are described in Paquette, Leo A.; “Principles of Modern Heterocyclic Chemistry” (W. A. Benjamin, New York, 1968), particularly Chapters 1, 3, 4, 6, 7, and 9; “The Chemistry of Heterocyclic Compounds, A series of Monographs” (John Wiley & Sons, New York, 1950 to present), in particular Volumes 13, 14, 16, 19, and 28; and J. Am. Chem. Soc. (1960) 82:5566.

Examples of heteroaryl groups include by way of example and not limitation pyridyl, thiazolyl, tetrahydrothiophenyl, pyrimidinyl, furanyl, thienyl, pyrrolyl, pyrazolyl, imidazolyl, tetrazolyl, benzofuranyl, thianaphthalenyl, indolyl, indolenyl, quinolinyl, isoquinolinyl, benzimidazolyl, isoxazolyl, pyrazinyl, pyridazinyl, indolizinyl, isoindolyl, 3H-indolyl, 1H-indazolyl, purinyl, 4H-quinolizinyl, phthalazinyl, naphthyridinyl, quinoxalinyl, quinazolinyl, cinnolinyl, pteridinyl, 4aH-carbazolyl, carbazolyl, phenanthridinyl, acridinyl, pyrimidinyl, phenanthrolinyl, phenazinyl, phenothiazinyl, furazanyl, phenoxazinyl, isochromanyl, chromanyl, imidazolidinyl, imidazolinyl, pyrazolidinyl, pyrazolinyl, benzotriazolyl, benzisoxazolyl, and isatinoyl.

Examples of heterocycloalkyls include by way of example and not limitation dihydroypyridyl, tetrahydropyridyl (piperidyl), tetrahydrothiophenyl, piperidinyl, 4-piperidonyl, pyrrolidinyl, 2-pyrrolidonyl, tetrahydrofuranyl, tetrahydropyranyl, bis-tetrahydropyranyl, tetrahydroquinolinyl, tetrahydroisoquinolinyl, decahydroquinolinyl, octahydroisoquinolinyl, piperazinyl, quinuclidinyl, and morpholinyl.

By way of example and not limitation, carbon bonded heteroaryls and heterocycloalkyls are bonded at position 2, 3, 4, 5, or 6 of a pyridine, position 3, 4, 5, or 6 of a pyridazine, position 2, 4, 5, or 6 of a pyrimidine, position 2, 3, 5, or 6 of a pyrazine, position 2, 3, 4, or 5 of a furan, tetrahydrofuran, thiofuran, thiophene, pyrrole or tetrahydropyrrole, position 2, 4, or 5 of an oxazole, imidazole or thiazole, position 3, 4, or 5 of an isoxazole, pyrazole, or isothiazole, position 2 or 3 of an aziridine, position 2, 3, or 4 of an azetidine, position 2, 3, 4, 5, 6, 7, or 8 of a quinoline or position 1, 3, 4, 5, 6, 7, or 8 of an isoquinoline. Still more typically, carbon bonded heterocycles include 2-pyridyl, 3-pyridyl, 4-pyridyl, 5-pyridyl, 6-pyridyl, 3-pyridazinyl, 4-pyridazinyl, 5-pyridazinyl, 6-pyridazinyl, 2-pyrimidinyl, 4-pyrimidinyl, 5-pyrimidinyl, 6-pyrimidinyl, 2-pyrazinyl, 3-pyrazinyl, 5-pyrazinyl, 6-pyrazinyl, 2-thiazolyl, 4-thiazolyl, or 5-thiazolyl.

By way of example and not limitation, nitrogen bonded heteroaryls and heterocycloalkyls are bonded at position 1 of an aziridine, azetidine, pyrrole, pyrrolidine, 2-pyrroline, 3-pyrroline, imidazole, imidazolidine, 2-imidazoline, 3-imidazoline, pyrazole, pyrazoline, 2-pyrazoline, 3-pyrazoline, piperidine, piperazine, indole, indoline, 1H-indazole, position 2 of a isoindole, or isoindoline, position 4 of a morpholine, and position 9 of a carbazole, or beta-carboline. Still more typically, nitrogen bonded heterocycles include 1-aziridyl, 1-azetedyl, 1-pyrrolyl, 1-imidazolyl, 1-pyrazolyl, and 1-piperidinyl.

“Substituted” as used herein and as applied to any of the above alkyl, alkenyl, alkynyl, aryl, arylalkyl, cycloalkyl, heteroaryl, heterocyclyl, and the like, means that one or more hydrogen atoms are each independently replaced with a substituent. Typical substituents include, but are not limited to, —X, —R, —OH, —OR, —SH, —SR, NH₂, —NHR, —N(R)₂, —N⁺(R)₃, —CX₃, —CN, —OCN, —SCN, —NCO, —NCS, —NO, —NO₂, —N₃, —NC(═O)H, —NC(═O)R, —C(═O)H, —C(═O)R, —C(═O)NH₂, —C(═O)N(R)₂, —SO₃—, —SO₃H, —S(═O)₂R, —OS(═O)₂OR, —S(═O)₂NH₂, —S(═O)₂N(R)₂, —S(═O)R, —OP(═O)(OH)₂, —OP(═O)(OR)₂, —P(═O)(OR)₂, —PO₃, —PO₃H₂, —C(═O)X, —C(═S)R, —CO₂H, —CO₂R, —CO₂—, —C(═S)OR, —C(═O)SR, —C(═S)SR, —C(═O)NH₂, —C(═O)N(R)₂, —C(═S)NH₂, —C(═S)N(R)₂, —C(═NH)NH₂, and —C(═NR)N(R)₂; wherein each X is independently selected for each occasion from F, Cl, Br, and I; and each R is independently selected for each occasion from C₁-C₁₂ alkyl, C₆-C₂₀ aryl, C₃-C₁₄ heterocycloalkyl or heteroaryl, protecting group and prodrug moiety. Wherever a group is described as “optionally substituted,” that group can be substituted with one or more of the above substituents, independently for each occasion. The substitution may include situations in which neighboring substituents have undergone ring closure, such as ring closure of vicinal functional substituents, to form, for instance, lactams, lactones, cyclic anhydrides, acetals, hemiacetals, thioacetals, aminals, and hemiaminals, formed by ring closure, for example, to furnish a protecting group.

It is to be understood that certain radical naming conventions can include either a mono-radical or a di-radical, depending on the context. For example, where a substituent requires two points of attachment to the rest of the molecule, it is understood that the substituent is a di-radical. For example, a substituent identified as alkyl that requires two points of attachment includes di-radicals such as —CH₂—, —CH₂CH₂—, —CH₂CH(CH₃)CH₂—, and the like. Other radical naming conventions clearly indicate that the radical is a di-radical such as “alkylene,” “alkenylene,” “arylene,” “heterocycloalkylene,” and the like.

“Isomerism” or “isomers” refers to compounds that have identical molecular formulae but differ in the sequence of bonding of their atoms or in the arrangement of their atoms in space. Isomers that differ in the arrangement of their atoms in space are termed “stereoisomers.” Stereoisomers that are not mirror images of one another are termed “diastereoisomers,” and stereoisomers that are non-superimposable mirror images of each other are termed “enantiomers,” or sometimes “optical isomers.”

A carbon atom bonded to four non-identical substituents is termed a “chiral center.” “Chiral isomer” means a compound with at least one chiral center. Compounds with more than one chiral center may exist either as an individual diastereomer or as a mixture of diastereomers, termed “diastereomeric mixture.” When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1966, 5, 385; errata 511; Cahn et al., Angew. Chem. 1966, 78, 413; Cahn and Ingold, J. Chem. Soc. 1951 (London), 612; Cahn et al., Experientia 1956, 12, 81; Cahn, J. Chem. Educ. 1964, 41, 116). A mixture containing equal amounts of individual enantiomeric forms of opposite chirality is termed a “racemic mixture.”

The compounds disclosed in this description and in the claims may comprise one or more asymmetric centers, and different diastereomers and/or enantiomers of each of the compounds may exist. The description of any compound in this description and in the claims is meant to include all enantiomers, diastereomers, and mixtures thereof, unless stated otherwise. In addition, the description of any compound in this description and in the claims is meant to include both the individual enantiomers, as well as any mixture, racemic or otherwise, of the enantiomers, unless stated otherwise. When the structure of a compound is depicted as a specific enantiomer, it is to be understood that the disclosure of the present application is not limited to that specific enantiomer. Accordingly, enantiomers, optical isomers, and diastereomers of each of the structural formulae of the present disclosure are contemplated herein. In the present specification, the structural formula of the compound represents a certain isomer for convenience in some cases, but the present disclosure includes all isomers, such as geometrical isomers, optical isomers based on an asymmetrical carbon, stereoisomers, tautomers, and the like, it being understood that not all isomers may have the same level of activity. The compounds may occur in different tautomeric forms. The compounds according to the disclosure are meant to include all tautomeric forms, unless stated otherwise. When the structure of a compound is depicted as a specific tautomer, it is to be understood that the disclosure of the present application is not limited to that specific tautomer.

The compounds of any formula described herein include the compounds themselves, as well as their salts, and their solvates, if applicable. A salt, for example, can be formed between an anion and a positively charged group (e.g., amino) on a compound of the disclosure. Suitable anions include chloride, bromide, iodide, sulfate, bisulfate, sulfamate, nitrate, phosphate, citrate, methanesulfonate, trifluoroacetate, glutamate, glucuronate, glutarate, malate, maleate, succinate, fumarate, tartrate, tosylate, salicylate, lactate, naphthalenesulfonate, and acetate (e.g., trifluoroacetate). The term “pharmaceutically acceptable anion” refers to an anion suitable for forming a pharmaceutically acceptable salt. Likewise, a salt can also be formed between a cation and a negatively charged group (e.g., carboxylate) on a compound of the disclosure. Suitable cations include sodium ion, potassium ion, magnesium ion, calcium ion, and an ammonium cation such as tetramethylammonium ion. Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. The compounds of the disclosure also include those salts containing quaternary nitrogen atoms.

Examples of suitable inorganic anions include, but are not limited to, those derived from the following inorganic acids: hydrochloric, hydrobromic, hydroiodic, sulfuric, sulfurous, nitric, nitrous, phosphoric, and phosphorous. Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Additionally, the compounds of the present disclosure, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc. “Solvate” means solvent addition forms that contain either stoichiometric or non-stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O. A hydrate refers to, for example, a mono-hydrate, a di-hydrate, a tri-hydrate, etc.

In addition, a crystal polymorphism may be present for the compounds or salts thereof represented by the formulae disclosed herein. It is noted that any crystal form, crystal form mixture, or anhydride or hydrate thereof, is included in the scope of the present disclosure.

Wherever a substituent is depicted as a di-radical (i.e., has two points of attachment to the rest of the molecule), it is to be understood that the substituent can be attached in any directional configuration unless otherwise indicated.

The sections that follow provide a description of methods based on the administration of an anti-CD45 ADCs to a human patient to promote acceptance of an immune cell expressing a CAR in CAR therapy.

### II. Anti-CD45 Antibody Drug Conjugates (ADCs) and Chimeric Antigen Receptor (CAR) Cell Methods of Treatment

A challenge of chimeric antigen receptor (CAR) therapy is determining a means by which the engineered CAR expressing cells, e.g., CAR-T cells, can be accepted by a human recipient. Such acceptance of the engineered immune cells can impact the efficacy of the treatment and also results in adverse side effects to the patient.

Lymphodepleting chemotherapy is a traditional way of suppressing the recipient's immune system to improve acceptance, but commonly has adverse side effects. Described herein are methods of promoting acceptance of CAR expressing immune cells in human patients who are receiving CAR therapy. The methods described herein specifically target CD45+ cells, e.g., CD45+ lymphocytes (e.g., T cells), in the human patient who is undergoing CAR therapy and ablates the CD45+ cells. The methods disclosed herein are more targeted than lymphodepleting chemotherapy and provide a means by which either autologous or allogeneic cells can be used. An advantage of the methods disclosed herein is that the treatment is lymphodepleting to the patient in need thereof (i.e., a patient in need of CAR therapy), but not substantially depleting of HSCs. For example, the methods disclosed herein may be capable of lymphodepleting a patient in need thereof without inducing myeloablation, e.g., myelosuppression that requires an HSC transplant to restore the patient's hematopoietic system.

Described herein are methods of administering anti-CD45 antibody-drug conjugates (ADCs) to deplete a population of CD45 specific cells (e.g., lymphocytes) within the patient receiving CAR therapy in order to facilitate the acceptance and efficacy of CAR-expressing immune cells. This selective depletion of specific CD45 expressing cells of the immune system improves overall and relapse-free patient survival while decreasing the risk of rejection of the CAR-expressing immune cell for treating autoimmune disorders or cancer.

The risk of rejection of a CAR expressing immune cell remains high following the administration of CAR cell therapies. The methods and compositions disclosed herein may be used to inhibit or prevent the rejection of a CAR cell in a human patient. The anti-CD45 ADCs may be used to selectively target lymphocytes in a patient who will be receiving a CAR cell therapy. Anti-CD45 ADCs, as described herein, may also be used to reduce the risk of the rejection of a CAR cell by targeting and depleting CD45 positive cells in a human patient who has already received a CAR cell therapy.

The compositions and methods described herein may be used to deplete CD45+ cells, e.g., lymphocytes, that are associated with CAR cell therapy rejection. The methods of the disclosure promote acceptance of an immune cell expressing a CAR in a human subject, e.g., a human subject having cancer or an autoimmune disease. In one embodiment, the method includes administering an anti-CD45 antibody drug conjugate (ADC) to a human subject who will be undergoing or has undergone CAR therapy, and administering a therapeutically effective amount of an immune cell expressing a CAR to the human subject. The CAR-expressing immune cell can be allogeneic or autologous.

The anti-CD45 ADC can be administered to the human patient in need prior to, concomitantly with, or following administration of CAR cell therapies. In one embodiment, an anti-CD45 ADC is administered to the human patient in need thereof prior to (e.g., about 1 to about 10 days before, about 1 to about 5 days before, about 1 to about 3 days before, about 3 days before, about 2 days before, about 12 hours after) administration of CAR cell therapies. A single dose of an anti-CD45 ADC may be administered to the human patient either prior to, after, or concomitantly with, administration of CAR cell therapies, where such single dose is sufficient to prevent or reduce the risk of depletion of the CAR expressing immune cell. In one embodiment, an anti-CD45 ADC is administered to the human patient in need thereof about 3 days prior to administration of CAR cell therapies. In one embodiment, an anti-CD45 ADC is administered to the human patient in need thereof about 2 days prior to administration of CAR cell therapies. In one embodiment, an anti-CD45 ADC is administered to the human patient in need thereof about 1 day prior to administration of CAR cell therapies. In one embodiment, an anti-CD45 ADC is administered to the human patient in need thereof about 20 hours prior to administration of CAR cell therapies. In one embodiment, an anti-CD45 ADC is administered to the human patient in need thereof about 18 hours prior to administration of CAR cell therapies. In one embodiment, an anti-CD45 ADC is administered to the human patient in need thereof about 15 hours prior to administration of CAR cell therapies. In one embodiment, an anti-CD45 ADC is administered to the human patient in need thereof about 12 hours prior to administration of CAR cell therapies. In one embodiment, an anti-CD45 ADC is administered to the human patient in need thereof about 6 hours prior to administration of CAR cell therapies. In one embodiment, an anti-CD45 ADC is administered to the human patient in need thereof about 4 hours prior to administration of CAR cell therapies. In one embodiment, an anti-CD45 ADC is administered to the human patient in need thereof about 2 hours prior to administration of CAR cell therapies. In one embodiment, an anti-CD45 ADC is administered to the human patient in need thereof concomitantly with the administration of CAR cell therapies. In one embodiment, an anti-CD45 ADC is administered to the human patient in need thereof about 2 hours after administration of CAR cell therapies. In one embodiment, an anti-CD45 ADC is administered to the human patient in need thereof about 4 hours after administration of CAR cell therapies. In one embodiment, an anti-CD45 ADC is administered to the human patient in need thereof about 6 hours after administration of CAR cell therapies. In one embodiment, an anti-CD45 ADC is administered to the human patient in need thereof about 12 hours after administration of CAR cell therapies.

In one embodiment, the anti-CD45 ADC is administered before the CAR expressing immune cells are administered to the human patient in need thereof. In one embodiment, the anti-CD45 ADC is administered to the human patient in combination with CAR therapy, where the anti-CD45 ADC is administered to the human subject about 12 hours to about 21 days before administration of the CAR expressing immune cells. In one embodiment, the anti-CD45 ADC is administered to the human patient in combination with CAR therapy, where the anti-CD45 ADC is administered to the human subject about 18 hours to about 20 days before administration of the CAR expressing immune cells. In one embodiment, the anti-CD45 ADC is administered to the human patient in combination with CAR therapy, where the anti-CD45 ADC is administered to the human subject about 20 hours to about 18 days before administration of the CAR expressing immune cells. In one embodiment, the anti-CD45 ADC is administered to the human patient in combination with CAR therapy, where the anti-CD45 ADC is administered to the human subject about 1 day to about 15 days before administration of the CAR expressing immune cells. In one embodiment, the anti-CD45 ADC is administered to the human patient in combination with CAR therapy, where the anti-CD45 ADC is administered to the human subject about 1 day to about 10 days before administration of the CAR expressing immune cells. In one embodiment, the anti-CD45 ADC is administered to the human patient in combination with CAR therapy, where the anti-CD45 ADC is administered to the human subject about 2 days to about 8 days before administration of the CAR expressing immune cells. In one embodiment, the anti-CD45 ADC is administered to the human patient in combination with CAR therapy, where the anti-CD45 ADC is administered to the human subject about 3 days to about 6 days before administration of the CAR expressing immune cells.

In one embodiment, a lymphodepleting amount of the anti-CD45 ADC is administered. Overall levels of lymphocytes in a biological sample from a human patient can be tested following administration of an anti-CD45 ADC, wherein a decrease in the overall number of lymphocytes in a human patient following administration of the anti-CD45 ADC relative to the level prior to administration indicates efficacy of the anti-CD45 ADC for preventing rejection of the CAR cell therapy. In one embodiment, the level of endogenous lymphocytes in a biological sample from the human patient is reduced by at least about 5%, at least about 10%, at least about 15%, or at least about 20%, relative to the level of lymphocytes in a biological sample (of the same type, e.g., blood) from the human patient just prior to administration of the anti-CD45 ADC. In one embodiment, the level of endogenous lymphocytes in a biological sample from the human patient is reduced by about 5% to 25%, by about 5% to 20%, by about 5% to 15%, or by about 5% to 10%, relative to the level of lymphocytes in a biological sample (of the same type, e.g., blood) from the human patient just prior to administration of the anti-CD45 ADC. In one embodiment, the level of endogenous lymphocytes is determined one day or less prior to administration of the anti-CD45 ADC.

Levels of lymphocytes can be determined according to standard methods known in the art, including, but not limited, to fluorescence-activated cell sorting (FACs) analysis or a hematology analyzer.

Normal levels of neutrophils are needed to prevent infections. Neutropenia occurs when there are is an abnormally low level of neutrophils in the blood, leading to increased susceptibility to infection (see, e.g., Schwartzberg, Lee S. “Neutropenia: etiology and pathogenesis.” Clinical cornerstone 8 (2006): S5-S11, which is hereby incorporated by reference in its entirety). Neutropenia is often caused by chemotherapy treatments, adverse drug reactions, or autoimmune disorders. Methods of measuring the absolute neutrophil count (ANC) in the blood of a subject are known in the art (see, e.g., Amundsen, Erik K., et al. American journal of clinical pathology. 137.6 (2012): 862-869), which is hereby incorporated by reference in its entirety).

In one embodiment of the methods disclosed herein, the human subject does not develop neutropenia following administration of an immune cell expressing a CAR. In certain embodiments, neutropenia is defined as the human subject having an absolute neutrophil count (ANC) of less than about 1500 per microliter of blood (e.g., less than about 1500/μL, less than about 1400/μL, less than about 1300/μL, less than about 1200/μL, less than about 1100/μL, less than about 1000/μL, less than about 900/μL, less than about 800/μL, less than about 700/μL, or less than about 600/μL).

Severe neutropenia or agranulocytosis, clinically defined as an absolute neutrophil count of less than 500/μL of blood, can cause morbidity and mortality from infections. In one embodiment, the human subject does not develop severe neutropenia following administration an immune cell expressing a CAR. In certain embodiments of the methods disclosed herein, the severe neutropenia is defined as an ANC of less than about 500/μL of blood (e.g., less than about 500/μL, less than about 450/μL, less than about 400/μL, less than about 350/μL, less than about 300/μL, less than about 250/μL, less than about 200/μL, less than about 150/μL, or less than about 100/μL).

In one embodiment, administration of the ADC (e.g., at a lymphodepleting dose) is effective to increase the levels of one or more CAR-T engrafting cytokines (i.e., cytokines beneficial for CAR-T engraftment and associated with CAR-T expansion and efficacy) in the human subject, e.g., relative to a reference level. For example, in certain embodiments, administration of the ADC is effective to increase the levels of the one or more CAR-T engrafting cytokines in the human subject relative to the level of the one or more CAR-T engrafting cytokines in the human subject prior to administration of the ADC or relative to a pre-determined threshold level. In certain embodiments, the levels of the CAR-T engrafting cytokine are equivalent to the levels of the CAR-T engrafting cytokines in a patient treated with fludarabine/cyclophosphamide chemical conditioning (see, e.g., patient data disclosed in Kochehnderfer et al. Clin Oncol. 35: 1803-13). In certain embodiments, the CAR-T engrafting cytokine is IL-15 and/or IL-7 (see, e.g., Example 4).

In one embodiment, administration of an anti-CD45 ADC (e.g., at a lymphodepleting dose) does not substantially increase the level(s) of one or more cytokine release syndrome (CRS)-cytokines in the human subject, e.g., relative to a reference level. CRS and associated CRS-cytokines are described, for example, in Lee, Daniel W., et al. Blood. 124.2 (2014): 188-195. In certain embodiments, administration of an anti-CD45 ADC does not substantially increase the levels of the one or more CRS-cytokines in the human subject relative to, for example, the level of the one or more CRS-cytokines in the human subject prior to administration of the ADC or relative to a pre-determined threshold level. In certain embodiments, the CRS-cytokine is IFNγ, IL-10, IL-6, IL-8, MIP-1α, MIP-1β, or IL-10.

In some embodiments, the administration of the ADC (e.g., at a lymphodepleting dose) is effective to increase the levels of one or more CAR-T engrafting cytokines (i.e., cytokines beneficial for CAR-T engraftment and associated with CAR-T expansion and efficacy) in the human subject but does not increase the levels of one or more cytokine release syndrome (CRS)-cytokines in the human subject.

As described above, one of the advantages of the methods described herein is that lymphodepleting chemotherapeutic agents can be reduced in amount or not included in the conditioning regimen administered to a human patient having or planning on having CAR therapy. Lymphodepleting chemotherapeutic agents such as, but not limited to, fludarabine, cyclophosphamide, bendamustine, and/or pentostatin are commonly used as anti-rejection agents to promote CAR expressing cell acceptance in a human receiving CAR therapy. In certain embodiments, a human patient is administered an anti-CD45 ADC in combination with, e.g., prior to, administration of a CAR expressing immune cell (e.g., T cell) such that the human patient does not receive lymphodepleting chemotherapeutic agent, e.g., fludarabine and/or cyclophosphamide, prior to, concomitantly with, or following prior to, concomitantly with, or following administration of the CAR expressing immune cell.

In certain embodiments, an anti-CD45 ADC is used in combination with another therapy in order to promote tolerance of the CAR expressing immune cells. The use of other immune depleting agents can also be avoided or reduced through the use of an anti-CD45 ADC as an agent to deplete a human subject's endogenous immune cells and reduce the risk of rejection of the CAR expressing immune cells. For example, alemtuzumab is commonly used as an anti-rejection agent in combination with CAR therapy to promote CAR expressing cell acceptance in the human receiving CAR therapy. In certain embodiments, a human patient is administered an anti-CD45 ADC in combination with, e.g., prior to, administration of a CAR expressing immune cell (e.g., T cell) such that the human patient does not receive alemtuzumab prior to, concomitantly with, or following administration of the CAR expressing immune cell.

The methods disclosed herein can be used both for autologous and allogeneic cells expressing CARs. Importantly, the anti-CD45 ADC conditioning methods described herein are useful for expanding the type of immune cell that can be used in CAR therapy by providing a means by which tolerance of an allogeneic cell can be provided. In one embodiment, the CAR expressing immune cell is an allogeneic cell or an autologous cell. Examples of the types of immune cells that may be engineered to express a CAR include, but are not limited to, an allogeneic T cell, an autologous T cell, an autologous NK cell, or an allogeneic NK cell.

In one embodiment, the anti-CD45 antibody-drug conjugate is used to deplete CD45 expressing donor cells, e.g., lymphocytes expressing CD45, by administering the anti-CD45 antibody-drug conjugate after the administration of CAR cell therapies. In one embodiment, the CAR cell therapies comprise allogeneic cells.

The methods disclosed herein are particularly useful for the treatment of cancer or an autoimmune disease in a human subject having one of these disorders.

In one embodiment, the methods disclosed herein are used to treat cancer. More specifically, an anti-CD45 ADC is administered to a human subject having cancer in combination with CAR therapy. Examples of the types of cancer that can be treated using the methods disclosed herein include, but are not limited to, adult advanced cancer, pancreatic cancer, non-resectable pancreatic cancer, colorectal cancer, metastatic colorectal cancer, ovarian cancer, triple-negative breast cancer, hematopoietic/lymphoid cancer, colon cancer liver metastasis, small cell lung cancer, non-small cell lung cancer, B-cell lymphoma, relapsed or refractory B-cell lymphoma, follicular lymphoma, mantle cell lymphoma, diffuse large cell lymphoma, relapsed or refractory diffuse large cell lymphoma, anaplastic large cell lymphoma, primary mediastinal B-cell lymphoma, recurrent mediastinal, refractory mediastinal large B-cell lymphoma, large B-cell lymphoma, Hodgkin lymphoma, non-Hodgkin lymphoma, relapsed or refractory non-Hodgkin lymphoma, refractory aggressive non-Hodgkin lymphoma, B-cell non-Hodgkin lymphoma, refractory non-Hodgkin lymphoma, colorectal carcinoma, gastric carcinoma, pancreatic carcinoma, triple-negative invasive breast carcinoma, renal cell carcinoma, lung squamous cell carcinoma, hepatocellularcarcinoma, urothelial carcinoma, leukemia, B-cell leukemia, B-cell acute lymphocytic leukemia, B-cell actue lymphoblastic leukemia, adult acute lymphoblastic leukemia, B-cell prolymphocytic leukemia, childhood acute lymphoblastic leukemia, refractory childhood actue lymphblastic leukemia, acute leukemia, acute lymphoblastic leukemia, acute lymphocytic leukemia, prolymphocytic leukemia, chronic lymphocytic leukemia, acute myeloid leukemia, recurrent plasma cell myeloma, refractory plasma cell myeloma, multiple myeloma, relapsed or refractory multiple myeloma, multiple myeloma of bone, malignant glioma of brain, myelodysplastic syndrome, EGFR-positive colorectal cancer, glioblastoma multiforme, neoplasms, blastic plasmacytoid dendritic cell neoplasms, liver metastases, solid tumors, advanced solid tumors, mesothelin positive tumors, hematological malignancies, and other advanced malignancies.

In one embodiment, the methods disclosed herein are used to treat an autoimmune disease. More specifically, an anti-CD45 ADC is administered to a human subject having an autoimmune disease in combination with CAR therapy. Examples of autoimmune diseases that can be treated using the combination methods disclosed herein include, but are not limited to, multiple sclerosis, Crohn's disease, ulcerative colitis, rheumatoid arthritis, type 1 diabetes, lupus, and psoriasis.

In certain embodiments, an anti-CD45 ADC is administered to a human patient in combination with a CAR-T cell therapy. In one embodiment, the anti-CD45 ADC is administered to the human patient prior to administration of the CAR-T therapy. Examples of CAR-T cells that could be used in combination with the anti-CD45 ADC therapy described herein include, but are not limited to, CD19 CAR-T (e.g., CART-19-01,02,03 (Fujian Medical University); daopeicart (Hebei Senlang Biotechnology Inc.); IM19CART/001, YMCART201702 (Beijing Immunochina Medical Science & Technology Co.); CART-CD19-02,03 (Wuhan Sian Medical Technology Co.); Universal CD19-CART/SHBYCL001,002 (Shanghai Bioray Laboratory Inc.); UnicarTherapy201701 (Shanghai Unicar-Therapy Biomedicine Technology Co.); Genechem/NCT02672501 (Shanghai GeneChem Co.); SenL_19 (Hebei Senlang Biotechnology Inc.); PCAR-019 (PersonGen BioTherapeutics (Suzhou); ICAR19 (Immune Cell, Inc.); WM-CART-02 (Sinobioway Cell Therapy Co.); HenanCH080,109,152 (Henan Cancer Hospital/The Pregene (ShenZhen) Biotechnology Co.); IM19-CD28 and IM19-41BB CAR-T cells (Beijing Immunochina Medical Science & Technology Company); CTL019/IT1601-CART19 (Beijing Sanwater Biological Technology Co.); CTL019/CCTL019C2201 (Novartis Pharmaceuticals); CD19:4-1BB:CD28:CD3/FirstShenzhen01 (Shenzhen Second People's Hospital/The Beijing Pregene Science and Technology Company); MB-CART19.1 (Shanghai Children's Medical Center/Miltenyi Biotec GmbH); PZ01 CAR-T cells (Pinze Lifetechnology Co.); YMCART201701 (Beijing Immunochina Medical Science & Technology Co.); 2016YJZ12 (Peking University/Marino Biotechnology Co.); EGFRt/19-28z/4-1BBL CAR T cells (Memorial Sloan Kettering Cancer Center/Juno Therapeutics, Inc.); Doing-002 (Beijing Doing Biomedical Co.); PCAR-019 (PersonGen BioTherapeutics (Suzhou) Co.); C-CAR011 (Peking Union Medical College Hospital/Cellular Biomedicine Group Ltd.); iPD1 CD19 eCAR T cells (Peking University/Marino Biotechnology Co.); 2013-1018/NCT02529813 (M.D. Anderson Cancer Center/Ziopharm/Intrexon Corp.); HenanCH CAR 2-1 (Henan Cancer Hospital/The Pregene (ShenZhen) Biotechnology Co.); JCAR015 (Juno Therapeutics, Inc.); JCAR017/017001,004,006 (Juno Therapeutics, Inc.); JCAR017 (Celgene); TBI-1501 (Takara Bio Inc.); JMU-CD19CAR (Jichi Medical University); KTE-C19 (Kite, A Gilead Company); TriCAR-T-CD19 (Timmune Biotech Inc.); PF-05175157 (Fred Hutchinson Cancer Research Center)); CD22/CD30/CD7/BCMA/CD123 (e.g., 2016040/NCT03121625 (Hebei Senlang Biotechnology Inc.)); CD22 (e.g., Ruijin-CAR-01 (Ruijin Hospital/Shanghai Unicar-Therapy Bio-medicine Technology Co.); AUTO-PA1,DB1 (Autolus Limited)), CD20 (e.g., Doing-006 (Beijing Doing Biomedical Co.)); or CD20/CD22/CD30 (e.g., SZ5601 (The First Affiliated Hospital of Soochow University Shanghai/Unicar-Therapy Bio-medicine Technology Co.)).

### CAR Construct

The present disclosure includes the use of CAR therapy in combination with an anti-CD45 immune suppressing ADC. The present disclosure is not generally limited to a specific CAR construct, e.g., a specific antigen binding region or intracellular signaling domain, as the present disclosure is based on the discovery that anti-CD45 ADCs can serve as a conditioning agent for CAR therapy by promoting acceptance of CAR expressing cells by ablating endogenous CD45+ immune cells, such as endogenous lymphocytes. Specific CARs, e.g., CD19 specific CARs, are contemplated herein and are included in the methods disclosed herein, but are not meant to be limiting.

CAR constructs are known in the art and generally contain (a) an extracellular region comprising an antigen binding domain, (b) a transmembrane domain and (c) a cytoplasmic signaling domain. Exemplary CAR configurations are known in the art, and any suitable configuration can be used in the methods described herein. For example, the CAR may be a first generation, a second generation, or a third generation CAR, e.g., as described in Guedan et al. Molecular Therapy-Methods & Clinical Development. 12: 145-156 (2019) or Sadelain et al. Cancer discovery 3.4: 388-398 (2013), the entire contents of which are hereby incorporated by reference. Briefly, a “first generation” CAR can comprise an (a) extracellular antigen binding domain, (b) a transmembrane domain, (c) one or more intracellular signaling domains, and optionally (d) a hinge region connecting the antigen binding domain to the transmembrane domain. A “second generation” CAR can comprise elements (a), (b), (c), and optionally (d), and further includes a co-stimulatory domain, for example, a co-stimulatory domain of CD28 or 4-1BB. A “third generation” CAR can comprise elements (a), (b), (c), and optionally (d), and further includes multiple co-stimulatory domains, for example, the co-stimulatory domains of CD28 and 4-1BB, or the co-stimulatory domains of CD28 and OX40. Each of the foregoing elements is described in detail below. It should be appreciated that in some embodiments, CAR molecules described by the following exemplary, non-limiting arrangements are from left to right, N-terminus to C-terminus of the CAR. A CAR as described by the disclosure may comprise or further comprise any other combination of elements as described herein. Other exemplary chimeric antigen receptor constructs are disclosed in U.S. Pat. Nos. 9,328,156; 9,783,591; 9,714,278; 9,765,156; 10,117,896; 9,573,988; 10,308,717; 10,221,245; 10,040,865; U.S. Patent Publication No. 2018/0256712A1; U.S. Patent Publication No. 2018/0271907A1; U.S. Patent Publication No. 2016/0046724A1; U.S. Patent Publication No. 2018/0044424A1; U.S. Patent Publication No. 2018/0258149A1; U.S. Patent Publication No. 2019/0151363A1; and U.S. Patent Publication No. 2018/0273601A1; the contents of each of the foregoing patents and patent publications are incorporated by reference herein in their entirety.

The CAR used in the methods disclosed herein includes an extracellular antigen binding domain. The extracellular antigen binding domain can be any molecule that binds to an antigen, including, but not limited to, a human antibody, a humanized antibody, or any a functional fragment thereof. In certain embodiments, the antigen binding domain is an scFv. In other embodiments, the extracellular antigen binding domain is a non-immunoglobulin scaffold protein. In other embodiments, the extracellular binding domain of the CAR comprises a single chain T cell receptor (scTCR). As described in U.S. Pat. Nos. 5,359,046, 5,686,281 and 6,103,521, the extracellular domain may also be obtained from any of the wide variety of extracellular domains or secreted proteins associated with ligand binding and/or signal transduction.

The choice of the molecular target (antigen) of the extracellular binding domain depends upon the type and number of ligands that define the surface of a target cell. For example, the antigen binding domain may be chosen to recognize a ligand that acts as a cell surface marker on target cells associated with a particular disease state. Thus, in one aspect, the CAR-mediated immune cell (e.g., T-cell) response can be directed to an antigen of interest by way of engineering an extracellular antigen binding domain that specifically binds a desired antigen into a CAR. For example, the antigen binding domain may be chosen to recognize a ligand that acts as a cell surface marker on target cells associated with a particular disease state, such as cancer or an autoimmune disease. Thus examples of cell surface markers that may act as ligands for the antigen binding domain in a CAR include those associated with cancer cells and other forms of diseased cells, for example, autoimmune disease cells and pathogen infected cells. In some embodiments, a CAR is engineered to target a tumor antigen of interest by way of engineering a desired antigen binding domain that specifically binds to an antigen on a tumor cell. In the context of the present disclosure, “tumor antigen” refers to antigens that are common to specific hyperproliferative disorders such as cancer. In one embodiment, the antigen is a tumor antigen, examples of which include, but are not limited to, CD19, CD22, CD30, CD7, BCMA, CD137, CD22, CD20, AFP, GPC3, MUC1, mesothelin, CD38, PD1, EGFR (e.g., EGFRvIII), MG7, BCMA, TACI, CEA, PSCA, CEA, HER2, MUC1, CD33, ROR2, NKR-2, PSCA, CD28, TAA, NKG2D, or CD123. In one embodiment, CAR comprises an scFv that binds to CD19, CD22, CD30, CD7, BCMA, CD137, CD22, CD20, AFP, GPC3, MUC1, mesothelin, CD38, PD1, EGFR (e.g., EGFRvIII), MG7, BCMA, TACI, CEA, PSCA, CEA, HER2, MUC1, CD33, ROR2, NKR-2, PSCA, CD28, TAA, NKG2D, or CD123.

In one embodiment, the CAR binds to BCMA, as described in US Patent Application Publication No. 20190388528 (Bluebird Bio), the contents of which relating to CARs are incorporated by reference herein.

In another aspect, the extracellular binding domain of the CAR binds to an antigen that is AFP (e.g., ETCH17AFPCAR01 (Aeon Therapeutics (Shanghai) Co./Eureka Therapeutics Inc.)), GPC3 (e.g., GeneChem GPC-3 CART (Shanghai GeneChem Co.); 302 GPC3-CART (Shanghai GeneChem Co.); CAR-T for liver cancer (Shanghai GeneChem Co.); CAR-GPC3 T cells (Carsgen Therapeutics)), MUC1 (e.g., PG-021-001,002 (PersonGen BioTherapeutics (Suzhou) Co.)), mesothelin (e.g., H2017-01-P01 (Ningbo Cancer Hospital); TAI-meso-CART (Shanghai GeneChem Co.); K16-4/NCT02930993 (China Meitan General Hospital/Marino Biotechnology Co.)), CD38 (e.g., Anti-CD38 A2 CAR-T/SOR-CART-MM-001 (Sorrento Therapeutics, Inc.)), herinCAR-PD1 (e.g., herinCAR-PD1/NBWYKY2016-06-001,002,003 (Ningbo Cancer Hospital); SIMC-20160101,02,03 (Shanghai International Medical Center)), BCMA (e.g., P-BCMA-101 autologous T stem cell memory (Tscm) CAR-T cells/P-BCMA-101-001 (Poseida Therapeutics, Inc.); HenanCH284 (Henan Cancer Hospital/The Pregene (ShenZhen) Biotechnology Company); LCAR-B38M CAR-T cells (Nanjing Legend Biotech Co.); 9762/NCT03338972 (Fred Hutchinson Cancer Research Center/Juno Therapeutics, Inc.); Descartes-08 (Cartesian Therapeutics); KITE-585 (Kite, A Gilead Company); bb21217 (bluebird bio); bb21217 (Celgene); JCARH125 (Juno Therapeutics, Inc.)), CD30 (e.g., ICAR30 T cells (Immune Cell, Inc.)), EGFR (e.g., EGFR:4-1BB:CD28:CD3 modified T cells/First Shenzhen02 (Shenzhen Sceond People's Hospital/The Beijing Pregene Science and Technology Company); EGFR-IL12-CART (Shenzhen Second People's Hospital/The Pregene (ShenZhen) Biotechnology Co.); SBNK-2016-015-01 (Beijing Sanbo Brain Hospital/Marino Biotechnology Co.)), MG7 (e.g., MG7-CART (Xijing Hospital/Shanghai GeneChem Co.)), BCMA/TACI (e.g., AUTO2-MM1 (Autolus Limited)), CEA (e.g., 383-74/NCT02416466 (Roger Williams Medical Center/Sirtex Medical)), mesothelin/PSCA/CEA/HER2/MUC1/EGFRvIII (e.g., NCT03267173 (First Affiliated Hospital of Harbin Medical University/Shanghai Unicar-Therapy Bio-medicine Technology Co.)), CD20 (e.g., EY201605-19 (Beijing Biohealthcare Biotechnology Co.)), CD33 (e.g., 2016-0341/NCT03126864 (M.D. Anderson Cancer Center/Intrexon Corp./Ziopharm)), EGFR/BCMA (e.g., EGFRt/BCMA-41BBz CAR T cell (Memorial Sloan Kettering Cancer Center/Juno Therapeutics, Inc.)), ROR2 (e.g., autologous CCT301-38 or CCT301-59 T cells (Shanghai Sinobioway Sunterra Biotech)), NKR-2 (e.g., CYAD-N2T-002,003,004 (Celyad)), PSCA (e.g., BP-012 (Bellicum Pharmaceuticals)), CD28 (e.g., autologous CSR T cells (Beijing Sanbo Brain Hospital/Marino Biotechnology Co.)), TAA (e.g., AMG 119 (Amgen)), NKG2D (e.g., CM-CS1 (Celyad)), or CD123 (e.g., UCART123 (Cellectis S.A.)). The foregoing sentence further provides examples of CARs that bind said antigens (e.g., AMG 119 (Amgen)). These CAR constructs may be used in the conditioning methods disclosed herein with an anti-CD45 ADC.

A CAR construct further contains a transmembrane domain that connects (either literally or by general proximity, e.g., with spacers) the extracellular antigen binding domain and the cytoplasmic signaling domain. Generally, a CAR may comprise an scFv, Fab or other antibody moiety, generally with a hinge or other linker between the scFv (or extracellular antigen binding domain) and a transmembrane domain. The transmembrane domain will be attached to an intracellular signaling domain, such as CD28 or CD3-ζ and typically will include one or more co-stimulatory domains as discussed below. Often, a spacer or hinge is introduced between the extracellular antigen binding domain and the transmembrane domain to provide flexibility which allows the antigen-binding domain to orient in different directions to facilitate antigen recognition and binding.

Thus, in certain embodiments, the CAR can further comprise a hinge region. The hinge region can be derived from the hinge region of IgG1, IgG2, IgG3, IgG4, IgA, IgD, IgE, IgM, CD28, or CD8 alpha. In one particular embodiment, the hinge region is derived from the hinge region of IgG4. In another embodiment, the hinge of a CAR between the extracellular binding domain and the transmembrane domain is a CD8 hinge domain (see SwissProt/GenBank Acc. No. P01732).

In one embodiment, a CAR comprises an extracellular antigen binding domain and a transmembrane domain connected via a CD8 hinge: AKPTTTPAPR PPTPAPTIAS QPLSLRPEAC RPAAGGAVHT RGLDFA (SEQ ID NO: 9).

In one embodiment, a CAR comprises an extracellular antigen binding domain and a transmembrane domain connected via a hybrid CD8-CD28 hinge: AKPTTTPAPR PPTPAPTIAS QPLSLRPEAC RPAAGGAVHT RGLDFAPRKI EVMYPPPYLD NEKSNGTIIH VKGKHLCPSP LFPGPSKP (SEQ ID NO: 10).

The transmembrane domain may be contributed by a protein contributing the extracellular antigen binding domain, a protein contributing the effector function signaling domain, a protein contributing the proliferation signaling portion, or by a totally different protein. For the most part it will be convenient to have the transmembrane domain naturally associated with one of the other domains of a CAR. In one embodiment, the transmembrane and cytoplasmic domains used would be contiguous portions of the CD28 sequence. Thus, any transmembrane domain is contemplated for use herein as long as the domain is capable of anchoring a CAR comprising the antigen binding domain to a cell membrane.

The transmembrane domain may be derived either from a natural or from a synthetic source. Where the source is natural, the domain may be derived from any membrane-bound or transmembrane protein. Transmembrane domains of particular use in this disclosure may be derived from (e.g., comprise at least the transmembrane domain(s) of) the alpha, beta or zeta chain of the T-cell receptor, CD28, CD3 epsilon, CD45, CD4, CD2, CD8, CD9, CD16, CD22, CD33, CD37, CD64, CD80, CD86, CD134, CD137, CD154, LFA-1 T-cell co-receptor, CD2 T-cell co-receptor/adhesion molecule, CD8 alpha, and fragments thereof. Transmembrane domains can be identified using any method known in the art or described herein, e.g., by using the UniProt Database.

In some embodiments, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of a CAR. A glycine-serine doublet provides a particularly suitable linker.

In some embodiments, the transmembrane domain in the CAR of the disclosure is the CD8 transmembrane domain. Sequences of CD8 for this purposes are taught in PCT pub no. W02014/055771.

In some embodiments, the transmembrane domain in the CAR is the CD8 transmembrane domain, or a functional portion thereof. For example, a CAR can comprise a CD3 transmembrane domain having an amino acid sequence of LDPKLCYLLD GILFIYGVIL TALFLRVK (SEQ ID NO: 11), or a functional portion thereof, such as LCYLLDGILF IYGVILTALF L (SEQ ID NO: 12).

In some embodiments, the transmembrane domain in the CAR of the disclosure is a CD28 transmembrane domain. An exemplary sequence of CD28 is provided below, as well as an exemplary transmembrane domain sequence. In some embodiments, the CD28 transmembrane domain comprises the exemplary transmembrane domain sequence below, or a fragment or variant thereof that is capable of anchoring a CAR comprising the sequence to a cell membrane. Thus, in some embodiments, the transmembrane domain of the CAR is a CD28 transmembrane domain containing the following amino acid sequence: FWVLVVVGGVLACYSLLVTVAFIIFWV (SEQ ID NO: 13). In one embodiment, the transmembrane domain of the CAR is a CD28 transmembrane domain containing the following amino acid sequence: IEVMYPPPYL DNEKSNGTII HVKGKHLCPS PLFPGPSKPF WVLVVVGGVL ACYSLLVTVA FIIFWV (SEQ ID NO: 14), or a functional fragment thereof, e.g., SEQ ID NO: 13.

In addition to an extracellular antigen binding domain and a transmembrane domain, a CAR further comprises an intracellular (or cytoplasmic) signaling domain.

It is known that signals generated through the endogenous TCR alone are insufficient for full activation of the T cell and that a secondary or co-stimulatory signal may also be required. Thus, T cell activation can be mediated by two distinct classes of cytoplasmic signaling sequences: those that initiate antigen-dependent primary activation through the TCR (primary cytoplasmic signaling sequences) and those that act in an antigen-independent manner to provide a secondary or co-stimulatory signal (secondary cytoplasmic signaling sequences).

An “intracellular signaling domain” or “cytoplasmic signaling domain” as the terms are used herein, refers to an intracellular portion of a molecule. The intracellular signaling domain can generate a signal that promotes an immune effector function of the CAR containing immune cell, e.g., a CAR-T cell or CAR-expressing NK cell. Examples of immune effector function, e.g., in a CART cell or CAR-expressing NK cell, include cytolytic activity and helper activity, including the secretion of cytokines. In embodiments, the intracellular signal domain transduces the effector function signal and directs the cell to perform a specialized function. While the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

In one embodiment, the intracellular signaling domain of the CAR contains a CD3 zeta signaling region as described in SEQ ID NO: 15, or a signaling portion thereof.

(SEQ ID NO: 15)
RVKFSRSADAPAYQQGQNQLYNELNLGRREEYDVLDKRRGRDPEMGGK
PRRKNPQEGLYNELQKDKMAEAYSEIGMKGERRRGKGHDGLYQGLSTA
TKDTYDALHMQALPPR

Cytoplasmic signaling domains further can include, but are not limited to, those derived from CD3 zeta, FcR gamma, FcR beta, CD3 gamma, CD3 delta, CD3 epsilon, CDS, CD22, CD79a, CD79b, CD278 (“ICOS”), Fc.epsilon.RI, CD66d, DAP10, and DAP12.

A CAR may further contain an “intracellular costimulatory domain” which is a polypeptide chain derived from an intracellular signaling domain of a costimulatory protein or proteins, such as CD28 and 4-1BB, that enhance cytokine production.

Exemplary co-stimulatory signaling regions include 4-1BB, CD21, CD28, CD27, CD127, ICOS, IL-15Ra, and OX40.

In certain embodiments, the cytoplasmic costimulatory domain of a CAR comprises the 4-1BB signaling domain by itself or combined with any other desired cytoplasmic domain(s) useful in the context of a CAR. 4-1BB is a member of the TNFR superfamily with an amino acid sequence provided as GenBank Acc. No. AAA62478.2, or the equivalent residues from a non-human species, e.g., mouse, rodent, monkey, ape and the like; and a “4-1BB costimulatory domain” is defined as amino acid residues 214-255 of GenBank acc no. AAA62478.2, or the equivalent residues from a non-human species, e.g., mouse, rodent, monkey, ape and the like.

In one embodiment, the intracellular costimulatory signaling domain of the CAR is 4-1BB (CD137) co-stimulatory signaling region, or a signaling portion thereof:

(SEQ ID NO: 16)
KRGRKKLLYIFKQPFMRPVQTTQEEDGCSCRFPEEEEGGCEL

In one embodiment, the costimulatory signaling domain of the CAR is the CD28 co-stimulatory signaling region sequence of the CAR is the following:

Intracellular domain: CD28 co-stimulatory signaling region, or a signaling portion thereof:

(SEQ ID NO: 17)
RSKRSRLLHSDYMNMTPRRPGPTRKHYQPYAPPRDFAAYRS

Thus, the cytoplasmic domain of the CAR contains a CD3-zeta signaling domain combined with any other desired cytoplasmic domain(s) useful in the context of the CAR of the disclosure. In certain embodiments, the cytoplasmic domain of the CAR can comprise a CD3 zeta domain and a costimulatory signaling region, including, but not limited to, 4-1BB, CD28, and CD27.

The cytoplasmic signaling sequences within the cytoplasmic signaling portion of the CAR of the disclosure may be linked to each other in a random or specified order. Optionally, a short oligo- or polypeptide linker or spacer, preferably between 5 and 20 amino acids in length may be inserted between cytoplasmic domains. A GGGGS (SEQ ID NO: 18) or (GGGGS)×3 (SEQ ID NO: 19) provides a particularly suitable linker.

In one embodiment, a CAR used herein includes an extracellular domain containing a single chain variable domain of an anti-CD19 monoclonal antibody, a transmembrane domain containing a hinge and transmembrane domain of CD8α, and a cytoplasmic domain containing the signaling domain of CD3ζ and the signaling domain of 4-1BB. An exemplary CAR includes an extracellular domain include the anti-CD19 monoclonal antibody which is described in Nicholson I C, et al., Mol Immunol 34:1157-1165 (1997) plus the 21 amino acid signal peptide of CD8α (translated from 63 nucleotides at positions 26-88 of GenBank Accession No. NM_001768). The CD8α hinge and transmembrane domain consists of 69 amino acids translated from the 207 nucleotides at positions 815-1021 of GenBank Accession No. NM_001768. The CD3ζ signaling domain of the preferred embodiment contains 112 amino acids translated from 339 nucleotides at positions 1022-1360 of GenBank Accession No. NM_000734.

Between the extracellular domain (comprising the antigen binding domain) and the transmembrane domain of the CAR, or between the cytoplasmic domain and the transmembrane domain of the CAR, there may be incorporated a spacer or hinge domain. As used herein, the term “spacer domain” generally means any oligo- or polypeptide that functions to link the transmembrane domain to the extracellular domain and/or the cytoplasmic domain in the polypeptide chain. As used herein, a hinge domain generally means any oligo- or polypeptide that functions to provide flexibility to the CAR, or domains thereof, and/or prevent steric hindrance of the CAR, or domains thereof. In some embodiments, a spacer or hinge domain may comprise up to 300 amino acids, preferably 10 to 100 amino acids and most preferably 5 to 20 amino acids. It also should be appreciated that one or more spacer domains may be included in other regions of a CAR, as aspects of the disclosure are not limited in this respect.

It is to be understood that a CAR can include a region (e.g., an antigen binding domain, a transmembrane domain, a cytoplasmic domain, a signaling domain, a safety domain, and/or a linker, or any combination thereof) having a sequence provided herein or a variant thereof or a fragment of either one thereof (e.g., a variant and/or fragment that retains the function required for the CAR activity) can be included in a CAR protein as described herein. In some embodiments, a variant has 1, 2, 3, 4, 5, 6, 7, 8, 9, or 10 amino acid changes relative to the illustrated sequence. In some embodiments, a variant has a sequence that is at least 80%, at least 85%, at least 90%, 90%-95%, at least 95% or at least 99% identical to the illustrated sequence. In some embodiments, a fragment is 1-5, 5-10, 10-20, 20-30, 30-40, or 40-50 amino acids shorter than a sequence provided herein. In some embodiments, a fragment is shorter at the N-terminal, C-terminal, or both terminal regions of the sequence provided. In some embodiments, a fragment contains 80%-85%, 85%-90%, 90%-95%, or 95%-99% of the number of amino acids in a sequence provided herein.

In other embodiments, the present disclosure comprises nucleic acid sequences that encode for the amino acid sequences disclosed herein.

In some embodiments, the above exemplary, non-limiting arrangements are from left to right, N-terminus to C-terminus of the CAR. The CAR may comprise or further comprise any other combination of elements as described herein.

Once the CAR construct is identified with its various parts, a CAR expressing immune cell is produced whereby the immune cell expresses the CAR. The method includes introducing into, e.g., transducing, the immune cell with a nucleic acid molecule described herein (e.g., an RNA molecule, e.g., an mRNA), or a vector comprising a nucleic acid molecule encoding a CAR, e.g., a CAR described herein. The present disclosure also provides a method of generating a population of cells (e.g., RNA-engineered cells transiently expressing an exogenous RNA). The method includes introducing into the cell an RNA as described herein (e.g., an in vitro transcribed RNA or synthetic RNA; an mRNA sequence encoding a CAR polypeptide as described herein). In embodiments, the RNA expresses the CAR polypeptide transiently. In one embodiment, the cell is a cell as described herein, e.g., an immune effector cell (e.g., T cells or NK cells, or cell population).

CAR expressing-immune cells can be administered as a dose based on cells per kilogram (cells/kg) of body weight of the subject to which the cells are administered. For example, in some embodiments, the subject is administered about 1×10⁶ to about 1×10⁸ cells/kg (e.g., about 1×10⁶ to about 2×10⁶, about 2×10⁶ to about 3×10⁶ about, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 9×10⁶, about 9×10⁶ to about 1×10⁷, about 1×10⁷ to about 2×10⁷, about 2×10⁷ to about 3×10⁷, about 3×10⁷ to about 4×10⁷, about 4×10⁷ to about 5×10⁷, about 5×10⁷ to about 6×10⁷, about 6×10⁷ to about 76×10⁷, about 8×10⁷ to about 9×10⁷, about 9×10⁷ to about 1×10⁸, about 1×10⁶, about 1×10⁷, or about 1×10⁸ cells/kg). In one embodiment, the subject is administered about 1×10⁶ to about 2×10⁶ cells/kg of engineered CART cells (e.g., about 1×10⁶, about 1.1×10⁶, about 1.2×10⁶, about 1.3×10⁶, about 1.4×10⁶, about 1.5×10⁶, about 1.6×10⁶, about 1.7×10⁶, about 1.8×10⁶, about 1.9×10⁶, or about 2×10⁶ cells/kg).

In some embodiments, a CAR expressing-immune cell dose is in the range of about 10⁴ to about 10¹⁰ cells/kg of body weight, for example, about 10⁵ to about 10⁹, about 10⁵ to about 10⁸, about 10⁵ to about 10⁷, or about 10⁵ to 10⁶, depending on the mode and location of administration. In general, in the case of systemic administration, a higher dose is used than in regional administration, where the immune cells of the invention are administered in the region of a tumor. Exemplary dose ranges include, but are not limited to, 1×10⁴ to 1×10⁸, 2×10⁴ to 1×10⁸, 3×10⁴ to 1×10⁸, 4×10⁴ to 1×10⁸, 5×10⁴ to 1×10⁸, 6×10⁴, to 1×10⁸, 7×10⁴ to 1×10⁸, 8×10⁴ to 1×10⁸, 9×10⁴ to 1×10⁸, 1×10⁵ to 1×10⁸, for example, 1×10⁵ to 9×10⁷, 1×10⁵ to 8×10⁷, 1×10⁵ to 7×10⁷, 1×10⁵ to 6×10⁷, 1×10⁵ to 5×10⁷, 1×10⁵ to 4×10⁷, 1×10⁵ to 3×10⁷, 1×10⁵ to 2×10⁷, 1×10⁵ to 1×10⁷, 1×10⁵ to 9×10⁶, 1×10⁵ to 8×10⁶, 1×10⁵ to 7×10⁶, 1×10⁵ to 6×10⁶, 1×10⁵ to 5×10⁶, 1×10⁵ to 4×10⁶, 1×10⁵ to 3×10⁶, 1×10⁵ to 2×10⁶, 1×10⁵ to 1×10⁶, 2×10⁵ to 9×10⁷, 2×10⁵ to 8×10⁷, 2×10⁵ to 7×10⁷, 2×10⁵ to 6×10⁷, 2×10⁵ to 5×10⁷, 2×10⁵ to 4×10⁷, 2×10⁵ to 3×10⁷, 2×10⁵ to 2×10⁷, 2×10⁵ to 1×10⁷, 2×10⁵ to 9×10⁶, 2×10⁵ to 8×10⁶, 2×10⁵ to 7×10⁶, 2×10⁵ to 6×10⁶, 2×10⁵ to 5×10⁶, 2×10⁵ to 4×10⁶, 3×10⁵ to 3×10⁶ cells/kg, and the like. Exemplary dose ranges also can include, but are not limited to, 5×10⁵ to 1×10⁸, for example, 6×10⁵ to 1×10⁸, 7×10⁵ to 1×10⁸, 8×10⁵ to 1×10⁸, 9×10⁵ to 1×10⁸, 1×10⁶ to 1×10⁸, 1×10⁶ to 9×10⁷, 1×10⁶ to 8×10⁷, 1×10⁶ to 7×10⁷, 1×10⁶ to 6×10⁷, 1×10⁶ to 5×10⁷, 1×10⁶ to 4×10⁷, 1×10⁶ to 3×10⁷ cells/kg, and the like. Exemplary cell doses include, but are not limited to, a dose of about 1×10⁴, about 2×10⁴, about 3×10⁴, about 4×10⁴, about 5×10⁴, about 6×10⁴, about 7×10⁴, about 8×10⁴, about 9×10⁴, about 1×10⁵, about 2×10⁵, about 3×10⁵, about 4×10⁵, about 5×10⁵, about 6×10⁵, about 7×10⁵, about 8×10⁵, about 9×10⁵, about 1×10⁶, about 2×10⁶, about 3×10⁶, about 4×10⁶, about 5×10⁶, about 6×10⁶, about 7×10⁶, about 8×10⁶, about 9×10⁷, about 1×10⁷, about 2×10⁷, about 3×10⁷, about 4×10⁷, about 5×10⁷, about 6×10⁷, about 7×10⁷, about 8×10⁷, about 9×10⁷, about 1×10⁸, about 2×10⁸, about 3×10⁸, about 4×10⁸, about 5×10⁸, about 6×10⁸, about 7×10⁸, about 8×10⁸, about 9×10⁸, about 1×10⁹ and so forth in the range of about 10⁴ to about 10¹⁰ cells/kg.

In some embodiments, the dose of CAR expressing-immune cells, e.g., CAR-T cells, is a non-weight based determination and is instead based on the total number of cells administered. For example, in some embodiments, the subject is administered a total dose of about 1×10⁷ to about 9×10⁸ cells (e.g., about 1×10⁷ to about 9×10⁸, about 1×10⁷ to about 8×10⁸, about 1×10⁷ to about 7×10⁸, about 1×10⁷ to about 6×10⁸, about 1×10⁷ to about 5×10⁸, about 1×10⁷ to about 4×10⁸, about 1×10⁷ to about 3×10⁸, about 1×10⁷ to about 2×10⁸, about 1×10⁷ to about 1×10⁸, about 2×10⁷ to about 9×10⁸, 3×10⁷ to about 8×10⁸, about 4×10⁷ to about 7×10⁸, about 5×10⁷ to about 6×10⁸, about 6×10⁷ to about 6×10⁸ cells). In some embodiments, the subject is administered a total dose of about 9×10⁸ cells or less (e.g., about 9×10⁸ or less, about 8×10⁸ or less, about 7×10⁸ or less, about 6×10⁸ or less, about 5×10⁸ or less, about 4×10⁸ or less, about 3×10⁸ or less, about 2×10⁸ or less, about 1×10⁸ or less, about 9×10⁷ or less, about 8×10⁷ or less, about 7×10⁷ or less, about 6×10⁷ or less, about 5×10⁷ or less, about 4×10⁷ or less, about 3×10⁷ or less, about 2×10⁷ or less, or about 1×10⁷ or less cells).

In one embodiment, the CAR expressing-immune cell is axicabtagene ciloleucel, a CD19-directed genetically modified autologous T cell immunotherapy. Accordingly, in some embodiments, the subject is pre-treated with a lymphodepleting dose of an anti-CD45 antibody drug conjugate (ADC), wherein the anti-CD45 ADC comprises an anti-CD45 antibody, or antigen-binding fragment thereof, conjugated to a cytotoxin via a linker before administration (e.g., by infusion) of a therapeutically effective amount of axicabtagene ciloleucel. In one embodiment, the subject is pre-medicated with acetaminophen (e.g., 650 mg PO) and an H1-antihistamine (e.g., diphenhydramine 12.5 mg intravenously or PO) approximately 1 hour before administration of axicabtagene ciloleucel. In certain embodiments, the subject is not administered systemic corticosteroids.

In some embodiments, the dosing of axicabtagene ciloleucel is based on the number of chimeric antigen receptor (CAR)-positive viable T cells. In certain embodiments, the subject is administered a dose of axicabtagene ciloleucel comprising 2×10⁶ CAR-positive viable T cells per kg body weight, with a maximum of 2×10⁸ CAR-positive viable T cells

In some embodiments, the subject administered axicabtagene ciloleucel is an adult subject with relapsed or refractory large B-cell lymphoma. In certain embodiments, the B-cell lymphoma is diffuse large B-cell lymphoma (DLBCL) not otherwise specified, primary mediastinal large B-cell lymphoma, high grade B-cell lymphoma, and DLBCL arising from follicular lymphoma. In some embodiments, the subject has previously received two or more lines of systemic therapy. In some embodiments, the subject does not have primary central nervous system lymphoma.

In another embodiment, the human subject is not administered a lymphodepleting chemotherapeutic agent, such as fludarabine or cyclophosphamide, prior to administration of axicabtagene ciloleucel.

In one embodiment, the CAR expressing-immune cell is tisagenlecleucel, a CD19-directed genetically modified autologous T cell immunotherapy. Accordingly, in some embodiments, the subject is pre-treated with a lymphodepleting dose of an anti-CD45 antibody drug conjugate (ADC), wherein the anti-CD45 ADC comprises an anti-CD45 antibody, or antigen-binding fragment thereof, conjugated to a cytotoxin via a linker before administration (e.g., by infusion) of a therapeutically effective amount of tisagenlecleucel. In one embodiment, the subject is pre-medicated with acetaminophen and an H1-antihistamine (e.g., diphenhydramine) approximately 30 minutes to 60 minutes before administration of tisagenlecleucel. In certain embodiments, the subject is not administered systemic corticosteroids.

In some embodiments, the dosing of tisagenlecleucel is based on the number of chimeric antigen receptor (CAR)-positive viable T cells. In certain embodiments, the subject has pediatric or young adult B-cell ALL and is up to 25 years in age. In some such embodiments, the subject having pediatric or young adult B-cell ALL is administered a dose of tisagenlecleucel comprising (i) 0.2 to 5.0×10⁶ CAR-positive viable T cells per kg body weight intravenously if the subject is 50 kg or less in weight, or (ii) 0.1 to 2.5×10⁸ total CAR positive viable T cells (non-weight based) intravenously if the patient is above 50 kg in weight.

In some embodiments, the subject administered tisagenlecleucel is an adult subject with relapsed or refractory large B-cell lymphoma. In some embodiments, a subject having adult relapsed or refractory diffuse large B-cell lymphoma is administered a dose of tisagenlecleucel comprising 0.6 to 6.0×10⁸ CAR-positive viable T cells intravenously. In certain embodiments, the B-cell lymphoma is diffuse large B-cell lymphoma (DLBCL) not otherwise specified, primary mediastinal large B-cell lymphoma, high grade B-cell lymphoma, and DLBCL arising from follicular lymphoma. In some embodiments, the subject has previously received two or more lines of systemic therapy. In some embodiments, the subject does not have primary central nervous system lymphoma.

In another embodiment, the human subject is not administered a lymphodepleting chemotherapeutic agent, such as fludarabine, cyclophosphamide, or bendamustine, prior to, administration of tisagenlecleucel.

The dose of the CAR expressing-immune cell can also be adjusted to account for whether a single dose is being administered or whether multiple doses are being administered. The precise determination of what would be considered an effective dose can be based on factors individual to each subject, including their size, age, sex, weight, and condition of the particular subject, as described above. Dosages can be readily determined by those skilled in the art based on the disclosure herein and knowledge in the art.

The administration of the CAR expressing-immune cells to the subject may be carried out in any suitable manner. In some embodiments, the cells are administered to a patient subcutaneously, intradermally, intratumorally, intranodally, intramedullary, intramuscularly, intravenously (e.g., by infusion), or intraperitoneally. In one embodiment, the cells are administered to a patient by subcutaneous injection. In another embodiment, the cells are administered intravenously. In certain embodiments, the cells may be injected directly into a tumor, lymph node, or site of infection. Optionally, expansion and/or differentiation agents can be administered to the subject prior to, during or after administration of cells to increase production of the cells in vivo.

### III. Anti-CD45 Antibody Drug Conjugates (ADCs)

As described herein, anti-CD45 ADCs can be used in combination with CAR therapy to treat cancer or an autoimmune disease in a human patient. More specifically, anti-CD45 ADCs can be used to deplete CD45+ cells (e.g., CD45+ lymphocytes) in a human subject who is also receiving CAR therapy. Anti-CD45 ADCs target endogenous lymphocytes and kill these cells such that the patient's immune system will not attack the CAR expressing immune cells (autologous or allogeneic) administered to the subject. Thus, anti-CD45 ADCs are used as a conditioning step in combination with CAR therapy to promote acceptance of the engineered CAR expressing immune cells in the recipient patient. One advantage of using anti-CD45 ADCs as a conditioning regimen is that endogenous lymphocytes expressing CD45 can be specifically targeted for depletion versus more traditional methods of conditioning for CAR therapy where general lymphodepleting chemotherapeutic agents are administered to the subject.

### Anti-CD45 Antibodies

ADCs capable of binding CD45 can be used as therapeutic agents to promote acceptance in a human patient of immune cells expressing CARs by preventing or reducing the risk of rejection of the immune cells expressing CARs.

The anti-CD45 ADCs described herein include an anti-CD45 antibody or antigen binding portion thereof, linked to a cytotoxin.

CD45 is a hematopoietic cell-specific transmembrane protein tyrosine phosphatase essential for T and B cell antigen receptor-mediated signaling. CD45 includes a large extracellular domain, and a phosphatase containing cytosolic domain. CD45 may act as both a positive and negative regulator depending on the nature of the stimulus and the cell type involved. Although there are a large number of permutations possible in the CD45 gene, only six isoforms are traditionally identified in humans. The isoforms are RA (Uniprot Accession No: P08575-8; SEQ ID NO: 20), RO (NCBI Accession No: NP_563578.2; SEQ ID NO: 21), RB (NCBI Accession No: XP_006711537.1; SEQ ID NO: 22), RAB (NCBI Accession No: XP_006711535.1; SEQ ID NO: 23), RBC (NCBI Accession No: XP_006711536.1; SEQ ID NO: 24) and RABC (NCBI Accession No. NP_002829.3; SEQ ID NO: 25) (Hermiston et al. 2003 “CD45: a critical regulator of signaling thresholds in immune cells.” Annu Rev Immunol. 2:107-137.). CD45RA is expressed on naïve T cells, and CD45RO is expressed on activated and memory T cells, some B cell subsets, activated monocytes/macrophages, and granulocytes. CD45RB is expressed on peripheral B cells, naïve T cells, thymocytes, weakly on macrophages, and dendritic cells.

In one embodiment, provided herein is an anti-CD45 antibody, or antigen-binding fragment thereof, comprising binding regions, e.g., CDRs, variable regions, corresponding to those of Ab1. The heavy chain variable region (VH) amino acid sequence of Ab1 is set forth in SEQ ID NO: 7 (see Table 4). The VH CDR domain amino acid sequences of Ab1 are set forth in SEQ ID NO: 1 (CDR-H1); SEQ ID NO: 2 (CDR-H2), and SEQ ID NO: 3 (CDR-H3). The light chain variable region (VL) amino acid sequence of Ab1 is described in SEQ ID NO: 8 (see Table 4). The VL CDR domain amino acid sequences of Ab1 are set forth in SEQ ID NO: 4 (CDR-L1); SEQ ID NO: 5 (CDR-L2), and SEQ ID NO: 6 (CDR-L3). Accordingly, in one embodiment, the present disclosure provides an anti-CD45 antibody, or antigen-binding fragment thereof, that may be used in conjunction with the compositions and methods described herein include those that have one or more, or all, of the following CDRs:

a.
(SEQ ID NO: 1)
a CDR-H1 having the amino acid sequence FTFNNYWMT;
b.
(SEQ ID NO: 2)
a CDR-H2 having the amino acid sequence
SISSSGGSIYYPDSVKG;
c.
(SEQ ID NO: 3)
a CDR-H3 having the amino acid sequence
ARDERWAGAMDA;
d.
(SEQ ID NO: 4)
a CDR-L1 having the amino acid sequence
KASQNINKNLD;
e.
(SEQ ID NO: 5)
a CDR-L2 having the amino acid sequence
ETNNLQT; and
f.
(SEQ ID NO: 6)
a CDR-L3 having the amino acid sequence YQHNSRFT.

In certain embodiments, the present disclosure provides an anti-CD45 antibody, or antigen-binding fragment thereof, that may be used in conjunction with the compositions and methods described herein include those that have one or more, or all, of the following heavy chain and light chain variable regions:

Ab 1 Heavy chain (HC) variable region
(CDRs underlined):
(SEQ ID NO: 7)
EVQLVESGGDRVQPGRSLTLSCVTSGFTFNNYWMTWIRQVPGKGLEWVA
SISSSGGSIYYPDSVKGRFTISRDNAKNTLYLQMNSLRSEDTATYYCAR
DERWAGAMDAWGQGTSVTVSS;
and
Ab 1 Light chain (LC) variable region
(CDRs underlined):
(SEQ ID NO: 8)
DIQMTQSPPVLSASVGDRVTLSCKASQNINKNLDWYQQKHGEAPKLLIY
ETNNLQTGIPSRFSGSGSGTDYTLTISSLQPEDVATYYCYQHNSRFTFG
SGTKLEIK.

In certain embodiments, an antibody comprises a modified heavy chain (HC) variable region comprising an HC variable domain comprising SEQ ID NO: 7, or a variant of SEQ ID NO: 7, which variant (i) differs from SEQ ID NO: 7 in 1, 2, 3, 4 or 5 amino acids substitutions, additions or deletions; (ii) differs from SEQ ID NO: 7 in at most 5, 4, 3, 2, or 1 amino acids substitutions, additions or deletions; (iii) differs from SEQ ID NO: 7 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO: 7, wherein in any of (i)-(iv), an amino acid substitution may be a conservative amino acid substitution or a non-conservative amino acid substitution; and wherein the modified heavy chain variable region has an enhanced biological activity relative to that of SEQ ID NO: 7.

In certain embodiments, an antibody comprises a modified light chain (LC) variable region comprising an LC variable domain comprising SEQ ID NO: 8, or a variant of SEQ ID NO: 8, which variant (i) differs from SEQ ID NO: 8 in 1, 2, 3, 4 or 5 amino acids substitutions, additions or deletions; (ii) differs from SEQ ID NO: 8 in at most 5, 4, 3, 2, or 1 amino acids substitutions, additions or deletions; (iii) differs from SEQ ID NO: 8 in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to SEQ ID NO: 8, wherein in any of (i)-(iv), an amino acid substitution may be a conservative amino acid substitution or a non-conservative amino acid substitution; and wherein the modified light chain variable region has an enhanced biological activity relative to that of SEQ ID NO: 8.

In certain embodiments, an anti-CD45 antibody comprises the CDRs described herein (SEQ ID Nos: 1 to 3 and 4 to 6) wherein the CDR comprises a conservative amino acid substitution (or 2, 3, 4, or 5 amino acid substitutions).

Anti-human CD45 antibodies, or fragments thereof, that bind to the epitope on human CD45 bound by Ab1 (or antibodies having the binding regions of Ab1) are also contemplated herein. Further contemplated are anti-human CD45 antibodies, or antigen binding fragments thereof, that compete with Ab1 (or antibodies having the binding regions of Ab1).

In some embodiments, an anti-CD45 antibody, or antigen-binding fragment thereof, specifically binds to human CD45 at a region comprising the amino acid sequence RNGPHERYHLEVEAGNT (SEQ ID NO: 27). For example, in certain embodiments, the anti-CD45 antibody, or antigen-binding fragment thereof, specifically binds to human CD45 at amino acid residues 486R, 493Y, and 502T of SEQ ID NO: 26 (fragment of CD45 isoform corresponding to NP_002829.3), or at residues corresponding thereto in a region comprising the sequence RNGPHERYHLEVEAGNT (SEQ ID NO: 27; bold residues indicate binding site) in other human CD45 isoforms. In some embodiments, the anti-CD45 antibody, or antigen-binding fragment thereof, specifically binds to a fibronectin domain (e.g., fibronectin d4 domain) of human CD45.

In one embodiment, an isolated anti-CD45 antibody, or an antigen binding portion thereof, specifically binds to an epitope of human CD45 comprising residues 486R, 493Y, and 502T of SEQ ID NO: 26, and also binds to cynomolgus and/or rhesus CD45.

In one embodiment, an isolated anti-CD45 antibody, or an antigen binding portion thereof, specifically binds to an epitope of human CD45 comprising the amino acid sequence RNGPHERYHLEVEAGNT (SEQ ID NO: 27), and also binds to cynomolgus and rhesus CD45.

In one embodiment, an isolated anti-CD45 antibody, or an antigen binding portion thereof, specifically binds to an epitope of human CD45 comprising the amino acid sequence CRPPRDRNGPHERYHLEVEAGNTLVRNESHK (SEQ ID NO: 28), and binds to cynomolgus and rhesus CD45.

In one embodiment, an isolated anti-CD45 antibody, or an antigen binding portion thereof, specifically binds to an epitope of human CD45 comprising residues 486R, 493Y, and 502T of SEQ ID NO: 26; binds to at least one additional amino acid, at least two additional amino acids, at least three additional amino acids, at least four additional amino acids, or at least five additional amino acids in a peptide comprising RNGPHERYHLEVEAGNT (SEQ ID NO: 27), wherein the additional amino acid residues are not residues 486R, 493Y, and 502T of SEQ ID NO: 26; and also binds to cynomolgus and rhesus CD45.

In some embodiments, the anti-CD45 antibody is able to bind the extracellular domains of the various isoforms of human CD45. Accordingly, in certain embodiments, the antibody herein is a pan-specific anti-CD45 antibody (i.e., an antibody that binds all six human CD45 isoforms). Further, Ab1 (or antibodies having the binding regions or specificity of this antibody) can also bind to cynomolgus CD45.

In exemplary embodiments, the anti-CD45 antibody used in conjunction with the conditioning methods described herein can be a monoclonal antibody or antigen-binding fragment thereof, a polyclonal antibody or antigen-binding fragment thereof, a humanized antibody or antigen-binding fragment thereof, a fully human antibody or antigen-binding fragment thereof, a chimeric antibody or antigen-binding fragment thereof, a bispecific antibody or antigen-binding fragment thereof, a dual-variable immunoglobulin domain, a single-chain Fv molecule (scFv), a diabody, a triabody, a nanobody, an antibody-like protein scaffold, a Fv fragment, a Fab fragment, a F(ab′)2 molecule, or a tandem di-scFv. Other exemplary anti-CD45 antibodies which may be used in whole or in part in the ADCs or methods described herein are provided below.

In one embodiment, the anti-CD45 antibody is or is derived from clone HI30, which is commercially available from BIOLEGEND® (San Diego, Calif.), or a humanized variant thereof. Humanization of antibodies can be performed by replacing framework residues and constant region residues of a non-human antibody with those of a germline human antibody according to procedures known in the art (as described, for instance, in Example 7, below). Additional anti-CD45 antibodies that can be used in conjunction with the methods described herein include the anti-CD45 antibodies ab10558, EP322Y, MEM-28, ab10559, 0.N.125, F10-89-4, HIe-1, 2B11, YTH24.5, PD7/26/16, F10-89-4, 1B7, ab154885, B-All, phosphor 51007, ab170444, EP350, Y321, GA90, D3/9, X1 6/99, and LT45, which are commercially available from ABCAM® (Cambridge, Mass.), as well as humanized variants thereof. Further anti-CD45 antibodies that may be used in conjunction with the patient conditioning procedures described herein include anti-CD45 antibody HPA000440, which is commercially available from SIGMA-ALDRICH® (St. Louis, Mo.), and humanized variants thereof. Additional anti-CD45 antibodies that can be used in conjunction with the patient conditioning methods described herein include murine monoclonal antibody BC8, which is described, for instance, in Matthews et al., Blood 78:1864-1874, 1991, the disclosure of which is incorporated herein by reference as it pertains to anti-CD45 antibodies, as well as humanized variants thereof. Further anti-CD45 antibodies that can be used in conjunction with the methods described herein include monoclonal antibody YAML568, which is described, for instance, in Glatting et al., J. Nucl. Med. 8:1335-1341, 2006, the disclosure of which is incorporated herein by reference as it pertains to anti-CD45 antibodies, as well as humanized variants thereof. Additional anti-CD45 antibodies that can be used in conjunction with the patient conditioning procedures described herein include monoclonal antibodies YTH54.12 and YTH25.4, which are described, for instance, in Brenner et al., Ann. N.Y. Acad. Sci. 996:80-88, 2003, the disclosure of which is incorporated herein by reference as it pertains to anti-CD45 antibodies, as well as humanized variants thereof. Additional anti-CD45 antibodies for use with the patient conditioning methods described herein include UCHL1, 2H4, SN130, MD4.3, MBI, and MT2, which are described, for instance, in Brown et al., Immunology 64:331-336, 1998, the disclosure of which is incorporated herein by reference as it pertains to anti-CD45 antibodies, as well as humanized variants thereof. Additional anti-CD45 antibodies that can be used in conjunction with the methods described herein include those produced and released from American Type Culture Collection (ATCC) Accession Nos. RA3-6132, RA3-2C2, and TIB122, as well as monoclonal antibodies C363.16A, and 13/2, which are described, for instance, in Johnson et al., J. Exp. Med. 169:1179-1184, 1989, the disclosure of which is incorporated herein by reference as it pertains to anti-CD45 antibodies, as well as humanized variants thereof. Further anti-CD45 antibodies that can be used in conjunction with the patient conditioning methods described herein include the monoclonal antibodies AHN-12.1, AHN-12, AHN-12.2, AHN-12.3, AHN-12.4, HLe-1, and KC56(T200), which are described, for instance, in Harvath et al., J. Immunol. 146:949-957, 1991, the disclosure of which is incorporated herein by reference as it pertains to anti-CD45 antibodies, as well as humanized variants thereof.

Additional anti-CD45 antibodies that can be used in conjunction with the patient conditioning methods described herein include those described, for example, in U.S. Pat. No. 7,265,212 (which describes, e.g., anti-CD45 antibodies 39E11, 16C9, and 1G10, among other clones); U.S. Pat. No. 7,160,987 (which describe, e.g., anti-CD45 antibodies produced and released by ATCC Accession No. HB-11873, such as monoclonal antibody 6G3); and U.S. Pat. No. 6,099,838 (which describes, e.g., anti-CD45 antibody MT3, as well as antibodies produced and released by ATCC Accession Nos. HB220 (also designated MB23G2) and HB223), as well as US 2004/0096901 and US 2008/0003224 (which describes, e.g., anti-CD45 antibodies produced and released by ATCC Accession No. PTA-7339, such as monoclonal antibody 17.1), the disclosures of each of which are incorporated herein by reference as they pertain to anti-CD45 antibodies.

Further anti-CD45 antibodies that can be used in conjunction with the patient conditioning methods described herein include antibodies produced and released from ATCC Accession Nos. MB4B4, MB23G2, 14.8, GAP 8.3, 74-9-3, I/24.D6, 9.4, 4B2, M1/9.3.4.HL.2, as well as humanized and/or affinity-matured variants thereof. Affinity maturation can be performed, for instance, using in vitro display techniques described herein or known in the art, such as phage display, as described in Example 6, below.

Additional anti-CD45 antibodies that can be used in conjunction with the patient conditioning methods described herein include anti-CD45 antibody T29/33, which is described, for instance, in Morikawa et al., Int. J. Hematol. 54:495-504, 1991, the disclosure of which is incorporated herein by reference as it pertains to anti-CD45 antibodies.

In certain embodiments, the anti-CD45 antibody is selected from apamistamab (also known 90Y-BC8, Iomab-B, BC8; as described in, e.g., US20170326259, WO2017155937, and Orozco et al. Blood. 127.3 (2016): 352-359.) or BC8-B10 (as described, e.g., in Li et al. PloS one 13.10 (2018): e0205135.), each of which is incorporated by reference. Other anti-CD45 antibodies have been described, for example, in WO2003/048327, WO2016/016442, US2017/0226209, US2016/0152733, U.S. Pat. No. 9,701,756; US2011/0076270, or U.S. Pat. No. 7,825,222, each of which is incorporated by reference in its entirety.

For example, in one embodiment, the anti-CD45 antibody, or antigen-binding fragment thereof, comprising binding regions, e.g., CDRs, variable regions, corresponding to those of apamistamab. The heavy chain variable region (VH) amino acid sequence of apamistamab is set forth in SEQ ID NO: 31 (see Table 4). The light chain variable region (VL) amino acid sequence of apamistamab is described in SEQ ID NO: 32 (see Table 4). In other embodiments, an anti-CD45 antibody, or antigen-binding portion thereof, comprises a variable heavy chain comprising the amino acid residues set forth in SEQ ID NO: 31, and a light chain variable region as set forth in SEQ ID NO: 32. In one embodiment, the anti-CD45 antibody comprises a heavy chain comprising a CDR1, CDR2 and CDR3 of apamistamab, and a light chain variable region comprising a CDR1, CDR2 and CDR3 of apamistamab.

In one embodiment, the anti-CD45 antibody comprises a heavy chain of an anti-CD45 antibody described herein, and a light chain variable region of anti-CD45 antibody described herein. In one embodiment, the anti-CD45 antibody comprises a heavy chain comprising a CDR1, CDR2 and CDR3 of an anti-CD45 antibody described herein, and a light chain variable region comprising a CDR1, CDR2 and CDR3 of an anti-CD45 antibody described herein.

In another embodiment, the antibody, or antigen-binding fragment thereof, comprises a heavy chain variable region that comprises an amino acid sequence having at least 95% identity to an anti-CD45 antibody herein, e.g., at least 95%, 96%, 97%, 98%, 99%, or 100% identity to an anti-CD45 antibody herein. In certain embodiments, an antibody comprises a modified heavy chain (HC) variable region comprising an HC variable domain of an anti-CD45 antibody herein, or a variant thereof, which variant (i) differs from the anti-CD45 antibody in 1, 2, 3, 4 or 5 amino acids substitutions, additions or deletions; (ii) differs from the anti-CD45 antibody in at most 5, 4, 3, 2, or 1 amino acids substitutions, additions or deletions; (iii) differs from the anti-CD45 antibody in 1-5, 1-3, 1-2, 2-5 or 3-5 amino acids substitutions, additions or deletions and/or (iv) comprises an amino acid sequence that is at least about 75%, 80%, 85%, 90%, 95%, 96%, 97%, 98% or 99% identical to the anti-CD45 antibody, wherein in any of (i)-(iv), an amino acid substitution may be a conservative amino acid substitution or a non-conservative amino acid substitution; and wherein the modified heavy chain variable region can have an enhanced biological activity relative to the heavy chain variable region of the anti-CD45 antibody, while retaining the CD45 binding specificity of the antibody.

In one embodiment, the methods and compositions disclosed herein comprise an anti-CD45 antibody, or antigen binding fragment thereof, that specifically binds to human CD45 (and possibly CD45 from one or more non-human species) but does not substantially bind to non-CD45 proteins. In embodiment, the antibody, or fragment thereof, binds to human CD45 with a K_{D} of 1×10⁻⁷ M or less, a K_{D} of 5×10⁻⁸ M or less, a K_{D} of 3×10⁻⁸ M or less, a K_{D} of 1×10⁻⁸ M or less, a K_{D} of 5×10⁻⁹ M or less, a K_{D} of 1×10⁻¹⁰ M or less, or a K_{D} of 1×10⁻¹¹ M or less.

Further, in certain embodiments the anti-CD45 ADC has a serum half-life in a human subject of about 3 days or less. In certain embodiments, the anti-CD45 described herein has a half-life (e.g., in humans) equal to or less than about 24 hours, equal to or less than about 23 hours, equal to or less than about 22 hours, equal to or less than about 21 hours, equal to or less than about 20 hours, equal to or less than about 19 hours, equal to or less than about 18 hours, equal to or less than about 17 hours, equal to or less than about 16 hours, equal to or less than about 15 hours, equal to or less than about 14 hours, equal to or less than about 13 hours, equal to or less than about 12 hours, or equal to or less than about 11 hours.

In one embodiment, the anti-CD45 antibody described herein has a half-life (e.g., in humans) about 1-5 hours, about 5-10 hours, about 10-15 hours, about 15-20 hours, or about 20 to 25 hours.

Additional anti-CD45 antibodies that can be used in the ADCs described herein can be identified using techniques known in the art, such as hybridoma production. Hybridomas can be prepared using a murine system. Protocols for immunization and subsequent isolation of splenocytes for fusion are known in the art. Fusion partners and procedures for hybridoma generation are also known. Alternatively, anti-CD45 antibodies can be generated using the HuMAb-Mouse® or XenoMouse™. In making additional anti-CD45 antibodies, the CD45 antigen is isolated and/or purified. The CD45 antigen may be a fragment of CD45 from the extracellular domain of CD45. Immunization of animals can be performed by any method known in the art. See, e.g., Harlow and Lane, Antibodies: A Laboratory Manual, New York: Cold Spring Harbor Press, 1990. Methods for immunizing animals such as mice, rats, sheep, goats, pigs, cattle and horses are well known in the art. See, e.g., Harlow and Lane, supra, and U.S. Pat. No. 5,994,619. The CD45 antigen may be administered with an adjuvant to stimulate the immune response. Adjuvants known in the art include complete or incomplete Freund's adjuvant, RIBI (muramyl dipeptides) or ISCOM (immunostimulating complexes). After immunization of an animal with a CD45 antigen, antibody-producing immortalized cell lines are prepared from cells isolated from the immunized animal. After immunization, the animal is sacrificed and lymph node and/or splenic B cells are immortalized by methods known in the art (e.g., oncogene transfer, oncogenic virus transduction, exposure to carcinogenic or mutating compounds, fusion with an immortalized cell, e.g., a myeloma cell, and inactivating a tumor suppressor gene. See, e.g., Harlow and Lane, supra. Hybridomas can be selected, cloned and further screened for desirable characteristics, including robust growth, high antibody production and desirable antibody characteristics.

Anti-CD45 antibodies for use in the anti-CD45 ADCs described herein can also be identified using high throughput screening of libraries of antibodies or antibody fragments for molecules capable of binding CD45. Such methods include in vitro display techniques known in the art, such as phage display, bacterial display, yeast display, mammalian cell display, ribosome display, mRNA display, and cDNA display, among others. The use of phage display to isolate antibodies, antigen-binding fragments, or ligands that bind biologically relevant molecules has been reviewed, for example, in Felici et al., Biotechnol. Annual Rev. 1:149-183, 1995; Katz, Annual Rev. Biophys. Biomol. Struct. 26:27-45, 1997; and Hoogenboom et al., Immunotechnology 4:1-20, 1998, the disclosures of each of which are incorporated herein by reference as they pertain to in vitro display techniques. Randomized combinatorial peptide libraries have been constructed to select for polypeptides that bind cell surface antigens as described in Kay, Perspect. Drug Discovery Des. 2:251-268, 1995 and Kay et al., Mol. Divers. 1:139-140, 1996, the disclosures of each of which are incorporated herein by reference as they pertain to the discovery of antigen-binding molecules. Proteins, such as multimeric proteins, have been successfully phage-displayed as functional molecules (see, for example, EP 0349578; EP 4527839; and EP 0589877, as well as Chiswell and McCafferty, Trends Biotechnol. 10:80-84 1992, the disclosures of each of which are incorporated herein by reference as they pertain to the use of in vitro display techniques for the discovery of antigen-binding molecules. In addition, functional antibody fragments, such as Fab and scFv fragments, have been expressed in in vitro display formats (see, for example, McCafferty et al., Nature 348:552-554, 1990; Barbas et al., Proc. Natl. Acad. Sci. USA 88:7978-7982, 1991; and Clackson et al., Nature 352:624-628, 1991, the disclosures of each of which are incorporated herein by reference as they pertain to in vitro display platforms for the discovery of antigen-binding molecules).

In addition to in vitro display techniques, computational modeling techniques can be used to design and identify anti-CD45 antibodies or antibody fragments in silico, for instance, using the procedures described in US 2013/0288373, the disclosure of which is incorporated herein as it pertains to molecular modeling methods for identifying anti-CD45 antibodies. For example, using computational modeling techniques, one of skill in the art can screen libraries of antibodies or antibody fragments in silico for molecules capable of binding specific epitopes on CD45, such as extracellular epitopes of CD45.

In one embodiment, the anti-CD45 antibody used in the ADCs described herein are able to internalize into the cell. In identifying an anti-CD45 antibody (or fragment thereof) additional techniques can be used to identify antibodies or antigen-binding fragments that bind CD45 on the surface of a cell (e.g., a lymphocyte) and further are able to be internalized by the cell, for instance, by receptor-mediated endocytosis. For example, the in vitro display techniques described above can be adapted to screen for antibodies or antigen-binding fragments thereof that bind CD45 on the surface of a hematopoietic stem cell and that are subsequently internalized. Phage display represents one such technique that can be used in conjunction with this screening paradigm. To identify anti-CD45 antibodies or fragments thereof that bind CD45 and are subsequently internalized a CD45+ cell, one of skill in the art can use the phage display techniques described in Williams et al., Leukemia 19:1432-1438, 2005, the disclosure of which is incorporated herein by reference in its entirety.

The internalizing capacity of an anti-CD45 antibody or fragment thereof can be assessed, for instance, using radionuclide internalization assays known in the art. For example, an anti-CD45 antibody or fragment thereof, identified using in vitro display techniques described herein or known in the art can be functionalized by incorporation of a radioactive isotope, such as ¹⁸F, ⁷⁵Br, ⁷⁷Br, ¹²²I, ¹²³I, ¹²⁴I, ¹²⁵I, ¹²⁹I, ¹³¹I, ²¹¹At, ⁶⁷Ga, ¹¹¹In, ⁹⁹Tc, ¹⁶⁹Yb, ¹⁸⁶Re, ⁶⁴Cu, ⁶⁷Cu, ¹⁷⁷Lu, ⁷⁷As, ⁷²As, ⁸⁶Y, ⁹⁰Y, ⁸⁹Zr, ²¹²Bi, ²¹³Bi, or ²²⁵Ac. For instance, radioactive halogens, such as ¹⁸F, ⁷⁵Br, ⁷⁷Br, ¹²²I, ¹²³I, ¹²⁴I, ¹²⁵I, ¹²⁹I, ¹³¹I, ²¹¹At, can be incorporated into antibodies, fragments thereof, or ligands using beads, such as polystyrene beads, containing electrophilic halogen reagents (e.g., Iodination Beads, Thermo Fisher Scientific, Inc., Cambridge, Mass.). Radiolabeled antibodies, or fragments thereof, can be incubated with hematopoietic stem cells for a time sufficient to permit internalization. Internalized antibodies, or fragments thereof, can be identified by detecting the emitted radiation (e.g., y-radiation) of the resulting hematopoietic stem cells in comparison with the emitted radiation (e.g., y-radiation) of the recovered wash buffer. The foregoing internalization assays can also be used to characterize ADCs.

In some embodiments, the anti-CD45 antibody (or fragment thereof) has a defined serum half-life. For example, an anti-CD45 antibody (or fragment thereof) may have a serum half-life of about 1-24 hours in the human patient. ADCs containing such anti-CD45 antibodies can also, for example, have a serum half-life of about 1-24 hours in a human patient. Pharmacokinetic analysis by measurement of serum levels can be performed by assays known in the art.

For recombinant production of an anti-CD45 antibody, nucleic acid encoding an antibody, e.g., as described above, is isolated and inserted into one or more vectors for further cloning and/or expression in a host cell. Such nucleic acid may be readily isolated and sequenced using conventional procedures (e.g., by using oligonucleotide probes that are capable of binding specifically to genes encoding the heavy and light chains of the antibody).

Suitable host cells for cloning or expression of antibody-encoding vectors include prokaryotic or eukaryotic cells described herein. For example, antibodies may be produced in bacteria, in particular when glycosylation and Fc effector function are not needed. For expression of antibody fragments and polypeptides in bacteria, see, e.g., U.S. Pat. Nos. 5,648,237, 5,789,199, and 5,840,523. (See also Charlton, Methods in Molecular Biology, Vol. 248 (B. K. C. Lo, ed., Humana Press, Totowa, N.J., 2003), pp. 245-254, describing expression of antibody fragments in E. coli.) After expression, the antibody may be isolated from the bacterial cell paste in a soluble fraction and can be further purified.

Vertebrate cells may also be used as hosts. For example, mammalian cell lines that are adapted to grow in suspension may be useful. Other examples of useful mammalian host cell lines are monkey kidney CV1 line transformed by SV40 (COS-7); human embryonic kidney line (293 or 293 cells as described, e.g., in Graham et al., J. Gen Virol. 36:59 (1977)); baby hamster kidney cells (BHK); mouse sertoli cells (TM4 cells as described, e.g., in Mather, Biol. Reprod. 23:243-251 (1980)); monkey kidney cells (CV1); African green monkey kidney cells (VERO-76); human cervical carcinoma cells (HELA); canine kidney cells (MDCK; buffalo rat liver cells (BRL 3A); human lung cells (W138); human liver cells (Hep G2); mouse mammary tumor (MMT 060562); TRI cells, as described, e.g., in Mather et al., Annals N.Y. Acad. Sci. 383:44-68 (1982); MRC 5 cells; and FS4 cells. Other useful mammalian host cell lines include Chinese hamster ovary (CHO) cells, including DHFR-CHO cells (Urlaub et al., Proc. Natl. Acad. Sci. USA 77:4216 (1980)); and myeloma cell lines such as Y0, NS0 and Sp2/0. For a review of certain mammalian host cell lines suitable for antibody production, see, e.g., Yazaki and Wu, Methods in Molecular Biology, Vol. 248 (B. K. C. Lo, ed., Humana Press, Totowa, N.J.), pp. 255-268 (2003). In one embodiment, the host cell is eukaryotic, e.g. a Chinese Hamster Ovary (CHO) cell or lymphoid cell (e.g., Y0, NS0, Sp20 cell).

### Fc-Modified Antibodies

The present disclosure is based in part on the discovery that antibodies, or antigen-binding fragments thereof, having Fc modifications that allow Fc silencing capable of binding an antigen expressed by, e.g., lymphocytes, such as CD45, can be used as therapeutic agents alone or as ADCs to (i) treat cancers and autoimmune diseases; and (ii) facilitate the engraftment of transplanted hematopoietic stem cells in a patient in need of transplant therapy. These therapeutic activities can be caused, for instance, by the binding of an anti-CD45 antibody, or antigen-binding fragment thereof, which binds to CD45 expressed by a cell (e.g., a lymphocyte),

The antibodies or binding fragments described herein may also include modifications and/or mutations that alter the properties of the antibodies and/or fragments, such as those that increase half-life, or increase or decrease ADCC.

In one embodiment, the anti-CD45 antibody, or binding fragment thereof, comprises a modified Fc region, wherein said modified Fc region comprises at least one amino acid modification relative to a wild-type Fc region, such that said molecule has an altered affinity for or binding to an FcgammaR (FcγR). Certain amino acid positions within the Fc region are known through crystallography studies to make a direct contact with FcγR. Specifically amino acids 234-239 (hinge region), amino acids 265-269 (B/C loop), amino acids 297-299 (C′/E loop), and amino acids 327-332 (F/G) loop. (see Sondermann et al., 2000 Nature, 406: 267-273). In some embodiments, the antibodies described herein may comprise variant Fc regions comprising modification of at least one residue that makes a direct contact with an FcγR based on structural and crystallographic analysis. In one embodiment, the Fc region of the anti-CD45 antibody (or fragment thereof) comprises an amino acid substitution at amino acid 265 according to the EU index as in Kabat et al., Sequences of Proteins of Immunological Interest, 5th Ed. Public Health Service, NH1, MD (1991), expressly incorporated herein by reference. The “EU index as in Kabat” refers to the numbering of the human IgG1 EU antibody. In one embodiment, the Fc region comprises a D265A mutation. In one embodiment, the Fc region comprises a D265C mutation. In some embodiments, the Fc region of the antibody (or fragment thereof) comprises an amino acid substitution at amino acid 234 according to the EU index as in Kabat.

In one embodiment, the Fc region comprises a mutation at an amino acid position of D265, V205, H435, 1253, and/or H310. For example, specific mutations at these positions include D265C, V205C, H435A, I253A, and/or H310A.

In one embodiment, the Fc region comprises a L234A mutation. In some embodiments, the Fc region of the anti-CD45 antibody (or fragment thereof) comprises an amino acid substitution at amino acid 235 according to the EU index as in Kabat. In one embodiment, the Fc region comprises a L235A mutation. In yet another embodiment, the Fc region comprises a L234A and L235A mutation. In a further embodiment, the Fc region comprises a D265C, L234A, and L235A mutation. In yet a further embodiment, the Fc region comprises a D265C, L234A, L235A, and H435A mutation. In a further embodiment, the Fc region comprises a D265C and H435A mutation.

In yet another embodiment, the Fc region comprises a L234A and L235A mutation (also referred to herein as “L234A.L235A” or as “LALA”). In another embodiment, the Fc region comprises a L234A and L235A mutation, wherein the Fc region does not include a P329G mutation. In a further embodiment, the Fc region comprises a D265C, L234A, and L235A mutation (also referred to herein as “D265C.L234A.L235A”). In another embodiment, the Fc region comprises a D265C, L234A, and L235A mutation, wherein the Fc region does not include a P329G mutation. In yet a further embodiment, the Fc region comprises a D265C, L234A, L235A, and H435A mutation (also referred to herein as “D265C.L234A.L235A.H435A”). In another embodiment, the Fc region comprises a D265C, L234A, L235A, and H435A mutation, wherein the Fc region does not include a P329G mutation. In a further embodiment, the Fc region comprises a D265C and H435A mutation (also referred to herein as “D265C.H435A”). In yet another embodiment, the Fc region comprises a D265A, S239C, L234A, and L235A mutation (also referred to herein as “D265A.S239C.L234A.L235A”). In yet another embodiment, the Fc region comprises a D265A, S239C, L234A, and L235A mutation, wherein the Fc region does not include a P329G mutation. In another embodiment, the Fc region comprises a D265C, N297G, and H435A mutation (also referred to herein as “D265C.N297G.H435A”). In another embodiment, the Fc region comprises a D265C, N297Q, and H435A mutation (also referred to herein as “D265C.N297Q.H435A”). In another embodiment, the Fc region comprises a E233P, L234V, L235A and delG236 (deletion of 236) mutation (also referred to herein as “E233P.L234V.L235A.delG236” or as “EPLVLAdelG”). In another embodiment, the Fc region comprises a E233P, L234V, L235A and delG236 (deletion of 236) mutation, wherein the Fc region does not include a P329G mutation. In another embodiment, the Fc region comprises a E233P, L234V, L235A, delG236 (deletion of 236) and H435A mutation (also referred to herein as “E233P.L234V.L235A.delG236.H435A” or as “EPLVLAdelG.H435A”). In another embodiment, the Fc region comprises a E233P, L234V, L235A, delG236 (deletion of 236) and H435A mutation, wherein the Fc region does not include a P329G mutation. In another embodiment, the Fc region comprises a L234A, L235A, S239C and D265A mutation. In another embodiment, the Fc region comprises a L234A, L235A, S239C and D265A mutation, wherein the Fc region does not include a P329G mutation. In another embodiment, the Fc region comprises a H435A, L234A, L235A, and D265C mutation. In another embodiment, the Fc region comprises a H435A, L234A, L235A, and D265C mutation, wherein the Fc region does not include a P329G mutation.

In some embodiments, the antibody has a modified Fc region such that, the antibody decreases an effector function in an in vitro effector function assay with a decrease in binding to an Fc receptor (Fc R) relative to binding of an identical antibody comprising an unmodified Fc region to the FcR. In some embodiments, the antibody has a modified Fc region such that, the antibody decreases an effector function in an in vitro effector function assay with a decrease in binding to an Fc gamma receptor (FcγR) relative to binding of an identical antibody comprising an unmodified Fc region to the FcγR. In some embodiments, the FcγR is FcγR1. In some embodiments, the FcγR is FcγR2A. In some embodiments, the FcγR is FcγR2B. In other embodiments, the FcγR is FcγR2C. In some embodiments, the FcγR is FcγR3A. In some embodiments, the FcγR is FcγR3B. In other embodiments, the decrease in binding is at least a 70% decrease, at least a 80% decrease, at least a 90% decrease, at least a 95% decrease, at least a 98% decrease, at least a 99% decrease, or a 100% decrease in antibody binding to a FcγR relative to binding of the identical antibody comprising an unmodified Fc region to the FcγR. In other embodiments, the decrease in binding is at least a 70% to a 100% decrease, at least a 80% to a 100% decrease, at least a 90% to a 100% decrease, at least a 95% to a 100% decrease, or at least a 98% to a 100% decrease, in antibody binding to a FcγR relative to binding of the identical antibody comprising an unmodified Fc region to the FcγR

In some embodiments, the antibody has a modified Fc region such that, the antibody decreases cytokine release in an in vitro cytokine release assay with a decrease in cytokine release of at least 50% relative to cytokine release of an identical antibody comprising an unmodified Fc region. In some embodiments, the decrease in cytokine release is at least a 70% decrease, at least a 80% decrease, at least a 90% decrease, at least a 95% decrease, at least a 98% decrease, at least a 99% decrease, or a 100% decrease in cytokine release relative to cytokine release of the identical antibody comprising an unmodified Fc region. In some embodiments, the decrease in cytokine release is at least a 70% to a 100% decrease, at least an 80% to a 100% decrease, at least a 90% to a 100% decrease, at least a 95% to a 100% decrease in cytokine release relative to cytokine release of the identical antibody comprising an unmodified Fc region. In certain embodiments, cytokine release is by immune cells.

In some embodiments, the antibody has a modified Fc region such that, the antibody decreases mast cell degranulation in an in vitro mast cell degranulation assay with a decrease in mast cell degranulation of at least 50% relative to mast cell degranulation of an identical antibody comprising an unmodified Fc region. In some embodiments, the decrease in mast cell degranulation is at least a 70% decrease, at least a 80% decrease, at least a 90% decrease, at least a 95% decrease, at least a 98% decrease, at least a 99% decrease, or a 100% decrease in mast cell degranulation relative to mast cell degranulation of the identical antibody comprising an unmodified Fc region. In some embodiments, the decrease in mast cell degranulation is at least a 70% to a 100% decrease, at least a 80% to a 100% decrease, at least a 90% to a 100% decrease, or at least a 95% to a 100% decrease, in mast cell degranulation relative to mast cell degranulation of the identical antibody comprising an unmodified Fc region.

In some embodiments, the antibody has a modified Fc region such that, the antibody decreases or prevents antibody dependent cell phagocytosis (ADCP) in an in vitro antibody dependent cell phagocytosis assay, with a decrease in ADCP of at least 50% relative to ADCP of an identical antibody comprising an unmodified Fc region. In some embodiments, the decrease in ADCP is at least a 70% decrease, at least a 80% decrease, at least a 90% decrease, at least a 95% decrease, at least a 98% decrease, at least a 99% decrease, or a 100% decrease in cytokine release relative to cytokine release of the identical antibody comprising an unmodified Fc region.

In some embodiments, the anti-HC antibody (e.g., anti-CD45 antibody) described herein comprises an Fc region comprising one of the following modifications or combinations of modifications: D265A, D265C, D265C/H435A, D265C/LALA, D265C/LALA/H435A, D265A/S239C/L234A/L235A/H435A, D265A/S239C/L234A/L235A, D265C/N297G, D265C/N297G/H435A, D265C (EPLVLAdelG*), D265C (EPLVLAdelG)/H435A, D265C/N297Q/H435A, D265C/N297Q, EPLVLAdelG/H435A, EPLVLAdelG/D265C, EPLVLAdelG/D265A, N297A, N297G, or N297Q. In some embodiments, the anti-CD45 antibody herein comprises an Fc region comprising one of the following modifications or combinations of modifications: D265A, D265C, D265C/H435A, D265C/LALA, D265C/LALA/H435A, D265C/N297G, D265C/N297G/H435A, D265C (IgG2*), D265C (IgG2)/H435A, D265C/N297Q/H435A, D265C/N297Q, EPLVLAdelG/H435A, N297A, N297G, or N297Q.

Binding or affinity between a modified Fc region and a Fc gamma receptor can be determined using a variety of techniques known in the art, for example but not limited to, equilibrium methods (e.g., enzyme-linked immunoabsorbent assay (ELISA); KinExA, Rathanaswami et al. Analytical Biochemistry, Vol. 373:52-60, 2008; or radioimmunoassay (RIA)), or by a surface plasmon resonance assay or other mechanism of kinetics-based assay (e.g., BIACORE® analysis or Octet™ analysis (forteBlO)), and other methods such as indirect binding assays, competitive binding assays fluorescence resonance energy transfer (FRET), gel electrophoresis and chromatography (e.g., gel filtration). These and other methods may utilize a label on one or more of the components being examined and/or employ a variety of detection methods including but not limited to chromogenic, fluorescent, luminescent, or isotopic labels. A detailed description of binding affinities and kinetics can be found in Paul, W. E., ed., Fundamental Immunology, 4th Ed., Lippincott-Raven, Philadelphia (1999), which focuses on antibody-immunogen interactions. One example of a competitive binding assay is a radioimmuno assay comprising the incubation of labeled antigen with the antibody of interest in the presence of increasing amounts of unlabeled antigen, and the detection of the antibody bound to the labeled antigen. The affinity of the antibody of interest for a particular antigen and the binding off-rates can be determined from the data by scatchard plot analysis. Competition with a second antibody can also be determined using radioimmunoassays. In this case, the antigen is incubated with antibody of interest conjugated to a labeled compound in the presence of increasing amounts of an unlabeled second antibody.

In one embodiment, an antibody having the Fc modifications described herein (e.g., D265C, L234A, L235A, and/or H435A) has at least a 70% decrease, at least a 80% decrease, at least a 90% decrease, at least a 95% decrease, at least a 98% decrease, at least a 99% decrease, or a 100% decrease in binding to a Fc gamma receptor relative to binding of the identical antibody comprising an unmodified Fc region to the Fc gamma receptor (e.g., as assessed by biolayer interferometry (BLI)).

Without wishing to be bound by any theory, it is believed that Fc region binding interactions with a Fc gamma receptor are essential for a variety of effector functions and downstream signaling events including, but not limited to, antibody dependent cell-mediated cytotoxicity (ADCC) and complement dependent cytotoxicity (CDC). Accordingly, in certain aspects, an antibody comprising a modified Fc region (e.g., comprising a L234A, L235A, and/or a D265C mutation) has substantially reduced or abolished effector functions. Effector functions can be assayed using a variety of methods known in the art, e.g., by measuring cellular responses (e.g., mast cell degranulation or cytokine release) in response to the antibody of interest. For example, using standard methods in the art, the Fc-modified antibodies can be assayed for their ability to trigger mast cell degranulation in or for their ability to trigger cytokine release, e.g. by human peripheral blood mononuclear cells.

Thus, in one embodiment, the Fc region comprises a mutation resulting in a decrease in half-life (e.g., relative to an antibody having an unmodified Fc region). An antibody having a short half-life may be advantageous in certain instances where the antibody is expected to function as a short-lived therapeutic, e.g., the conditioning step described herein where the antibody is administered followed by a CAR therapy. Ideally, the antibody would be substantially cleared prior to delivery of the CAR therapy, which also generally express a target antigen (e.g., CD45) but are not the target of the anti-CD45 antibody unlike the endogenous stem cells. In one embodiment, the Fc regions comprises a mutation at position 435 (EU index according to Kabat). In one embodiment, the mutation is an H435A mutation.

In one embodiment, the anti-CD45 described herein has a half-life (e.g., in humans) equal to or less than about 24 hours, equal to or less than about 23 hours, equal to or less than about 22 hours, equal to or less than about 21 hours, equal to or less than about 20 hours, equal to or less than about 19 hours, equal to or less than about 18 hours, equal to or less than about 17 hours, equal to or less than about 16 hours, equal to or less than about 15 hours, equal to or less than about 14 hours, equal to or less than about 13 hours, equal to or less than about 12 hours, or equal to or less than about 11 hours.

In one embodiment, the anti-CD45 antibody described herein has a half-life (e.g., in humans) of about 1-5 hours, about 5-10 hours, about 10-15 hours, about 15-20 hours, or about 20 to 25 hours. In one embodiment, the half-life of the anti-HC antibody is about 5-7 hours; about 5-9 hours; about 5-11 hours; about 5-13 hours; about 5-15 hours; about 5-20 hours; about 5-24 hours; about 7-24 hours; about 9-24 hours; about 11-24 hours; about 12-22 hours; about 10-20 hours; about 8-18 hours; or about 14-24 hours.

In some aspects, the Fc region comprises two or more mutations that confer reduced half-life and reduce an effector function of the antibody. In some embodiments, the Fc region comprises a mutation resulting in a decrease in half-life and a mutation of at least one residue that can make direct contact with an FcγR (e.g., as based on structural and crystallographic analysis). In one embodiment, the Fc region comprises a H435A mutation, a L234A mutation, and a L235A mutation. In one embodiment, the Fc region comprises a H435A mutation and a D265C mutation. In one embodiment, the Fc region comprises a H435A mutation, a L234A mutation, a L235A mutation, and a D265C mutation.

In some embodiments, the antibody or antigen-binding fragment thereof is conjugated to a cytotoxin (e.g., amatoxin) by way of a cysteine residue in the Fc domain of the antibody or antigen-binding fragment thereof. In some embodiments, the cysteine residue is introduced by way of a mutation in the Fc domain of the antibody or antigen-binding fragment thereof. For instance, the cysteine residue may be selected from the group consisting of Cys118, Cys239, and Cys265. In one embodiment, the Fc region of the anti-CD45 antibody (or fragment thereof) comprises an amino acid substitution at amino acid 265 according to the EU index as in Kabat. In one embodiment, the Fc region comprises a D265C mutation. In one embodiment, the Fc region comprises a D265C and H435A mutation. In one embodiment, the Fc region comprises a D265C, a L234A, and a L235A mutation. In one embodiment, the Fc region comprises a D265C, a L234A, a L235A, and a H435A mutation. In one embodiment, the Fc region of the anti-CD45 antibody, or antigen-binding fragment thereof, comprises an amino acid substitution at amino acid 239 according to the EU index as in Kabat. In one embodiment, the Fc region comprises a S239C mutation. In one embodiment, the Fc region comprises a L234A mutation, a L235A mutation, a S239C mutation and a D265A mutation. In another embodiment, the Fc region comprises a S239C and H435A mutation. In another embodiment, the Fc region comprises a L234A mutation, a L235A mutation, and S239C mutation. In yet another embodiment, the Fc region comprises a H435A mutation, a L234A mutation, a L235A mutation, and S239C mutation. In yet another embodiment, the Fc region comprises a H435A mutation, a L234A mutation, a L235A mutation, a S239C mutation and D265A mutation.

Notably, Fc amino acid positions are in reference to the EU numbering index unless otherwise indicated.

Antibodies and antigen-binding fragments that may be used in conjunction with the compositions and methods described herein include the above-described antibodies and antigen-binding fragments thereof, as well as humanized variants of those non-human antibodies and antigen-binding fragments described above and antibodies or antigen-binding fragments that bind the same epitope as those described above, as assessed, for instance, by way of a competitive antigen binding assay.

The antibodies of the present disclosure may be further engineered to further modulate antibody half-life by introducing additional Fc mutations, such as those described for example in (Dall'Acqua et al. (2006) J Biol Chem 281: 23514-24), (Zalevsky et al. (2010) Nat Biotechnol 28: 157-9), (Hinton et al. (2004) J Biol Chem 279: 6213-6), (Hinton et al. (2006) J Immunol 176: 346-56), (Shields et al. (2001) J Biol Chem 276: 6591-604), (Petkova et al. (2006) Int Immunol 18: 1759-69), (Datta-Mannan et al. (2007) Drug Metab Dispos 35: 86-94), (Vaccaro et al. (2005) Nat Biotechnol 23: 1283-8), (Yeung et al. (2010) Cancer Res 70: 3269-77) and (Kim et al. (1999) Eur J Immunol 29: 2819-25), and include positions 250, 252, 253, 254, 256, 257, 307, 376, 380, 428, 434 and 435. Exemplary mutations that may be made singularly or in combination are T250Q, M252Y, 1253A, S254T, T256E, P2571, T307A, D376V, E380A, M428L, H433K, N434S, N434A, N434H, N434F, H435A and H435R mutations.

Methods of engineering antibodies to include any of the Fc modifications herein are well known in the art. These methods include, but are not limited to, preparation by site-directed (or oligonucleotide-mediated) mutagenesis, PCR mutagenesis, and cassette mutagenesis of a prepared DNA molecule encoding the antibody or at least the constant region of the antibody. Site-directed mutagenesis is well known in the art (see, e.g., Carter et al., Nucleic Acids Res., 13:4431-4443 (1985) and Kunkel et al., Proc. Natl. Acad. Sci. USA, 82:488 (1987)). PCR mutagenesis is also suitable for making amino acid sequence variants of the starting polypeptide. See Higuchi, in PCR Protocols, pp. 177-183 (Academic Press, 1990); and Vallette et al., Nuc. Acids Res. 17:723-733 (1989). Another method for preparing sequence variants, cassette mutagenesis, is based on the technique described by Wells et al., Gene, 34:315-323 (1985).

### Cytotoxins

Various cytotoxins can be conjugated to an anti-CD45 antibody via a linker for use in the combination therapies described herein. In particular, the anti-CD45 ADCs include an antibody (or an antigen-binding fragment thereof) conjugated (i.e., covalently attached by a linker) to a cytotoxic moiety (or cytotoxin). In various embodiments, the cytotoxic moiety exhibits reduced or no cytotoxicity when bound in a conjugate, but resumes cytotoxicity after cleavage from the linker. In various embodiments, the cytotoxic moiety maintains cytotoxicity without cleavage from the linker. In some embodiments, the cytotoxic molecule is conjugated to a cell internalizing antibody, or antigen-binding fragment thereof as disclosed herein, such that following the cellular uptake of the antibody, or fragment thereof, the cytotoxin may access its intracellular target and, e.g., mediate T cell death.

Antibodies, antigen-binding fragments thereof, and ligands described herein (e.g., antibodies, antigen-binding fragments thereof, and soluble ligands that recognize and bind CD45) can be conjugated (or linked) to a cytotoxin.

ADCs of the present disclosure therefore may be of the general formula Ab-(Z-L-D)ₙ wherein an antibody or antigen-binding fragment thereof (Ab) is conjugated (covalently linked) to linker (L), through a chemical moiety (Z), to a cytotoxic moiety (“drug,” D). “n” represents the number of drugs linked to the antibody, and generally ranges from 1 to 8.

Accordingly, the antibody or antigen-binding fragment thereof may be conjugated to a number of drug moieties as indicated by integer n, which represents the average number of cytotoxins per antibody, which may range, e.g., from about 1 to about 20. In some embodiments, n is from 1 to 4. In some embodiments, n is 1. The average number of drug moieties per antibody in preparations of ADC from conjugation reactions may be characterized by conventional means such as mass spectroscopy, ELISA assay, and HPLC. The quantitative distribution of ADC in terms of n may also be determined. In some instances, separation, purification, and characterization of homogeneous ADC where n is a certain value from ADC with other drug loadings may be achieved by means such as reverse phase HPLC or electrophoresis.

For some anti-CD45 ADCs, the average number of cytotoxins per antibody may be limited by the number of attachment sites on the antibody. For example, where the attachment is a cysteine thiol, an antibody may have only one or several cysteine thiol groups, or may have only one or several sufficiently reactive thiol groups through which a linker and chemical moiety may be attached. Generally, antibodies do not contain many free and reactive cysteine thiol groups which may be linked to a drug moiety; primarily, cysteine thiol residues in antibodies exist as disulfide bridges. In certain embodiments, an antibody may be reduced with a reducing agent such as dithiothreitol (DTT) or tricarbonylethylphosphine (TCEP), under partial or total reducing conditions, to generate reactive cysteine thiol groups. In certain embodiments, higher drug loading, e.g. n>5, may cause aggregation, insolubility, toxicity, or loss of cellular permeability of certain antibody-drug conjugates.

In certain embodiments, fewer than the theoretical maximum of drug moieties are conjugated to an antibody during a conjugation reaction. An antibody may contain, for example, lysine residues that do not react with the drug-linker intermediate or linker reagent, as discussed below. Only the most reactive lysine groups may react with an amine-reactive linker reagent. In certain embodiments, an antibody is subjected to denaturing conditions to reveal reactive nucleophilic groups such as lysine or cysteine.

The loading (drug/antibody ratio) of an ADC may be controlled in different ways, e.g., by: (i) limiting the molar excess of drug-linker intermediate or linker reagent relative to antibody, (ii) limiting the conjugation reaction time or temperature, (iii) partial or limiting reductive conditions for cysteine thiol modification, (iv) engineering by recombinant techniques the amino acid sequence of the antibody such that the number and position of cysteine residues is modified for control of the number and/or position of linker-drug attachments.

Cytotoxins suitable for use with the compositions and methods described herein include DNA-intercalating agents, (e.g., anthracyclines), agents capable of disrupting the mitotic spindle apparatus (e.g., vinca alkaloids, maytansine, maytansinoids, and derivatives thereof), RNA polymerase inhibitors (e.g., an amatoxin, such as a-amanitin, and derivatives thereof), and agents capable of disrupting protein biosynthesis (e.g., agents that exhibit rRNA N-glycosidase activity, such as saporin and ricin A-chain), among others known in the art.

In some embodiments, the cytotoxin is a microtubule-binding agent (for instance, maytansine or a maytansinoid), an amatoxin, pseudomonas exotoxin A, deBouganin, diphtheria toxin, saporin, an auristatin, an anthracycline, a calicheamicin, irinotecan, SN-38, a duocarmycin, a pyrrolobenzodiazepine, a pyrrolobenzodiazepine dimer, an indolinobenzodiazepine, an indolinobenzodiazepine dimer, an indolinobenzodiazepine pseudodimer, or a variant thereof, or another cytotoxic compound described herein or known in the art.

Additional details regarding cytotoxins that can be used in the anti-CD45 ADCs useful in the methods of the present disclosure are described below.

### Amatoxins

In some embodiments, the cytotoxin of the antibody-drug conjugate is an RNA polymerase inhibitor.

In some embodiments, the RNA polymerase inhibitor is an amatoxin or derivative thereof. In some embodiments, the cytotoxin of the antibody-drug conjugate as disclosed herein is an amatoxin or derivative thereof, such as an α-amanitin, β-amanitin, γ-amanitin, ε-amanitin, amanin, amaninamide, amanullin, amanullinic acid, proamanullin, or a derivative thereof. Structures of the various naturally occurring amatoxins are represented by formula II and accompanying Table 1, and are disclosed in, e.g., Zanotti et al., Int. J. Peptide Protein Res. 30, 1987, 450-459.

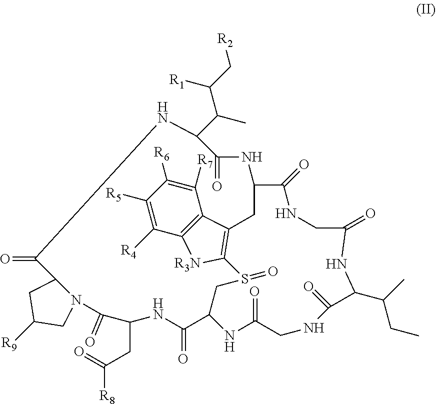

TABLE 1
Amatoxin structure table.

| Name | R₁ | R₂ | R₃, R₄ | R₅ | R₆, R₇ | R₈ | R₉ |
|---|---|---|---|---|---|---|---|
| α-amanitin | OH | OH | H | OH | H | NH₂ | OH |
| β-amanitin | OH | OH | H | OH | H | OH | OH |
| γ-amanitin | OH | H | H | OH | H | NH₂ | OH |
| ε-amanitin | OH | H | H | OH | H | OH | OH |
| Amanin | OH | OH | H | H | H | OH | OH |
| Amaninamide | OH | OH | H | H | H | NH₂ | OH |
| Amanullin | H | H | H | OH | H | NH₂ | OH |
| Amanullinic | H | H | H | OH | H | OH | OH |
| acid | | | | | | | |
| Proamanullin | H | H | H | OH | H | NH₂ | H |

Amatoxins may be isolated from a variety of mushroom species (e.g., Amanita phalloides, Galerina marginata, Lepiota brunneo-incarnata) or may be prepared semi-synthetically or synthetically. A member of this family, α-amanitin, is described in Wieland, Int. J. Pept. Protein Res. 1983, 22(3):257-276. A derivative of an amatoxin may be obtained by chemical modification of a naturally occurring compound (“semi-synthetic”), or may be obtained from an entirely synthetic source. Synthetic routes to various amatoxin derivatives are disclosed in, for example, U.S. Pat. No. 9,676,702 and in Perrin et al., J. Am. Chem. Soc. 2018, 140, p. 6513-6517, each of which is incorporated by reference herein in their entirety with respect to synthetic methods for preparing and derivatizing amatoxins.

Many positions on amatoxins or derivatives thereof can serve as the position to covalently bond the linking moiety L, and, hence the antibodies or antigen-binding fragments thereof. In some embodiments, the cytotoxin in the ADC's as disclosed herein is an amatoxin or derivative thereof represented by formula (III):

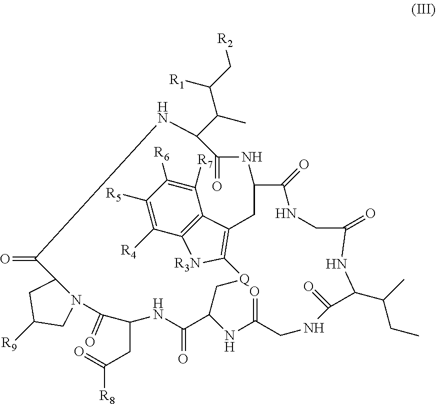

wherein R₁ is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₈ is OH, NH₂, or OR_{D};

R₉ is H, OH, or OR_{D};

X is —S—, —S(O)—, or —SO₂—; and

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl.

For instance, in one embodiment, amatoxins useful in conjunction with the compositions and methods described herein include compounds according to formula (IIIA)

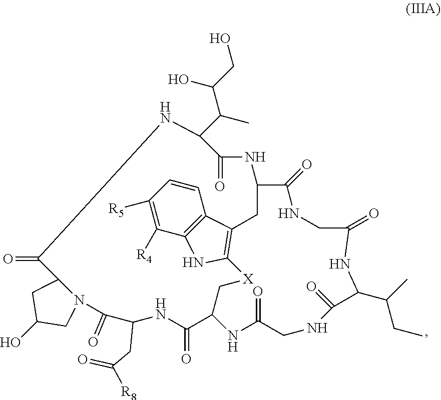

wherein R₄, R₅, X, and R₈ are each as defined above.

For instance, in one embodiment, amatoxins useful in conjunction with the compositions and methods described herein include compounds according to formula (IIIB), below:

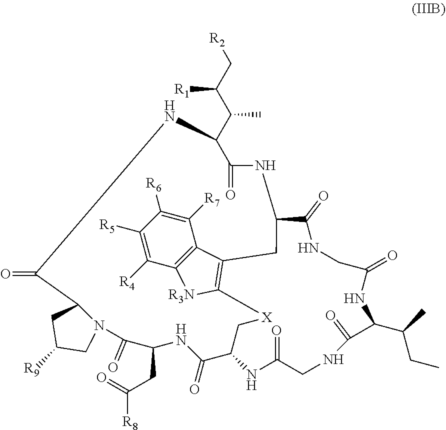

wherein R₁ is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₈ is OH, NH₂, or OR_{D};

R₉ is H, OH, or OR_{D};

X is —S—, —S(O)—, or —SO₂—; and

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl.

In one embodiment, amatoxins useful in conjunction with the compositions and methods described herein also include compounds according to formula (IIIC), below:

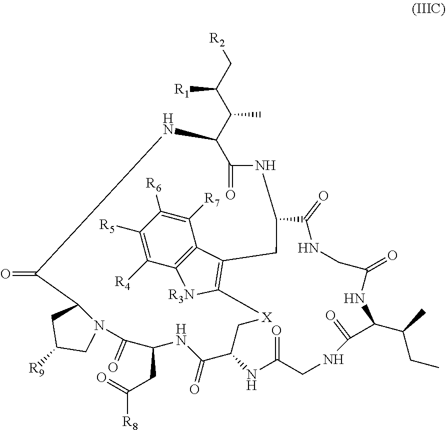

wherein R₁ is H, OH, or OR_{A};

R₂ is H, OH, or OR_{B};

R_{A} and R_{B}, when present, together with the oxygen atoms to which they are bound, combine to form an optionally substituted 5-membered heterocycloalkyl group;

R₃ is H or R_{D};

R₄ is H, OH, OR_{D}, or R_{D};

R₅ is H, OH, OR_{D}, or R_{D};

R₆ is H, OH, OR_{D}, or R_{D};

R₇ is H, OH, OR_{D}, or R_{D};

R₈ is OH, NH₂, or OR_{D};

R₉ is H, OH, or OR_{D};

X is —S—, —S(O)—, or —SO₂—; and

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl.

In one embodiment, the cytotoxin is an amanitin.

As described herein, amatoxins may be conjugated to an antibody, or an antigen-binding fragment thereof, for instance, by way of a linker moiety. Exemplary methods of amatoxin conjugation and linkers useful for such processes are described in the section entitled “Linkers for chemical conjugation,” as well as in Table 1, below. Exemplary linker-containing amatoxins useful for conjugation to an anti-CD45 antibody, or an antigen-binding fragment, in accordance with the compositions and methods described herein are shown in structural formulas (I), (IA), (IB), (IV), (IVA), and (IVB), recited herein.

For instance, the antibodies, or antigen-binding fragments, described herein may be bound to an amatoxin so as to form a conjugate represented by the formula Ab-Z-L-Am, wherein Ab is the antibody, or antigen-binding fragment thereof, L is a linker, Z is a chemical moiety and Am is an amatoxin. Many positions on amatoxins or derivatives thereof can serve as the position to covalently bond the linking moiety L, and, hence the antibodies or antigen-binding fragments thereof. In some embodiments, the amatoxin-linker conjugate Am-L-Z is represented by formula (I)

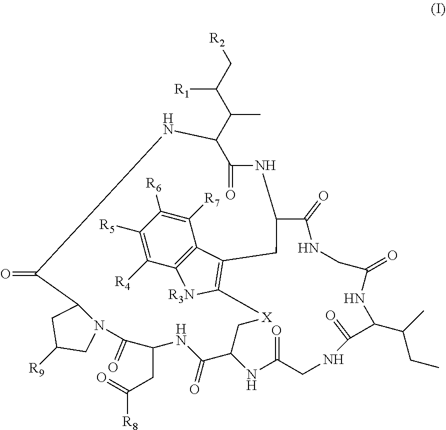

wherein R₁ is H, OH, OR_{A}, or OR_{C};

R₂ is H, OH, OR_{B}, or OR_{C};
R_{A} and R_{B}, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is L-Z;

R_{D} is optionally substituted C₁-C₆ alkyl, optionally substituted C₁-C₆ heteroalkyl, optionally substituted C₂-C₆ alkenyl, optionally substituted C₂-C₆ heteroalkenyl, optionally substituted C₂-C₆ alkynyl, optionally substituted C₂-C₆ heteroalkynyl, optionally substituted cycloalkyl, optionally substituted heterocycloalkyl, optionally substituted aryl, or optionally substituted heteroaryl;

L is a linker, such as optionally substituted C₁-C₆ alkylene, optionally substituted C₁-C₆ heteroalkylene, optionally substituted C₂-C₆ alkenylene, optionally substituted C₂-C₆ heteroalkenylene, optionally substituted C₂-C₆ alkynylene, optionally substituted C₂-C₆ heteroalkynylene, optionally substituted cycloalkylene, optionally substituted heterocycloalkylene, optionally substituted arylene, optionally substituted heteroarylene, a peptide (e.g., a dipeptide), —(C═O)—, a disulfide, a hydrazone, a —(CH₂CH₂O)ₚ— group, wherein p is an integer from 1-6, a ((CH₂)ₘO)ₙ(CH₂)ₘ— group, where n and each m are each independently selected from 1, 2, 3, 4, 5, 6, 7, 8, 9, and 10; or a combination thereof; and

Z is a chemical moiety that forms a coupling reaction between a reactive substituent present on L and a reactive substituent present within an antibody, antigen-binding fragment thereof, or soluble ligand that binds CD45.

In some embodiments, the cytotoxin contains one R_{C} substituent.

In some embodiments, R_{A} and R_{B}, together with the oxygen atoms to which they are bound, combine to form:

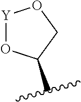

wherein Y is —(C═O)—, —(C═S)—, —(C═NR_{E})—, or —(CR_{E}R_{E′})—; and

R_{E} and R_{E′} are each independently optionally substituted C₁-C₆ alkylene-R_{C}, optionally substituted C₁-C₆ heteroalkylene-R_{C}, optionally substituted C₂-C₆ alkenylene-R_{C}, optionally substituted C₂-C₆ heteroalkenylene-R_{C}, optionally substituted C₂-C₆ alkynylene-R_{C}, optionally substituted C₂-C₆ heteroalkynylene-R_{C}, optionally substituted cycloalkylene-R_{C}, optionally substituted heterocycloalkylene-R_{C}, optionally substituted arylene-R_{C}, or optionally substituted heteroarylene-R_{C}.

In some embodiments, Am-L-Z is represented by formula (I), wherein

R₁ is H, OH, OR_{A}, or OR_{C};

R₂ is H, OH, OR_{B}, or OR_{C};

R_{A} and R_{B}, together with the oxygen atoms to which they are bound, combine to form:

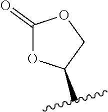 [SMILES: *[C@@H]1COC(=O)O1]

R₃ is H or R_{C};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, or NHR_{C};

R₉ is H or OH; and

wherein R_{C} and R_{D} are each as defined above.

In some embodiments, Am-L-Z is represented by formula (I),

wherein

R₁ is H, OH, OR_{A}, or OR_{C};

R₂ is H, OH, OR_{B}, or OR_{C};

R_{A} and R_{B}, together with the oxygen atoms to which they are bound, combine to form:

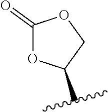 [SMILES: *[C@@H]1COC(=O)O1]

R₃ is H or R_{C};

R₄ and R₅ are each independently H, OH, OR_{C}, R_{C}, or OR_{D};

R₆ and R₇ are each H;

R₈ is OH, NH₂, OR_{C}, or NHR_{C};

R₉ is H or OH; and

wherein X and R_{C} are as defined above.

In some embodiments, Am-L-Z is represented by formula (I)

wherein R₁ is H, OH, or OR_{A};

R₂ is H, OH, or OR_{B};

R_{A} and R_{B}, together with the oxygen atoms to which they are bound, combine to form:

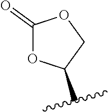 [SMILES: *[C@@H]1COC(=O)O1]

R₃, R₄, R₆, and R₇ are each H;

R₅ is OR_{C};

R₈ is OH or NH₂;

R₉ is H or OH; and

wherein R_{C} is as defined above. Such amatoxin conjugates are described, for example, in US Patent Application Publication No. 2016/0002298, the disclosure of which is incorporated herein by reference in its entirety.

In some embodiments, Am-L-Z is represented by formula (I), wherein:

R₁ and R₂ are each independently H or OH;

R₃ is R_{C};

R₄, R₆, and R₇ are each H;

R₅ is H, OH, or OC₁-C₆ alkyl;

R₈ is OH or NH₂;

R₉ is H or OH; and

wherein R_{C} is as defined above.

In some embodiments, Am-L-Z is represented by formula (I),

wherein

R₁ and R₂ are each independently H or OH;

R₃, R₆, and R₇ are each H;

R₄ and R₅ are each independently H, OH, OR_{C}, or R_{C};

R₈ is OH or NH₂;

R₉ is H or OH; and

wherein R_{C} is as defined above. Such amatoxin conjugates are described, for example, in US Patent Application Publication No. 2015/0218220, the disclosure of which is incorporated herein by reference in its entirety.

In some embodiments, Am-L-Z is represented by formula (I), wherein

R₁ and R₂ are each independently H or OH;

R₃, R₆, and R₇ are each H;

R₄ and R₅ are each independently H or OH;

R₈ is OH, NH₂, OR_{C}, or NHR_{C};

R₉ is H or OH; and

wherein R_{C} is as defined above. Such amatoxin conjugates are described, for example, in U.S. Pat. Nos. 9,233,173 and 9,399,681, the disclosures of each of which are incorporated herein by reference in their entirety.

In some embodiments, the linker L and the chemical moiety Z, taken together as L-Z, is

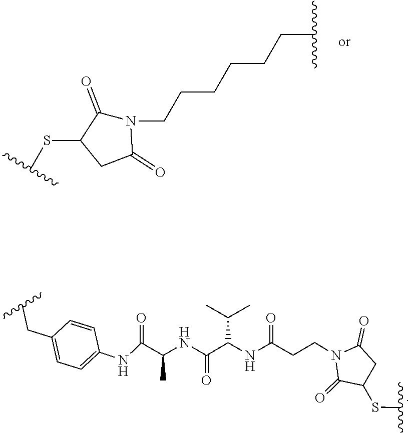

where S is a sulfur atom which represents the reactive substituent present within an antibody, or antigen-binding fragment thereof, that binds CD45 (e.g., from the —SH group of a cysteine residue).

In some embodiments, L-Z is

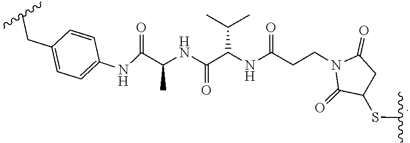

In some embodiments, L-Z is

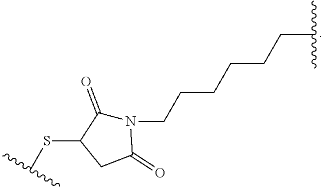 [SMILES: *CCCCCCN1C(=O)CC(S*)C1=O]

In some embodiments, Am-L-Z is represented by formula (IA)

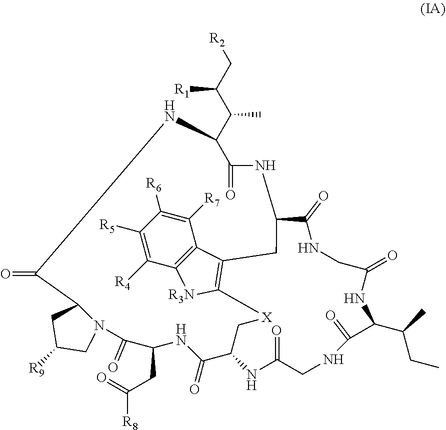

wherein:

R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, optionally substituted heteroarylene, a peptide (e.g., a dipeptide), -(C═O)—, a disulfide, a hydrazone, a —(CH₂CH₂O)ₚ— group, wherein p is an integer from 1-6, a ((CH₂)ₘO)ₙ(CH₂)ₘ— group, w here n and each m are each independently selected from 1, 2, 3, 4, 5, 6, 7, 8, 9, and 10; or a combination thereof;

Z is a chemical moiety formed from a coupling reaction between a reactive substituent Z′, present on L and a reactive substituent present within an antibody, or antigen-binding fragment thereof, that binds CD45; and

wherein Am contains exactly one R_{C} substituent.

In some embodiments, Am-L-Z is represented by formula (IB)

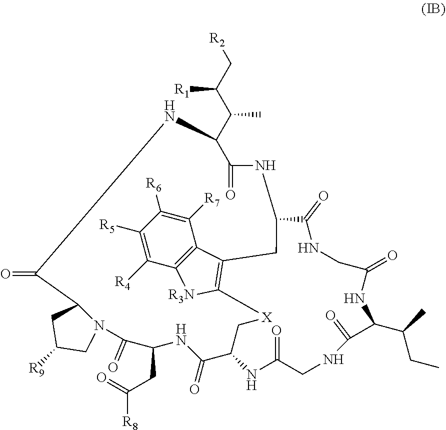

wherein:

R₁ is H, OH, OR_{A}, or OR_{C};

R₂ is H, OH, OR_{B}, or OR_{C};

R_{A} and R_{B}, when present, together with the oxygen atoms to which they are bound, combine to form an optionally substituted 5-membered heterocycloalkyl group;

R₃ is H, R_{C}, or R_{D};

R₄ is H, OH, OR_{C}, OR_{D}, R_{C}, or R_{D};

R₅ is H, OH, OR_{C}, OR_{D}, R_{C}, or R_{D};

R₆ is H, OH, OR_{C}, OR_{D}, R_{C}, or R_{D};

R₇ is H, OH, OR_{C}, OR_{D}, R_{C}, or R_{D};

R₈ is OH, NH₂, OR_{C}, OR_{D}, NHR_{C}, or NR_{C}R_{D};

R₉ is H, OH, OR_{C}, or OR_{D};

X is —S—, —S(O)—, or —SO₂—;

R_{C} is -L-Z;

R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl;

L is a linker, such as optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, optionally substituted heteroarylene, a dipeptide, —(C═O)—, a peptide, a disulfide, a hydrazone, a —(CH₂CH₂O)ₚ— group, wherein p is an integer from 1-6, a ((CH₂)ₘO)ₙ(CH₂)ₘ— group, where n and each m are each independently selected from 1, 2, 3, 4, 5, 6, 7, 8, 9, and 10; or a combination thereof;

Z is a chemical moiety formed from a coupling reaction between a reactive substituent Z′, present on L and a reactive substituent present within an antibody, or antigen-binding fragment thereof, that binds CD45; and

wherein Am contains exactly one R_{C} substituent.

In some embodiments, the linker comprises a —(CH)₂ₙ— unit, where n is an integer from 2-6. In some embodiments, the linker includes —((CH₂)ₙ where n is 6.

In some embodiments, the linker L and the chemical moiety Z, taken together as L-Z, is

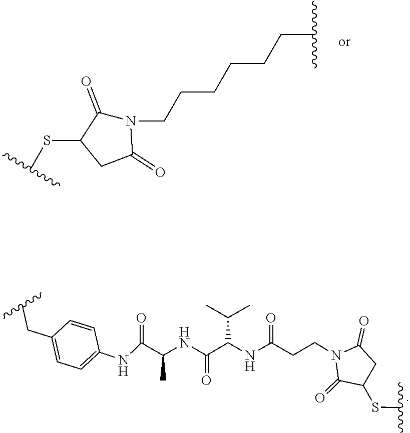

where S is a sulfur atom which represents the reactive substituent present within an antibody, or antigen-binding fragment thereof, that binds CD45 (e.g., from the —SH group of a cysteine residue).

In some embodiments, L-Z is

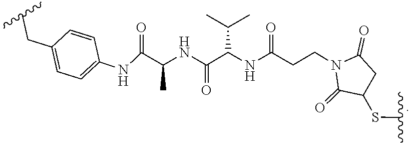

In some embodiments, L-Z is

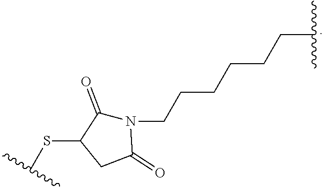 [SMILES: *CCCCCCN1C(=O)CC(S*)C1=O]

In some embodiments, the conjugate Am-L-Z-Ab is represented by any one of the following structural formulas:

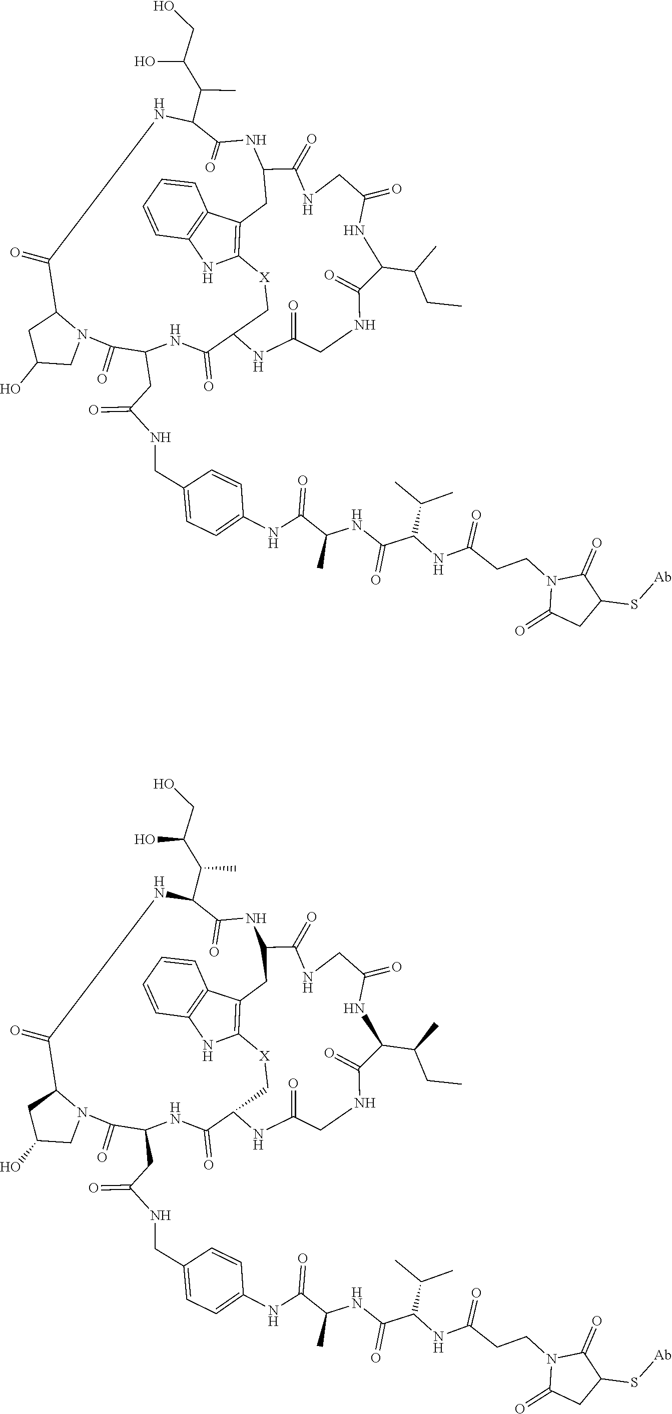

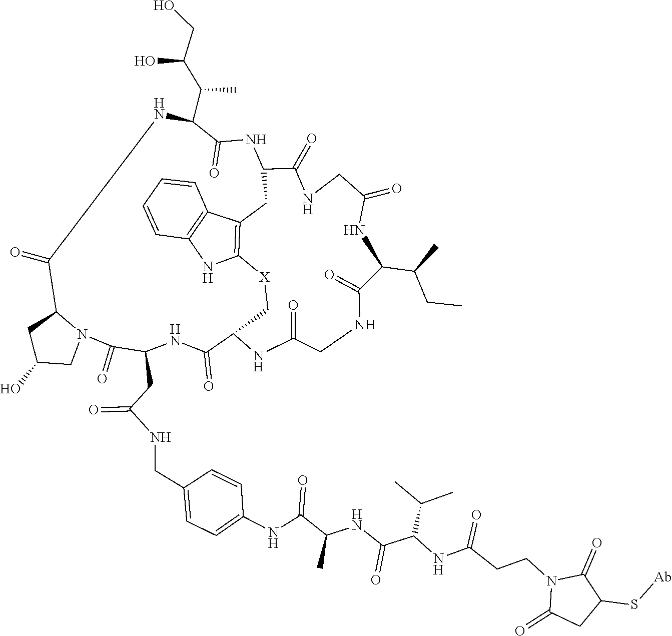

In some embodiments, Am-L-Z-Ab is represented by the structural formula

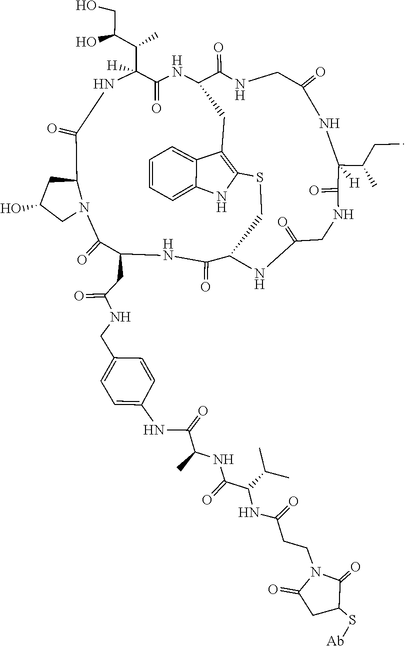

In some embodiments, Am-L-Z′, where Am-L-Z′ is the precursor to Am-L-Z-Ab, is

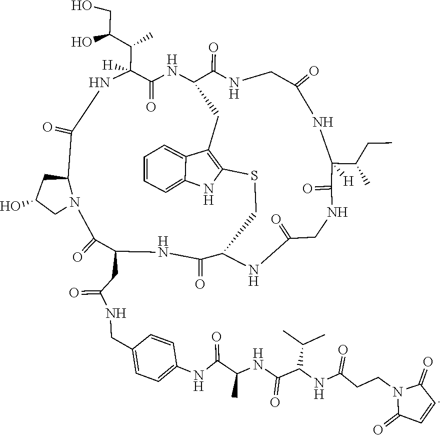

wherein the maleimide (reactive substituent Z′) reacts with a thiol group found on a cysteine in the antibody.

In some embodiments, Am-L-Z is represented by formula (IV), formula (IVA), or formula (IVB):

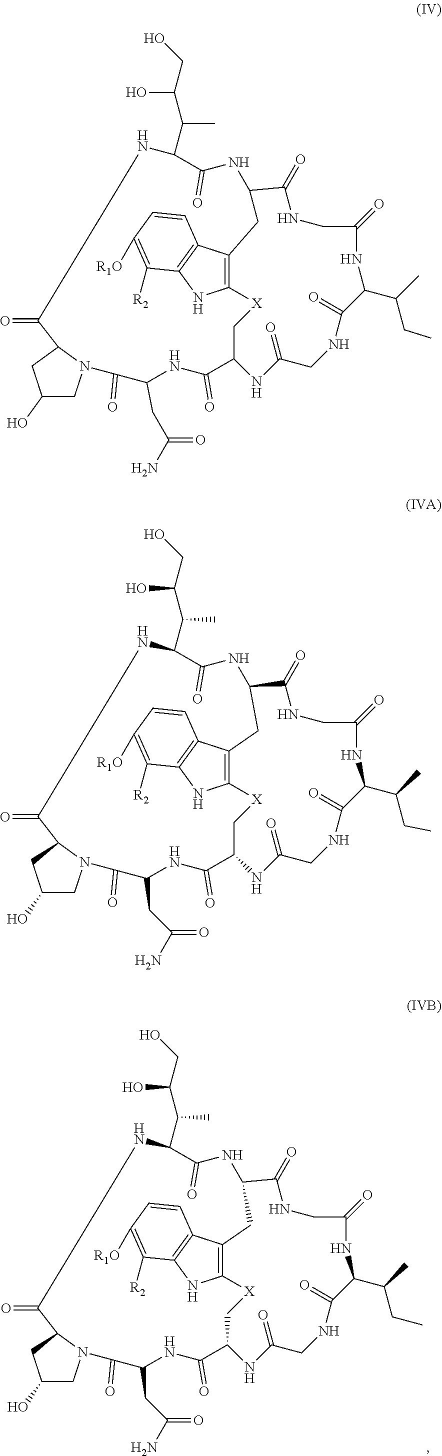

wherein X is S, SO, or SO₂; R₁ is H or a linker covalently bound to the antibody or antigen-binding fragment thereof through a chemical moiety Z, formed from a coupling reaction between a reactive substituent Z′ present on the linker and a reactive substituent present within an antibody, or antigen-binding fragment thereof; and R₂ is H or a linker covalently bound to the antibody or antigen-binding fragment thereof through a chemical moiety Z, formed from a coupling reaction between a reactive substituent Z′ present on the linker and a reactive substituent present within an antibody, or antigen-binding fragment thereof; wherein when R₁ is H, R₂ is the linker, and when R₂ is H, R₁ is the linker.

In some embodiments, the linker comprises a —(CH₂)ₙ— unit, where n is an integer from 2-6. In some embodiments, R₁ is the linker and R₂ is H, and the linker and chemical moiety, together as L-Z, is

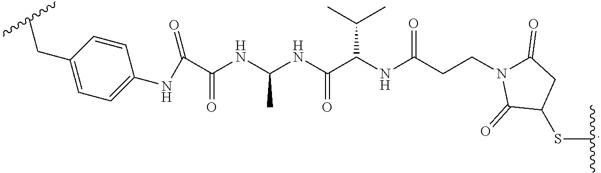

In some embodiments, R₁ is the linker and R₂ is H, and the linker and chemical moiety, together as L-Z, is

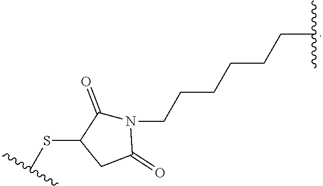 [SMILES: *CCCCCCN1C(=O)CC(S*)C1=O]

In some embodiments, Am-L-Z-Ab is

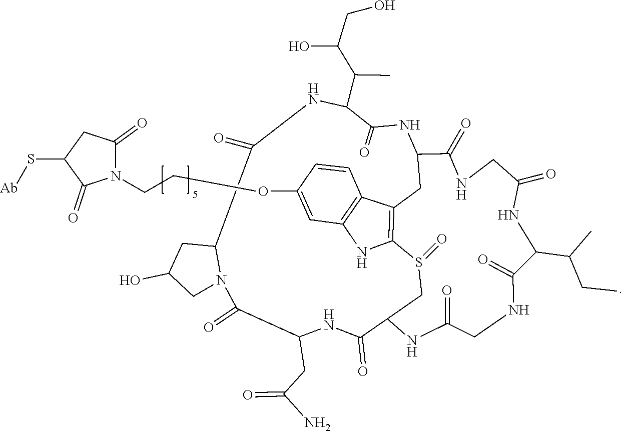

In some embodiments, Ab-Z-L-Am is

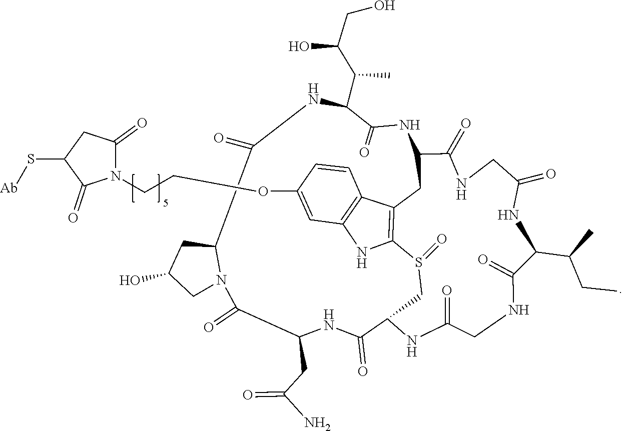

In some embodiments, Am-L-Z-Ab is:

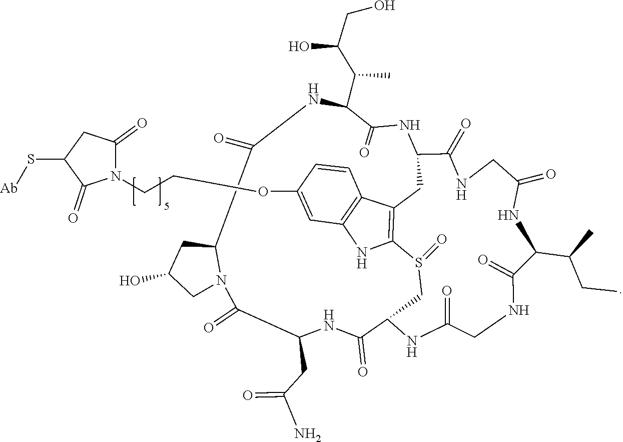

In some embodiments, the Am-L-Z-Ab precursor (i.e., Am-L-Z′) is one of:

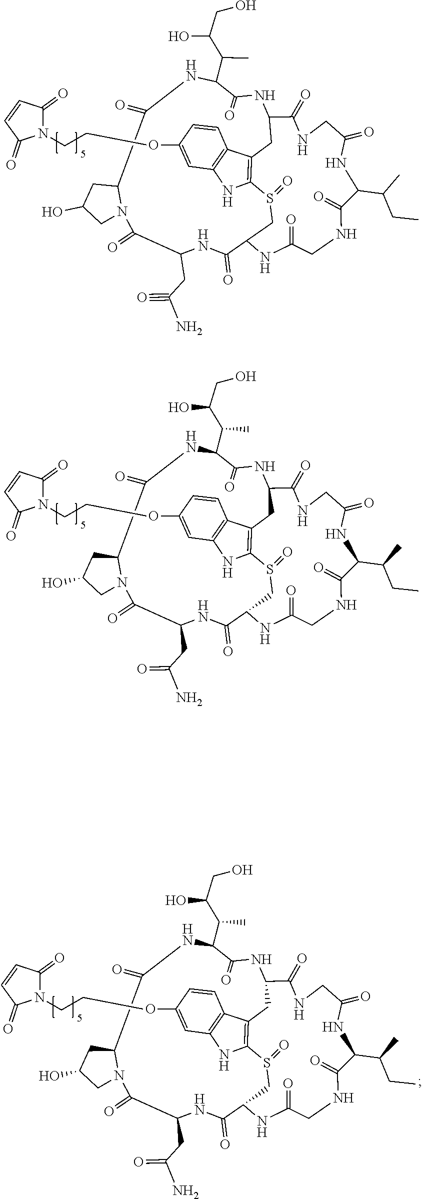

wherein the maleimide reacts with a thiol group found on a cysteine in the CD-45 antibody.

Additional amatoxins that may be used for conjugation to an antibody, or antigen-binding fragment thereof, in accordance with the compositions and methods described herein are described, for example, in WO 2016/142049; WO 2016/071856; and WO 2017/046658, the disclosures of each of which are incorporated herein by reference in their entirety. For instance, antibodies, antigen-binding fragments thereof, and ligands that recognize and bind CD45 can be conjugated to a-amanitin or a variant thereof, as described in US 2015/0218220, the disclosure of which is incorporated herein by reference as it pertains, for example, to amatoxins, such as a-amanitin and variants thereof, as well as covalent linkers that can be used for covalent conjugation. Synthetic methods of making amatoxins are described in, for example, U.S. Pat. No. 9,676,702, which is incorporated by reference herein with respect to the synthetic methods disclosed therein.

The linker L may be attached to the amatoxin (e.g., an amatoxin of formula III, IIIA, IIIB, or IIIC) at any one of several possible positions (e.g., any of R¹-R⁹) to provide an amatoxin-linker conjugate of formula I, IA, IB, IV, IVA, or IVB.

In some embodiments, the linker is attached at position R¹. In some embodiments, the linker is attached at position R². In some embodiments, the linker is attached at position R³. In some embodiments, the linker is attached at position R⁴. In some embodiments, the linker is attached at position R⁵. In some embodiments, the linker is attached at position R⁶. In some embodiments, the linker is attached at position R⁷. In some embodiments, the linker is attached at position R⁸. In some embodiments, the linker is attached at position R⁹.

In some embodiments, the cytotoxin is an a-amanitin. In some embodiments, the linker includes a hydrazine, a disulfide, a thioether or a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6.

In some embodiments, the linker includes a —(CH₂)ₙ— unit, where n is an integer from 2-6. In some embodiments, the linker is -PAB-Cit-Val-((C═O)(CH₂)ₙ—. In some embodiments, the linker is -PAB-Ala-Val-((C═O)(CH₂)ₙ—. In some embodiments, the linker L and the chemical moiety Z, taken together as L-Z, is

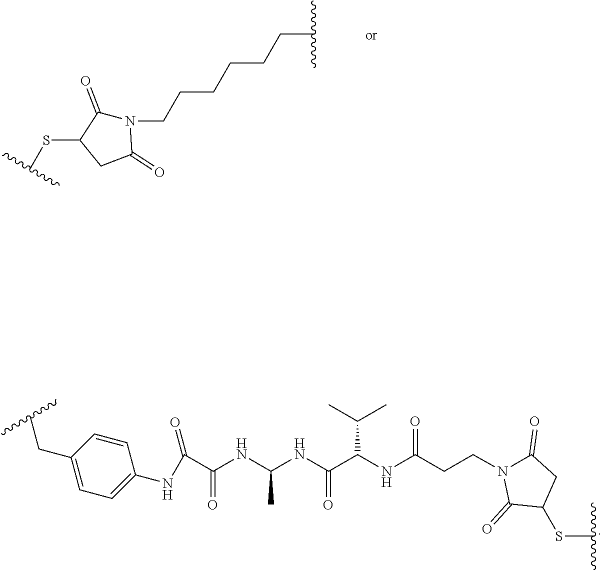

Auristatins

Anti-CD45 antibodies and antigen-binding fragments thereof described herein can be conjugated to a cytotoxin that is an auristatin (U.S. Pat. Nos. 5,635,483; 5,780,588). Auristatins are anti-mitotic agents that interfere with microtubule dynamics, GTP hydrolysis, and nuclear and cellular division (Woyke et al (2001) Antimicrob. Agents and Chemother. 45(12):3580-3584) and have anticancer (U.S. Pat. No. 5,663,149) and antifungal activity (Pettit et al (1998) Antimicrob. Agents Chemother. 42:2961-2965). (U.S. Pat. Nos. 5,635,483; 5,780,588). The auristatin drug moiety may be attached to the antibody through the N (amino) terminus or the C (carboxyl) terminus of the peptidic drug moiety (WO 02/088172).

Exemplary auristatin embodiments include the N-terminus linked monomethylauristatin drug moieties DE and DF (MMAE and MMAF, respectively), disclosed in Senter et al, Proceedings of the American Association for Cancer Research, Volume 45, Abstract Number 623, presented Mar. 28, 2004, the disclosure of which is expressly incorporated by reference in its entirety.

An exemplary auristatin embodiment is MMAE:

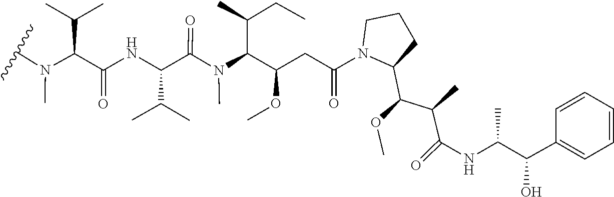 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@H](C)[C@@H](O)c1ccccc1)OC)C(C)C)C(C)C]

wherein the wavy line indicates the point of covalent attachment to the linker of an antibody-linker conjugate (-L-Z-Ab as described herein).

Another exemplary auristatin embodiment is MMAF:

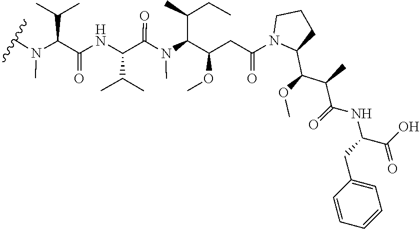 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)C(=O)O)OC)C(C)C)C(C)C]

wherein the wavy line indicates the point of covalent attachment to the linker of an antibody-linker conjugate (-L-Z-Ab as described herein), as disclosed in US 2005/0238649.

Auristatins may be prepared according to the methods of: U.S. Pat. Nos. 5,635,483; 5,780,588; Pettit et al (1989) J. Am. Chem. Soc. 111:5463-5465; Pettit et al (1998) Anti-Cancer Drug Design 13:243-277; Pettit, G. R., et al. Synthesis, 1996, 719-725; Pettit et al (1996) J. Chem. Soc. Perkin Trans. 15:859-863; and Doronina (2003) Nat. Biotechnol. 21(7):778-784.

Maytansinoids

Antibodies and antigen-binding fragments thereof described herein can be conjugated to a cytotoxin that is a microtubule binding agent. In some embodiments, the microtubule binding agent is a maytansine, a maytansinoid or a maytansinoid analog. Maytansinoids are mitototic inhibitors which bind microtubules and act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533. Maytansinoid drug moieties are attractive drug moieties in antibody drug conjugates because they are: (i) relatively accessible to prepare by fermentation or chemical modification, derivatization of fermentation products, (ii) amenable to derivatization with functional groups suitable for conjugation through the non-disulfide linkers to antibodies, (iii) stable in plasma, and (iv) effective against a variety of tumor cell lines.

Examples of suitable maytansinoids include esters of maytansinol, synthetic maytansinol, and maytansinol analogs and derivatives. Included herein are any cytotoxins that inhibit microtubule formation and that are highly toxic to mammalian cells, as are maytansinoids, maytansinol, and maytansinol analogs, and derivatives.

Examples of suitable maytansinol esters include those having a modified aromatic ring and those having modifications at other positions. Such suitable maytansinoids are disclosed in U.S. Pat. Nos. 4,137,230; 4,151,042; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,362,663; 4,364,866; 4,424,219; 4,450,254; 4,322,348; 4,362,663; 4,371,533; 5,208,020; 5,416,064; 5,475,092; 5,585,499; 5,846,545; 6,333,410; 7,276,497; and 7,473,796, the disclosures of each of which are incorporated herein by reference as they pertain to maytansinoids and derivatives thereof.

In some embodiments, the antibody-drug conjugates (ADCs) of the present disclosure utilize the thiol-containing maytansinoid (DM1), formally termed N²′-deacetyl-N²′-(3-mercapto-1-oxopropyl)-maytansine, as the cytotoxic agent. DM1 is represented by the following structural formula:

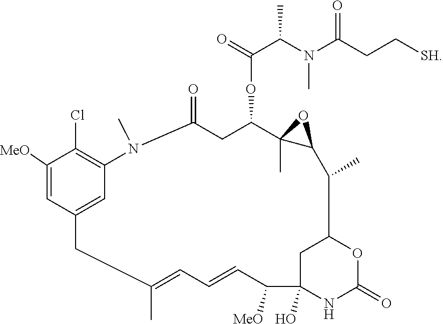 [SMILES: COc1cc2cc(c1Cl)N(C)C(=O)C[C@H](OC(=O)[C@H](C)N(C)C(=O)CCS)[C@]1(C)O[C@H]1[C@H](C)C1C[C@@](O)(NC(=O)O1)[C@H](OC)/C=C/C=C(\C)C2]

In another embodiment, the conjugates of the present disclosure utilize the thiol-containing maytansinoid N²′-deacetyl-N²′(4-methyl-4-mercapto-1-oxopentyl)-maytansine (e.g., DM4) as the cytotoxic agent. DM4 is represented by the following structural formula:

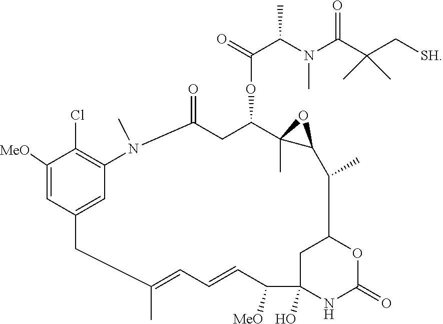 [SMILES: COc1cc2cc(c1Cl)N(C)C(=O)C[C@H](OC(=O)[C@H](C)N(C)C(=O)C(C)(C)CS)[C@]1(C)O[C@H]1[C@H](C)C1C[C@@](O)(NC(=O)O1)[C@H](OC)/C=C/C=C(\C)C2]

Another maytansinoid comprising a side chain that contains a sterically hindered thiol bond is N²′-deacetyl-N-²′(4-mercapto-1-oxopentyl)-maytansine (termed DM3), represented by the following structural formula:

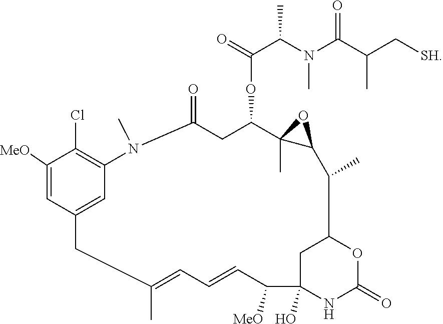 [SMILES: COc1cc2cc(c1Cl)N(C)C(=O)C[C@H](OC(=O)[C@H](C)N(C)C(=O)C(C)CS)[C@]1(C)O[C@H]1[C@H](C)C1C[C@@](O)(NC(=O)O1)[C@H](OC)/C=C/C=C(\C)C2]

Each of the maytansinoids taught in U.S. Pat. Nos. 5,208,020 and 7,276,497, can also be used in the conjugates of the present disclosure. In this regard, the entire disclosure of U.S. Pat. Nos. 5,208,020 and 7,276,697 is incorporated herein by reference.

Many positions on maytansinoids can serve as the position to covalently bond the linking moiety and, hence the antibodies or antigen-binding fragments thereof (-L-Z-Ab as described herein). For example, the C-3 position having a hydroxyl group, the C-14 position modified with hydroxymethyl, the C-15 position modified with hydroxy and the C-20 position having a hydroxy group are all expected to be useful. In some embodiments, the C-3 position serves as the position to covalently bond the linker moiety, and in some particular embodiments, the C-3 position of maytansinol serves as the position to covalently bond the linking moiety. There are many linking groups known in the art for making antibody-maytansinoid conjugates, including, for example, those disclosed in U.S. Pat. Nos. 5,208,020, 6,441,163, and EP Patent No. 0425235 B1; Chari et al., Cancer Research 52:127-131 (1992); and U.S. 2005/0169933 A1, the disclosures of which are hereby expressly incorporated by reference. Additional linking groups are described and exemplified herein.

The present disclosure also includes various isomers and mixtures of maytansinoids and conjugates. Certain compounds and conjugates of the present disclosure may exist in various stereoisomeric, enantiomeric, and diastereomeric forms. Several descriptions for producing such antibody-maytansinoid conjugates are provided in U.S. Pat. Nos. 5,208,020; 5,416,064; 6,333,410; 6,441,163; 6,716,821; and 7,368,565, each of which is incorporated herein in its entirety.

Anthracyclines

In other embodiments, the antibodies and antigen-binding fragments thereof described herein can be conjugated to a cytotoxin that is an anthracycline molecule. Anthracyclines are antibiotic compounds that exhibit cytotoxic activity. Studies have indicated that anthracyclines may operate to kill cells by a number of different mechanisms including: 1) intercalation of the drug molecules into the DNA of the cell thereby inhibiting DNA-dependent nucleic acid synthesis; 2) production by the drug of free radicals which then react with cellular macromolecules to cause damage to the cells or 3) interactions of the drug molecules with the cell membrane [see, e.g., C. Peterson et al., “Transport And Storage Of Anthracycline In Experimental Systems And Human Leukemia” in Anthracycline Antibiotics In Cancer Therapy; N. R. Bachur, “Free Radical Damage” id. at pp. 97-102]. Because of their cytotoxic potential anthracyclines have been used in the treatment of numerous cancers such as leukemia, breast carcinoma, lung carcinoma, ovarian adenocarcinoma and sarcomas [see e.g., P. H-Wiernik, in Anthracycline: Current Status And New Developments p 11]. Commonly used anthracyclines include doxorubicin, epirubicin, idarubicin and daunomycin. In some embodiments, the cytotoxin is an anthracycline selected from the group consisting of daunorubicin, doxorubicin, epirubicin, and idarubicin. Representative examples of anthracyclines include, but are not limited to daunorubicin (Cerubidine; Bedford Laboratories), doxorubicin (Adriamycin; Bedford Laboratories; also referred to as doxorubicin hydrochloride, hydroxy-daunorubicin, and Rubex), epirubicin (Ellence; Pfizer), and idarubicin (Idamycin; Pfizer Inc.)

The anthracycline analog, doxorubicin (ADRIAMYCINO) is thought to interact with DNA by intercalation and inhibition of the progression of the enzyme topoisomerase II, which unwinds DNA for transcription. Doxorubicin stabilizes the topoisomerase II complex after it has broken the DNA chain for replication, preventing the DNA double helix from being resealed and thereby stopping the process of replication. Doxorubicin and daunorubicin (DAUNOMYCIN) are prototype cytotoxic natural product anthracycline chemotherapeutics (Sessa et al., (2007) Cardiovasc. Toxicol. 7:75-79).

One non-limiting example of a suitable anthracycline for use herein is PNU-159682 (“PNU”). PNU exhibits greater than 3000-fold cytotoxicity relative to the parent nemorubicin (Quintieri et al., Clinical Cancer Research 2005, 11, 1608-1617). PNU is represented by structural formula:

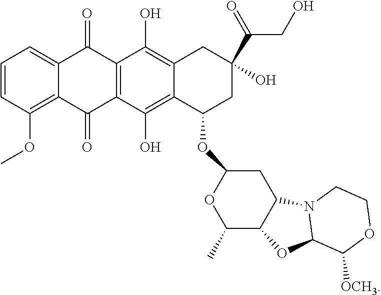

Multiple positions on anthracyclines such as PNU can serve as the position to covalently bond the linking moiety and, hence the anti-CD45 antibodies or antigen-binding fragments thereof as described herein. For example, linkers may be introduced through modifications to the hydroxymethyl ketone side chain.

In some embodiments, the cytotoxin is a PNU derivative represented by structural formula:

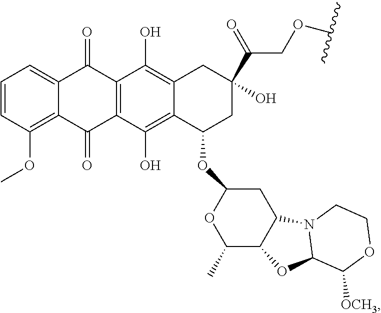

wherein the wavy line indicates the point of covalent attachment to the linker of the ADC as described herein.

In some embodiments, the cytotoxin is a PNU derivative represented by structural formula:

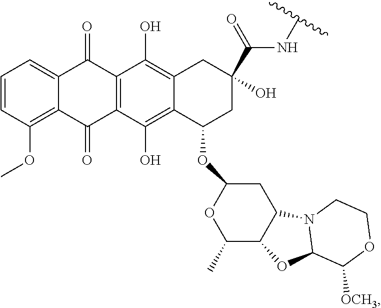

wherein the wavy line indicates the point of covalent attachment to the linker of the ADC as described herein.

Pyrrolobenzodiazepines (PBDs)

In other embodiments, the anti-CD45 antibodies or antigen-binding fragments thereof described herein can be conjugated to a cytotoxin that is a pyrrolobenzodiazepine (PBD) or a cytotoxin that comprises a PBD. PBDs are natural products produced by certain actinomycetes and have been shown to be sequence selective DNA alkylating compounds. PBD cytotoxins include, but are not limited to, anthramycin, dimeric PBDs, and those disclosed in, for example, Hartley, J A (2011) The development of pyrrolobenzodiazepines as antitumour agents. Expert Opin Inv Drug, 20(6), 733-744 and Antonow D, Thurston D E (2011) Synthesis of DNA-interactive pyrrolo[2,1-c][1,4]benzodiazepines (PBDs). Chem Rev 111: 2815-2864.

In some embodiments, the cytotoxin may be a pyrrolobenzodiazepine dimer represented by the formula:

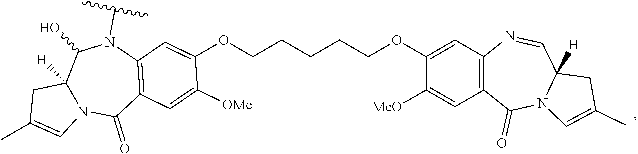

wherein the wavy line indicates the attachment point of the linker.

In some embodiments, the cytotoxin is conjugated to the antibody, or the antigen-binding fragment thereof, by way of a maleimidocaproyl linker.

In some embodiments, the linker comprises one or more of a peptide, oligosaccharide, —(CH₂)ₚ—, —(CH₂CH₂O)_{q}—, —(C═O)(CH₂)ᵣ—, —(C═O)(CH₂CH₂O)ₜ—, —(NHCH₂CH₂)ᵤ—, -PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB, wherein each of p, q, r, t, and u are integers from 1-12, selected independently for each occurrence.

In some embodiments, the linker has the structure of formula:

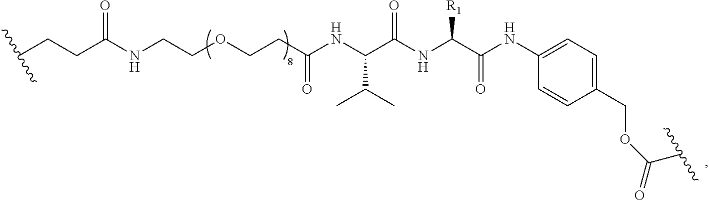

wherein R₁ is CH₃ (Ala) or (CH₂)₃NH(CO)NH₂ (Cit).

In some embodiments, the linker, prior to conjugation to the antibody and including the reactive substituent Z′, taken together as L-Z′, has the structure:

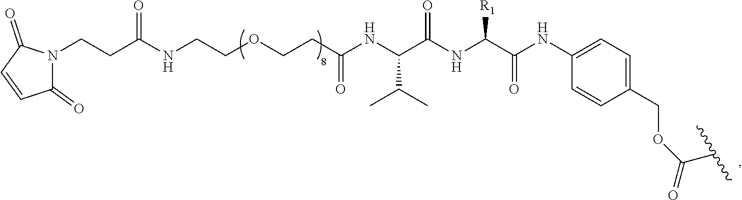

wherein the wavy line indicates the attachment point to the cytotoxin (e.g., a PBD). In certain embodiments, R₁ is CH₃.

In some embodiments, the cytotoxin-linker conjugate, prior to conjugation to the antibody and including the reactive substituent Z′, taken together as Cy-L-Z′, has the structure of formula:

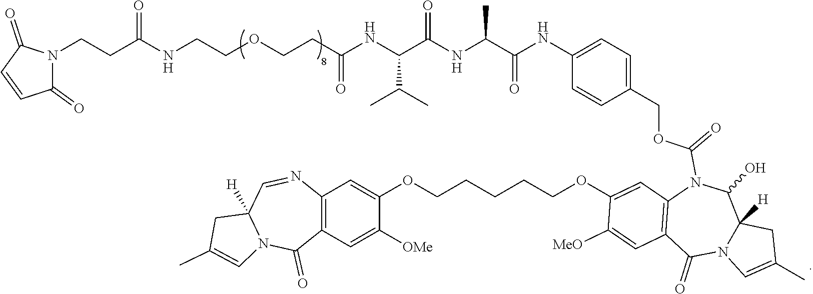

This particular cytotoxin-linker conjugate is known as tesirine (SG3249), and has been described in, for example, Howard et al., ACS Med. Chem. Lett. 2016, 7(11), 983-987, the disclosure of which is incorporated by reference herein in its entirety.

In some embodiments, the cytotoxin may be a pyrrolobenzodiazepine dimer represented by formula:

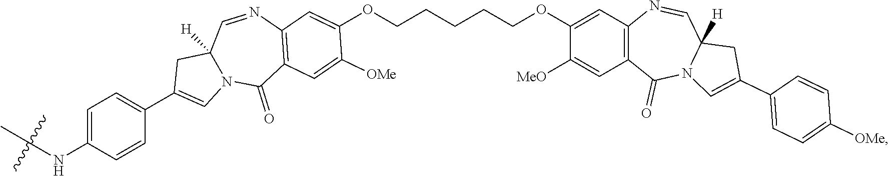 [SMILES: *Nc1ccc(C2=CN3C(=O)c4cc(OC)c(OCCCCCOc5cc6c(cc5OC)C(=O)N5C=C(c7ccc(OC)cc7)C[C@H]5C=N6)cc4N=C[C@@H]3C2)cc1]

wherein the wavy line indicates the attachment point of the linker.

In some embodiments, the cytotoxin-linker conjugate, prior to conjugation to the antibody and including the reactive substituent Z′, taken together as Cy-L-Z′, has the structure of formula:

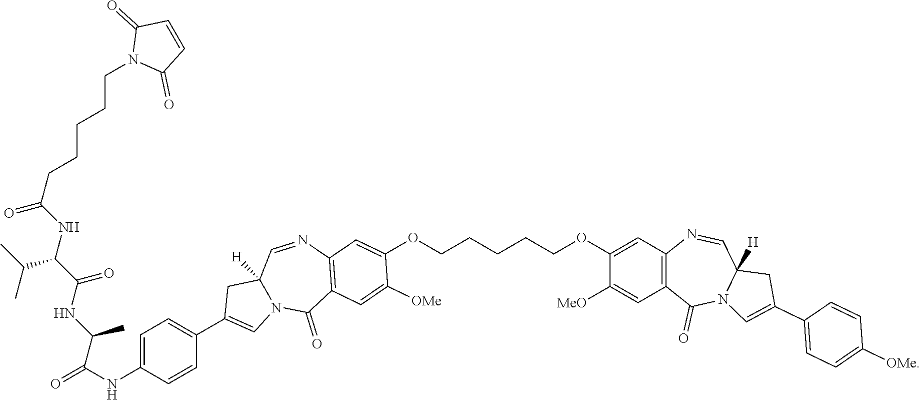 [SMILES: COc1ccc(C2=CN3C(=O)c4cc(OC)c(OCCCCCOc5cc6c(cc5OC)C(=O)N5C=C(c7ccc(NC(=O)[C@H](C)NC(=O)[C@@H](NC(=O)CCCCCN8C(=O)C=CC8=O)C(C)C)cc7)C[C@H]5C=N6)cc4N=C[C@@H]3C2)cc1]

This particular cytotoxin-linker conjugate is known as talirine, and has been described, for example, in connection with the ADC Vadastuximab talirine (SGN-CD33A), Mantaj et al., Angewandte Chemie International Edition English 2017,56, 462-488, the disclosure of which is incorporated by reference herein in its entirety.

In some embodiments, the cytotoxin may be an indolinobenzodiazepine pseudodimer having the structure of formula:

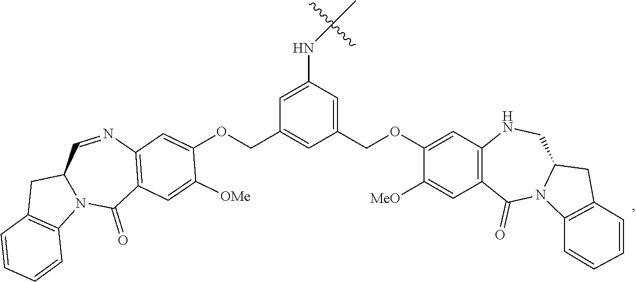 [SMILES: *Nc1cc(COc2cc3c(cc2OC)C(=O)N2c4ccccc4C[C@H]2C=N3)cc(COc2cc3c(cc2OC)C(=O)N2c4ccccc4C[C@H]2CN3)c1]

wherein the wavy line indicates the attachment point of the linker.

In some embodiments, the cytotoxin-linker conjugate, prior to conjugation to the antibody and including the reactive substituent Z′, taken together as Cy-L-Z′, has the structure of formula:

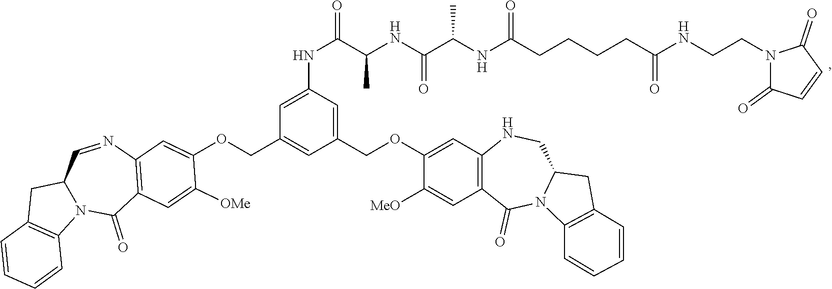 [SMILES: COc1cc2c(cc1OCc1cc(COc3cc4c(cc3OC)C(=O)N3c5ccccc5C[C@H]3CN4)cc(NC(=O)[C@H](C)NC(=O)[C@H](C)NC(=O)CCCCC(=O)NCCN3C(=O)C=CC3=O)c1)N=C[C@@H]1Cc3ccccc3N1C2=O]

which comprises the ADC IMGN632, disclosed in, for example, International Patent Application Publication No. WO2017004026, which is incorporated by reference herein.

Calicheamicin

In other embodiments, the antibodies and antigen-binding fragments thereof described herein can be conjugated to a cytotoxin that is an enediyne antitumor antibiotic (e.g., calicheamicins, ozogamicin). The calicheamicin family of antibiotics are capable of producing double-stranded DNA breaks at sub-picomolar concentrations. For the preparation of conjugates of the calicheamicin family, see U.S. Pat. Nos. 5,712,374; 5,714,586; 5,739,116; 5,767,285; 5,770,701; 5,770,710; 5,773,001; and 5,877,296 (all to American Cyanamid Company). Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents to American Cyanamid.

An exemplary calicheamicin is designated γ₁, which is herein referenced simply as gamma, and has the structural formula:

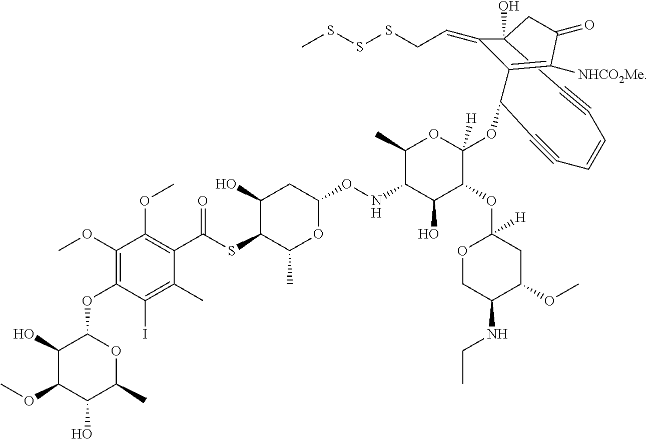

In some embodiments, the calicheamicin may be a gamma-calicheamicin derivative or an N-acetyl gamma-calicheamicin derivative. Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents. Calicheamicins contain a methyltrisulfide moiety that can be reacted with appropriate thiols to form disulfides, at the same time introducing a functional group that is useful in attaching a calicheamicin derivative to an anti-CD45 antibody or antigen-binding fragment thereof as described herein, via a linker. For the preparation of conjugates of the calicheamicin family, see U.S. Pat. Nos. 5,712,374; 5,714,586; 5,739,116; 5,767,285; 5,770,701; 5,770,710; 5,773,001; and 5,877,296 (all to American Cyanamid Company). Structural analogues of calicheamicin which may be used include, but are not limited to, those disclosed in, for example, Hinman et al., Cancer Research 53:3336-3342 (1993), Lode et al., Cancer Research 58:2925-2928 (1998), and the aforementioned U.S. patents to American Cyanamid.

In one embodiment, the cytotoxin of the ADC as disclosed herein may be a calicheamicin disulfide derivative represented by the formula:

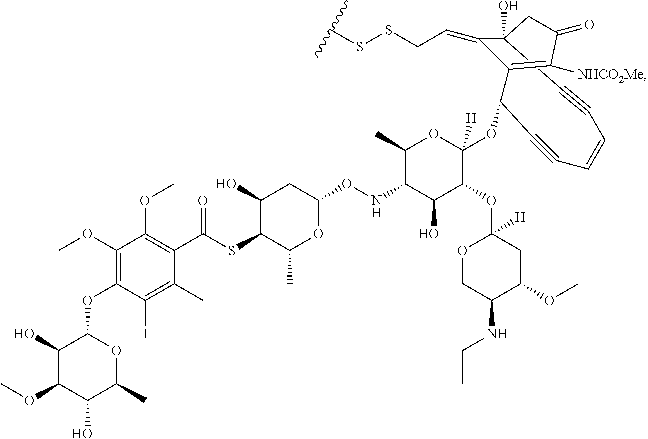

wherein the wavy line indicates the attachment point of the linker.

Ribosome Inactivating Proteins (RIPs)

In some embodiments, the cytotoxin conjugated to an anti-CD45 antibody is a ribosome-inactivating protein (RIP). Ribosome inactivating proteins are protein synthesis inhibitors that act on ribosomes, usually irreversibly. RIPs are found in plants, as well as bacteria. Examples of RIPs include, but are not limited to, saporin, ricin, abrin, gelonin, Pseudomonas exotoxin (or exotoxin A), trichosanthin, luffin, agglutinin and the diphtheria toxin.

Another example of an RIP that may be used in the ADCs and methods disclosed herein are a Shiga toxin (Stx) or a Shiga-like toxins (SLT). Shiga toxin (Stx) is a potent bacterial toxin found in Shigella dysenteriae 1 and in some serogroups (including serotypes O157:H7, and O104:H4) of Escherichia coli (called Stx1 in E. coli). In addition to Stx1, some E. coli strains produce a second type of Stx (Stx2) that has the same mode of action as Stx/Stx1 but is antigenically distinct. SLT is a historical term for similar or identical toxins produced by Escherichia coli. Because subtypes of each toxin have been identified, the prototype toxin for each group is now designated Stx1a or Stx2a. Stx1a and Stx2a exhibit differences in cytotoxicity to various cell types, bind dissimilarly to receptor analogs or mimics, induce differential chemokine responses, and have several distinctive structural characteristics.

A member of the Shiga toxin family refers to any member of a family of naturally occurring protein toxins which are structurally and functionally related, notably, toxins isolated from S. dysenteriae and E. coli (Johannes L, Romer W, Nat Rev Microbiol 8: 105-16 (2010)). For example, the Shiga toxin family encompasses true Shiga toxin (Stx) isolated from S. dysenteriae serotype 1, Shiga-like toxin 1 variants (SLT1 or Stx1 or SLT-1 or Slt-I) isolated from serotypes of enterohemorrhagic E. coli, and Shiga-like toxin 2 variants (SLT2 or Stx2 or SLT-2) isolated from serotypes of enterohemorrhagic E. coli. SLT1 differs by only one residue from Stx, and both have been referred to as Verocytotoxins or Verotoxins (VTs) (O'Brien A et al., Curr Top Microbiol Immunol 180: 65-94 (1992)). Although SLT1 and SLT2 variants are reported to be only about 53-60% similar to each other at the amino acid sequence level, they share mechanisms of enzymatic activity and cytotoxicity common to the members of the Shiga toxin family (Johannes, Nat Rev Microbiol 8: 105-16 (2010)).

Members of the Shiga toxin family have two subunits; A subunit and a B subunit. The B subunit of the toxin binds to a component of the cell membrane known as glycolipid globotriaosylceramide (Gb3). Binding of the subunit B to Gb3 causes induction of narrow tubular membrane invaginations, which drives formation of inward membrane tubules for the bacterial uptake into the cell. The Shiga toxin (a non-pore forming toxin) is transferred to the cytosol via Golgi network and ER. From the Golgi toxin is trafficked to the ER. Shiga toxins act to inhibit protein synthesis within target cells by a mechanism similar to that of ricin (Sandvig and van Deurs (2000) EMBO J 19(220:5943). After entering a cell the A subunit of the toxin cleaves a specific adenine nucleobase from the 28S RNA of the 60S subunit of the ribosome, thereby halting protein synthesis (Donohue-Rolfe et al. (2010) Reviews of Infectious Diseases 13 Suppl. 4(7): S293-297).

As used herein, reference to Shiga family toxin refers to any member of the Shiga toxin family of naturally occurring protein toxins (e.g., toxins isolated from S. dysenteriae and E. coli) which are structurally and functionally related. For example, the Shiga toxin family encompasses true Shiga toxin (Stx) isolated from S. dysenteriae serotype 1, Shiga-like toxin 1 variants (SLT1 or Stx1 or SLT-1 or Slt-I) isolated from serotypes of enterohemorrhagic E. coli, and Shiga-like toxin 2 variants (SLT2 or Stx2 or SLT-2) isolated from serotypes of enterohemorrhagic E. coli. As used herein, “subunit A from a Shiga family toxin” or “Shiga family toxin subunit A” refers to a subunit A from any member of the Shiga toxin family, including Shiga toxins or Shiga-like toxins.

In one embodiment, an anti-CD45 ADC comprises an anti-CD45 antibody conjugated to a Shiga family toxin subunit A, or a portion of a Shiga family toxin subunit A having cytotoxic activity, i.e., ribosome inhibiting activity. Shiga toxin subunit A cytotoxic activities include, for example, ribosome inactivation, protein synthesis inhibition, N-glycosidase activity, polynucleotide:adenosine glycosidase activity, RNAase activity, and DNAase activity. Non-limiting examples of assays for Shiga toxin effector activity measure protein synthesis inhibitory activity, depurination activity, inhibition of cell growth, cytotoxicity, supercoiled DNA relaxation activity, and nuclease activity.

In certain embodiments, an anti-CD45 antibody, or an antigen binding fragment thereof, is conjugated to Shiga family toxin A subunit, or a fragment thereof having ribosome inhibiting activity. An example of a Shiga family toxin subunit A is Shiga-like toxin 1 subunit A (SLT-1A), the amino acid sequence of which is provided below

(SEQ ID NO: 33)
KEFTLDFSTAKTYVDSLNVIRSAIGTPLQTISSGGTSLLMIDSGSGDN
LFAVDVRGIDPEEGRFNNLRLIVERNNLYVTGFVNRTNNVFYRFADFS
HVTFPGTTAVTLSGDSSYTTLQRVAGISRTGMQINRHSLTTSYLDLMS
HSGTSLTQSVARAMLRFVTVTAEALRFROIORGFRTTLDDLSGRSYVM
TAEDVDLTLNWGRLSSVLPDYHGQDSVRVGRISFGSINAILGSVALIL
NCHHHASRVARMASDEFPSMCPADGRVRGITHNKILWDSSTLGAILMR
RTISS.

Another example of a Shiga family toxin subunit A is Shiga toxin subunit A (StxA), the amino acid sequence of which is provided below

(SEQ ID NO: 34)
KEFTLDFSTAKTYVDSLNVIRSAIGTPLQTISSGGTSLLMIDSGTGDN
LFAVDVRGIDPEEGRFNNLRLIVERNNLYVTGFVNRTNNVFYRFADFS
HVTFPGTTAVTLSGDSSYTTLQRVAGISRTGMQINRHSLTTSYLDLMS
HSGTSLTQSVARAMLRFVTVTAEALRFRQIQRGFRTTLDDLSGRSYVM
TAEDVDLTLNWGRLSSVLPDYHGQDSVRVGRISFGSINAILGSVALIL
NCHHHASRVARMASDEFPSMCPADGRVRGITHNKILWDSSTLGAILMR
RTISS.

Another example of a Shiga family toxin subunit A is Shiga-like toxin 2 subunit A (SLT-2A), the amino acid sequence of which is provided below

(SEQ ID NO: 35)
DEFTVDFSSQKSYVDSLNSIRSAISTPLGNISQGGVSVSVINHVLGGN
YISLNVRGLDPYSERFNHLRLIMERNNLYVAGFINTETNIFYRFSDFS
HISVPDVITVSMTTDSSYSSLQRIADLERTGMQIGRHSLVGSYLDLME
FRGRSMTRASSRAMLRFVTVIAEALRFRQIQRGFRPALSEASPLYTMT
AQDVDLTLNWGRISNVLPEYRGEEGVRIGRISFNSLSAILGSVAVILN
CHSTGSYSVRSVSQKQKTECQIVGDRAAIKVNNVLWEANTIAALLNRK
PQDLTEPNQ.

In certain circumstances, naturally occurring Shiga family toxin subunits A may comprise precursor forms containing signal sequences of about 22 amino acids at their amino-terminals which are removed to produce mature Shiga family toxin A subunits and are recognizable to the skilled worker. Cytotoxic fragments or truncated versions of Shiga family toxin subunit A may also be used in the ADCs and methods disclosed herein.

In certain embodiments, a Shiga family toxin subunit A differs from a naturally occurring Shiga toxin A subunit by up to 1, 2, 3, 4, 5, 6, 7, 8, 9, 10, 15, 20, 25, 30, 35, 40 or more amino acid residues (but by no more than that which retains at least 85%, 90%, 95%, 99%, or more amino acid sequence identity). In some embodiments, the Shiga family toxin subunit A differs from a naturally occurring Shiga family toxin A subunit by up to 1, 2, 3, 4, 5, 6, 7, 8, 9, 10, 15, 20, 25, 30, 35, 40 or more amino acid residues (but by no more than that which retains at least 85%, 90%, 95%, 99% or more amino acid sequence identity). Thus, a polypeptide region derived from an A Subunit of a member of the Shiga toxin family may comprise additions, deletions, truncations, or other alterations from the original sequence as long as at least 85%, 90%, 95%, 99% Of more amino acid sequence identity is maintained to a naturally occurring Shiga family toxin subunit A.

Accordingly, in certain embodiments, the Shiga family toxin subunit A comprises or consists essentially of amino acid sequences having at least 55%, 60%, 65%, 70%, 75%, 80%, 85%, 90%, 95%, 97%, 98%, 99%, 99.5% or 99.7% overall sequence identity to a naturally occurring Shiga family toxin subunit A. such as SIT-1A (SEQ ID NO: 33), StxA (SEQ ID NO: 34), and/or SLT-2A (SEQ ID NO: 35).

Suitable Shiga toxins and RIPs suitable as cytotoxins are disclosed in, for example, US20180057544, which is incorporated by reference herein in its entirety.

Additional Cytotoxins

In other embodiments, the antibodies and antigen-binding fragments thereof described herein can be conjugated to a cytotoxin other than or in addition to those cytotoxins disclosed herein above. Additional cytotoxins suitable for use with the compositions and methods described herein include, without limitation, 5-ethynyluracil, abiraterone, acylfulvene, adecypenol, adozelesin, aldesleukin, altretamine, ambamustine, amidox, amifostine, aminolevulinic acid, amrubicin, amsacrine, anagrelide, anastrozole, andrographolide, angiogenesis inhibitors, antarelix, anti-dorsalizing morphogenetic protein-1, antiandrogen, prostatic carcinoma, antiestrogen, antineoplaston, antisense oligonucleotides, aphidicolin glycinate, apoptosis gene modulators, apoptosis regulators, apurinic acid, asulacrine, atamestane, atrimustine, axinastatin 1, axinastatin 2, axinastatin 3, azasetron, azatoxin, azatyrosine, baccatin III derivatives, balanol, batimastat, BCR/ABL antagonists, benzochlorins, benzoylstaurosporine, beta lactam derivatives, beta-alethine, betaclamycin B, betulinic acid, bFGF inhibitors, bicalutamide, bisantrene, bisaziridinylspermine, bisnafide, bistratene A, bizelesin, breflate, bleomycin A2, bleomycin B2, bropirimine, budotitane, buthionine sulfoximine, calcipotriol, calphostin C, camptothecin derivatives (e.g., 10-hydroxy-camptothecin), capecitabine, carboxamide-amino-triazole, carboxyamidotriazole, carzelesin, casein kinase inhibitors, castanospermine, cecropin B, cetrorelix, chlorins, chloroquinoxaline sulfonamide, cicaprost, cis-porphyrin, cladribine, clomifene and analogues thereof, clotrimazole, collismycin A, collismycin B, combretastatin A4, combretastatin analogues, conagenin, crambescidin 816, crisnatol, cryptophycin 8, cryptophycin A derivatives, curacin A, cyclopentanthraquinones, cycloplatam, cypemycin, cytarabine ocfosfate, cytolytic factor, cytostatin, dacliximab, decitabine, dehydrodidemnin B, 2′deoxycoformycin (DCF), deslorelin, dexifosfamide, dexrazoxane, dexverapamil, diaziquone, didemnin B, didox, diethylnorspermine, dihydro-5-azacytidine, dihydrotaxol, dioxamycin, diphenyl spiromustine, discodermolide, docosanol, dolasetron, doxifluridine, droloxifene, dronabinol, duocarmycin SA, ebselen, ecomustine, edelfosine, edrecolomab, eflornithine, elemene, emitefur, epothilones, epithilones, epristeride, estramustine and analogues thereof, etoposide, etoposide 4′-phosphate (also referred to as etopofos), exemestane, fadrozole, fazarabine, fenretinide, filgrastim, finasteride, flavopiridol, flezelastine, fluasterone, fludarabine, fluorodaunorunicin hydrochloride, forfenimex, formestane, fostriecin, fotemustine, gadolinium texaphyrin, gallium nitrate, galocitabine, ganirelix, gelatinase inhibitors, gemcitabine, glutathione inhibitors, hepsulfam, homoharringtonine (HHT), hypericin, ibandronic acid, idoxifene, idramantone, ilmofosine, ilomastat, imidazoacridones, imiquimod, immunostimulant peptides, iobenguane, iododoxorubicin, ipomeanol, irinotecan, iroplact, irsogladine, isobengazole, jasplakinolide, kahalalide F, lamellarin-N triacetate, lanreotide, leinamycin, lenograstim, lentinan sulfate, leptolstatin, letrozole, lipophilic platinum compounds, lissoclinamide 7, lobaplatin, lometrexol, lonidamine, losoxantrone, loxoribine, lurtotecan, lutetium texaphyrin, lysofylline, masoprocol, maspin, matrix metalloproteinase inhibitors, menogaril, rnerbarone, meterelin, methioninase, metoclopramide, MIF inhibitor, ifepristone, miltefosine, mirimostim, mithracin, mitoguazone, mitolactol, mitomycin and analogues thereof, mitonafide, mitoxantrone, mofarotene, molgramostim, mycaperoxide B, myriaporone, N-acetyldinaline, N-substituted benzamides, nafarelin, nagrestip, napavin, naphterpin, nartograstim, nedaplatin, nemorubicin, neridronic acid, nilutamide, nisamycin, nitrullyn, octreotide, okicenone, onapristone, ondansetron, oracin, ormaplatin, oxaliplatin, oxaunomycin, paclitaxel and analogues thereof, palauamine, palmitoylrhizoxin, pamidronic acid, panaxytriol, panomifene, parabactin, pazelliptine, pegaspargase, peldesine, pentosan polysulfate sodium, pentostatin, pentrozole, perflubron, perfosfamide, phenazinomycin, picibanil, pirarubicin, piritrexim, podophyllotoxin, porfiromycin, purine nucleoside phosphorylase inhibitors, raltitrexed, rhizoxin, rogletimide, rohitukine, rubiginone B1, ruboxyl, safingol, saintopin, sarcophytol A, sargramostim, sobuzoxane, sonermin, sparfosic acid, spicamycin D, spiromustine, stipiamide, sulfinosine, tallimustine, tegafur, temozolomide, teniposide, thaliblastine, thiocoraline, tirapazamine, topotecan, topsentin, triciribine, trimetrexate, veramine, vinorelbine, vinxaltine, vorozole, zeniplatin, and zilascorb, among others.

### Linkers

A variety of linkers can be used to conjugate antibodies, antigen-binding fragments, and ligands described herein (e.g., antibodies, antigen-binding fragments thereof, and soluble ligands that recognize and bind CD45) with a cytotoxic molecule.

The term “Linker” as used herein means a divalent chemical moiety comprising a covalent bond or a chain of atoms that covalently attaches an antibody or fragment thereof (Ab) to a drug moiety (D) to form antibody-drug conjugates of the present disclosure (ADCs; Ab-Z-L-D, where D is a cytotoxin). Suitable linkers have two reactive termini, one for conjugation to an antibody and the other for conjugation to a cytotoxin. The antibody conjugation reactive terminus of the linker (reactive moiety, Z) is typically a site that is capable of conjugation to the antibody through a cysteine thiol or lysine amine group on the antibody, and so is typically a thiol-reactive group such as a double bond (as in maleimide) or a leaving group such as a chloro, bromo, iodo, or an R-sulfanyl group, or an amine-reactive group such as a carboxyl group; while the cytotoxin conjugation reactive terminus of the linker is typically a site that is capable of conjugation to the cytotoxin. Non-limiting examples for linker-cytotoxin conjugation include, for example, formation of an amide bond with a basic amine or carboxyl group on the cytotoxin, via a carboxyl or basic amine group on the linker, respectively, or formation of an ether or the like, via alkylation of an OH group on the cytotoxin, via e.g., a leaving group on the linker. In some embodiments, cytotoxin-linker conjugation is through formation of an amide bond with a basic amine or carboxyl group on the cytotoxin, and so the reactive substituent on the linker is respectively a carboxyl or basic amine group. When the term “linker” is used in describing the linker in conjugated form, one or both of the reactive termini will be absent (such as reactive moiety Z, having been converted to chemical moiety Z) or incomplete (such as being only the carbonyl of the carboxylic acid) because of the formation of the bonds between the linker and/or the cytotoxin, and between the linker and/or the antibody or antigen-binding fragment thereof. Such conjugation reactions are described further herein below.

In some embodiments, the linker is cleavable under intracellular conditions, such that cleavage of the linker releases the drug unit from the antibody in the intracellular environment. In yet other embodiments, the linker unit is not cleavable and the drug is released, for example, by antibody degradation. The linkers useful for the present ADCs are preferably stable extracellularly, prevent aggregation of ADC molecules and keep the ADC freely soluble in aqueous media and in a monomeric state. Before transport or delivery into a cell, the ADC is preferably stable and remains intact, i.e. the antibody remains linked to the drug moiety. The linkers are stable outside the target cell and may be cleaved at some efficacious rate inside the cell. An effective linker will: (i) maintain the specific binding properties of the antibody; (ii) allow intracellular delivery of the conjugate or drug moiety; (iii) remain stable and intact, i.e. not cleaved, until the conjugate has been delivered or transported to its targeted site; and (iv) maintain a cytotoxic, cell-killing effect or a cytostatic effect of the cytotoxic moiety. Stability of the ADC may be measured by standard analytical techniques such as mass spectroscopy, HPLC, and the separation/analysis technique LC/MS. Covalent attachment of the antibody and the drug moiety requires the linker to have two reactive functional groups, i.e. bivalency in a reactive sense. Bivalent linker reagents which are useful to attach two or more functional or biologically active moieties, such as peptides, nucleic acids, drugs, toxins, antibodies, haptens, and reporter groups are known, and methods have been described their resulting conjugates (Hermanson, G. T. (1996) Bioconjugate Techniques; Academic Press: New York, p. 234-242).

Linkers include those that may be cleaved, for instance, by enzymatic hydrolysis, photolysis, hydrolysis under acidic conditions, hydrolysis under basic conditions, oxidation, disulfide reduction, nucleophilic cleavage, or organometallic cleavage (see, for example, Leriche et al., Bioorg. Med. Chem., 20:571-582, 2012, the disclosure of which is incorporated herein by reference as it pertains to linkers suitable for covalent conjugation).

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation.

Linkers hydrolyzable under acidic conditions include, for example, hydrazones, semicarbazones, thiosemicarbazones, cis-aconitic amides, orthoesters, acetals, ketals, or the like. (See, e.g., U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661, the disclosure of each of which is incorporated herein by reference in its entirety as it pertains to linkers suitable for covalent conjugation. Such linkers are relatively stable under neutral pH conditions, such as those in the blood, but are unstable at below pH 5.5 or 5.0, the approximate pH of the lysosome.

Linkers cleavable under reducing conditions include, for example, a disulfide. A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene), SPDB and SMPT (See, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935, the disclosure of each of which is incorporated herein by reference in its entirety as it pertains to linkers suitable for covalent conjugation.

Linkers susceptible to enzymatic hydrolysis can be, e.g., a peptide-containing linker that is cleaved by an intracellular peptidase or protease enzyme, including, but not limited to, a lysosomal or endosomal protease. One advantage of using intracellular proteolytic release of the therapeutic agent is that the agent is typically attenuated when conjugated and the serum stabilities of the conjugates are typically high. In some embodiments, the peptidyl linker is at least two amino acids long or at least three amino acids long. Exemplary amino acid linkers include a dipeptide, a tripeptide, a tetrapeptide or a pentapeptide. Examples of suitable peptides include those containing amino acids such as Valine, Alanine, Citrulline (Cit), Phenylalanine, Lysine, Leucine, and Glycine. Amino acid residues which comprise an amino acid linker component include those occurring naturally, as well as minor amino acids and non-naturally occurring amino acid analogs, such as citrulline. Exemplary dipeptides include valine-citrulline (vc or val-cit) and alanine-phenylalanine (af or ala-phe). Exemplary tripeptides include glycine-valine-citrulline (gly-val-cit) and glycine-glycine-glycine (gly-gly-gly). In some embodiments, the linker comprises a dipeptide selected from the group consisting of Phe-Lys, Val-Lys, Phe-Ala, Phe-Cit, Val-Ala, Val-Cit, and Val-Arg. In some embodiments, the linker includes a dipeptide such as Val-Cit, Ala-Val, or Phe-Lys, Val-Lys, Ala-Lys, Phe-Cit, Leu-Cit, Ile-Cit, Phe-Arg, or Trp-Cit. Linkers containing dipeptides such as Val-Cit or Phe-Lys are disclosed in, for example, U.S. Pat. No. 6,214,345, the disclosure of which is incorporated herein by reference in its entirety as it pertains to linkers suitable for covalent conjugation. In some embodiments, the linker includes a dipeptide selected from Val-Ala and Val-Cit.

Linkers suitable for conjugating the antibodies, antigen-binding fragments, and ligands described herein to a cytotoxic molecule include those capable of releasing a cytotoxin by a 1,6-elimination process. Chemical moieties capable of this elimination process include the p-aminobenzyl (PAB) group, 6-maleimidohexanoic acid, pH-sensitive carbonates, and other reagents as described in Jain et al., Pharm. Res. 32:3526-3540, 2015, the disclosure of which is incorporated herein by reference in its entirety as it pertains to linkers suitable for covalent conjugation.

In some embodiments, the linker includes a “self-immolative” group such as the afore-mentioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and U.S. Pat. No. 7,223,837. In some embodiments, a dipeptide is used in combination with a self-immolative linker.

In some embodiments, the linker includes a self-immolative group such as the afore-mentioned PAB or PABC (para-aminobenzyloxycarbonyl), which are disclosed in, for example, Carl et al., J. Med. Chem. (1981) 24:479-480; Chakravarty et al (1983) J. Med. Chem. 26:638-644; U.S. Pat. No. 6,214,345; US20030130189; US20030096743; U.S. Pat. No. 6,759,509; US20040052793; U.S. Pat. Nos. 6,218,519; 6,835,807; 6,268,488; US20040018194; WO98/13059; US20040052793; U.S. Pat. Nos. 6,677,435; 5,621,002; US20040121940; WO2004/032828). Other such chemical moieties capable of this process (“self-immolative linkers”) include methylene carbamates and heteroaryl groups such as aminothiazoles, aminoimidazoles, aminopyrimidines, and the like. Linkers containing such heterocyclic self-immolative groups are disclosed in, for example, U.S. Patent Publication Nos. 20160303254 and 20150079114, and U.S. Pat. No. 7,754,681; Hay et al. (1999) Bioorg. Med. Chem. Lett. 9:2237; US 2005/0256030; de Groot et al (2001) J. Org. Chem. 66:8815-8830; and U.S. Pat. No. 7,223,837.

Linkers suitable for use herein further may include one or more groups selected from C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, heteroarylene, and combinations thereof, each of which may be optionally substituted. Non-limiting examples of such groups include (CH₂)ₚ, (CH₂CH₂O)ₚ, and —(C═O)(CH₂)ₚ— units, wherein p is an integer from 1-6, independently selected for each occasion.

In some embodiments, each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

In some embodiments, each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be interrupted by one or more heteroatoms selected from O, S and N.

In some embodiments, each C₁-C₆ alkylene, C₁-C₆ heteroalkylene, C₂-C₆ alkenylene, C₂-C₆ heteroalkenylene, C₂-C₆ alkynylene, C₂-C₆ heteroalkynylene, C₃-C₆ cycloalkylene, heterocycloalkylene, arylene, or heteroarylene may optionally be interrupted by one or more heteroatoms selected from O, S and N and may be optionally substituted with from 1 to 5 substituents independently selected for each occasion from the group consisting of alkyl, alkenyl, alkynyl, cycloalkyl, heterocycloalkyl, alkaryl, alkyl heteroaryl, amino, ammonium, acyl, acyloxy, acylamino, aminocarbonyl, alkoxycarbonyl, ureido, carbamate, aryl, heteroaryl, sulfinyl, sulfonyl, hydroxyl, alkoxy, sulfanyl, halogen, carboxy, trihalomethyl, cyano, hydroxy, mercapto, and nitro.

Suitable linkers may contain groups having solubility enhancing properties. Linkers including the (CH₂CH₂O)ₚ unit (polyethylene glycol, PEG, for example, wherein p is an integer from 1-6), and ((CH₂)ₘO)ₙ(CH₂)ₘ— unit, where n and each m are each independently selected from 1, 2, 3, 4, 5, 6, 7, 8, 9, and 10), for example, can enhance solubility, as can alkyl chains substituted with amino, sulfonic acid, phosphonic acid or phosphoric acid residues. Linkers including such moieties are disclosed in, for example, U.S. Pat. Nos. 8,236,319 and 9,504,756, the disclosure of each of which is incorporated herein by reference in its entirety as it pertains to linkers suitable for covalent conjugation. Further solubility enhancing groups include, for example, acyl and carbamoyl sulfamide groups, having the structure:

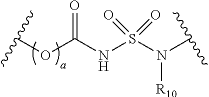

wherein a is 0 or 1; and

R¹⁰ is selected from the group consisting of hydrogen, C₁-C₂₄ alkyl groups, C₃-C₂₄ cycloalkyl groups, C₁-C₂₄ (hetero)aryl groups, C₁-C₂₄ alkyl(hetero)aryl groups and C₁-C₂₄ (hetero)arylalkyl groups, the C₁-C₂₄ alkyl groups, C₃-C₂₄ cycloalkyl groups, C₂-C₂₄ (hetero)aryl groups, C₃-C₂₄ alkyl(hetero)aryl groups and C₃-C₂₄ (hetero)arylalkyl groups, each of which may be optionally substituted and/or optionally interrupted by one or more heteroatoms selected from O, S and NR¹¹R¹², wherein R¹¹ and R¹² are independently selected from the group consisting of hydrogen and C₁-C₄ alkyl groups; or R¹⁰ is a cytotoxin, wherein the cytotoxin is optionally connected to N via a spacer moiety. Linkers containing such groups are described, for example, in U.S. Pat. No. 9,636,421 and U.S. Patent Application Publication No. 2017/0298145, the disclosures of which are incorporated herein by reference in their entirety as they pertain to linkers suitable for covalent conjugation to cytotoxins and antibodies or antigen-binding fragments thereof.

In some embodiments, the linker may include one or more of a hydrazine, a disulfide, a thioether, a dipeptide, a p-aminobenzyl (PAB) group, a heterocyclic self-immolative group, an optionally substituted C₁-C₆ alkyl, an optionally substituted C₁-C₆ heteroalkyl, an optionally substituted C₂-C₆ alkenyl, an optionally substituted C₂-C₆ heteroalkenyl, an optionally substituted C₂-C₆ alkynyl, an optionally substituted C₂-C₆ heteroalkynyl, an optionally substituted C₃-C₆ cycloalkyl, an optionally substituted heterocycloalkyl, an optionally substituted aryl, an optionally substituted heteroaryl, a solubility enhancing group, acyl, —(C═O)—, or —(CH₂CH₂O)ₚ— group, wherein p is an integer from 1-6. One of skill in the art will recognize that one or more of the groups listed may be present in the form of a bivalent (diradical) species, e.g., C₁-C₆ alkylene and the like. In some embodiments, the linker L comprises the moiety *-L₁L₂-**, wherein:

L₁ is absent or is —(CH₂)ₘNR¹³C(═O)—, —(CH₂)ₘNR¹³—, —(CH₂)ₘX₃(CH₂)ₘ—,

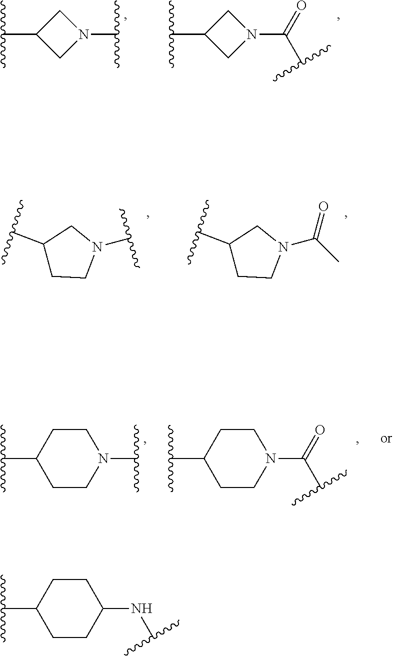

L₂ is absent or is —(CH₂)ₘ—, —NR¹³(CH₂)ₘ—, —(CH₂)ₘNR¹³C(═O)(CH₂)ₘ—, —X₄, —(CH₂)ₘNR¹³C(═O)X₄, —(CH₂)ₘNR¹³C(═O)—, —((CH₂)ₘO)ₙ(CH₂)ₘ—, —((CH₂)ₘO)ₙ(CH₂)ₘX₃(CH₂)ₘ—, —NR¹³((CH₂)ₘO)ₙX₃(CH₂)ₘ—, —NR¹³((CH₂)ₘO)ₙ(CH₂)ₘX₃(CH₂)ₘ—, —X₁X₂C(═O)(CH₂)ₘ—, —(CH₂)ₘ(O(CH₂)ₘ)ₙ—, —(CH₂)ₘNR¹³(CH₂)ₘ—, —(CH₂)ₘNR¹³C(═O)(CH₂)ₘX₃(CH₂)ₘ—, —(CH₂)ₘC(═O)NR¹³(CH₂)ₘNR¹³C(═O)(CH₂)ₘ—, —(CH₂)ₘC(═O)—, —(CH₂)ₘNR¹³(CH₂)ₘC(═O)X₂X₁C(═O)—, —(CH₂)ₘX₃(CH₂)ₘC(═O)X₂X₁C(═O)—, —(CH₂)ₘC(═O)NR¹³(CH₂)ₘ—, —(CH₂)ₘC(═O)NR¹³(CH₂)ₘX₃(CH₂)ₘ—, —(CH₂)ₘX₃(CH₂)ₘNR¹³C(═O)(CH₂)ₘ—, —(CH₂)ₘX₃(CH₂)ₘC(═O)NR¹³(CH₂)ₘ—, —(CH₂)ₘO)ₙ(CH₂)ₘNR¹³C(═O)(CH₂)ₘ—, —(CH₂)ₘC(═O)NR¹³(CH₂)ₘ(O(CH₂)ₘ)ₙ—, —(CH₂)ₘ(O(CH₂)ₘ)ₙC(═O)—, —(CH₂)ₘNR¹³(CH₂)ₘC(═O)—, —(CH₂)ₘC(═O)NR¹³(CH₂)ₘNR¹³C(═O)—, —(CH₂)ₘ(O(CH₂)ₘ)ₙX₃(CH₂)ₘ—, —(CH₂)_{m×3}((CH₂)ₘ0)ₙ(CH₂)ₘ—, —(CH₂)ₘX₃(CH₂)ₘC(═O)—, —(CH₂)ₘ(═O)NR¹³(CH₂)ₘO)ₙ(CH₂)ₘX₃(CH₂)ₘ—, —(CH₂)ₘX₃(CH₂)ₘ(O(CH₂)ₘO)ₙNR¹³C(═O)(CH₂)ₘ—, —(CH₂)ₘX₃(CH₂)ₘ(O(CH₂)ₘ)ₙC(═O)—, —(CH₂)ₘX₃(CH₂)ₘ(O(CH₂)ₘ)ₙ—, —(CH₂)ₘC(═O)NR¹³(CH₂)ₘC(═O)—, —(CH₂)ₘC(═O)NR¹³(CH₂)ₘ(O(CH₂)ₘ)ₙC(═O)—, —((CH₂)ₘO)ₘ(CH₂)ₘNR¹³C(═O)(CH₂)ₘ—, —(CH₂)ₘC(═O)NR¹³(CH₂)ₘC(═O)NR¹³(CH₂)ₘ—, —(CH₂)ₘNR¹³C(═O)(CH₂)ₘNR¹³C(═O)(CH₂)—(CH₂)ₘX₃(CH₂)ₘC(═O)NR¹³—, —(CH₂)ₘC(═O)NR¹³—, —(CH₂)ₘX₃—, —C(R¹³)₂(CH₂)ₘ—, —(CH₂)ₘC(R¹³)₂NR¹³—, —(CH₂)ₘC(═O)NR¹³(CH₂)ₘNR¹³—, —(CH₂)ₘC(═O)NR¹³(CH₂)ₘNR¹³C(═O)NR¹³—, —(CH₂)ₘC(═O)X₂X₁C(═O)—, —C(R¹³)₂(CH₂)ₘNR¹³C(═O)(CH₂)ₘ—, —(CH₂)ₘC(═O)NR¹³(CH₂)ₘC(R¹³)₂NR¹³—, —C(R¹³)₂(CH₂)ₘX₃(CH₂)ₘ—, —(CH₂)ₘX₃(CH₂)ₘC(R¹³)₂NR¹³—, —C(R¹³)₂(CH₂)ₘOC(═O)NR¹³(CH₂)ₘ—, —(CH₂)ₘNR¹³C(═O)O(CH₂)ₘC(R¹³)₂NR¹³—, —(CH₂)ₘX₃(CH₂)ₘNR¹³—, —(CH₂)ₘX₃(CH₂)ₘ(O(CH₂)ₘ)ₙNR¹³—, —(CH₂)ₘNR¹³—, —(CH₂)ₘC(═O)NR¹³(CH₂)ₘ(O(CH₂)ₘ)ₙNR¹³—, —(CH₂)ₘ(O(CH₂)ₘ)ₙNR¹³—, —(CH₂CH₂O)ₙ(CH₂)ₘ—, —(CH₂)ₘ(OCH₂CH₂)ₙ, —(CH₂)ₘO(CH₂)ₘ—, —(CH₂)ₘS(═O)₂—, —(CH₂)ₘC(═O)NR¹³(CH₂)ₘS(═O)₂—, —(CH₂)ₘX₃(CH₂)ₘS(═O)₂—, —(CH₂)ₘX₂X₁C(═O)—, —(CH₂)ₘ(O(CH₂)ₘ)ₙC(═O)X₂X₁C(═O)—, —(CH₂)ₘ(O(CH₂)ₘ)ₙX₂X₁C(═O)—, —(CH₂)ₘX₃(CH₂)ₘX₂X₁C(═O)—, —(CH₂)ₘX₃(CH₂)ₘ(O(CH₂)ₘ)ₙX₂X₁C(═O)—, —(CH₂)ₘX₃(CH₂)ₘC(═O)NR¹³(CH₂)ₘNR¹³C(═O)—, —(CH₂)ₘX₃(CH₂)ₘC(═O)NR¹³(CH₂)ₘC(═O)—, —(CH₂)ₘX₃(CH₂)ₘC(═O)NR¹³(CH₂)ₘ(O(CH₂)ₘ)ₙC(═O)—, —(CH₂)ₘC(═O)X₂X₁C(═O)NR¹³(CH₂)ₘ—, —(CH₂)ₘX₃(O(CH₂)ₘ)ₙC(═O)—, —(CH₂)ₘNR¹³C(═O)((CH₂)ₘO)ₙ(CH₂)ₘ—, —(CH₂)ₘ(O(CH₂)ₘ)ₙC(═O)NR¹³(CH₂)ₘ—, —(CH₂)ₘNR¹³C(═O)NR¹³(CH₂)ₘ— or —(CH₂)ₘX₃(CH₂)ₘNR¹³C(═O)—;

wherein

X₁ is

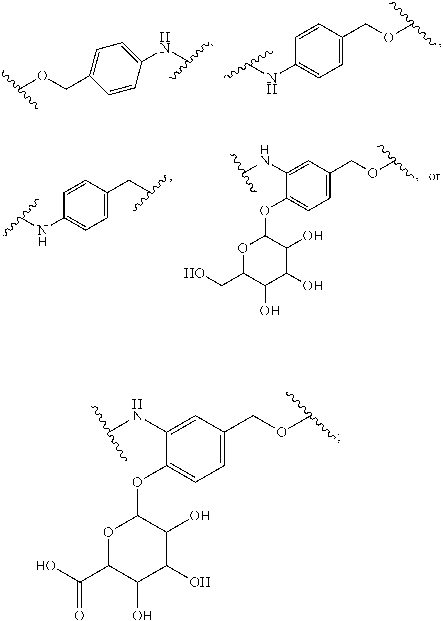 [SMILES: *Nc1ccc(CO*)cc1 ; *Nc1ccc(CO*)cc1 ; *Cc1ccc(N*)cc1 ; *Nc1cc(CO*)ccc1OC1OC(CO)C(O)C(O)C1O ; *Nc1cc(CO*)ccc1OC1OC(C(=O)O)C(O)C(O)C1O]

X₂ is

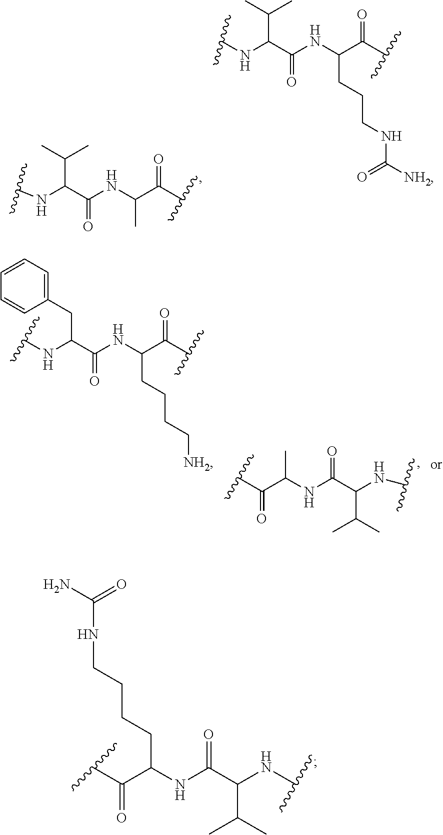

X₃ is

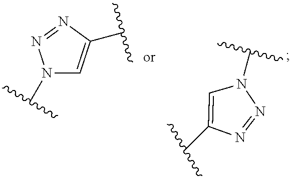 [SMILES: *c1cn(*)nn1 ; *c1cn(*)nn1]

and

X₄ is

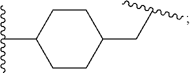

wherein

R¹³ is independently selected for each occasion from H and C₁-C₆ alkyl;

m is independently selected for each occasion from 1, 2, 3, 4, 5, 6, 7, 8, 9 and 10;

n is independently selected for each occasion from 1, 2, 3, 4, 5, 6, 7, 8, 9, 10, 11, 12, 13 and 14; and

wherein the single asterisk (*) indicates the attachment point to the cytotoxin (e.g., an amatoxin), and the double asterisk (**) indicates the attachment point to the reactive substituent Z′ or chemical moiety Z, with the proviso that L₁ and L₂ are not both absent.

In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a combination of one or more of a peptide, oligosaccharide, —(CH₂)ₚ—, —(CH₂CH₂O)ₚ—, PAB, Val-Cit-PAB, Val-Ala-PAB, Val-Lys(Ac)-PAB, Phe-Lys-PAB, Phe-Lys(Ac)-PAB, D-Val-Leu-Lys, Gly-Gly-Arg, Ala-Ala-Asn-PAB, or Ala-PAB.

In some embodiments, the linker comprises a ((CH₂)ₘO)ₙ(CH₂)ₘ— group where n and each m are each independently selected from 1, 2, 3, 4, 5, 6, 7, 8, 9, and 10; and a heteroaryl group, wherein the heteroaryl group is a triazole. In some embodiments, the ((CH₂)ₘO)ₙ(CH₂)ₘ— group and triazole together comprise

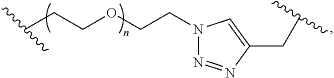

where n is from 1 to 10, and the wavy lines indicate attachment points to additional linker components, the chemical moiety Z, or the amatoxin.

In some embodiments, the linker includes a dipeptide selected from Val-Ala and Val-Cit. In some embodiments, a dipeptide is used in combination with a self-immolative linker. In some embodiments, the linker includes a p-aminobenzyl group (PAB). In one embodiment, the p-aminobenzyl group is disposed between the cytotoxic drug and a protease cleavage site in the linker. In one embodiment, the p-aminobenzyl group is part of a p-aminobenzyloxycarbonyl unit. In one embodiment, the p-aminobenzyl group is part of a p-aminobenzylamido unit.

In some embodiments, the linker comprises a —(C═O)(CH₂)ₚ— unit, wherein p is an integer from 1-6.

In some embodiments, the linker comprises a —(CH₂)ₙ— unit, wherein n is an integer from 2 to 6.

In one specific embodiment, the linker comprises the structure

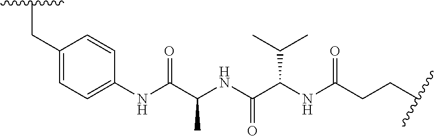 [SMILES: *CCC(=O)N[C@H](C(=O)N[C@@H](C)C(=O)Nc1ccc(C*)cc1)C(C)C]

wherein the wavy lines indicate attachment points to the cytotoxin and the reactive moiety Z. In another specific embodiment, the linker comprises the structure

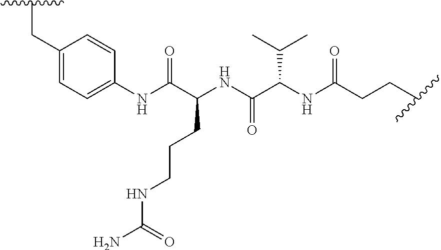 [SMILES: *CCC(=O)N[C@H](C(=O)N[C@@H](CCCNC(N)=O)C(=O)Nc1ccc(C*)cc1)C(C)C]

wherein the wavy lines indicate attachment points to the cytotoxin and the reactive moiety Z. Such PAB-dipeptide-propionyl linkers are disclosed in, e.g., Patent Application Publication No. WO2017/149077, which is incorporated by reference herein in its entirety. Further, the cytotoxins disclosed in WO2017/149077 are incorporated by reference herein.

In certain embodiments, the linker of the ADC includes N-beta-maleimidopropyl-Val-Ala-para-aminobenzyl (BMP-Val-Ala-PAB. In certain embodiments, the linker of the ADC is N-beta-maleimidopropyl-Val-Ala-para-aminobenzyl (BMP-Val-Ala-PAB).

In certain embodiments, the linker of the ADC is maleimidocaproyl-Val-Ala-para-aminobenzyl (mc-Val-Ala-PAB).

In certain embodiments, the linker of the ADC is maleimidocaproyl-Val-Cit-para-aminobenzyl (mc-vc-PAB).

In some embodiments, the linker comprises one of:

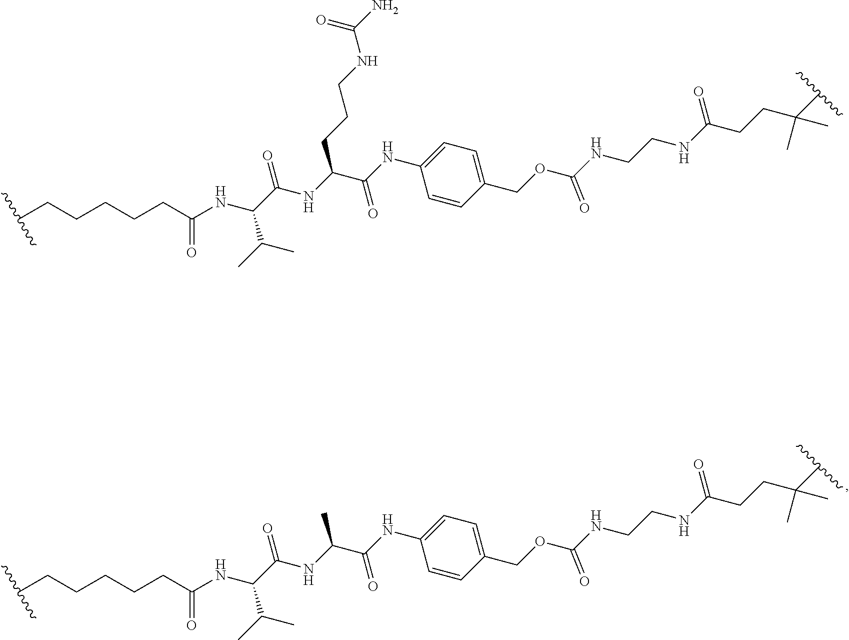 [SMILES: *CCCCCC(=O)N[C@H](C(=O)N[C@@H](CCCNC(N)=O)C(=O)Nc1ccc(COC(=O)NCCNC(=O)CCC(*)(C)C)cc1)C(C)C ; *CCCCCC(=O)N[C@H](C(=O)N[C@@H](C)C(=O)Nc1ccc(COC(=O)NCCNC(=O)CCC(*)(C)C)cc1)C(C)C]

where the gem-dimethyl terminus of the linker is attached to, for example, the disulfide moiety of a calicheamicin derivative, produced by reduction of the calicheamicin trisulfide group. Such linkers are disclosed in, for example, International Patent Application Publication No. WO2016/172273, the disclosure of which is incorporated by reference herein in its entirety.

In some embodiments, the linker comprises a 4-(4′-acetylphenoxy)butanoic acid moiety. In some embodiments, the linker comprises a hydrazone. In some embodiments, the linker comprises a 4-(4′-acetylphenoxy)butanoic acid moiety and a hydrazone, represented by the formula:

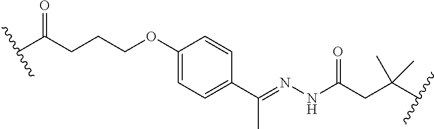 [SMILES: *C(=O)CCCOc1ccc(/C(C)=N/NC(=O)CC(*)(C)C)cc1]

where the dimethyl terminus of the linker is attached to, for example, the disulfide moiety of a calicheamicin derivative, produced by reduction of the calicheamicin trisulfide group. Such linkers are disclosed in, for example, U.S. Pat. No. 5,606,040, the disclosure of which is incorporated by reference herein in its entirety.

In some embodiments, the linker comprises MCC (4-[N-maleimidomethyl]cyclohexane-1-carboxylate).

Linkers that can be used to conjugate an antibody, antigen-binding fragment thereof, or ligand to a cytotoxic agent include those that are covalently bound to the cytotoxic agent on one end of the linker and, on the other end of the linker, contain a chemical moiety formed from a coupling reaction between a reactive substituent present on the linker and a reactive substituent present within the antibody, antigen-binding fragment thereof, or ligand that binds CD45. Reactive substituents that may be present within an antibody, antigen-binding fragment thereof, or ligand that binds CD45 include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. Examples of linkers useful for the synthesis of ADCs include those that contain electrophiles, such as Michael acceptors (e.g., maleimides), activated esters, electron-deficient carbonyl compounds, and aldehydes, among others, suitable for reaction with nucleophilic substituents present within antibodies or antigen-binding fragments, such as amine and thiol moieties. For instance, linkers suitable for the synthesis of ADCs include, without limitation, succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, for instance, Liu et al., 18:690-697, 1979, the disclosure of which is incorporated herein by reference as it pertains to linkers for chemical conjugation.

It will be recognized by one of skill in the art that any one or more of the chemical groups, moieties and features disclosed herein may be combined in multiple ways to form linkers useful for conjugation of the antibodies and cytotoxins as disclosed herein. Further linkers useful in conjunction with the compositions and methods described herein, are described, for example, in U.S. Patent Application Publication No. 2015/0218220, the disclosure of which is incorporated herein by reference in its entirety.

In certain embodiments, an intermediate, which is the precursor of the linker, is reacted with the drug moiety under appropriate conditions. In certain embodiments, reactive groups are used on the drug and/or the intermediate or linker. The product of the reaction between the drug and the intermediate, or the derivatized drug, is subsequently reacted with the antibody or antigen-binding fragment under appropriate conditions. Alternatively, the linker or intermediate may first be reacted with the antibody or a derivatized antibody, and then reacted with the drug or derivatized drug. Such conjugation reactions will now be described more fully.

A number of different reactions are available for covalent attachment of linkers or drug-linker conjugates to the antibody or antigen-binding fragment thereof. Suitable attachment points on the antibody molecule include the amine groups of lysine, the free carboxylic acid groups of glutamic acid and aspartic acid, the sulfhydryl groups of cysteine, and the various moieties of the aromatic amino acids. For instance, non-specific covalent attachment may be undertaken using a carbodiimide reaction to link a carboxy (or amino) group on a compound to an amino (or carboxy) group on an antibody moiety. Additionally, bifunctional agents such as dialdehydes or imidoesters may also be used to link the amino group on a compound to an amino group on an antibody moiety. Also available for attachment of drugs to binding agents is the Schiff base reaction. This method involves the periodate oxidation of a drug that contains glycol or hydroxy groups, thus forming an aldehyde which is then reacted with the binding agent. Attachment occurs via formation of a Schiff base with amino groups of the binding agent. Isothiocyanates may also be used as coupling agents for covalently attaching drugs to binding agents. Other techniques are known to the skilled artisan and within the scope of the present disclosure.

Linkers useful in for conjugation to the antibodies or antigen-binding fragments as described herein include, without limitation, linkers containing chemical moieties Z formed by coupling reactions as depicted in Table 2, below. Curved lines designate points of attachment to the antibody or antigen-binding fragment, and the cytotoxic molecule, respectively.

TABLE 2
Exemplary chemical moieties Z formed by coupling reactions in the formation of
antibody-drug conjugates

| Exemplary | |
|---|---|
| Coupling Reactions | Chemical Moiety Z Formed by Coupling Reactions |
| [3 + 2] Cycloaddition | 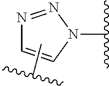 |
| [3 + 2] Cycloaddition | 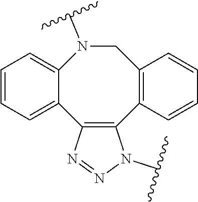 [SMILES: *N1Cc2ccccc2-c2c(nnn2*)-c2ccccc21] |
| [3 + 2] Cycloaddition, Esterification | 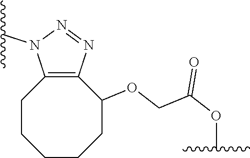 [SMILES: *OC(=O)COC1CCCCCc2c1nnn2*] |
| [3 + 2] Cycloaddition, Esterification | 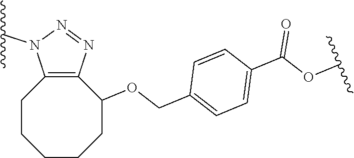 [SMILES: *OC(=O)c1ccc(COC2CCCCCc3c2nnn3*)cc1] |
| [3 + 2] Cycloaddition, Esterification | 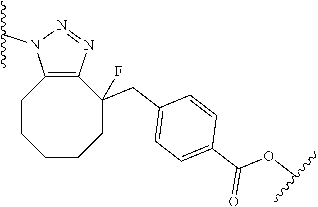 [SMILES: *OC(=O)c1ccc(CC2(F)CCCCCc3c2nnn3*)cc1] |
| [3 + 2] Cycloaddition, Esterification | 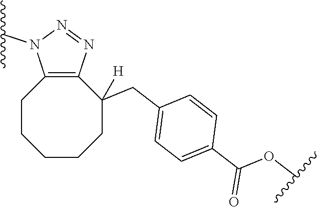 [SMILES: *OC(=O)c1ccc(CC2CCCCCc3c2nnn3*)cc1] |
| [3 + 2] Cycloaddition, Esterification | 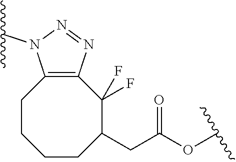 [SMILES: *OC(=O)CC1CCCCc2c(nnn2*)C1(F)F] |
| [3 + 2] Cycloaddition, Esterification | 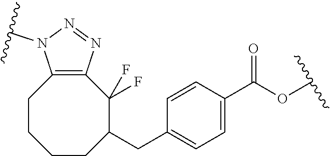 [SMILES: *OC(=O)c1ccc(CC2CCCCc3c(nnn3*)C2(F)F)cc1] |
| [3 + 2] Cycloaddition, Esterification | 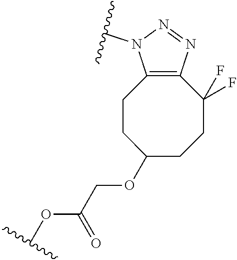 [SMILES: *OC(=O)COC1CCc2c(nnn2*)C(F)(F)CC1] |
| [3 + 2] Cycloaddition, Esterification | 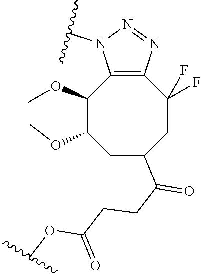 [SMILES: *OC(=O)CCC(=O)C1C[C@H](OC)[C@@H](OC)c2c(nnn2*)C(F)(F)C1] |
| [3 + 2] Cycloaddition, Esterification | 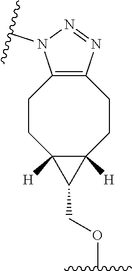 [SMILES: *OC[C@@H]1[C@@H]2CCc3c(nnn3*)CC[C@@H]21] |
| [3 + 2] Cycloaddition, Esterification | 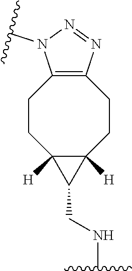 [SMILES: *NC[C@@H]1[C@@H]2CCc3c(nnn3*)CC[C@@H]21] |
| [3 + 2] Cycloaddition, Esterification | 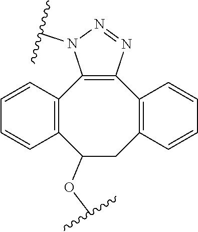 [SMILES: *OC1Cc2ccccc2-c2nnn(*)c2-c2ccccc21] |
| [3 + 2] Cycloaddition, Esterification | 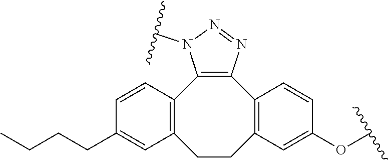 [SMILES: *Oc1ccc2c(c1)CCc1cc(CCCC)ccc1-c1c-2nnn1*] |
| [3 + 2] Cycloaddition | 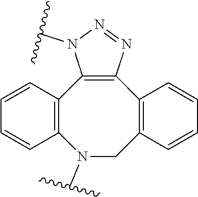 [SMILES: *N1Cc2ccccc2-c2nnn(*)c2-c2ccccc21] |
| Michael addition | 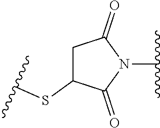 [SMILES: *SC1CC(=O)N(*)C1=O] |
| Michael addition | 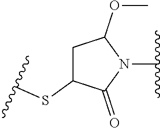 [SMILES: *SC1CC(OC)N(*)C1=O] |
| Imine condensation, Amidation | 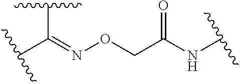 [SMILES: *NC(=O)CON=C(*)*] |
| Imine condensation | 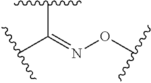 [SMILES: *ON=C(*)*] |
| Disulfide formation | 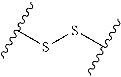 [SMILES: *SS*] |
| Thiol alkylation | 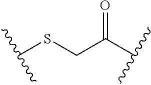 [SMILES: *SCC(*)=O] |
| Condensation, Michael addition | 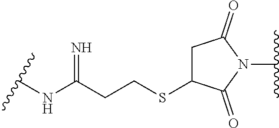 [SMILES: *NC(=N)CCSC1CC(=O)N(*)C1=O] |

One of skill in the art will recognize that a reactive substituent Z attached to the linker and a reactive substituent on the antibody or antigen-binding fragment thereof, are engaged in the covalent coupling reaction to produce the chemical moiety Z, and will recognize the reactive moiety Z′. Therefore, antibody-drug conjugates useful in conjunction with the methods described herein may be formed by the reaction of an antibody, or antigen-binding fragment thereof, with a linker or cytotoxin-linker conjugate, as described herein, the linker or cytotoxin-linker conjugate including a reactive substituent Z′, suitable for reaction with a reactive substituent on the antibody, or antigen-binding fragment thereof, to form the chemical moiety Z.

In some embodiments, Z′ is —NR¹³C(═O)CH═CH₂, —N₃, —SH, —S(═O)₂(CH═CH₂), —(CH₂)₂S(═O)₂(CH═CH₂), —NR¹³S(═O)₂(CH═CH₂), —NR¹³C(═O)CH₂R¹⁴, —NR¹³C(═O)CH₂Br, —NR¹³C(═O)CH₂I, —NHC(═O)CH₂Br, —NHC(═O)CH₂I, —ONH₂, —C(O)NHNH₂, —CO₂H, —NH₂, —NH(C═O), —NC(═S),

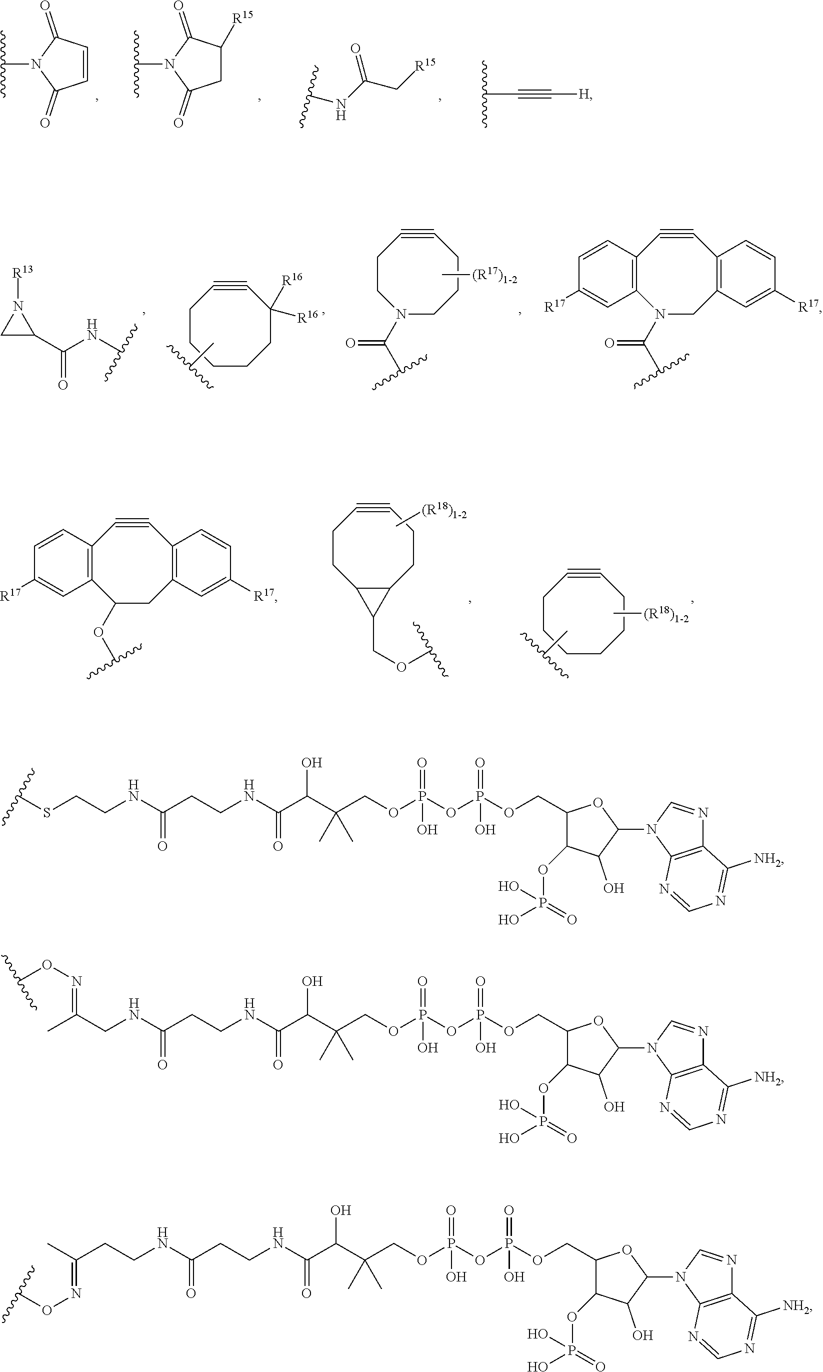

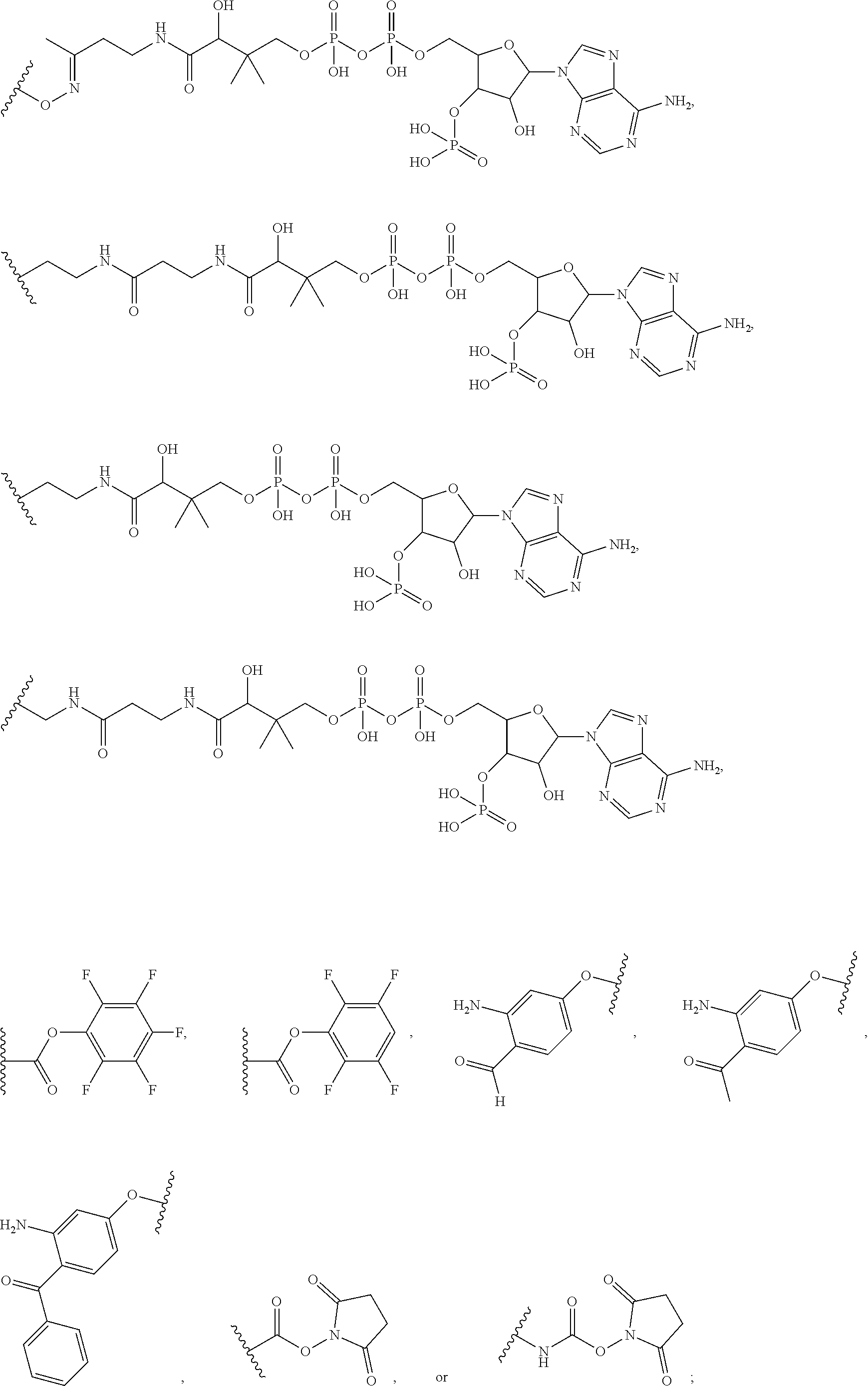

wherein

R¹³ is independently selected for each occasion from H and C₁-C₆ alkyl;

R¹⁴ is —S(CH₂)ₙCHR¹⁵NHC(═O)R¹³;

R¹⁵ is R¹³ or —C(═O)OR¹³;

R¹⁶ is independently selected for each occasion from H, C₁-C₆ alkyl, F, Cl, and —OH;

R¹⁷ is independently selected for each occasion from H, C₁-C₆ alkyl, F, Cl, —NH₂, —OCH₃, —OCH₂CH₃, —N(CH₃)₂, —CN, —NO₂ and —OH; and

R¹⁸ is independently selected for each occasion from H, C₁-C₆ alkyl, F, benzyloxy substituted with —C(═O)OH, benzyl substituted with —C(═O)OH, C₁-C₄ alkoxy substituted with —C(═O)OH, and C₁-C₄ alkyl substituted with —C(═O)OH;

m is independently selected for each occasion from 1, 2, 3, 4, 5, 6, 7, 8, 9 and 10; and

n is independently selected for each occasion from 1, 2, 3, 4, 5, 6, 7, 8, 9, 10, 11, 12, 13 and 14.

As depicted in Table 2, examples of suitably reactive substituents on the linker and antibody or antigen-binding fragment thereof include a nucleophile/electrophile pair (e.g., a thiol/haloalkyl pair, an amine/carbonyl pair, or a thiol/α,β-unsaturated carbonyl pair, and the like), a diene/dienophile pair (e.g., an azide/alkyne pair, or a diene/α,β-unsaturated carbonyl pair, among others), and the like. Coupling reactions between the reactive substitutents to form the chemical moiety Z include, without limitation, thiol alkylation, hydroxyl alkylation, amine alkylation, amine or hydroxylamine condensation, hydrazine formation, amidation, esterification, disulfide formation, cycloaddition (e.g., [4+2] Diels-Alder cycloaddition, [3+2] Huisgen cycloaddition, among others), nucleophilic aromatic substitution, electrophilic aromatic substitution, and other reactive modalities known in the art or described herein. Preferably, the linker contains an electrophilic functional group for reaction with a nucleophilic functional group on the antibody, or antigen-binding fragment thereof.

Reactive substituents that may be present within an antibody, or antigen-binding fragment thereof, as disclosed herein include, without limitation, nucleophilic groups such as (i) N-terminal amine groups, (ii) side chain amine groups, e.g. lysine, (iii) side chain thiol groups, e.g. cysteine, and (iv) sugar hydroxyl or amino groups where the antibody is glycosylated. Reactive substituents that may be present within an antibody, or antigen-binding fragment thereof, as disclosed herein include, without limitation, hydroxyl moieties of serine, threonine, and tyrosine residues; amino moieties of lysine residues; carboxyl moieties of aspartic acid and glutamic acid residues; and thiol moieties of cysteine residues, as well as propargyl, azido, haloaryl (e.g., fluoroaryl), haloheteroaryl (e.g., fluoroheteroaryl), haloalkyl, and haloheteroalkyl moieties of non-naturally occurring amino acids. In some embodiments, the reactive substituents present within an antibody, or antigen-binding fragment thereof as disclosed herein include, are amine or thiol moieties. Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by introducing one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

In some embodiments, the reactive substituent Z′ attached to the linker is a nucleophilic group which is reactive with an electrophilic group present on an antibody. Useful electrophilic groups on an antibody include, but are not limited to, aldehyde and ketone carbonyl groups. The heteroatom of a nucleophilic group can react with an electrophilic group on an antibody and form a covalent bond to the antibody. Useful nucleophilic groups include, but are not limited to, hydrazide, oxime, amino, hydroxyl, hydrazine, thiosemicarbazone, hydrazine carboxylate, and arylhydrazide.

In some embodiments, Z is the product of a reaction between reactive nucleophilic substituents present within the antibodies, or antigen-binding fragments thereof, such as amine and thiol moieties, and a reactive electrophilic substituent Z′. For instance, Z′ may be a Michael acceptor (e.g., maleimide), activated ester, electron-deficient carbonyl compound, and aldehyde, among others.

Several representative and non-limiting examples of reactive substituents and the resulting chemical moieties are provided in Table 3.

TABLE 3
Complementary reactive substituents and chemical moieties

| | Functional Group on | | |
|---|---|---|---|
| | Antibody | Z′ group | Z group |
| Naturally Occurring | 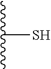 [SMILES: *S] | 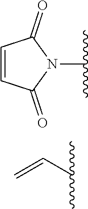 | 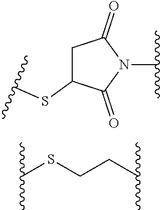 |
| | 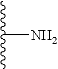 [SMILES: *N] | 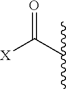 | 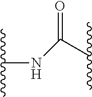 [SMILES: *NC(*)=O] |
| Synthetically Introduced | 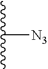 [SMILES: *N=[N+]=[N-]] | 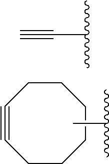 | 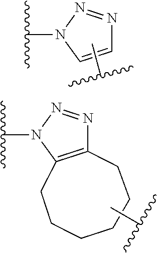 |
| | 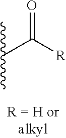 | 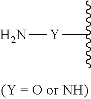 | 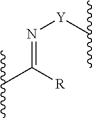 |

For instance, linkers suitable for the synthesis of ADCs include, without limitation, reactive substituents Z such as maleimide or haloalkyl groups. These may be attached to the linker by reagents such as succinimidyl 4-(N-maleimidomethyl)-cyclohexane-L-carboxylate (SMCC), N-succinimidyl iodoacetate (SIA), sulfo-SMCC, m-maleimidobenzoyl-N-hydroxysuccinimidyl ester (MBS), sulfo-MBS, and succinimidyl iodoacetate, among others described, in for instance, Liu et al., 18:690-697, 1979, the disclosure of which is incorporated herein by reference as it pertains to linkers for chemical conjugation.

In some embodiments, the reactive substituent Z′ attached to linker L is a maleimide, azide, or alkyne. An example of a maleimide-containing linker is the non-cleavable maleimidocaproyl-based linker, which is particularly useful for the conjugation of microtubule-disrupting agents such as auristatins. Such linkers are described by Doronina et al., Bioconjugate Chem. 17:14-24, 2006, the disclosure of which is incorporated herein by reference as it pertains to linkers for chemical conjugation.

In some embodiments, the reactive substituent Z′ is —(C═O)— or —NH(C═O)—, such that the linker may be joined to the antibody, or antigen-binding fragment thereof, by an amide or urea moiety, respectively, resulting from reaction of the —(C═O)— or —NH(C═O)— group with an amino group of the antibody or antigen-binding fragment thereof.

In some embodiments, the reactive substituent Z′ is an N-maleimidyl group, halogenated N-alkylamido group, sulfonyloxy N-alkylamido group, carbonate group, sulfonyl halide group, thiol group or derivative thereof, alkynyl group comprising an internal carbon-carbon triple bond, (het-ero)cycloalkynyl group, bicyclo[6.1.0]non-4-yn-9-yl group, alkenyl group comprising an internal carbon-carbon double bond, cycloalkenyl group, tetrazinyl group, azido group, phosphine group, nitrile oxide group, nitrone group, nitrile imine group, diazo group, ketone group, (O-alkyl)hydroxylamino group, hydrazine group, halogenated N-maleimidyl group, 1,1-bis (sulfonylmethyl)methylcarbonyl group or elimination derivatives thereof, carbonyl halide group, or an allenamide group, each of which may be optionally substituted. In some embodiments, the reactive substituent comprises a cycloalkene group, a cycloalkyne group, or an optionally substituted (hetero)cycloalkynyl group.

Exemplary antibody-drug conjugates and ligand-drug conjugates useful in conjunction with the methods described herein may be formed by the reaction of an antibody, antigen-binding fragment thereof, or ligand with an amatoxin that is conjugated to a linker containing a substituent suitable for reaction with a reactive residue on the antibody, antigen-binding fragment thereof, or ligand. Non-limiting examples of amatoxin-linker conjugates containing a reactive substituent Z′ suitable for reaction with a reactive residue on the antibody or antigen-binding fragment thereof include, without limitation, 7′C-(4-(6-(maleimido)hexanoyl)piperazin-1-yl)-amatoxin; 7′C-(4-(6-(maleimido)hexanamido)piperidin-1-yl)-amatoxin; 7′C-(4-(6-(6-(maleimido)hexanamido)hexanoyl)piperazin-1-yl)-amatoxin; 7′C-(4-(4-((maleimido)methyl)cyclohexanecarbonyl)piperazin-1-yl)-amatoxin; 7′C-(4-(6-(4-((maleimido)methyl)cyclohexanecarboxamido)hexanoyl)piperazin-1-yl)-amatoxin; 7′C-(4-(2-(6-(maleimido)hexanamido)ethyl)piperidin-1-yl)-amatoxin; 7′C-(4-(2-(6-(6-(maleimido)hexanamido)hexanamido)ethyl)piperidin-1-yl)-amatoxin; 7′C-(4-(2-(4-((maleimido)methyl)cyclohexanecarboxamido)ethyl)piperidin-1-yl)-amatoxin; 7′C-(4-(2-(6-(4-((maleimido)methyl)cyclohexanecarboxamido)hexanamido)ethyl)piperidin-1-yl)-amatoxin; 7′C-(4-(2-(3-carboxypropanamido)ethyl)piperidin-1-yl)-amatoxin; 7′C-(4-(2-(2-bromoacetamido)ethyl)piperidin-1-yl)-amatoxin; 7′C-(4-(2-(3-(pyridin-2-yldisulfanyl)propanamido)ethyl)piperidin-1-yl)-amatoxin; 7′C-(4-(2-(4-(maleimido)butanamido)ethyl)piperidin-1-yl)-amatoxin; 7′C-(4-(2-(maleimido)acetyl)piperazin-1-yl)-amatoxin; 7′C-(4-(3-(maleimido)propanoyl)piperazin-1-yl)-amatoxin; 7′C-(4-(4-(maleimido)butanoyl)piperazin-1-yl)-amatoxin; 7′C-(4-(2-(6-(4-((maleimido)methyl)cyclohexanecarboxamido)hexanamido)ethyl)piperidin-1-yl)-amatoxin; 7′C-(3-((6-(maleimido)hexanamido)methyl)pyrrolidin-1-yl)-amatoxin; 7′C-(3-((6-(6-(maleimido)hexanamido)hexanamido)methyl)pyrrolidin-1-yl)-amatoxin; 7′C-(3-((4-((maleimido)methyl)cyclohexanecarboxamido)methyl)pyrrolidin-1-yl)-amatoxin; 7′C-(3-((6-((4-(maleimido)methyl)cyclohexanecarboxamido)hexanamido)methyl)pyrrolidin-1-yl)-amatoxin; 7′C-(4-(2-(6-(2-(aminooxy)acetamido)hexanamido)ethyl)piperidin-1-yl)-amatoxin; 7′C-(4-(2-(4-(2-(aminooxy)acetamido)butanamido)ethyl)piperidin-1-yl)-amatoxin; 7′C-(4-(4-(2-(aminooxy)acetamido)butanoyl)piperazin-1-yl)-amatoxin; 7′C-(4-(6-(2-(aminooxy)acetamido)hexanoyl)piperazin-1-yl)-amatoxin; 7′C-((4-(6-(maleimido)hexanamido)piperidin-1-yl)methyl)-amatoxin; 7′C-((4-(2-(6-(maleimido)hexanamido)ethyl)piperidin-1-yl)methyl)-amatoxin; 7′C-((4-(6-(maleimido)hexanoyl)piperazin-1-yl)methyl)-amatoxin; (R)-7′C-((3-((6-(maleimido)hexanamido)methyl)pyrrolidin-1-yl)methyl)-amatoxin; (S)-7′C-((3-((6-(maleimido)hexanamido)methyl)pyrrolidin-1-yl)methyl)-amatoxin; 7′C-((4-(2-(6-(6-(maleimido)hexanamido)hexanamido)ethyl)piperidin-1-yl)methyl)-amatoxin; 7′C-((4-(2-(4-((maleimido)methyl)cyclohexanecarboxamido)ethyl)piperidin-1-yl)methyl)-amatoxin; 7′C-((4-(2-(6-(4-((maleimido)methyl)cyclohexanecarboxamido)hexanamido)ethyl)piperidin-1-yl)methyl)-amatoxin; 7′C-((4-(2-(6-(maleimido)hexanamido)ethyl)piperazin-1-yl)methyl)-amatoxin; 7′C-((4-(2-(6-(6-(maleimido)hexanamido)hexanamido)ethyl)piperazin-1-yl)methyl)-amatoxin; 7′C-((4-(2-(4-((maleimido)methyl)cyclohexanecarboxamido)ethyl)piperazin-1-yl)methyl)-amatoxin; 7′C-((4-(2-(6-(4-((maleimido)methyl)cyclohexanecarboxamido)hexanamido)ethyl)piperazin-1-yl)methyl)-amatoxin; 7′C-((3-((6-(6-(maleimido)hexanamido)hexanamido)-S-methyl)pyrrolidin-1-yl)methyl)-amatoxin; 7′C-((3-((6-(6-(maleimido)hexanamido)hexanamido)-R-methyl)pyrrolidin-1-yl)methyl)-amatoxin; 7′C-((3-((4-((maleimido)methyl)cyclohexanecarboxamido)-S-methyl)pyrrolidin-1-yl)methyl)-amatoxin; 7′C-((3-((4-((maleimido)methyl)cyclohexanecarboxamido)-R-methyl)pyrrolidin-1-yl)methyl)-amatoxin; 7′C-((3-((6-(4-((maleimido)methyl)cyclohexanecarboxamido)hexanamido)methyl)pyrrolidin-1-yl)methyl)-amatoxin; 7′C-((4-(2-(3-carboxypropanamido)ethyl)piperazin-1-yl)methyl)-amatoxin; 7′C-((4-(6-(6-(maleimido)hexanamido)hexanoyl)piperazin-1-yl)methyl)-amatoxin; 7′C-((4-(6-(4-((maleimido)methyl)cyclohexanecarboxamido)hexanoyl)piperazin-1-yl)methyl)-amatoxin; 7′C-((4-(2-(maleimido)acetyl)piperazin-1-yl)methyl)-amatoxin; 7′C-((4-(3-(maleimido)propanoyl)piperazin-1-yl)methyl)-amatoxin; 7′C-((4-(4-(maleimido)butanoyl)piperazin-1-yl)methyl)-amatoxin; 7′C-((4-(2-(2-(maleimido)acetamido)ethyl)piperidin-1-yl)methyl)-amatoxin; 7′C-((4-(2-(4-(maleimido)butanamido)ethyl)piperidin-1-yl)methyl)-amatoxin; 7′C-((4-(2-(6-(4-((maleimido)methyl)cyclohexanecarboxamido)hexanamido)ethyl)piperidin-1-yl)methyl)-amatoxin; 7′C-((3-((6-(maleimido)hexanamido)methyl)azetidin-1-yl)methyl)-amatoxin; 7′C-((3-(2-(6-(maleimido)hexanamido)ethyl)azetidin-1-yl)methyl)-amatoxin; 7′C-((3-((4-((maleimido)methyl)cyclohexanecarboxamido)methyl)azetidin-1-yl)methyl)-amatoxin; 7′C-((3-(2-(4-((maleimido)methyl)cyclohexanecarboxamido)ethyl)azetidin-1-yl)methyl)-amatoxin; 7′C-((3-(2-(6-(4-((maleimido)methyl)cyclohexanecarboxamido)hexanamido)ethyl)azetidin-1-yl)methyl)-amatoxin; 7′C-(((2-(6-(maleimido)-N-methylhexanamido)ethyl)(methyl)amino)methyl)-amatoxin; 7′C-(((4-(6-(maleimido)-N-methylhexanamido)butyl(methyl)amino)methyl)-amatoxin; 7′C-((2-(2-(6-(maleimido)hexanamido)ethyl)aziridin-l-yl)methyl)-amatoxin; 7′C-((2-(2-(6-(4-((maleimido)methyl)cyclohexanecarboxamido)hexanamido)ethyl)aziridin-l-yl)methyl)-amatoxin; 7′C-((4-(6-(6-(2-(aminooxy)acetamido)hexanamido)hexanoyl)piperazin-1-yl)methyl)-amatoxin; 7′C-((4-(1-(aminooxy)-2-oxo-6,9,12,15-tetraoxa-3-azaheptadecan-17-oyl)piperazin-1-yl)methyl)-amatoxin; 7′C-((4-(2-(2-(aminooxy)acetamido)acetyl)piperazin-1-yl)methyl)-amatoxin; 7′C-((4-(3-(2-(aminooxy)acetamido)propanoyl)piperazin-1-yl)methyl)-amatoxin; 7′C-((4-(4-(2-(aminooxy)acetamido)butanoyl)piperazin-1-yl)methyl)-amatoxin; 7′C-((4-(2-(6-(2-(aminooxy)acetamido)hexanamido)ethyl)piperidin-1-yl)methyl)-amatoxin; 7′C-((4-(2-(2-(2-(aminooxy)acetamido)acetamido)ethyl)piperidin-1-yl)methyl)-amatoxin; 7′C-((4-(2-(4-(2-(aminooxy)acetamido)butanamido)ethyl)piperidin-1-yl)methyl)-amatoxin; 7′C-((4-(20-(aminooxy)-4,19-dioxo-6,9,12,15-tetraoxa-3,18-diazaicosyl)piperidin-1-yl)methyl)-amatoxin; 7′C-(((2-(6-(2-(aminooxy)acetamido)-N-methylhexanamido)ethyl)(methyl)amino)methyl)-amatoxin; 7′C-(((4-(6-(2-(aminooxy)acetamido)-N-methylhexanamido)butyl)(methyl)amino)methyl)-amatoxin; 7′C-((3-((6-(4-((maleimido)methyl)cyclohexanecarboxamido)hexanamido)methyl)pyrrolidin-1-yl)-S-methyl)-amatoxin; 7′C-((3-((6-(4-((maleimido)methyl)cyclohexanecarboxamido)hexanamido)-R-methyl)pyrrolidin-1-yl)methyl)-amatoxin; 7′C-((4-(2-(2-bromoacetamido)ethyl)piperazin-1-yl)methyl)-amatoxin; 7′C-((4-(2-(2-bromoacetamido)ethyl)piperidin-1-yl)methyl)-amatoxin; 7′C-((4-(2-(3-(pyridine-2-yldisulfanyl)propanamido)ethyl)piperidin-1-yl)methyl)-amatoxin; 6′O-(6-(6-(maleimido)hexanamido)hexyl)-amatoxin; 6′O-(5-(4-((maleimido)methyl)cyclohexanecarboxamido)pentyl)-amatoxin; 6′O-(2-((6-(maleimido)hexyl)oxy)-2-oxoethyl)-amatoxin; 6′O-((6-(maleimido)hexyl)carbamoyl)-amatoxin; 6′O-((6-(4-((maleimido)methyl)cyclohexanecarboxamido)hexyl)carbamoyl)-amatoxin; 6′O-(6-(2-bromoacetamido)hexyl)-amatoxin; 7′C-(4-(6-(azido)hexanamido)piperidin-1-yl)-amatoxin; 7′C-(4-(hex-5-ynoylamino)piperidin-1-yl)-amatoxin; 7′C-(4-(2-(6-(maleimido)hexanamido)ethyl)piperazin-1-yl)-amatoxin; 7′C-(4-(2-(6-(6-(maleimido)hexanamido)hexanamido)ethyl)piperazin-1-yl)-amatoxin; 6′O-(6-(6-(11,12-didehydro-5,6-dihydro-dibenz[b,f]azocin-5-yl)-6-oxohexanamido)hexyl)-amatoxin; 6′O-(6-(hex-5-ynoylamino)hexyl)-amatoxin; 6′O-(6-(2-(aminooxy)acetylamido)hexyl)-amatoxin; 6′O-((6-aminooxy)hexyl)-amatoxin; and 6′O-(6-(2-iodoacetamido)hexyl)-amatoxin.

In some embodiments, the ADC comprises an anti-CD45 antibody conjugated to an amatoxin of any of formulae III, IIIA, IIIB, or IIIC as disclosed herein, via a linker L and a chemical moiety Z, wherein the linker includes a hydrazine, a disulfide, a thioether or a peptide. In some embodiments, the linker includes a dipeptide. In some embodiments, the linker includes a dipeptide selected from Val-Ala and Val-Cit. In some embodiments, the linker includes a para-aminobenzyl group (PAB). In some embodiments, the linker includes the moiety PAB-Cit-Val. In some embodiments, the linker includes the moiety PAB-Ala-Val. In some embodiments, the linker includes a —((C═O)(CH₂)ₙ— unit, wherein n is an integer from 1-6. In some embodiments, the linker is -PAB-Cit-Val-((C═O)(CH₂)ₙ—.

In some embodiments, the linker includes a —(CH₂)ₙ— unit, where n is an integer from 2-6. In some embodiments, the linker is -PAB-Cit-Val-((C═O)(CH₂)ₙ. In some embodiments, the linker is -PAB-Ala-Val-((C═O)(CH₂)ₙ—. In some embodiments, the linker is —(CH₂)ₙ—. In some embodiments, the linker is —((CH₂)ₙ—, wherein n is 6.

In some embodiments, the chemical moiety Z is selected from Table 2. In some embodiments, the chemical moiety Z is

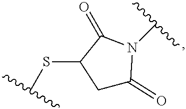

where S is a sulfur atom which represents the reactive substituent present within an antibody, or an antigen-binding fragment thereof, that binds CD45 (e.g., from the —SH group of a cysteine residue).

In some embodiments, the linker L and the chemical moiety Z, taken together as L-Z, is

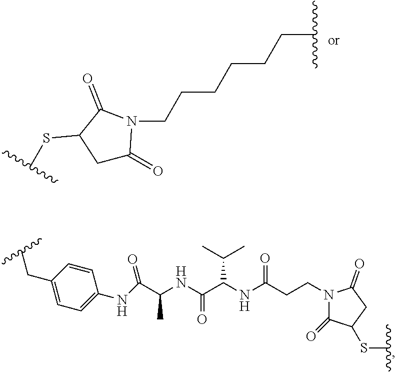

One of skill in the art will recognize the linker-reactive substituent group structure, prior to conjugation with the antibody or antigen binding fragment thereof, includes a maleimide as the group Z. The foregoing linker moieties and amatoxin-linker conjugates, among others useful in conjunction with the compositions and methods described herein, are described, for example, in U.S. Patent Application Publication No. 2015/0218220 and Patent Application Publication No. WO2017/149077, the disclosure of each of which is incorporated herein by reference in its entirety.

In some embodiments, the linker-reactive substituent group structure L-Z′, prior to conjugation with the antibody or antigen binding fragment thereof, is:

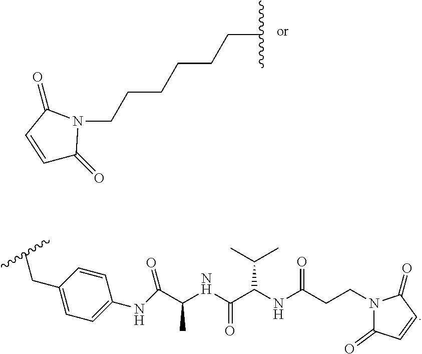

In some embodiments, an amatoxin as disclosed herein is conjugated to a linker-reactive moiety -L-Z′ having the following formula:

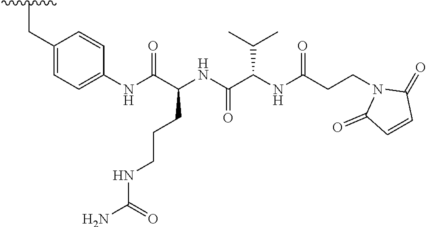 [SMILES: *Cc1ccc(NC(=O)[C@H](CCCNC(N)=O)NC(=O)[C@@H](NC(=O)CCN2C(=O)C=CC2=O)C(C)C)cc1]

where the wavy line indicates the attachment point to the amatoxin.

In some embodiments, an amatoxin as disclosed herein is conjugated to a linker-reactive substituent -L-Z′ having the following formula:

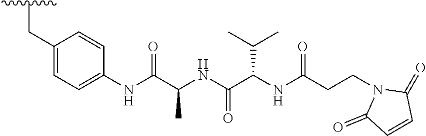 [SMILES: *Cc1ccc(NC(=O)[C@H](C)NC(=O)[C@@H](NC(=O)CCN2C(=O)C=CC2=O)C(C)C)cc1]

where the wavy line indicates the attachment point to the amatoxin.

The foregoing linker moieties and amatoxin-linker conjugates, among others useful in conjunction with the compositions and methods described herein, are described, for example, in

U.S. Patent Application Publication No. 2015/0218220 and Patent Application Publication No. WO2017/149077, the disclosure of each of which is incorporated herein by reference in its entirety.

### Preparation of Antibody-Drug Conjugates

In the ADCs of Formula (I) and (II) as disclosed herein, an anti-CD45 antibody or antigen binding fragment thereof is conjugated to one or more cytotoxic drug moieties (D), e.g. about 1 to about 20 drug moieties per antibody, through a linker L and a chemical moiety Z as disclosed herein. The ADCs of the present disclosure may be prepared by several routes, employing organic chemistry reactions, conditions, and reagents known to those skilled in the art, including: (1) reaction of a reactive substituent of an antibody or antigen binding fragment thereof with a bivalent linker reagent to form Ab-Z-L as described herein above, followed by reaction with a drug moiety D; or (2) reaction of a reactive substituent of a drug moiety with a bivalent linker reagent to form D-L-Z, followed by reaction with a reactive substituent of an antibody or antigen binding fragment thereof as described herein above. Additional methods for preparing ADC are described herein.

In another aspect, the anti-CD45 antibody or antigen binding fragment thereof has one or more lysine residues that can be chemically modified to introduce one or more sulfhydryl groups. The ADC is then formed by conjugation through the sulfhydryl group's sulfur atom as described herein above. The reagents that can be used to modify lysine include, but are not limited to, N-succinimidyl S-acetylthioacetate (SATA) and 2-Iminothiolane hydrochloride (Traut's Reagent).

In another aspect, the anti-CD45 antibody or antigen binding fragment thereof can have one or more carbohydrate groups that can be chemically modified to have one or more sulfhydryl groups. The ADC is then formed by conjugation through the sulfhydryl group's sulfur atom as described herein above.

In yet another aspect, the anti-CD45 antibody can have one or more carbohydrate groups that can be oxidized to provide an aldehyde (—CHO) group (see, for e.g., Laguzza, et al., J. Med. Chem. 1989, 32(3), 548-55). The ADC is then formed by conjugation through the corresponding aldehyde as described herein above. Other protocols for the modification of proteins for the attachment or association of cytotoxins are described in Coligan et al., Current Protocols in Protein Science, vol. 2, John Wiley & Sons (2002), incorporated herein by reference.

Methods for the conjugation of linker-drug moieties to cell-targeted proteins such as antibodies, immunoglobulins or fragments thereof are found, for example, in U.S. Pat. Nos. 5,208,020; 6,441,163; WO2005037992; WO2005081711; and WO2006/034488, all of which are hereby expressly incorporated by reference in their entirety.

Alternatively, a fusion protein comprising the antibody and cytotoxic agent may be made, e.g., by recombinant techniques or peptide synthesis. The length of DNA may comprise respective regions encoding the two portions of the conjugate either adjacent one another or separated by a region encoding a linker peptide which does not destroy the desired properties of the conjugate.

Therapeutic Uses

As described above, the amount of ADC administered should be sufficient to deplete lymphocytes which reject CAR cell therapy. In one embodiment, a therapeutically effective dose of the anti-CD45 ADC will be lower than doses used for anti-CD45ADC conditioning. The determination of a therapeutically effective dose is within the capability of practitioners in this art, however, as an example, in embodiments of the method described herein utilizing systemic administration of an ADC for the treatment of an immune disease or cancer, an effective human dose may be in the range of about 0.001-about 150 mg/kg, e.g., about 0.1-about 150 mg/kg (e.g., about 5 mg/kg, about 10 mg/kg, about 25 mg/kg, about 50 mg/kg, about 75 mg/kg, about 100 mg/kg, about 150 mg/kg etc.). In one embodiment, a therapeutically effective dose of an anti-CD45 ADC for treatment prior to CAR therapy in a human patient will be a dose amount that will deplete lymphocytes in the subject while generally not depleting HSCs in the subject. At higher doses, e.g., conditioning for stem cell transplantation therapy, anti-CD45 ADCs may be used to deplete human HSCs (see, for example, WO 2017/219025). As described in the examples below, anti-CD45 ADCs may be used deplete lymphocytes, where at higher doses anti-CD45 ADCs may be used to deplete both lymphocytes and HSCs. Thus, therapeutically effective doses of an anti-CD45 ADC for lymphodepletion prior to CAR therapy in a subject are doses that maintain overall HSC survival in the patient while depleting lymphocytes. For example, in some embodiments, therapeutically effective doses of an anti-CD45 ADC for lymphodepletion prior to CAR therapy in a subject may be doses that substantially maintain overall HSC survival in the patient while substantially depleting lymphocytes.

The effective dose of an anti-CD45 ADC described herein can range, for example from about 0.001 to about 100 mg/kg of body weight per single (e.g., bolus) administration, multiple administrations, or continuous administration, or to achieve an optimal serum concentration (e.g., a serum concentration of about 0.0001-about 5000 μg/mL) of the anti-CD45 ADC. A dose of the anti-CD45 ADC may be administered one or more times (e.g., 2-10 times) per day, week, or month to a human subject who has had, is concomitantly receiving, or will be receiving CAR therapy at a time point following delivery of the anti-CD45 ADC. An anti-CD45 ADC may be administered to the human patient one time or as multiple doses. In one embodiment, the anti-CD45 ADC can be administered in an amount sufficient to reduce the quantity of host-reactive lymphocytes, for example, by about 10%, about 20%, about 30%, about 40%, about 50%, about 60%, about 70%, about 80%, about 90%, about 95%, or more prior to CAR therapy.

In one embodiment, the dose of the anti-CD45 antibody conjugated via a linker to a cytotoxin administered to the human patient is about 0.1 mg/kg to about 0.3 mg/kg.

In one embodiment, the dose of the anti-CD45 antibody conjugated via a linker to a cytotoxin administered to the human patient is about 0.15 mg/kg to about 0.3 mg/kg.

In one embodiment, the dose of the anti-CD45 antibody conjugated via a linker to a cytotoxin administered to the human patient is about 0.15 mg/kg to about 0.25 mg/kg.

In one embodiment, the dose of the anti-CD45 antibody conjugated via a linker to a cytotoxin administered to the human patient is about 0.2 mg/kg to about 0.3 mg/kg.

In one embodiment, the dose of the anti-CD45 antibody conjugated via a linker to a cytotoxin administered to the human patient is about 0.25 mg/kg to about 0.3 mg/kg.

In one embodiment, the dose of the anti-CD45 antibody conjugated via a linker to a cytotoxin administered to the human patient is about 0.1 mg/kg.

In one embodiment, the dose of the anti-CD45 antibody conjugated via a linker to a cytotoxin administered to the human patient is about 0.2 mg/kg.

In one embodiment, the dose of the anti-CD45 antibody conjugated via a linker to a cytotoxin administered to the human patient is about 0.3 mg/kg.

In one embodiment, the dose of the anti-CD45 ADC described herein administered to the human patient is about 0.001 mg/kg to 10 mg/kg, about 0.01 mg/kg to 9.5 mg/kg, about 0.1 mg/kg to 9 mg/kg, about 0.1 mg/kg to 8.5 mg/kg, about 0.1 mg/kg to 8 mg/kg, about 0.1 mg/kg to 7.5 mg/kg, about 0.1 mg/kg to 7 mg/kg, about 0.1 mg/kg to 6.5 mg/kg, about 0.1 mg/kg to 6 mg/kg, about 0.1 mg/kg to 5.5 mg/kg, about 0.1 mg/kg to 5 mg/kg, about 0.1 mg/kg to 4.5 mg/kg, about 0.1 mg/kg to 4 mg/kg, about 0.5 mg/kg to 3.5 mg/kg, about 0.5 mg/kg to 3 mg/kg, about 1 mg/kg to 10 mg/kg, about 1 mg/kg to 9 mg/kg, about 1 mg/kg to 8 mg/kg, about 1 mg/kg to 7 mg/kg, about 1 mg/kg to 6 mg/kg, about 1 mg/kg to 5 mg/kg, about 1 mg/kg to 4 mg/kg, or about 1 mg/kg to 3 mg/kg.

In one embodiment, the anti-CD45 ADC described herein that is administered to a human patient for treatment or conditioning has a half-life of equal to or less than 24 hours, equal to or less than 22 hours, equal to or less than 20 hours, equal to or less than 18 hours, equal to or less than 16 hours, equal to or less than 14 hours, equal to or less than 13 hours, equal to or less than 12 hours, equal to or less than 11 hours, equal to or less than 10 hours, equal to or less than 9 hours, equal to or less than 8 hours, equal to or less than 7 hours, equal to or less than 6 hours, or equal to or less than 5 hours. In one embodiment, the half life of the anti-HC ADC is 5 hours to 7 hours; is 5 hours to 9 hours; is 15 hours to 11 hours; is 5 hours to 13 hours; is 5 hours to 15 hours; is 5 hours to 20 hours; is 5 hours to 24 hours; is 7 hours to 24 hours; is 9 hours to 24 hours; is 11 hours to 24 hours; 12 hours to 22 hours; 10 hours to 20 hours; 8 hours to 18 hours; or 14 hours to 24 hours.

In one embodiment, the methods disclosed herein minimize liver toxicity in the patient receiving the ADC for conditioning. For example, in certain embodiments, the methods disclosed herein result in a liver marker level remaining below a known toxic level in the patient for more than about 24 hours, about 48 hours, about 72 hours, or about 96 hours. In other embodiments, the methods disclosed herein result in a liver marker level remaining within a reference range in the patient for more than about 24 hours, about 48 hours, about 72 hours, or about 96 hours. In certain embodiments, the methods disclosed herein result in a liver marker level rising not more than about 1.5-fold above a reference range, not more than about 3-fold above a reference range, not more than about 5-fold above a reference range, or not more than about 10-fold above a reference range for more than about 24 hours, about 48 hours, about 72 hours, or about 96 hours. Examples of liver markers that can be used to test for toxicity include alanine aminotransaminase (ALT), lactate dehydrogenase (LDH), and aspartate aminotransaminase (AST). In certain embodiments, administration of an ADC as described herein, i.e., where two doses are administered instead of a single dose, results in a transient increase in a liver marker, e.g., AST, LDH, and/or ALT. In some instances, an elevated level of a liver marker indicating toxicity may be reached, but within a certain time period, e.g., about 12 hours, about 18 hours, about 24 hours, about 36 hours, about 48 hours, about 72 hours, about 3 days, about 3.5 days, about 4 days, about 4.5 days, about 5 days, about 5.5 days, about 6 days, about 6.5 days, about 7 days, about 7.5 days, or less than a week, the liver marker level returns to a normal level not associated with liver toxicity. For example, in a human (average adult male), a normal, non-toxic level of ALT is 7 to 55 units per liter (U/L); and a normal, non-toxic level of AST is 8 to 48 U/L. In certain embodiments, at least one of the patient's blood AST, ALT, or LDH levels does not reach a toxic level between administration of a first dose of the ADC and 14 days after administration of the first dose to the patient. For example, the patient may be administered a first dose and subsequently a second dose, a third dose, a fourth dose, or more doses within, e.g., 5, 10, or 14 days of being administered the first dose, yet at least one of the patient's blood AST, ALT, or LDH levels does not reach a toxic level between administration of a first dose of the ADC and 14 days after administration of the first dose to the patient.

In certain embodiments, at least one of the patient's blood AST, ALT, or LDH levels does not rise above normal levels, does not rise more than 1.5-fold above normal levels, does not rise more than 3-fold above normal levels, does not rise more than 5-fold above normal levels, or does not rise more than 10-fold above normal levels.

The route of administration may affect the recommended dose. Repeated systemic doses are contemplated in order to maintain an effective level, e.g., to reduce the risk of CAR-T cell rejection, depending on the mode of administration adopted.

The anti-CD45 ADCs described herein may be administered by a variety of routes, such as orally, transdermally, subcutaneously, intranasally, intravenously, intramuscularly, intraocularly, or parenterally. The most suitable route for administration in any given case will depend on the particular ADC, the patient, pharmaceutical formulation methods, administration methods (e.g., administration time and administration route), the patient's age, body weight, sex, severity of the diseases being treated, the patient's diet, and the patient's excretion rate.

ADCs described herein can be administered to a patient, as described above, (e.g., a human patient suffering from an immune disease or cancer) in a variety of dosage forms. For instance, ADCs described herein can be administered to a patient suffering from an immune disease or cancer in the form of an aqueous solution, such as an aqueous solution containing one or more pharmaceutically acceptable excipients. Suitable pharmaceutically acceptable excipients for use with the compositions and methods described herein include viscosity-modifying agents. The aqueous solution may be sterilized using techniques known in the art.

Pharmaceutical formulations comprising anti-CD45 ADCs as described herein are prepared by mixing such ADC with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG).

### EXAMPLES

The following examples are put forth so as to provide those of ordinary skill in the art with a description of how the compositions and methods described herein may be used, made, and evaluated, and are intended to be purely exemplary of the invention and are not intended to limit the scope of what the inventors regard as their invention.

### Example 1. In Vitro Analysis of an Anti-CD45-Amatoxin Antibody Drug Conjugate (ADC) Using an In Vitro Cell Killing Assay

The anti-CD45 ADC used in the following Example is Ab1 conjugated to amanitin (i.e., anti-CD45-AM; fast half-life variant D265C, H435A at a DAR of 2; i.e., ADC 1). The sequences of Ab1 are set forth in SEQ ID Nos: 1 to 7. The amatoxin used in this example is represented by Formula (I) with an R8 linkage.

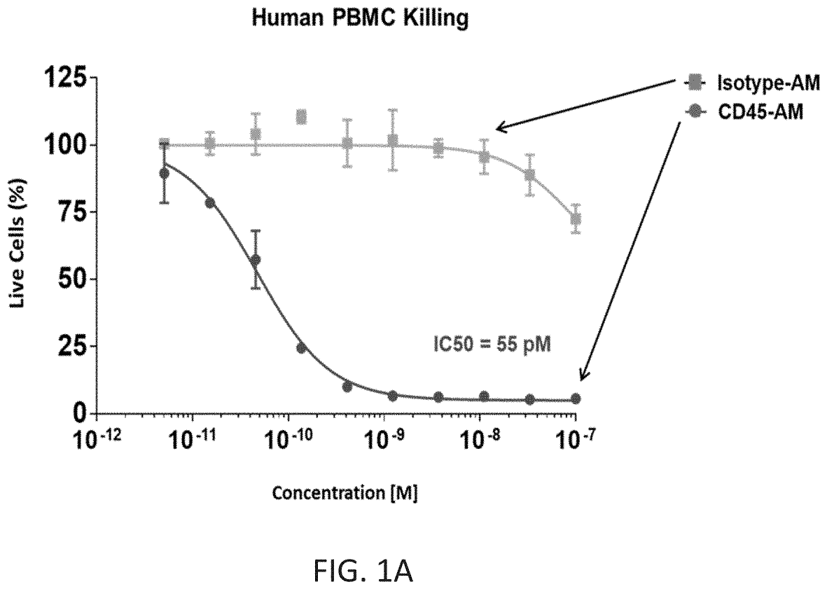

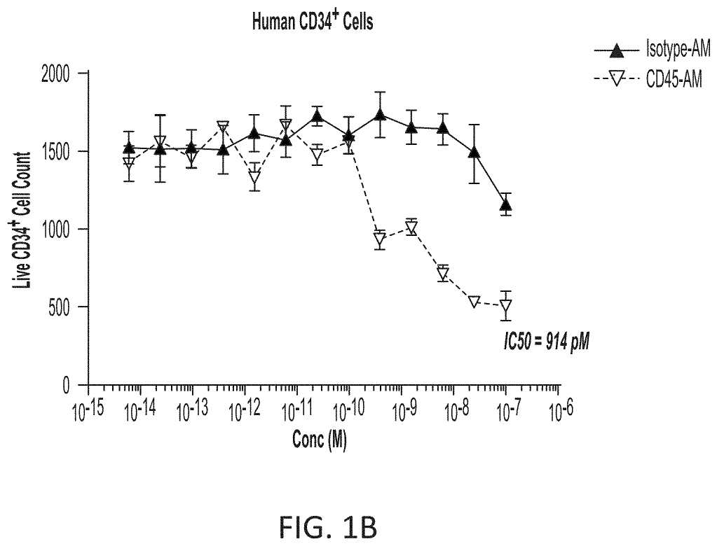

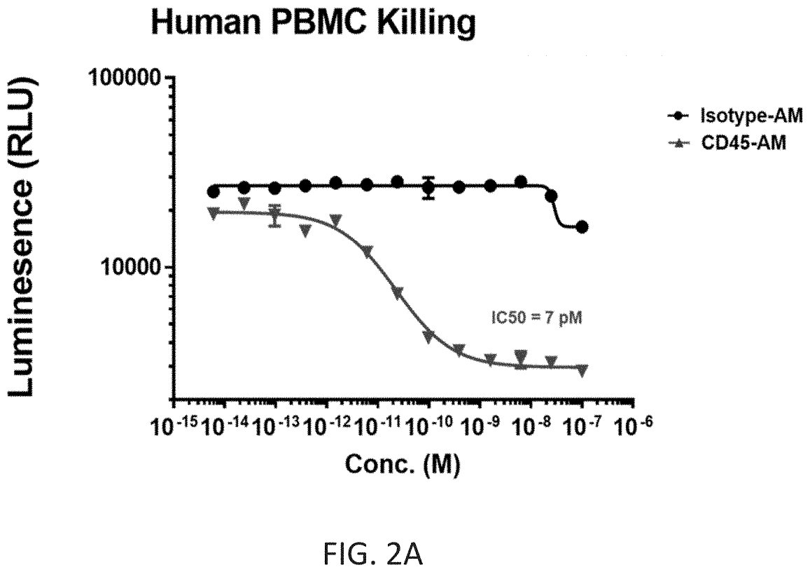

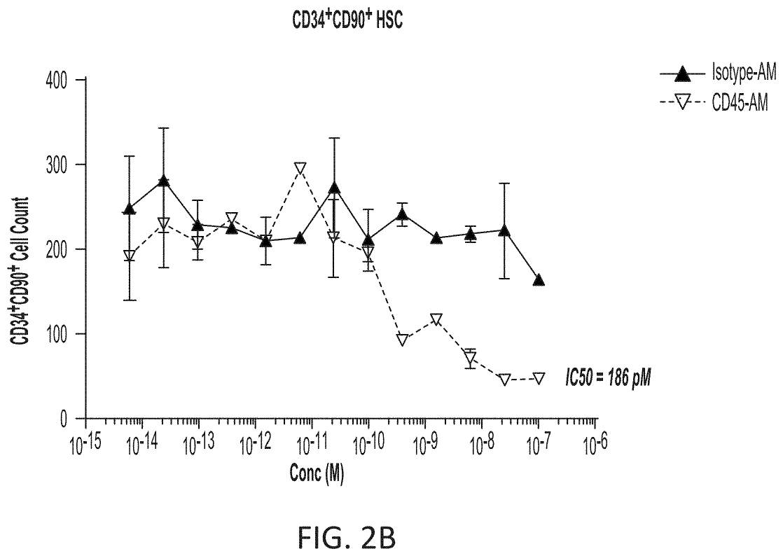

For in vitro killing assays using human PBMCs, the PBMCs were cultured in the presence of anti-CD45-AM or the controls. Cell viability was measured by (FIGS. 1A and 2A). For in vitro killing assays using human HSCs (i.e., CD34+ or CD34+ CD90+ cells), the human BMCs were cultured with anti-CD45-AM or the controls. Live cell counts were determined by flow cytometry. Killing of cells was measured and the results were shown in FIGS. 1B and 2B.

The results in FIGS. 1A and 2A indicate that the anti-CD45-AM is highly effective at killing human PBMCs (FIG. 1A; IC₅₀=55 pM; FIG. 2A; IC₅₀=7 pM). Anti-CD45-AM demonstrates similar efficiency in killing both the human and cyno PBMCs (˜2-fold difference; results not shown). In addition, the anti-CD45-AM is also effective at killing human bone marrow CD34+ cells (FIG. 1B; IC₅₀=914 pM) and human bone marrow CD34+CD90+ cells (FIG. 2B; IC₅₀=186 pM). However, these data indicate a differential toxicity against human lymphocytes (FIG. 2A; IC₅₀=7 pM) compared to human HSCs (FIG. 2B; IC₅₀=186 pM), which results in preferential depletion of human lymphocytes.

### Example 2. An In Vivo Study Using an Anti-CD45-Amatoxin Antibody Drug Conjugate (ADC)

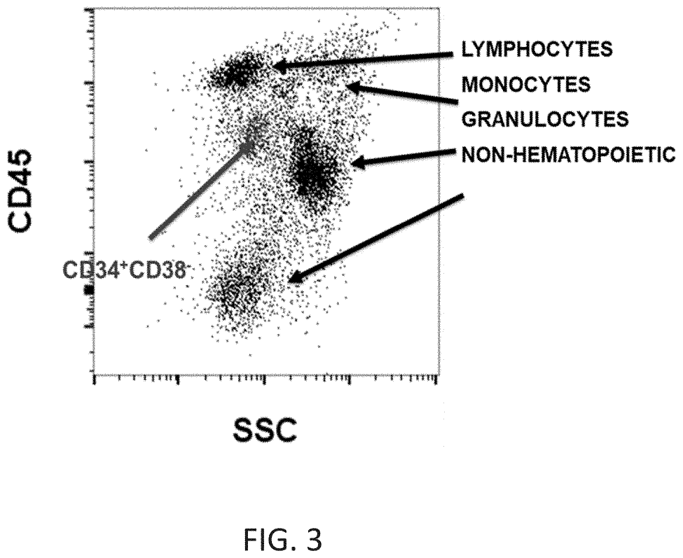

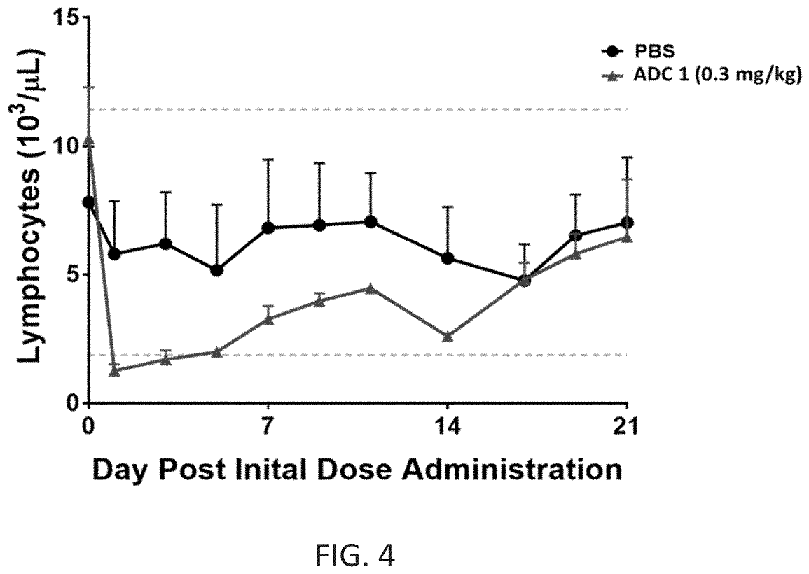

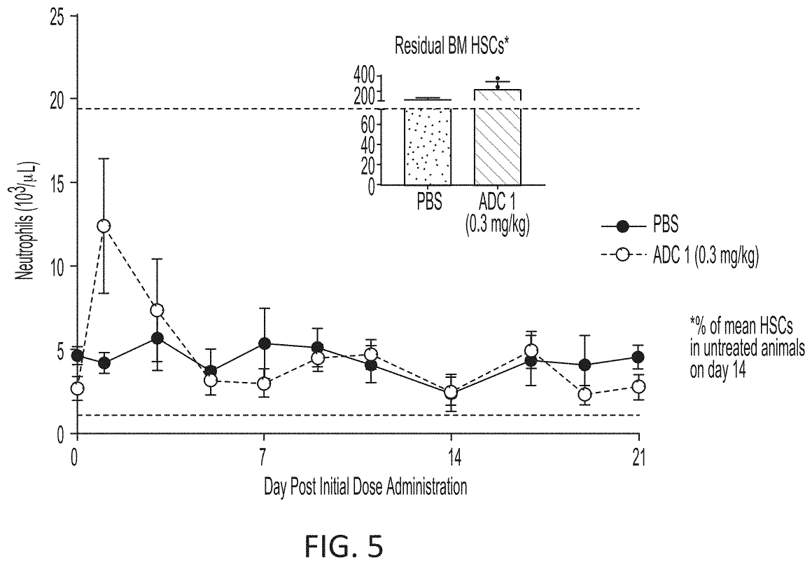

The anti-CD45 ADC used in the following Example is Ab1 conjugated to an amatoxin represented by Formula (I) with an R8 linkage (same ADC as described in Example 1). Cohorts of monkeys were administered the anti-CD45 ADC (0.3 mg/kg) or a control (PBS) at T=0. Cells were analyzed thereafter using flow cytometry. FIG. 3 depicts the phenotypic analysis of the cells. FIG. 4 graphically depicts the depletion of peripheral lymphocytes in monkeys treated with the ADC 1 or control. FIG. 5 graphically depicts the levels of neutrophils in monkeys treated with the ADC 1 or control.

The results in FIG. 3 indicate that the target expression profile for ADC 1 allows dosing to deplete lymphocytes while sparing HSCs. The results in FIGS. 4 and 5 indicate that a single dose of ADC 1 (0.3 mg/kg) results in the rapid depletion of peripheral lymphocytes (i.e., achieves rapid and deep lymphodepletion; FIG. 4), while no neutropenia was observed in the monkeys at a lymphodepleting dose; FIG. 5).

### Example 3. Pharmacokinetic Analysis of an Anti-CD45-Amatoxin Antibody Drug Conjugate (ADC)

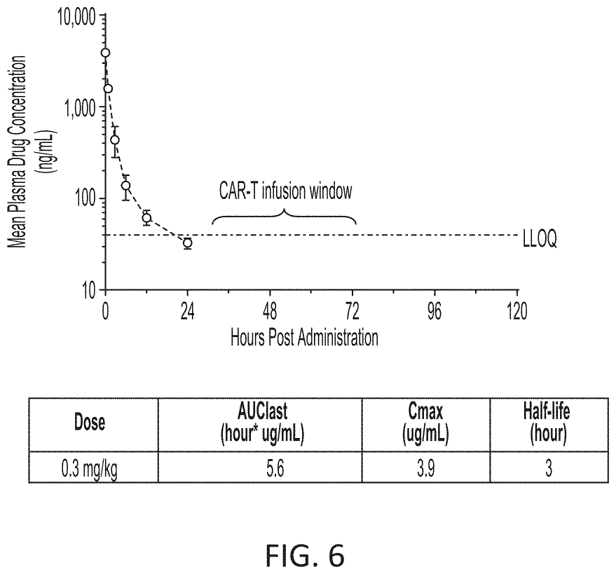

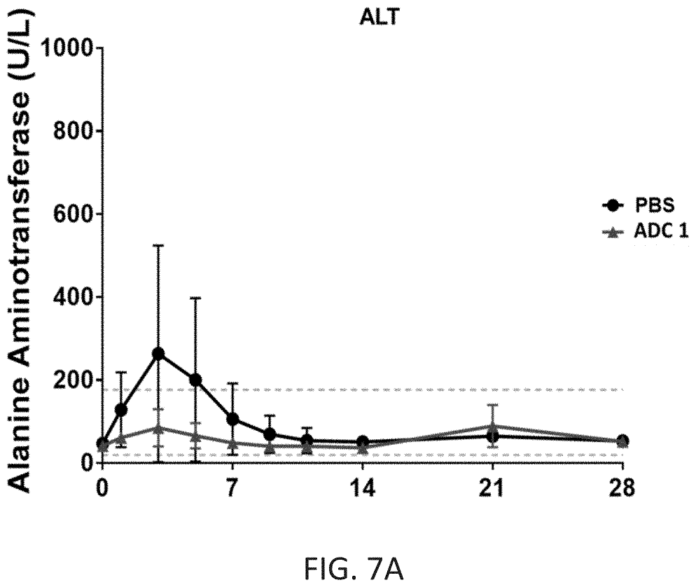

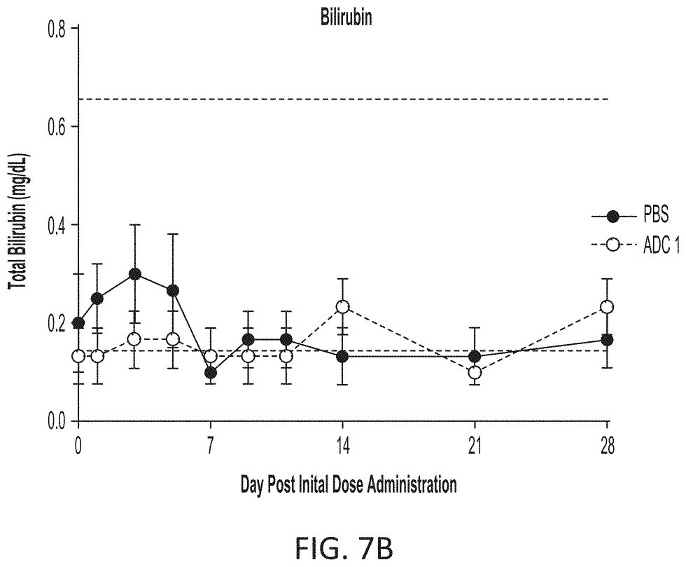

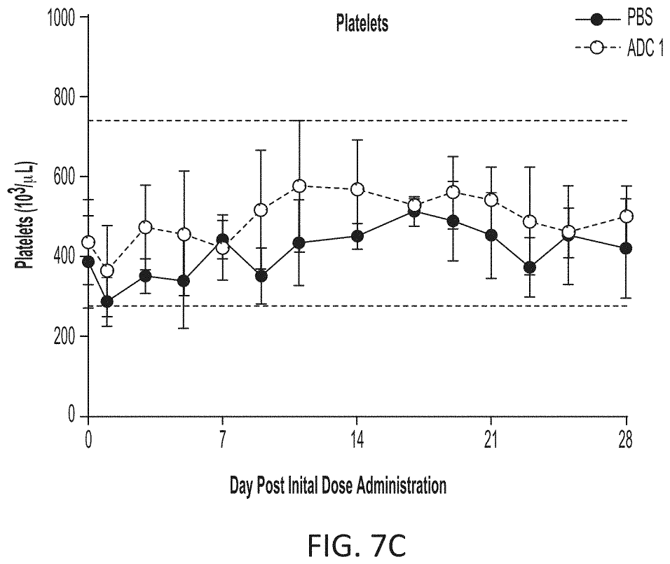

The anti-CD45 ADC used in the following Example is the same ADC as described above in Example 1. Cohorts of monkeys were administered the anti-CD45 ADC (0.3 mg/kg) or a control (PBS) at T=0. The mean plasma concentration of the anti-CD45 ADC was measured and graphically depicted as a function of time (i.e., hours post administration) (FIG. 6). Plasma levels of ALT (alanine aminotransferase; FIG. 7A) and bilirubin FIG. 7B) were measured using a hematology analyzer and was graphically represented as a function of days post dose administration as shown in FIGS. 7A and 7B. Platelet cell count was measured using a hematology analyzer and was graphically represented as a function of days post dose administration as shown in FIG. 7C. These results indicate that the ADC had no effect on platelet cell count or plasma levels of ALT or bilirubin.

The results in FIG. 6 indicate that a lymphodepleting dose of the ADC 1 (fast half-life) is cleared by 48 hours post dose administration, such that the ADC is not detectable during the potential window for CAR-T infusion. The results in FIGS. 7A-7C indicate that a lymphodepleting dose of the ADC 1 (fast half-life) is well tolerated, with no observation of thrombocytopenia, where the clinical chemistry values for liver and kidney function are all within control parameters at the lymphodepleting dose.

### Example 4. Analysis of Cytokine Levels Upon Administration of a Lymphodepleting Dose of an Anti-CD45-Amatoxin Antibody Drug Conjugate (ADC)

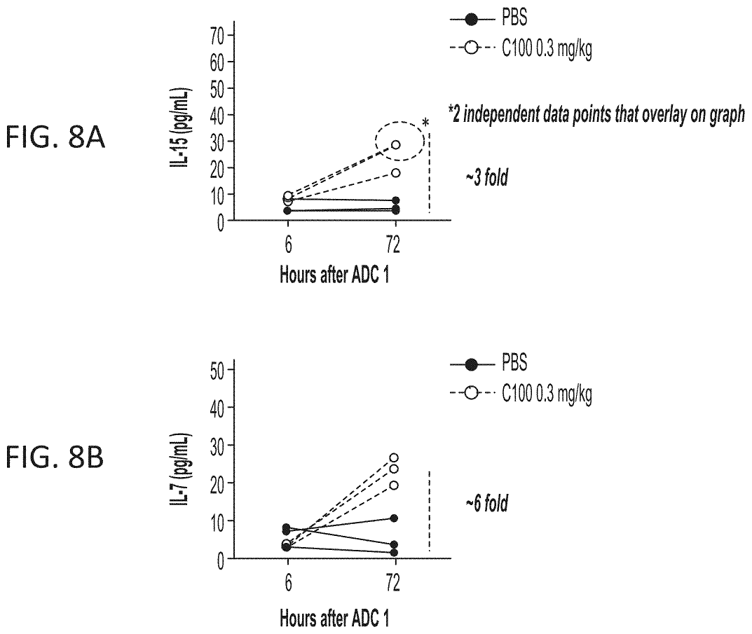

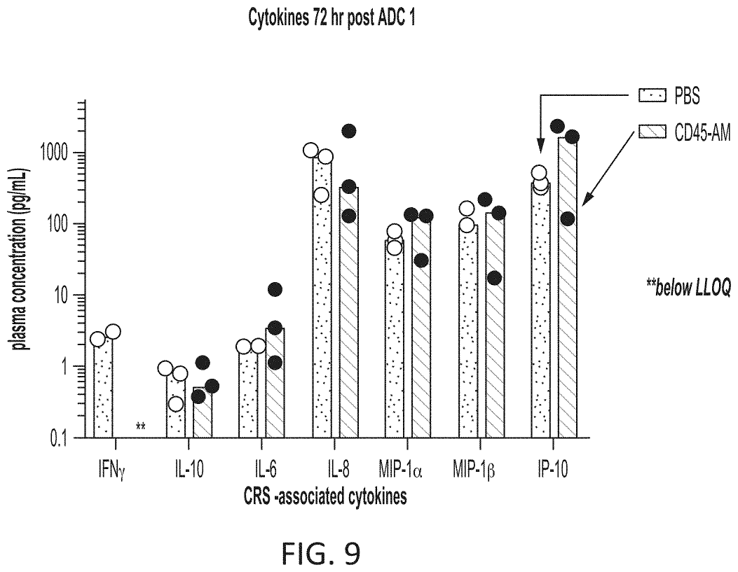

The anti-CD45 ADC used in the following Example is the same ADC as described above in Example 1. Cohorts of monkeys were administered the anti-CD45 ADC (0.3 mg/kg) or a control (PBS) at T=0. Levels of IL-15 (pg/mL; FIG. 8A) and levels of IL-7 (pg/mL; FIG. 8A) were measured and graphically depicted as a function of time (hours after ADC 1 administration). The levels of certain other cytokine release syndrome (CRS)-associated cytokines were also measured at 72 hours after ADC 1 administration and graphically depicted in FIG. 9.

The results in FIG. 8A, and FIG. 8B indicate that a lymphodepleting dose of the ADC 1 increases IL-15 levels (FIG. 8A) and IL-7 levels (FIG. 8B) and provides equivalent levels of CAR-T-engrafting cytokines (i.e., IL-15 and IL-7) as compared to fludarabine/cyclophosphamide chemical conditioning (see, e.g., patient data disclosed in Kochehnderfer et al. Clin Oncol. 35: 1803-13). Increases in IL-15 and IL-7 are associated with CAR-T expansion and efficacy. The results also indicate that a lymphodepleting dose of the ADC 1 does not elevate the levels of key CRS-cytokines (FIG. 9), e.g., IFNγ, IL-10, IL-6, IL-8, MIP-1α, MIP-1β, and IL-10.

### Example 5. Combination Therapy of Anti-CD45 ADC and T Cell Depletion for Allogeneic Transplant

Allogeneic transplantation (2×10⁷ Balb/c CD45.1 TCR BM→B6) was performed in B6 mice after conditioning with a CD45 ADC (CD45 conjugated to PBD) or irradiation as a control. Conditioning was performed by either administering total body irradiation (TBI), a CD45-PBD ADC at 3 mg/kg dose, a CD45-PBD ADC at a dose of 1 mg/kg, a combination of 3 mg/kg CD45-PBD ADC and T cell depletion therapy (anti-CD4 and CD8 antibodies), or a naïve control.

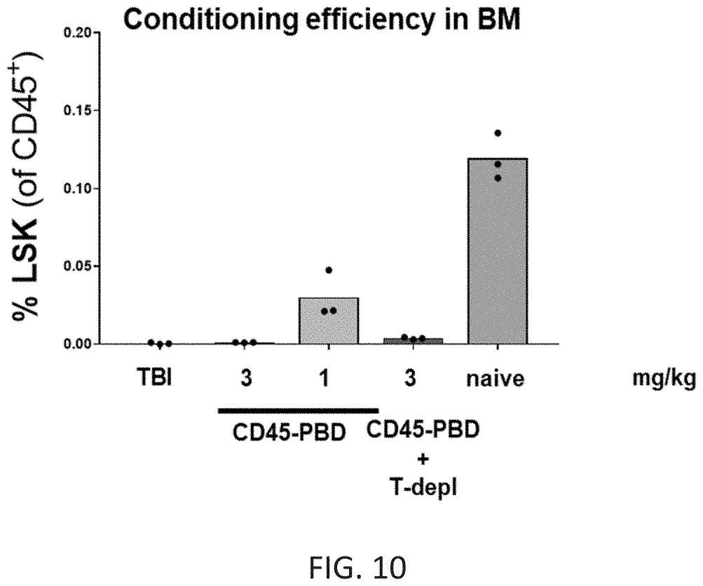

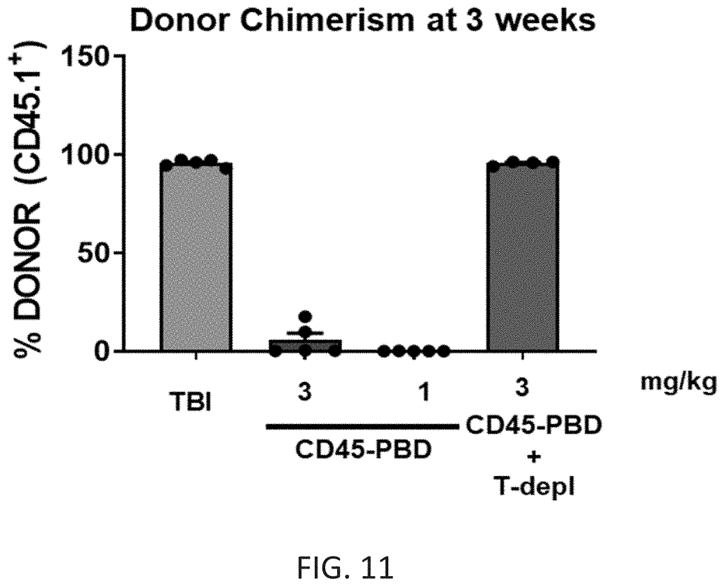

The results of the experiment are provided in FIGS. 10 and 11. Less than 10% donor chimerism was observed following conditioning with a CD45-PBD ADC as a single agent (survived out to 3 wks before rejection). By contrast, full donor chimerism was achieved following conditioning with a CD45-PBD in combination with a T cell depleting therapy (e.g., anti-CD4 and CD8 mAbs). The results provided in FIGS. 10 and 11 suggest that the level of T cell depletion with CD45-PBD as single agent can be enhanced by combining the CD45 ADC therapy with a T cell depleting therapy, such as an anti-CD4 antibody, an anti-CD8 antibody, anti-thymocyte globulin (“ATG”) (e.g., rabbit ATG, equine ATG, and combinations thereof), an anti-CD52 antibody (e.g., alemtuzumab), TBI, and combinations thereof. In some embodiments, the T cell depleting therapy may be a monoclonal antibody.

### Example 6. In Vivo Analysis of Lymphodepletion and Myeloid Depletion in hNSG Mice Upon Administration of an Anti-CD45 ADC

The anti-CD45 ADC used in the following Example is Ab1 conjugated to one of two amatoxins (referred to in this example as “A” and “B”) represented by Formula (I) with an R8 linkage (A) or an R5 linkage (B). Isotype and anti-CD45 antibodies conjugated to amanitin (“CD45-AM”) were administered to humanized NSG (hNSG) mice at the indicated dose levels (1 mg/kg, 3 mg/kg, or 6 mg/kg). To measure the levels of T cells, B cells, and myeloid cells in mice post-ADC administration, peripheral blood (day 7 and day 14) and bone marrow (day 14) were sampled and analyzed via flow cytometry.

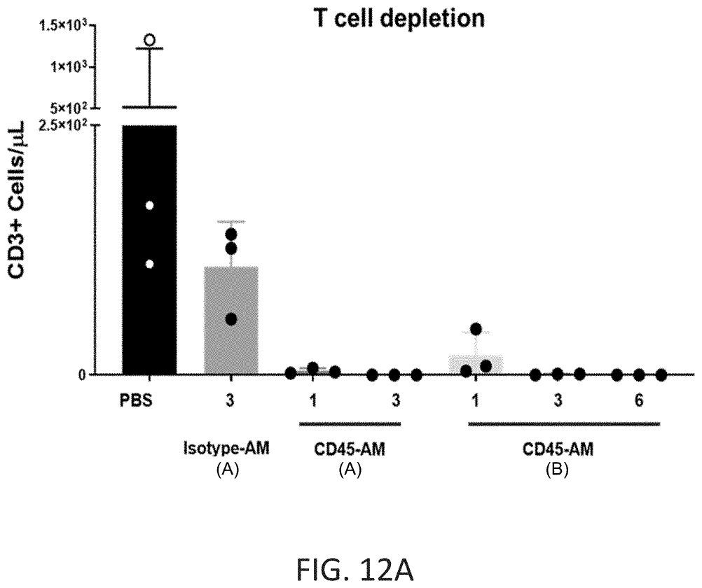

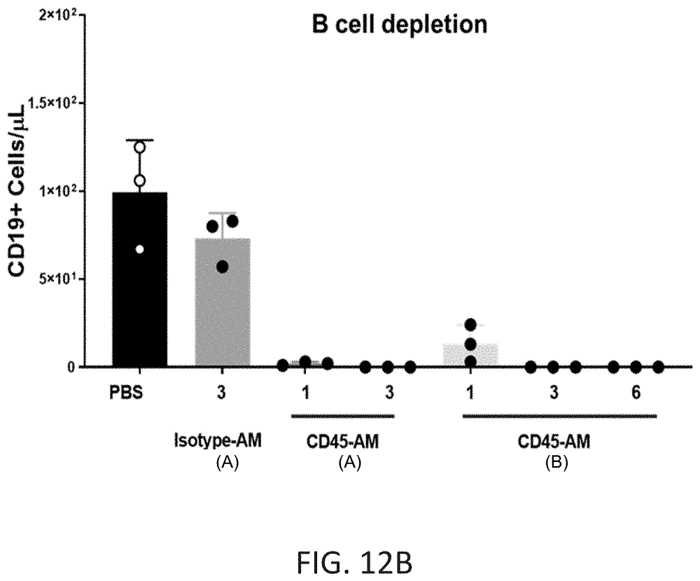

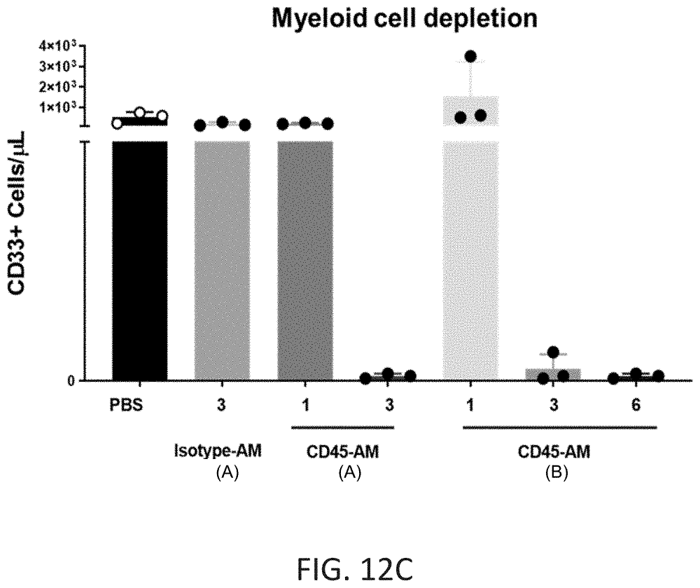

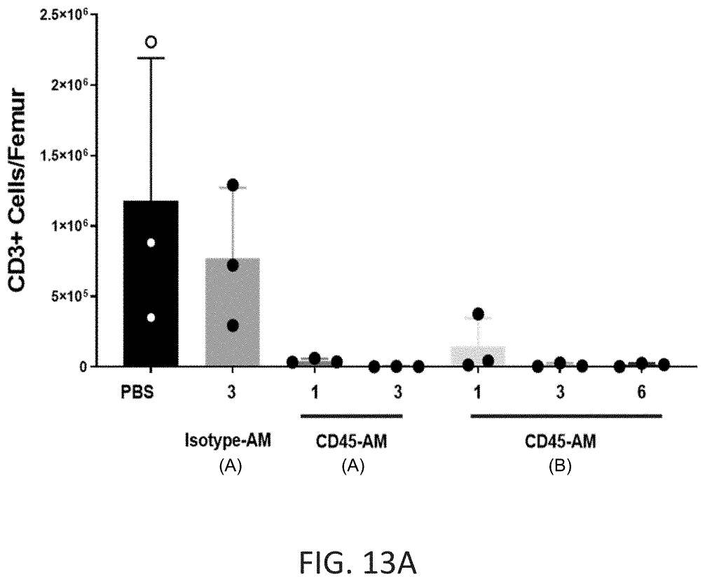

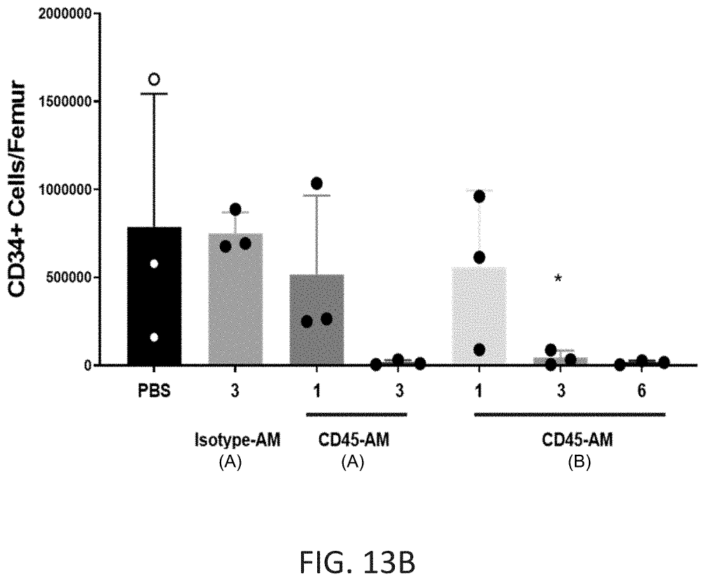

As shown in FIGS. 12A-12C, 14 days after administration at the 1 mg/kg dose level, CD45-AM ADCs mediated extended depletion of human lymphocytes (T and B) with only transient depletion of human myeloid lineages. Further, at all dose levels, CD45-AM ADCs mediated substantial depletion of human T cells in bone marrow (FIG. 13A). In contrast, at 1 mg/kg, no effect of CD45-AM ADCs was observed on HSCs in BM of hNSG mice (FIG. 13B). At >3 mg/kg, CD45-AM ADCs mediated substantial depletion of HSCs in BM of hNSG mice (FIG. 13B).

These results indicate that extended lymphodepletion can be achieved in peripheral blood and bone marrow at non-myeloablative doses of CD45-AM in hNSG mice.

TABLE 4
Sequence Summary

| Sequence | | |
|---|---|---|
| Identifier | Description | Sequence |
| SEQ ID NO: 1 | Ab1 CDR-H1 | FTFNNYWMT |
| SEQ ID NO: 2 | Ab1 CDR-H2 | SISSSGGSIYYPDSVKG |
| SEQ ID NO: 3 | Ab1 CDR-H3 | ARDERWAGAMDA |
| SEQ ID NO: 4 | Ab1 CDR-L1 | KASQNINKNLD |
| SEQ lD NO: 5 | Ab1 CDR-L2 | ETNNLQT |
| SEQ lD NO: 6 | Ab1 CDR-L3 | YQHNSRFT |
| SEQ ID NO: 7 | Ab1 Heavy chain | EVQLVESGGDRVQPGRSLTLSCVTSGFTFNNY |
| | variable region | WMTWIRQVPGKGLEWVASISSSGGSIYYPDSV |
| | (CDRs underlined) | KGRFTISRDNAKNTLYLQMNSLRSEDTATYYC |
| | | ARDERWAGAMDAWGQGTSVTVSS |
| SEQ ID NO: 8 | Ab1 Light chain | DIQMTQSPPVLSASVGDRVTLSCKASQNINKN |
| | variable region | LDWYQQKHGEAPKLLIYETNNLQTGIPSRFSG |
| | (CDRs underlined) | SGSGTDYTLTISSLQPEDVATYYCYQHNSRFTF |
| | | GSGTKLEIK |
| SEQ ID NO: 9 | CD8 hinge | AKPTTTPAPR PPTPAPTIAS QPLSLRPEAC |
| | | RPAAGGAVHT RGLDFA |
| SEQ ID NO: | hybrid CD8 - CD28 | AKPTTTPAPR PPTPAPTIAS QPLSLRPEAC |
| 10 | hinge | RPAAGGAVHT RGLDFAPRKI EVMYPPPYLD |
| | | NEKSNGTIIH VKGKHLCPSP LFPGPSKP |
| SEQ ID NO: | CD3 transmembrane | LDPKLCYLLD GILFIYGVIL TALFLRVK |
| 11 | domain fragment | |
| SEQ ID NO: | CD3 transmembrane | LCYLLDGILF IYGVILTALF L |
| 12 | domain fragment | |
| SEQ ID NO: | CD28 | FWVLVVVGGVLACYSLLVTVAFIIFWV |
| 13 | transmembrane | |
| | domain fragment | |
| SEQ ID NO: | CD28 | IEVMYPPPYL DNEKSNGTII HVKGKHLCPS |
| 14 | transmembrane | PLFPGPSKPF WVLVVVGGVL ACYSLLVTVA |
| | domain fragment | FIIFWV |
| SEQ ID NO: | CD3 zeta signaling | RVKFSRSADAPAYQQGQNQLYNELNLGRREE |
| 15 | region | YDVLDKRRGRDPEMGGKPRRKNPQEGLYNEL |
| | | QKDKMAEAYSEIGMKGERRRGKGHDGLYQG |
| | | LSTATKDTYDALHMQALPPR |
| SEQ ID NO: | 4-1BB (CD137) co- | KRGRKKLLYIFKQPFMRPVQTTQEEDGCSCRF |
| 16 | stimulatory signaling | PEEEEGGCEL |
| | region | |
| SEQ ID NO: | CD28 co-stimulatory | RSKRSRLLHSDYMNMTPRRPGPTRKHYQPYA |
| 17 | signaling region | PPRDFAAYRS |
| SEQ ID NO: | Peptide linker | GGGGS |
| 18 | | |
| SEQ ID NO: | Peptide Linker | GGGGS GGGGS GGGGS |
| 19 | | |
| SEQ ID NO: | Human CD45RA | MTMYLWLKLLAFGFAFLDTEVFVTGQSPTPSP |
| 20 | Isoform (Uniprot | TGLTTAKMPSVPLSSDPLPTHTTAFSPASTFER |
| | Accession No: | ENDFSETTTSLSPDNTSTQVSPDSLDNASAFNT |
| | P08575-8) | TDAYLNASETTTLSPSGSAVISTTTIATTPSKPT |
| | | CDEKYANITVDYLYNKETKLFTAKLNVNENV |
| | | ECGNNTCTNNEVHNLTECKNASVSISHNSCTA |
| | | PDKTLILDVPPGVEKFQLHDCTQVEKADTTIC |
| | | LKWKNIETFTCDTQNITYRFQCGNMIFDNKEI |
| | | KLENLEPEHEYKCDSEILYNNHKFTNASKIIKT |
| | | DFGSPGEPQIIFCRSEAAHQGVITWNPPQRSFH |
| | | NFTLCYIKETEKDCLNLDKNLIKYDLQNLKPY |
| | | TKYVLSLHAYIIAKVQRNGSAAMCHFTTKSAP |
| | | PSQVWNMTVSMTSDNSMHVKCRPPRDRNGP |
| | | HERYHLEVEAGNTLVRNESHKNCDFRVKDLQ |
| | | YSTDYTFKAYFHNGDYPGEPFILHHSTSYNSK |
| | | ALIAFLAFLIIVTSIALLVVLYKIYDLHKKRSCN |
| | | LDEQQELVERDDEKQLMNVEPIHADILLETYK |
| | | RKIADEGRLFLAEFQSIPRVFSKFPIKEARKPFN |
| | | QNKNRYVDILPYDYNRVELSEINGDAGSNYIN |
| | | ASYIDGFKEPRKYIAAQGPRDETVDDFWRMI |
| | | WEQKATVIVMVTRCEEGNRNKCAEYWPSME |
| | | EGTRAFGDVVVKINQHKRCPDYIIQKLNIVNK |
| | | KEKATGREVTHIQFTSWPDHGVPEDPHLLLKL |
| | | RRRVNAFSNFFSGPIVVHCSAGVGRTGTYIGID |
| | | AMLEGLEAENKVDVYGYVVKLRRQRCLMVQ |
| | | VEAQYILIHQALVEYNQFGETEVNLSELHPYL |
| | | HNMKKRDPPSEPSPLEAEFQRLPSYRSWRTQH |
| | | IGNQEENKSKNRNSNVIPYDYNRVPLKHELEM |
| | | SKESEHDSDESSDDDSDSEEPSKYIN1ASFIMSY |
| | | WKPEVMIAAQGPLKETIGDFWQMIFQRKVKV |
| | | IVMLTELKHGDQEICAQYWGEGKQTYGDIEV |
| | | DLKDTDKSSTYTLRVFELRHSKRKDSRTVYQ |
| | | YQYTNWSVEQLPAEPKELISMIQVVKQKLPQK |
| | | NSSEGNKHHKSTPLLIHCRDGSQQTGIFCALLN |
| | | LLESAETEEVVDIFQVVKALRKARPGMVSTFE |
| | | QYQFLYDVIASTYPAQNGQVKKNNHQEDKIE |
| | | FDNEVDKVKQDANCVNPLGAPEKLPEAKEQA |
| | | EGSEPTSGTEGPEHSVNGPASPALNQGS |
| SEQ ID NO: | Human CD45R0 | MTMYLWLKLLAFGFAFLDTEVFVTGQSPTPSP |
| 21 | Isoform (NCBI | TDAYLNASETTTLSPSGSAVISTTTIATTPSKPT |
| | Accession No: | CDEKYANITVDYLYNKETKLFTAKLNVNENV |
| | NP_563578.2) | ECGNNTCTNNEVHNLTECKNASVSISHNSCTA |
| | | PDKTLILDVPPGVEKFQLHDCTQVEKADTTIC |
| | | LKWKNIETFTCDTQNITYRFQCGNMIFDNKEI |
| | | KLENLEPEHEYKCDSEILYNNHKFTNASKIIKT |
| | | DFGSPGEPQIIFCRSEAAHQGVITWNPPQRSFH |
| | | NFTLCYIKETEKDCLNLDKNLIKYDLQNLKPY |
| | | TKYVLSLHAYIIAKVQRNGSAAMCHFTTKSAP |
| | | PSQVWNMTVSMTSDNSMHVKCRPPRDRNGP |
| | | HERYHLEVEAGNTLVRNESHKNCDFRVKDLQ |
| | | YSTDYTFKAYFHNGDYPGEPFILHHSTSYNSK |
| | | ALIAFLAFLIIVTSIALLVVLYKIYDLHKKRSCN |
| | | LDEQQELVERDDEKQLMNVEPIHADILLETYK |
| | | RKIADEGRLFLAEFQSIPRVFSKFPIKEARKPFN |
| | | QNKNRYVDILPYDYNRVELSEINGDAGSNYIN |
| | | ASYIDGFKEPRKYIAAQGPRDETVDDFWRMI |
| | | WEQKATVIVMVTRCEEGNRNKCAEYWPSME |
| | | EGTRAFGDVVVKINQHKRCPDYIIQKLNIVNK |
| | | KEKATGREVTHIQFTSWPDHGVPEDPHLLLKL |
| | | RRRVNAFSNFFSGPIVVHCSAGVGRTGTYIGID |
| | | AMLEGLEAENKVDVYGYVVKLRRQRCLMVQ |
| | | VEAQYILIHQALVEYNQFGETEVNLSELHPYL |
| | | HNMKKRDPPSEPSPLEAEFQRLPSYRSWRTQH |
| | | IGNQEENKSKNRNSNVIPYDYNRVPLKHELEM |
| | | SKESEHDSDESSDDDSDSEEPSKYINTASFIMSY |
| | | WKPEVMIAAQGPLKETIGDFWQMIFQRKVKV |
| | | IVMLTELKHGDQEICAQYWGEGKQTYGDIEV |
| | | DLKDTDKSSTYTLRVFELRHSKRKDSRTVYQ |
| | | YQYTNWSVEQLPAEPKELISMIQVVKQKLPQK |
| | | NSSEGNKHHKSTPLLIHCRDGSQQTGIFCALLN |
| | | LLESAETEEVVDIFQVVKALRKARPGMVSTFE |
| | | QYQFLYDVIASTYPAQNGQVKKNNHQEDKIE |
| | | FDNEVDKVKQDANCVNPLGAPEKLPEAKEQA |
| | | EGSEPTSGTEGPEHSVNGPASPALNQGS |
| SEQ ID NO: | Human CD45RB | MTMYLWLKLLAFGFAFLDTEVFVTGQSPTPSP |
| 22 | Isoform (NCBI | TGVSSVQTPHLPTHADSQTPSAGTDTQTFSGS |
| | Accession No: | AANAKLNPTPGSNAISDAYLNASETTTLSPSGS |
| | XP_006711537.1) | AVISTTTIATTPSKPTCDEKYANITVDYLYNKE |
| | | TKLFTAKLNVNENVECGNNTCTNNEVHNLTE |
| | | CKNASVSISHNSCTAPDKTLILDVPPGVEKFQL |
| | | HDCTQVEKADTTICLKWKNIETFTCDTQNITY |
| | | RFQCGNMIFDNKEIKLENLEPEHEYKCDSEILY |
| | | NNHKFTNASKIIKTDFGSPGEPQIIFCRSEAAHQ |
| | | GVITWNPPQRSFHNFTLCYIKETEKDCLNLDK |
| | | NLIKYDLQNLKPYTKYVLSLHAYIIAKVQRNG |
| | | SAAMCHFTTKSAPPSQVWNMTVSMTSDNSM |
| | | HVKCRPPRDRNGPHERYHLEVEAGNTLVRNE |
| | | SHKNCDFRVKDLQYSTDYTFKAYFHNGDYPG |
| | | EPFILHHSTSYNSKALIAFLAFLIIVTSIALLVVL |
| | | YKIYDLHKKRSCNLDEQQELVERDDEKQLMN |
| | | VEPIHADILLETYKRKIADEGRLFLAEFQSIPRV |
| | | FSKFPIKEARKPFNQNKNRYVDILPYDYNRVE |
| | | LSEINGDAGSNYINASYIDGFKEPRKYIAAQGP |
| | | RDETVDDFWRMIWEQKATVIVMVTRCEEGN |
| | | RNKCAEYWPSMEEGTRAFGDVVVKINQHKR |
| | | CPDYIIQKLNIVNKKEKATGREVTHIQFTSWPD |
| | | HGVPEDPHLLLKLRRRVNAFSNFFSGPIVVHC |
| | | SAGVGRTGTYIGIDAMLEGLEAENKVDVYGY |
| | | VVKLRRQRCLMVQVEAQYILIHQALVEYNQF |
| | | GETEVNLSELHPYLHNMKKRDPPSEPSPLEAE |
| | | FQRLPSYRSWRTQHIGNQEENKSKNRNSNVIP |
| | | YDYNRVPLKHELEMSKESEHDSDESSDDDSDS |
| | | EEPSKYINASFIMSYWKPEVMIAAQGPLKETIG |
| | | DFWQMIFQRKVKVIVMLTELKHGDQEICAQY |
| | | WGEGKQTYGDIEVDLKDTDKSSTYTLRVFEL |
| | | RHSKRKDSRTVYQYQYTNWSVEQLPAEPKEL |
| | | ISMIQVVKQKLPQKNSSEGNKHHKSTPLLIHC |
| | | RDGSQQTGIFCALLNLLESAETEEVVDIFQVV |
| | | KALRKARPGMVSTFEQYQFLYDVIASTYPAQ |
| | | NGQVKKNNHQEDKIEFDNEVDKVKQDANCV |
| | | NPLGAPEKLPEAKEQAEGSEPTSGTEGPEHSV |
| | | NGPASPALNQGS |
| SEQ ID NO: | Human CD45RAB | MTMYLWLKLLAFGFAFLDTEVFVTGQSPTPSP |
| 23 | Isoform (NCBI | TGLTTAKMPSVPLSSDPLPTHTTAFSPASTFER |
| | Accession No: | ENDFSETTTSLSPDNTSTQVSPDSLDNASAFNT |
| | XP_006711535.1) | TGVSSVQTPHLPTHADSQTPSAGTDTQTFSGS |
| | | AANAKLNPTPGSNAISDAYLNASETTTLSPSGS |
| | | AVISTTTIATTPSKPTCDEKYANITVDYLYNKE |
| | | TKLFTAKLNVNENVECGNNTCTNNEVHNLTE |
| | | CKNASVSISHNSCTAPDKTLILDVPPGVEKFQL |
| | | HDCTQVEKADTTICLKWKNIETFTCDTQNITY |
| | | RFQCGNMIFDNKEIKLENLEPEHEYKCDSEILY |
| | | NNHKFTNASKIIKTDFGSPGEPQIIFCRSEAAHQ |
| | | GVITWNPPQRSFHNFTLCYIKETEKDCLNLDK |
| | | NLIKYDLQNLKPYTKYVLSLHAYIIAKVQRNG |
| | | SAAMCHFTTKSAPPSQVWNMTVSMTSDNSM |
| | | HVKCRPPRDRNGPHERYHLEVEAGNTLVRNE |
| | | SHKNCDFRVKDLQYSTDYTFKAYFHNGDYPG |
| | | EPFILHHSTSYNSKALIAFLAFLIIVTSIALLVVL |
| | | YKIYDLHKKRSCNLDEQQELVERDDEKQLMN |
| | | VEPIHADILLETYKRKIADEGRLFLAEFQSIPRV |
| | | FSKFPIKEARKPFNQNKNRYVDILPYDYNRVE |
| | | LSEINGDAGSNYINASYIDGFKEPRKYIAAQGP |
| | | RDETVDDFWRMIWEQKATVIVMVTRCEEGN |
| | | RNKCAEYWPSMEEGTRAFGDVVVKINQHKR |
| | | CPDYIIQKLNIVNKKEKATGREVTHIQFTSWPD |
| | | HGVPEDPHLLLKLRRRVNAFSNFFSGPIVVHC |
| | | SAGVGRTGTYIGIDAMLEGLEAENKVDVYGY |
| | | VVKLRRQRCLMVQVEAQYILIHQALVEYNQF |
| | | GETEVNLSELHPYLHNMKKRDPPSEPSPLEAE |
| | | FQRLPSYRSWRTQHIGNQEENKSKNRNSNVIP |
| | | YDYNRVPLKHELEMSKESEHDSDESSDDDSDS |
| | | EEPSKYINASFIMSYWKPEVMIAAQGPLKETIG |
| | | DFWQMIFQRKVKVIVMLTELKHGDQEICAQY |
| | | WGEGKQTYGDIEVDLKDTDKSSTYTLRVFEL |
| | | RHSKRKDSRTVYQYQYTNWSVEQLPAEPKEL |
| | | ISMIQVVKQKLPQKNSSEGNKHHKSTPLLIHC |
| | | RDGSQQTGIFCALLNLLESAETEEVVDIFQVV |
| | | KALRKARPGMVSTFEQYQFLYDVIASTYPAQ |
| | | NGQVKKNNHQEDKIEFDNEVDKVKQDANCV |
| | | NPLGAPEKLPEAKEQAEGSEPTSGTEGPEHSV |
| | | NGPASPALNQGS |
| SEQ ID NO: | Human CD45RBC | MTMYLWLKLLAFGFAFLDTEVFVTGQSPTPSP |
| 24 | Isoform (NCBI | TGVSSVQTPHLPTHADSQTPSAGTDTQTFSGS |
| | Accession No: | AANAKLNPTPGSNAISDVPGERSTASTFPTDPV |
| | XP_006711536.1) | SPLTTTLSLAHHSSAALPARTSNTTITANTSDA |
| | | YLNASETTTLSPSGSAVISTTTIATTPSKPTCDE |
| | | KYANITVDYLYNKETKLFTAKLNVNENVECG |
| | | NNTCTNNEVHNLTECKNASVSISHNSCTAPDK |
| | | TLILDVPPGVEKFQLHDCTQVEKADTTICLKW |
| | | KNIETFTCDTQNITYRFQCGNMIFDNKEIKLEN |
| | | LEPEHEYKCDSEILYNNHKFTNASKIIKTDFGS |
| | | PGEPQIIFCRSEAAHQGVITWNPPQRSFHNFTL |
| | | CYIKETEKDCLNLDKNLIKYDLQNLKPYTKYV |
| | | LSLHAYIIAKVQRNGSAAMCHFTTKSAPPSQV |
| | | WNMTVSMTSDNSMHVKCRPPRDRNGPHERY |
| | | HLEVEAGNTLVRNESHKNCDFRVKDLQYSTD |
| | | YTFKAYFHNGDYPGEPFILHHSTSYNSKALIAF |
| | | LAFLIIVTSIALLVVLYKIYDLHKKRSCNLDEQ |
| | | QELVERDDEKQLMNVEPIHADILLETYKRKIA |
| | | DEGRLFLAEFQSIPRVFSKFPIKEARKPFNQNK |
| | | NRYVDILPYDYNRVELSEINGDAGSNYINASYI |
| | | DGFKEPRKYIAAQGPRDETVDDFWRMIWEQK |
| | | ATVIVMVTRCEEGNRNKCAEYWPSMEEGTRA |
| | | FGDVVVKINQHKRCPDYIIQKLNIVNKKEKAT |
| | | GREVTHIQFTSWPDHGVPEDPHLLLKLRRRVN |
| | | AFSNFFSGPIVVHCSAGVGRTGTYIGIDAMLEG |
| | | LEAENKVDVYGYVVKLRRQRCLMVQVEAQY |
| | | ILIHQALVEYNQFGETEVNLSELHPYLHNMKK |
| | | RDPPSEPSPLEAEFQRLPSYRSWRTQHIGNQEE |
| | | NKSKNRNSNVIPYDYNRVPLKHELEMSKESEH |
| | | DSDESSDDDSDSEEPSKYINASFIMSYWKPEV |
| | | MIAAQGPLKETIGDFWQMIFQRKVKVIVMLTE |
| | | LKHGDQEICAQYWGEGKQTYGDIEVDLKDTD |
| | | KSSTYTLRVFELRHSKRKDSRTVYQYQYTNW |
| | | SVEQLPAEPKELISMIQVVKQKLPQKNSSEGN |
| | | KHHKSTPLLIHCRDGSQQTGIFCALLNLLESAE |
| | | TEEVVDIFQVVKALRKARPGMVSTFEQYQFL |
| | | YDVIASTYPAQNGQVKKNNHQEDKIEFDNEV |
| | | DKVKQDANCVNPLGAPEKLPEAKEQAEGSEP |
| | | TSGTEGPEHSVNGPASPALNQGS |
| SEQ ID NO: | Human CD45RABC | MTMYLWLKLLAFGFAFLDTEVFVTGQSPTPSP |
| 25 | Isoform (NCBI | TGLTTAKMPSVPLSSDPLPTHTTAFSPASTFER |
| | Accession No. | ENDFSETTTSLSPDNTSTQVSPDSLDNASAFNT |
| | NP_002829.3) | TGVSSVQTPHLPTHADSQTPSAGTDTQTFSGS |
| | | AANAKLNPTPGSNAISDVPGERSTASTFPTDPV |
| | | SPLTTTLSLAHHSSAALPARTSNTTITANTSDA |
| | | YLNASETTTLSPSGSAVISTTTIATTPSKPTCDE |
| | | KYANITVDYLYNKETKLFTAKLNVNENVECG |
| | | NNTCTNNEVHNLTECKNASVSISHNSCTAPDK |
| | | TLILDVPPGVEKFQLHDCTQVEKADTTICLKW |
| | | KNIETFTCDTQNITYRFQCGNMIFDNKEIKLEN |
| | | LEPEHEYKCDSEILYNNHKFTNASKIIKTDFGS |
| | | PGEPQIIFCRSEAAHQGVITWNPPQRSFHNFTL |
| | | CYIKETEKDCLNLDKNLIKYDLQNLKPYTKYV |
| | | LSLHAYIIAKVQRNGSAAMCHFTTKSAPPSQV |
| | | WNMTVSMTSDNSMHVKCRPPRDRNGPHERY |
| | | HLEVEAGNTLVRNESHKNCDFRVKDLQYSTD |
| | | YTFKAYFHNGDYPGEPFILHHSTSYNSKALIAF |
| | | LAFLIIVTSIALLVVLYKIYDLHKKRSCNLDEQ |
| | | QELVERDDEKQLMNVEPIHADILLETYKRKIA |
| | | DEGRLFLAEFQSIPRVFSKFPIKEARKPFNQNK |
| | | NRYVDILPYDYNRVELSEINGDAGSNYINASYI |
| | | DGFKEPRKYIAAQGPRDETVDDFWRMIWEQK |
| | | ATVIVMVTRCEEGNRNKCAEYWPSMEEGTRA |
| | | FGDVVVKINQHKRCPDYIIQKLNIVNKKEKAT |
| | | GREVTHIQFTSWPDHGVPEDPHLLLKLRRRVN |
| | | AFSNFFSGPIVVHCSAGVGRTGTYIGIDAMLEG |
| | | LEAENKVDVYGYVVKLRRQRCLMVQVEAQY |
| | | ILIHQALVEYNQFGETEVNLSELHPYLHNMKK |
| | | RDPPSEPSPLEAEFQRLPSYRSWRTQHIGNQEE |
| | | NKSKNRNSNVIPYDYNRVPLKHELEMSKESEH |
| | | DSDESSDDDSDSEEPSKYINASFIMSYWKPEV |
| | | MIAAQGPLKETIGDFWQMIFQRKVKVIVMLTE |
| | | LKHGDQEICAQYWGEGKQTYGDIEVDLKDTD |
| | | KSSTYTLRVFELRHSKRKDSRTVYQYQYTNW |
| | | SVEQLPAEPKELISMIQVVKQKLPQKNSSEGN |
| | | KHHKSTPLLIHCRDGSQQTGIFCALLNLLESAE |
| | | TEEVVDIFQVVKALRKARPGMVSTFEQYQFL |
| | | YDVIASTYPAQNGQVKKNNHQEDKIEFDNEV |
| | | DKVKQDANCVNPLGAPEKLPEAKEQAEGSEP |
| | | TSGTEGPEHSVNGPASPALNQGS |
| SEQ ID NO: | Human CD45RABC | QSPTPSPTGLTTAKMPSVPLSSDPLPTHTTAFSP |
| 26 | Antigen (Fragment | ASTFERENDFSETTTSLSPDNTSTQVSPDSLDN |
| | of Human | ASAFNTTGVSSVQTPHLPTHADSQTPSAGTDT |
| | CD45RABC | QTFSGSAANAKLNPTPGSNAISDVPGERSTAST |
| | Isoform) | FPTDPVSPLTTTLSLAHHSSAALPARTSNTTITA |
| | | NTSDAYLNASETTTLSPSGSAVISTTTIATTPSK |
| | | PTCDEKYANITVDYLYNKETKLFTAKLNVNE |
| | | NVECGNNTCTNNEVHNLTECKNASVSISHNSC |
| | | TAPDKTLILDVPPGVEKFQLHDCTQVEKADTT |
| | | ICLKWKNIETFTCDTQNITYRFQCGNMIFDNK |
| | | EIKLENLEPEHEYKCDSEILYNNHKFTNASKII |
| | | KTDFGSPGEPQIIFCRSEAAHQGVITWNPPQRS |
| | | FHNFTLCYIKETEKDCLNLDKNLIKYDLQNLK |
| | | PYTKYVLSLHAYIIAKVQRNGSAAMCHFTTKS |
| | | APPSQVWNMTVSMTSDNSMHVKCRPPRDRN |
| | | GPHERYHLEVEAGNTLVRNESHKNCDFRVKD |
| | | LQYSTDYTFKAYFHNGDYPGEPFILHHSTSYN |
| | | SK |
| SEQ ID NO: | CD45 Fragment | RNGPHERYHLEVEAGNT |
| 27 | | |
| SEQ ID NO: | CD45 Fragment | CRPPRDRNGPHERYHLEVEAGNTLVRNESHK |
| 28 | | |
| SEQ ID NO: | Apamistamab Heavy | EVKLLESGGGLVQPGGSLKLSCAASGFDFSRY |
| 29 | Chain | WMSWVRQAPGKGLEWIGEINPTSSTINFTPSL |
| | | KDKVFISRDNAKNTLYLQMSKVRSEDTALYY |
| | | CARGNYYRYGDAMDYWGQGTSVTVSSAKTT |
| | | PPSVYPLAPGSAAQTNSMVTLGCLVKGYFPEP |
| | | VTVTWNSGSLSSGVHTFPAVLQSDLYTLSSSV |
| | | TVPSSTWPSETVTCNVAHPASSTKVDKKIVPR |
| | | DCGCKPCICTVPEVSSVFIFPPKPKDVLTITLTP |
| | | KVTCVVVDISKDDPEVQFSWFVDDVEVHTAQ |
| | | TQPREEQFNSTERSVSELPIMHQDWLNGKEFK |
| | | CRVNSAAFPAPIEKTISKTKGRPKAPQVYTIPPP |
| | | KEQMAKDKVSLTCMITDFFPEDITVEWQWNG |
| | | QPAENYKNTQPIMDTDGSYFVYSKLNVQKSN |
| | | WEAGNTFTCSVLHEGLHNHHTEKSLSHSPGK |
| SEQ ID NO: | Apamistamab Light | DIALTQSPASLAVSLGQRATISCRASKSVSTSG |
| 30 | Chain | YSYLHWYQQKPGQPPKLLIYLASNLESGVPAR |
| | | FSGSGSGTDFTLNIHPVEEEDAATYYCQHSRE |
| | | LPFTFGSGTKLEIKRADAAPTVSIFPPSSEQLTS |
| | | GGASVVCFLNNEYPKDINVKWKIDGSERQNG |
| | | VLNSWTDQDSKDSTYSMSSTLTLTKDEYERH |
| | | NSYTCEATHKTSTSPIVKSFNRNEC |
| SEQ ID NO: | Apamistamab Heavy | EVKLLESGGGLVQPGGSLKLSCAASGFDFSRY |
| 31 | Chain Variable | WMSWVRQAPGKGLEWIGEINPTSSTINFTPSL |
| | Region | KDKVFISRDNAKNTLYLQMSKVRSEDTALYY |
| | | CARGNYYRYGDAMDYWGQGTSVTVSSA |
| SEQ ID NO: | Apamistamab Light | DIALTQSPASLAVSLGQRATISCRASKSVSTSG |
| 32 | Chain Variable | YSYLHWYQQKPGQPPKLLIYLASNLESGVPAR |
| | Region | FSGSGSGTDFTLNIHPVEEEDAATYYCQHSRE |
| | | LPFTFGSGTKLEIKR |
| SEQ ID NO: | SLT-1A | KEFTLDFSTAKTYVDSLNVIRSAIGTPLQTISSG |
| 33 | | GTSLLMIDSGSGDNLFAVDVRGIDPEEGRFNN |
| | | LRLIVERNNLYVTGFVNRTNNVFYRFADFSHV |
| | | TFPGTTAVTLSGDSSYTTLQRVAGISRTGMQIN |
| | | RHSLTTSYLDLMSHSGTSLTQSVARAMLRFVT |
| | | VTAEALRFRQIQRGFRTTLDDLSGRSYVMTAE |
| | | DVDLTLNWGRLSSVLPDYHGQDSVRVGRISF |
| | | GSINAILGSVALILNCHHHASRVARMASDEFPS |
| | | MCPADGRVRGITHNKILWDSSTLGAILMRRTI |
| | | SS |
| SEQ ID NO: | StxA | KEFTLDFSTAKTYVDSLNVIRSAIGTPLQTISSG |
| 34 | | GTSLLMIDSGTGDNLFAVDVRGIDPEEGRFNN |
| | | LRLIVERNNLYVTGFVNRTNNVFYRFADFSHV |
| | | TFPGTTAVTLSGDSSYTTLQRVAGISRTGMQIN |
| | | RHSLTTSYLDLMSHSGTSLTQSVARAMLRFVT |
| | | VTAEALRFRQIQRGFRTTLDDLSGRSYVMTAE |
| | | DVDLTLNWGRLSSVLPDYHGQDSVRVGRISF |
| | | GSINAILGSVALILNCHHHASRVARMASDEFPS |
| | | MCPADGRVRGITHNKILWDSSTLGAILMRRTI |
| | | SS |
| SEQ ID NO: | SLT-2A | DEFTVDFSSQKSYVDSLNSIRSAISTPLGNISQG |
| 35 | | GVSVSVINHVLGGNYISLNVRGLDPYSERFNH |
| | | LRLIMERNNLYVAGFINTETNIFYRFSDFSHISV |
| | | PDVITVSMTTDSSYSSLQRIADLERTGMQIGRH |
| | | SLVGSYLDLMEFRGRSMTRASSRAMLRFVTVI |
| | | AEALRFRQIQRGFRPALSEASPLYTMTAQDVD |
| | | LTLNWGRISNVLPEYRGEEGVRIGRISFNSLSAI |
| | | LGSVAVILNCHSTGSYSVRSVSQKQKTECQIV |
| | | GDRAAIKVNNVLWEANTIAALLNRKPQDLTE |
| | | PNQ |

### OTHER EMBODIMENTS

All publications, patents, and patent applications mentioned in this specification are incorporated herein by reference to the same extent as if each independent publication or patent application was specifically and individually indicated to be incorporated by reference.

While the invention has been described in connection with specific embodiments thereof, it will be understood that it is capable of further modifications and this application is intended to cover any variations, uses, or adaptations of the invention following, in general, the principles of the invention and including such departures from the invention that come within known or customary practice within the art to which the invention pertains and may be applied to the essential features hereinbefore set forth, and follows in the scope of the claims.

1. A method of promoting acceptance of an immune cell expressing a chimeric antigen receptor (CAR) in a human subject having cancer or an autoimmune disease, the method comprising
(a) administering a therapeutically effective amount of an anti-CD45 antibody drug conjugate (ADC) to a human subject having cancer or an autoimmune disease, wherein the anti-CD45 ADC comprises an anti-CD45 antibody, or antigen-binding fragment thereof, conjugated to a cytotoxin via a linker; and
(b) administering a therapeutically effective amount of an immune cell expressing a CAR to the human subject, wherein the CAR comprises an extracellular domain that binds to a tumor antigen or an antigen associated with an autoimmune disease, a transmembrane domain, and a cytoplasmic domain.

2. The method of claim 1, wherein the human subject is not administered alemtuzumab or a lymphodepleting chemotherapeutic agent prior to, concomitantly with, or following step (b).

3. (canceled)

4. The method of claim 2, wherein the lymphodepleting chemotherapeutic agent is fludarabine, cyclophosphamide, bendamustine, and/or pentostatin.

5. The method of claim 1, further comprising administering an anti-CD45 ADC to the human subject prior to step (b).

6. (canceled)

7. The method of claim 1, wherein the immune cell is an allogeneic cell or an autologous cell.

8. The method of claim 7, wherein the allogeneic cell is an allogeneic T cell or an allogeneic NK cell and/or wherein the therapeutically effective amount of the allogeneic cell expressing the CAR is about 1×10⁴ to about 7.0×10⁸ cells/kg.

9. (canceled)

10. A method of treating a human patient having a tumor comprising (i) administering a therapeutically effective amount of an anti-CD45 ADC to a human patient, wherein the anti-CD45 ADC comprises an anti-CD45 antibody, or antigen-binding fragment thereof, conjugated to a cytotoxin via a linker, and (ii) administering to the human patient a therapeutically effective amount of from about 1×10⁶ to about 7×10⁸ CAR T cells/kg.

11. The method of claim 10, wherein the therapeutically effective amount of the CAR T cells is about 1×10⁶ to about 1×10⁸ cells/kg.

12. The method of claim 1, wherein the anti-CD45 ADC is administered to the patient as a single dose or as multiple doses.

13. The method of claim 1, wherein the human patient does not develop neutropenia following administration of the immune cell expressing the CAR.

14. The method of claim 13, wherein neutropenia is defined as the human patient having an absolute neutrophil count (ANC) of less than about 1500 per microliter (1500/microL)

15. The method of claim 1, wherein the human subject does not develop severe neutropenia following administration of the immune cell expressing the CAR.

16. The method of claim 15, wherein the severe neutropenia is defined as an ANC of less than 500/microL.

17. A method of lymphodepleting a human patient selected for CAR-T therapy comprising administering a therapeutically effective amount of an anti-CD45 ADC to the human patient prior to administration of CAR-T cells to the human patient.

18. The method of claim 17, wherein:
(i) the human patient is not administered cyclophosphamide and/or fludarabine as a lymphodepleting regimen as a pre-treatment for the CAR-T therapy; or
(ii) the human patient is not administered a lymphodepleting chemotherapy as a lymphodepleting regimen as a pre-treatment for the CAR-T therapy.

19. (canceled)

20. The method of claim 17, further comprising administering CAR-T therapy to the human patient.

21. The method of claim 1, wherein the anti-CD45 antibody, or antigen-binding fragment thereof, comprises a heavy chain variable region comprising a CDR1, a CDR2, and a CDR3 having an amino acid sequence as set forth in SEQ ID NOs: 1, 2, and 3, respectively, and comprises a light chain variable region comprising a CDR1, a CDR2, and a CDR3 having an amino acid sequence as set forth in SEQ ID NOs: 4, 5, and 6, respectively.

22. The method of claim 21, wherein the anti-CD45 antibody, or antigen-binding fragment thereof, is chimeric or humanized and/or is an IgG1 isotype or an IgG4 isotype.

23. (canceled)

24. The method of claim 1, wherein the cytotoxin is an antimitotic agent, a ribosome inactivating protein (RIP), or an RNA polymerase inhibitor.

25. The method of claim 24, wherein the RNA polymerase inhibitor is an amatoxin.

26. The method of claim 24, wherein the RNA polymerase inhibitor is an amanitin.

27. The method of claim 26, wherein the amanitin is selected from the group consisting of α-amanitin, β-amanitin, γ-amanitin, ε-amanitin, amanin, amaninamide, amanullin, amanullinic acid, proamanullin, and derivatives thereof.

28. The method of claim 1, wherein the anti-CD45 ADC is represented by formula (I) 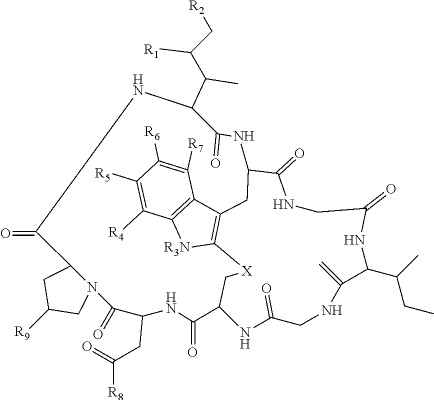
wherein R₁ is H, OH, OR_{A}, or OR_{C};
R₂ is H, OH, OR_{B}, or OR_{C};
R_{A} and R_{B}, when present, together with the oxygen atoms to which they are bound, combine to form an optionally substituted 5-membered heterocycloalkyl group;
R₃ is H, R_{C}, or R_{D};
R₄, R₅, R₆, and R₇ are each independently H, OH, OR_{C}, OR_{D}, R_{C}, or R_{D};
R₈ is OH, NH₂, OR_{C}, OR_{D}, NHR_{C}, or NR_{C}R_{D};
R₉ is H, OH, OR_{C}, or OR_{D};
X is —S—, —S(O)—, or —SO₂—;
R_{C} is -L-Z;
R_{D} is optionally substituted alkyl (e.g., C₁-C₆ alkyl), optionally substituted heteroalkyl (e.g., C₁-C₆ heteroalkyl), optionally substituted alkenyl (e.g., C₂-C₆ alkenyl), optionally substituted heteroalkenyl (e.g., C₂-C₆ heteroalkenyl), optionally substituted alkynyl (e.g., C₂-C₆ alkynyl), optionally substituted heteroalkynyl (e.g., C₂-C₆ heteroalkynyl), optionally substituted cycloalkyl, optionally substituted heterocycloalkyl, optionally substituted aryl, or optionally substituted heteroaryl;
L is a linker selected from the group consisting of optionally substituted alkylene (e.g., C₁-C₆ alkylene), optionally substituted heteroalkylene (C₁-C₆ heteroalkylene), optionally substituted alkenylene (e.g., C₂-C₆ alkenylene), optionally substituted heteroalkenylene (e.g., C₂-C₆ heteroalkenylene), optionally substituted alkynylene (e.g., C₂-C₆ alkynylene), optionally substituted heteroalkynylene (e.g., C₂-C₆ heteroalkynylene), optionally substituted cycloalkylene, optionally substituted heterocycloalkylene, optionally substituted arylene, optionally substituted heteroarylene, a dipeptide, —(C═O)—, a peptide, a disulfide, a hydrazone, a —(CH₂CH₂O)ₚ— group, wherein p is an integer from 1-6, a ((CH₂)ₘO)ₙ(CH₂)ₘ— group, where n and each m are each independently selected from 1, 2, 3, 4, 5, 6, 7, 8, 9, and 10; and combinations thereof; and
Z is a chemical moiety formed from a coupling reaction between a reactive substituent Z′ present on L and a reactive substituent present within the anti-CD45 antibody or antigen-binding fragment thereof.

29. The method of claim 26, wherein the antimitotic agent is a maytansine, an auristatin, a pyrrolobenzodiazepine (PBD) or a calicheamicin.

30-31. (canceled)

32. The method of claim 28, wherein the linker, together with the reactive substituent Z′ of the ADC, is N-beta-maleimidopropyl-Val-Ala-para-aminobenzyl (BMP-Val-Ala-PAB).

33. The method of claim 1, wherein the ADC has a serum half-life of 3 days or less.

34. The method of claim 1, wherein the extracellular domain of the CAR comprises an scFv antibody, a single chain T cell receptor (scTCR), or a non-immunoglobulin scaffold protein.

35. (canceled)

36. The method of claim 1, wherein the tumor antigen is an antigen selected from the group consisting of CD19, CD22, CD30, CD7, BCMA, CD137, CD22, CD20, AFP, GPC3, MUC1, mesothelin, CD38, PD1, EGFR (e.g., EGFRvIII), MG7, BCMA, TACI, CEA, PSCA, CEA, HER2, MUC1, CD33, ROR2, NKR-2, PSCA, CD28, TAA, NKG2D, or CD123.

37. The method of claim 1, wherein the cytoplasmic domain of the CAR comprises a CD28 cytoplasmic signaling domain, a CD3 zeta cytoplasmic signaling domain, an OX40 cytoplasmic signaling domain, and/or a CD137 (4-1BB) cytoplasmic signaling domain.

38. (canceled)

39. The method of claim 1, wherein the human subject having cancer has a cancer selected from the group consisting of leukemia, adult advanced cancer, pancreatic cancer, non-resectable pancreatic cancer, colorectal cancer, metastatic colorectal cancer, ovarian cancer, triple-negative breast cancer, hematopoietic/lymphoid cancer, colon cancer liver metastasis, small cell lung cancer, non-small cell lung cancer, B-cell lymphoma, relapsed or refractory B-cell lymphoma, follicular lymphoma, mantle cell lymphoma, diffuse large cell lymphoma, relapsed or refractory diffuse large cell lymphoma, anaplastic large cell lymphoma, primary mediastinal B-cell lymphoma, recurrent mediastinal, refractory mediastinal large B-cell lymphoma, large B-cell lymphoma, Hodgkin lymphoma, non-Hodgkin lymphoma, relapsed or refractory non-Hodgkin lymphoma, refractory aggressive non-Hodgkin lymphoma, B-cell non-Hodgkin lymphoma, refractory non-Hodgkin lymphoma, colorectal carcinoma, gastric carcinoma, pancreatic carcinoma, triple-negative invasive breast carcinoma, renal cell carcinoma, lung squamous cell carcinoma, hepatocellularcarcinoma, urothelial carcinoma, leukemia, B-cell leukemia, B-cell acute lymphocytic leukemia, B-cell acute lymphoblastic leukemia, adult acute lymphoblastic leukemia, B-cell prolymphocytic leukemia, childhood acute lymphoblastic leukemia, refractory childhood acute lymphoblastic leukemia, acute leukemia, acute lymphoblastic leukemia, acute lymphocytic leukemia, prolymphocytic leukemia, chronic lymphocytic leukemia, acute myeloid leukemia, recurrent plasma cell myeloma, refractory plasma cell myeloma, multiple myeloma, relapsed or refractory multiple myeloma, multiple myeloma of bone, malignant glioma of brain, myelodysplastic syndrome, EGFR-positive colorectal cancer, glioblastoma multiforme, neoplasms, blastic plasmacytoid dendritic cell neoplasms, liver metastases, solid tumors, advanced solid tumors, mesothelin positive tumors, hematological malignancies, and other advanced malignancies.

40. The method of claim 1, wherein the anti-CD45 ADC is administered to the subject in a therapeutically effective amount such that hematopoietic stem cell (HSC) levels are maintained in the patient.

41. The method of claim 40, wherein the level of HSCs in the subject is 70% or more, 80% or more, or 90% or more relative to the level of HSCs prior to anti-CD45 ADC treatment in the subject.

42-43. (canceled)

44. The method of claim 1, wherein the anti-CD45 ADC treatment is administered in combination with a T cell depleting therapy.

45. (canceled)

46. The method of claim 44, wherein the T cell depleting therapy comprises an agent that binds to an antigen expressed on the cell surface of a human T cell or an agent that binds to an antigen expressed on the cell surface of an activated human T cell.

47. (canceled)

48. The method of claim 44, wherein:
(i) the T cell depleting therapy comprises an anti-CD4 antibody, an anti-CD8 antibody, an anti-CD137 antibody, or an anti-CD52 antibody;
(ii) the T cell depleting therapy comprises anti-thymocyte globublin (ATG); or
(iii) the T cell depleting therapy comprises total body irradiation (TBI).

49-51. (canceled)

52. The method of claim 48, wherein the anti-CD52 antibody is alemtuzumab.

53-56. (canceled)

57. The method of claim 1, wherein the level of one or more CAR-T engrafting cytokines in the human subject increases following administration of the anti-CD45 ADC.

58. The method of claim 57, wherein the CAR-T engrafting cytokine is IL-15 or IL-7.

59. The method of claim 1, wherein the level of one or more cytokine release syndrome (CRS)-cytokines does not substantially increase in the human patient following administration of the anti-CD45 ADC.

60. The method of claim 59, wherein the one or more CRS-cytokines is IFNγ, IL-10, IL-6, IL-8, MIP-1α, MIP-1β, or IL-10.


SEQUENCE LISTING (35 sequences)

SEQ ID NO: 1 - protein, 9 aa, Artificial Sequence
  Description of Artificial Sequence Synthetic peptide
FTFNNYWMT

SEQ ID NO: 2 - protein, 17 aa, Artificial Sequence
  Description of Artificial Sequence Synthetic peptide
SISSSGGSIYYPDSVKG

SEQ ID NO: 3 - protein, 12 aa, Artificial Sequence
  Description of Artificial Sequence Synthetic peptide
ARDERWAGAMDA

SEQ ID NO: 4 - protein, 11 aa, Artificial Sequence
  Description of Artificial Sequence Synthetic peptide
KASQNINKNLD

SEQ ID NO: 5 - protein, 7 aa, Artificial Sequence
  Description of Artificial Sequence Synthetic peptide
ETNNLQT

SEQ ID NO: 6 - protein, 8 aa, Artificial Sequence
  Description of Artificial Sequence Synthetic peptide
YQHNSRFT

SEQ ID NO: 7 - protein, 119 aa, Artificial Sequence
  Description of Artificial Sequence Synthetic polypeptide
EVQLVESGGDRVQPGRSLTLSCVTSGFTFNNYWMTWIRQVPGKGLEWVASISSSGGSIYYPDSVKGRFTISRDNAKNTLYLQMNSLRSEDTATYYCARDERWAGAMDAWGQGTSVTVSS

SEQ ID NO: 8 - protein, 106 aa, Artificial Sequence
  Description of Artificial Sequence Synthetic polypeptide
DIQMTQSPPVLSASVGDRVTLSCKASQNINKNLDWYQQKHGEAPKLLIYETNNLQTGIPSRFSGSGSGTDYTLTISSLQPEDVATYYCYQHNSRFTFGSGTKLEIK

SEQ ID NO: 9 - protein, 46 aa, Artificial Sequence
  Description of Artificial Sequence Synthetic polypeptide
AKPTTTPAPRPPTPAPTIASQPLSLRPEACRPAAGGAVHTRGLDFA

SEQ ID NO: 10 - protein, 88 aa, Artificial Sequence
  Description of Artificial Sequence Synthetic polypeptide
AKPTTTPAPRPPTPAPTIASQPLSLRPEACRPAAGGAVHTRGLDFAPRKIEVMYPPPYLDNEKSNGTIIHVKGKHLCPSPLFPGPSKP

SEQ ID NO: 11 - protein, 28 aa, Artificial Sequence
  Description of Artificial Sequence Synthetic peptide
LDPKLCYLLDGILFIYGVILTALFLRVK

SEQ ID NO: 12 - protein, 21 aa, Artificial Sequence
  Description of Artificial Sequence Synthetic peptide
LCYLLDGILFIYGVILTALFL

SEQ ID NO: 13 - protein, 27 aa, Artificial Sequence
  Description of Artificial Sequence Synthetic peptide
FWVLVVVGGVLACYSLLVTVAFIIFWV

SEQ ID NO: 14 - protein, 66 aa, Artificial Sequence
  Description of Artificial Sequence Synthetic polypeptide
IEVMYPPPYLDNEKSNGTIIHVKGKHLCPSPLFPGPSKPFWVLVVVGGVLACYSLLVTVAFIIFWV

SEQ ID NO: 15 - protein, 112 aa, Artificial Sequence
  Description of Artificial Sequence Synthetic polypeptide
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 16 - protein, 42 aa, Artificial Sequence
  Description of Artificial Sequence Synthetic polypeptide
KRGRKKLLYIFKQPFMRPVQTTQEEDGCSCRFPEEEEGGCEL

SEQ ID NO: 17 - protein, 41 aa, Artificial Sequence
  Description of Artificial Sequence Synthetic polypeptide
RSKRSRLLHSDYMNMTPRRPGPTRKHYQPYAPPRDFAAYRS

SEQ ID NO: 18 - protein, 5 aa, Artificial Sequence
  Description of Artificial Sequence Synthetic peptide
GGGGS

SEQ ID NO: 19 - protein, 15 aa, Artificial Sequence
  Description of Artificial Sequence Synthetic peptide
GGGGSGGGGSGGGGS

SEQ ID NO: 20 - protein, 1211 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 21 - protein, 1145 aa, Homo sapiens
MTMYLWLKLLAFGFAFLDTEVFVTGQSPTPSPTDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 22 - protein, 1192 aa, Homo sapiens
MTMYLWLKLLAFGFAFLDTEVFVTGQSPTPSPTGVSSVQTPHLPTHADSQTPSAGTDTQTFSGSAANAKLNPTPGSNAISDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 23 - protein, 1258 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 24 - protein, 1240 aa, Homo sapiens
MTMYLWLKLLAFGFAFLDTEVFVTGQSPTPSPTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 25 - protein, 1306 aa, Homo sapiens
MTMYLWLKLLAFGFAFLDTEVFVTGQSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSKALIAFLAFLIIVTSIALLVVLYKIYDLHKKRSCNLDEQQELVERDDEKQLMNVEPIHADILLETYKRKIADEGRLFLAEFQSIPRVFSKFPIKEARKPFNQNKNRYVDILPYDYNRVELSEINGDAGSNYINASYIDGFKEPRKYIAAQGPRDETVDDFWRMIWEQKATVIVMVTRCEEGNRNKCAEYWPSMEEGTRAFGDVVVKINQHKRCPDYIIQKLNIVNKKEKATGREVTHIQFTSWPDHGVPEDPHLLLKLRRRVNAFSNFFSGPIVVHCSAGVGRTGTYIGIDAMLEGLEAENKVDVYGYVVKLRRQRCLMVQVEAQYILIHQALVEYNQFGETEVNLSELHPYLHNMKKRDPPSEPSPLEAEFQRLPSYRSWRTQHIGNQEENKSKNRNSNVIPYDYNRVPLKHELEMSKESEHDSDESSDDDSDSEEPSKYINASFIMSYWKPEVMIAAQGPLKETIGDFWQMIFQRKVKVIVMLTELKHGDQEICAQYWGEGKQTYGDIEVDLKDTDKSSTYTLRVFELRHSKRKDSRTVYQYQYTNWSVEQLPAEPKELISMIQVVKQKLPQKNSSEGNKHHKSTPLLIHCRDGSQQTGIFCALLNLLESAETEEVVDIFQVVKALRKARPGMVSTFEQYQFLYDVIASTYPAQNGQVKKNNHQEDKIEFDNEVDKVKQDANCVNPLGAPEKLPEAKEQAEGSEPTSGTEGPEHSVNGPASPALNQGS

SEQ ID NO: 26 - protein, 552 aa, Homo sapiens
QSPTPSPTGLTTAKMPSVPLSSDPLPTHTTAFSPASTFERENDFSETTTSLSPDNTSTQVSPDSLDNASAFNTTGVSSVQTPHLPTHADSQTPSAGTDTQTFSGSAANAKLNPTPGSNAISDVPGERSTASTFPTDPVSPLTTTLSLAHHSSAALPARTSNTTITANTSDAYLNASETTTLSPSGSAVISTTTIATTPSKPTCDEKYANITVDYLYNKETKLFTAKLNVNENVECGNNTCTNNEVHNLTECKNASVSISHNSCTAPDKTLILDVPPGVEKFQLHDCTQVEKADTTICLKWKNIETFTCDTQNITYRFQCGNMIFDNKEIKLENLEPEHEYKCDSEILYNNHKFTNASKIIKTDFGSPGEPQIIFCRSEAAHQGVITWNPPQRSFHNFTLCYIKETEKDCLNLDKNLIKYDLQNLKPYTKYVLSLHAYIIAKVQRNGSAAMCHFTTKSAPPSQVWNMTVSMTSDNSMHVKCRPPRDRNGPHERYHLEVEAGNTLVRNESHKNCDFRVKDLQYSTDYTFKAYFHNGDYPGEPFILHHSTSYNSK

SEQ ID NO: 27 - protein, 17 aa, Homo sapiens
RNGPHERYHLEVEAGNT

SEQ ID NO: 28 - protein, 31 aa, Homo sapiens
CRPPRDRNGPHERYHLEVEAGNTLVRNESHK

SEQ ID NO: 29 - protein, 445 aa, Artificial Sequence
  Description of Artificial Sequence Synthetic polypeptide
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSAKTTPPSVYPLAPGSAAQTNSMVTLGCLVKGYFPEPVTVTWNSGSLSSGVHTFPAVLQSDLYTLSSSVTVPSSTWPSETVTCNVAHPASSTKVDKKIVPRDCGCKPCICTVPEVSSVFIFPPKPKDVLTITLTPKVTCVVVDISKDDPEVQFSWFVDDVEVHTAQTQPREEQFNSTERSVSELPIMHQDWLNGKEFKCRVNSAAFPAPIEKTISKTKGRPKAPQVYTIPPPKEQMAKDKVSLTCMITDFFPEDITVEWQWNGQPAENYKNTQPIMDTDGSYFVYSKLNVQKSNWEAGNTFTCSVLHEGLHNHHTEKSLSHSPGK

SEQ ID NO: 30 - protein, 218 aa, Artificial Sequence
  Description of Artificial Sequence Synthetic polypeptide
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKRADAAPTVSIFPPSSEQLTSGGASVVCFLNNEYPKDINVKWKIDGSERQNGVLNSWTDQDSKDSTYSMSSTLTLTKDEYERHNSYTCEATHKTSTSPIVKSFNRNEC

SEQ ID NO: 31 - protein, 122 aa, Artificial Sequence
  Description of Artificial Sequence Synthetic polypeptide
EVKLLESGGGLVQPGGSLKLSCAASGFDFSRYWMSWVRQAPGKGLEWIGEINPTSSTINFTPSLKDKVFISRDNAKNTLYLQMSKVRSEDTALYYCARGNYYRYGDAMDYWGQGTSVTVSSA

SEQ ID NO: 32 - protein, 112 aa, Artificial Sequence
  Description of Artificial Sequence Synthetic polypeptide
DIALTQSPASLAVSLGQRATISCRASKSVSTSGYSYLHWYQQKPGQPPKLLIYLASNLESGVPARFSGSGSGTDFTLNIHPVEEEDAATYYCQHSRELPFTFGSGTKLEIKR

SEQ ID NO: 33 - protein, 293 aa, Escherichia coli
KEFTLDFSTAKTYVDSLNVIRSAIGTPLQTISSGGTSLLMIDSGSGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 34 - protein, 293 aa, Shigella dysenteriae
KEFTLDFSTAKTYVDSLNVIRSAIGTPLQTISSGGTSLLMIDSGTGDNLFAVDVRGIDPEEGRFNNLRLIVERNNLYVTGFVNRTNNVFYRFADFSHVTFPGTTAVTLSGDSSYTTLQRVAGISRTGMQINRHSLTTSYLDLMSHSGTSLTQSVARAMLRFVTVTAEALRFRQIQRGFRTTLDDLSGRSYVMTAEDVDLTLNWGRLSSVLPDYHGQDSVRVGRISFGSINAILGSVALILNCHHHASRVARMASDEFPSMCPADGRVRGITHNKILWDSSTLGAILMRRTISS

SEQ ID NO: 35 - protein, 297 aa, Escherichia coli
DEFTVDFSSQKSYVDSLNSIRSAISTPLGNISQGGVSVSVINHVLGGNYISLNVRGLDPYSERFNHLRLIMERNNLYVAGFINTETNIFYRFSDFSHISVPDVITVSMTTDSSYSSLQRIADLERTGMQIGRHSLVGSYLDLMEFRGRSMTRASSRAMLRFVTVIAEALRFRQIQRGFRPALSEASPLYTMTAQDVDLTLNWGRISNVLPEYRGEEGVRIGRISFNSLSAILGSVAVILNCHSTGSYSVRSVSQKQKTECQIVGDRAAIKVNNVLWEANTIAALLNRKPQDLTEPNQ